SEQUENCE LISTING (190 sequences)

SEQ ID NO: 1 - protein, 126 aa, synthetic construct
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 2 - protein, 61 aa, synthetic construct
GDRSRLLRDLRVDTKSRAAWARLLQEHPNARKYKGANKKGLSKGCFGLKLDRIGSMSGLGC

SEQ ID NO: 3 - protein, 53 aa, synthetic construct
DLRVDTKSRAAWARLLQEHPNARKYKGANKKGLSKGCFGLKLDRIGSMSGLGC

SEQ ID NO: 4 - protein, 36 aa, synthetic construct
EHPNARKYKGANKKGLSKGCFGLKLDRIGSMSGLGC

SEQ ID NO: 5 - protein, 22 aa, synthetic construct
GLSKGCFGLKLDRIGSMSGLGC

SEQ ID NO: 6 - protein, 19 aa, synthetic construct
MEFGLSWLFLVAILKGVQC

SEQ ID NO: 7 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 233 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 54 aa, synthetic construct
MEFGLWLFLVAILKGVQCEHPNARKYKGANKKGLSKGCFGLKLDRIGSMSGLGC

SEQ ID NO: 10 - protein, 39 aa, synthetic construct
MEFGLWLFLVAILKGVQCLSKGCFGLKLDRIGSMSGLGC

SEQ ID NO: 11 - protein, 302 aa, synthetic construct
MEFGLSWLFLVAILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEHPNARKYKGANKKGLSKGCFGLKLDRIGSMSGLGC

SEQ ID NO: 12 - protein, 287 aa, synthetic construct
MEFGLSWLFLVAILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSLSKGCFGLKLDRIGSMSGLGC

SEQ ID NO: 13-20 - skipped (8 entries)

SEQ ID NO: 21 - protein, 123 aa, synthetic construct [sequence omitted]

SEQ ID NO: 22 - protein, 112 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 23 - protein, 255 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 24 - protein, 231 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 25 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 255 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 27 - protein, 252 aa, synthetic construct
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 28-30 - skipped (3 entries)

SEQ ID NO: 31 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 204 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 33 - protein, 431 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 431 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35-41 - skipped (7 entries)

SEQ ID NO: 42 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
LALGXXXX

SEQ ID NO: 43 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
LKLGXXXX

SEQ ID NO: 44 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
VKLGXXXX

SEQ ID NO: 45 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
VKLGXXXX

SEQ ID NO: 46 - protein, 8 aa, synthetic construct
  VARIANT 5: note = E, S, or Q
  VARIANT 6: note = A, N, Q, S, T, E, D, K, R or H
  VARIANT 8: note = K, R, E, or A
VKLGXXTX

SEQ ID NO: 47 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
VKLGXXXK

SEQ ID NO: 48 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
LALGXXXX

SEQ ID NO: 49 - protein, 8 aa, synthetic construct
  VARIANT 5: note = E, S, or Q
  VARIANT 6: note = A, N, Q, S, T, E, D, K, R or H
  VARIANT 8: note = K, R, E, or A
LALGXXTX

SEQ ID NO: 50 - protein, 8 aa, synthetic construct
  VARIANT 5: note = E, S, or Q
  VARIANT 6: note = A, N, Q, S, T, E, D, K, R or H
  VARIANT 8: note = K, R, E, or A
LALGXXSX

SEQ ID NO: 51 - protein, 8 aa, synthetic construct
  VARIANT 5: note = E, S, or Q
  VARIANT 6: note = A, N, Q, S, T, E, D, K, R or H
LALGXXTR

SEQ ID NO: 52 - protein, 8 aa, synthetic construct
  VARIANT 5: note = E, S, or Q
  VARIANT 6: note = A, N, Q, S, T, E, D, K, R or H
LALGXXTK

SEQ ID NO: 53 - protein, 8 aa, synthetic construct
  VARIANT 5: note = E, S, or Q
  VARIANT 6: note = A, N, Q, S, T, E, D, K, R or H
LALGXXTE

SEQ ID NO: 54 - protein, 8 aa, synthetic construct
  VARIANT 5: note = E, S, or Q
  VARIANT 6: note = A, N, Q, S, T, E, D, K, R or H
LALGXXTA

SEQ ID NO: 55 - skipped

SEQ ID NO: 56 - protein, 8 aa, synthetic construct
  VARIANT 5: note = E, S, or Q
  VARIANT 6: note = A, N, Q, S, T, E, D, K, R or H
LALGXXSK

SEQ ID NO: 57 - protein, 8 aa, synthetic construct
  VARIANT 5: note = E, S, or Q
  VARIANT 6: note = A, N, Q, S, T, E, D, K, R or H
  VARIANT 7: note = A, N, Q, S, T, E, D, K, R or H
  VARIANT 8: note = K, R, E, or A
LKLGXXXX

SEQ ID NO: 58 - protein, 8 aa, synthetic construct
  VARIANT 5: note = E, S, or Q
  VARIANT 6: note = A, N, Q, S, T, E, D, K, R or H
  VARIANT 8: note = K, R, E, or A
LKLGXXTX

SEQ ID NO: 59 - protein, 8 aa, synthetic construct
  VARIANT 5: note = E, S, or Q
  VARIANT 6: note = A, N, Q, S, T, E, D, K, R or H
LKLGXXTK

SEQ ID NO: 60 - skipped

SEQ ID NO: 61 - protein, 10 aa, synthetic construct
LALGETTRPA

SEQ ID NO: 62 - protein, 10 aa, synthetic construct
LALGETTKPA

SEQ ID NO: 63 - protein, 8 aa, synthetic construct
ALGETTKP

SEQ ID NO: 64 - protein, 8 aa, synthetic construct
ALGETTKP

SEQ ID NO: 65 - protein, 10 aa, synthetic construct
LALGETTKPA

SEQ ID NO: 66 - protein, 10 aa, synthetic construct
LKLGQTTKPK

SEQ ID NO: 67 - protein, 10 aa, synthetic construct
LALGQTTKPK

SEQ ID NO: 68 - protein, 10 aa, synthetic construct
LKLGQTTKPA

SEQ ID NO: 69 - protein, 10 aa, synthetic construct
LALGQTTKPA

SEQ ID NO: 70 - protein, 10 aa, synthetic construct
LALGQTTKPA

SEQ ID NO: 71 - protein, 10 aa, synthetic construct
LALGQTTEPA

SEQ ID NO: 72 - protein, 10 aa, synthetic construct
LALGQTTKPA

SEQ ID NO: 73 - protein, 10 aa, synthetic construct
LALGQTTKPA

SEQ ID NO: 74 - protein, 10 aa, synthetic construct
VALGQTTKPA

SEQ ID NO: 75 - protein, 10 aa, synthetic construct
LALGESTARG

SEQ ID NO: 76 - protein, 10 aa, synthetic construct
LALGETSKRA

SEQ ID NO: 77 - protein, 10 aa, synthetic construct
LALGQSTKPA

SEQ ID NO: 78 - protein, 10 aa, synthetic construct
LALGQTTKPA

SEQ ID NO: 79 - protein, 10 aa, synthetic construct
LALGQTTKPA

SEQ ID NO: 80 - protein, 10 aa, synthetic construct
LALGQTTKPA

SEQ ID NO: 81 - protein, 20 aa, synthetic construct
LALGQTTKPALALGQTTKPA

SEQ ID NO: 82 - protein, 10 aa, synthetic construct
VKLGQTTKPA

SEQ ID NO: 83-90 - skipped (8 entries)

SEQ ID NO: 91 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGETTRPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 92 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 93 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 94 - protein, 753 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 95 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 96 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 97 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 98 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 99 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 100 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTEPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 101 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRLALGQTTKPAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 102 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRLALGQTTKPAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 103 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 104 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGESTARGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 105 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGETSKRANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 106 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGQSTKPANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 107 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSLALGQTTKPAGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 108 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 109 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPALALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 110 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVAKGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 111-120 - skipped (10 entries)

SEQ ID NO: 121 - protein, 735 aa, Adeno associated virus 2 [sequence omitted]

SEQ ID NO: 122 - protein, 734 aa, Adeno associated virus 2
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 123 - protein, 724 aa, Adeno associated virus 2
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 124 - protein, 736 aa, Adeno associated virus 2
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 125 - protein, 733 aa, Adeno associated virus 2
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 126 - protein, 742 aa, Adeno associated virus 2
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 127-130 - skipped (4 entries)

SEQ ID NO: 131 - protein, 254 aa, synthetic construct
GLSKGCFGLKLDRIGSMSGLGCVGGRKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 283 aa, synthetic construct
EHPNARKYKGANKKGLSKGCFGLKLDRIGSMSGLGCGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 283 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEHPNARKYKGANKKGLSKGCFGLKLDRIGSMSGLGC

SEQ ID NO: 134 - protein, 336 aa, synthetic construct
EHPNARKYKGANKKGLSKGCFGLKLDRIGSMSGLGCRRKRAPVKQTLNFDLLKLAGDVESNPGPMEFGLSWLFLVAILKGVQCEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 135 - protein, 289 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 136 - protein, 275 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGLSKGCFGLKLDRIGSMSGLGC

SEQ ID NO: 137 - protein, 275 aa, synthetic construct
GLSKGCFGLKLDRIGSMSGLGCGGGGSGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 289 aa, synthetic construct; identical to SEQ ID NO: 135

SEQ ID NO: 139 - protein, 494 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 140 - protein, 311 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAGGGGSGGGGSGGGGSGGGGSEHPNARKYKGANKKGLSKGCFGLKLDRIGSMSGLGC

SEQ ID NO: 141 - protein, 100 aa, synthetic construct
  VARIANT 1..100: note = GGGGS repeats may be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 142 - protein, 50 aa, synthetic construct
  VARIANT 1..50: note = GGGGS repeats may be deleted
GGGGSGGGGSGGGGSGGGGSGGGGSGGGGSGGGGSGGGGSGGGGSGGGGS

SEQ ID NO: 143 - protein, 40 aa, synthetic construct
  VARIANT 1..40: note = GS repeats may be deleted
GSGSGSGSGSGSGSGSGSGSGSGSGSGSGSGSGSGSGSGS

SEQ ID NO: 144 - protein, 4 aa, synthetic construct
GSGS

SEQ ID NO: 145 - protein, 4 aa, synthetic construct
GGGS

SEQ ID NO: 146 - protein, 5 aa, synthetic construct
GGGGS

SEQ ID NO: 147 - protein, 6 aa, synthetic construct
GGGGGS

SEQ ID NO: 148 - protein, 7 aa, synthetic construct
GGGGGGS

SEQ ID NO: 149 - protein, 25 aa, synthetic construct
  VARIANT 1..25: note = GGGGS repeats may be deleted
GGGGSGGGGSGGGGSGGGGSGGGGS

SEQ ID NO: 150 - protein, 20 aa, synthetic construct
GGGGSGGGGSGGGGSGGGGS

SEQ ID NO: 151 - protein, 25 aa, synthetic construct
GGGGSGGGGSGGGGSGGGGSGGGGS

SEQ ID NO: 152 - DNA, 40 bp, synthetic construct
  source 1..40: mol_type = other DNA
tggggttgatctctccccagcatgccacacaaaaaaccaa

SEQ ID NO: 153 - DNA, 24 bp, synthetic construct
  source 1..24: mol_type = other DNA
gggactttcctacttggcagtaca

SEQ ID NO: 154 - DNA, 59 bp, synthetic construct
  source 1..59: mol_type = other DNA
gttgcctttacttctaggcctgccgccaccatggagttcggcctgagctggctgttcct

SEQ ID NO: 155 - DNA, 59 bp, synthetic construct
  source 1..59: mol_type = other DNA
gagctggctgttcctggtggccatccttaagggcgtgcagtgcgagcaccccaacgcgc

SEQ ID NO: 156 - DNA, 59 bp, synthetic construct
  source 1..59: mol_type = other DNA
ccgcctccgccagatccgcctccgccgcttccgcctccgccacatcccaggccgctcat

SEQ ID NO: 157 - DNA, 59 bp, synthetic construct
  source 1..59: mol_type = other DNA
gaggcggatctggcggaggcggcagcggcggcggcggctctgacaaaactcacacatgc

SEQ ID NO: 158 - DNA, 55 bp, synthetic construct
  source 1..55: mol_type = other DNA
tgttcctggtggccatccttaagggcgtgcagtgcgacaaaactcacacatgccc

SEQ ID NO: 159 - DNA, 59 bp, synthetic construct
  source 1..59: mol_type = other DNA
ccgcctccgccagatccgcctccgccgcttccgcctccgcctttacccggagacaggga

SEQ ID NO: 160 - DNA, 59 bp, synthetic construct
  source 1..59: mol_type = other DNA
ggcggatctggcggaggcggcagcggcggcggcggctctgagcaccccaacgcgcgcaa

SEQ ID NO: 161 - DNA, 49 bp, synthetic construct
  source 1..49: mol_type = other DNA
ttgtaatccagaggttgattctcgagttaacatcccaggccgctcatgg

SEQ ID NO: 162 - DNA, 49 bp, synthetic construct
  source 1..49: mol_type = other DNA
gaatcggctccatgagcggcctgggatgttagggaggcggaagcggcgg

SEQ ID NO: 163 - DNA, 49 bp, synthetic construct
  source 1..49: mol_type = other DNA
tactccttgccattcagctagtcctggtgcaggacggtgaggacgctga

SEQ ID NO: 164 - DNA, 49 bp, synthetic construct
  source 1..49: mol_type = other DNA
gttgcctttacttctaggcctgccgccacctaggagttcggcctgagct

SEQ ID NO: 165 - DNA, 49 bp, synthetic construct
  source 1..49: mol_type = other DNA
ctaggagttcggcctgagctggctgttcctggtggccatccttaagggc

SEQ ID NO: 166 - DNA, 48 bp, synthetic construct
  source 1..48: mol_type = other DNA
tggccatccttaagggcgtgcagtgctagcaccccaacgcgcgcaaat

SEQ ID NO: 167 - DNA, 6552 bp, synthetic construct
  source 1..6552: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattgctagtgcggccatcgtgatggcaggttgggcgtcgcttggtcggtcatttcgaaccccagagtcccgctcatttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcctttctctgctgcacacatcccaggccgctcatggagccgattcggtccagcttgaggccgaagcagcccttggacaagcccttcttgttggctcctttgtatttgcgcgcgttggggtgctcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatctgcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 168 - DNA, 6932 bp, synthetic construct
  source 1..6932: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttatcatttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcagagccgccgccgccgctgccgcctccgccagatccgcctccgccgcttccgcctccgccacatcccaggccgctcatggagccgattcggtccagcttgaggccgaagcagcccttggacaagcccttcttgttggctcctttgtatttgcgcgcgttggggtgctcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 169 - DNA, 6929 bp, synthetic construct
  source 1..6929: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagcccttcttgttggctcctttgtatttgcgcgcgttggggtgctcagagccgccgccgccgctgccgcctccgccagatccgcctccgccgcttccgcctccgcctttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 170 - DNA, 6693 bp, synthetic construct
  source 1..6693: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaatcaggcggccacggttctcttgatctccaccttggtgccctggccgaaggtccagggcacggtgctgtactgctggcagtagtaggtggcgaagtcctcgggctgcaggctgctgatggtcagggtgaagtcggtgccgctgccgctgccgctgaatctgctgggcacgccgctgtgcaggctgctggtgaagtagatcagcaccttgggggccttgccgggcttctgctggtaccagttcaggtagttgctgatgtcctggctggcgctgcaggtgatggtcactctgtcgcccacgctggcgctcaggctgctggggctctgggtcagctggatgtcagagccgccgccgccgctgccgcctccgccagatccgcctccgccgcttccgcctccgcccacggtcaccagggtgccctggccccacacgtcgaagtaccagtggctggtgccgtagtagtaggggtacttggcgcagtagtacacggcggtgtcctcggctctcaggctgttcatctgcaggtaggcggtgctcttgctggtgtccaggctgaaggtgaatcttctcttgaagtcggcggcgtaggtgggctcgccggtgtaggtgttgatccagcccacccactccaggcccttgccgggggcctgtctcacccagttcatgccgtagtgggtgaagtcgtagccgctggcggcgcagctcagtctcaggctgccgccgggctgcaccaggccgccgccgctctccaccagctgcacctcgcactgcacgcccttaaggatggccaccaggaacagccagctcaggccgaactccatggggccggggttgctctccacgtcgccggccagcttcagcaggtcgaagttcagggtctgcttcacgggggctctctttcttctacatcccaggccgctcatggagccgattcggtccagcttgaggccgaagcagcccttggacaagcccttcttgttggctcctttgtatttgcgcgcgttggggtgctcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 171 - DNA, 6932 bp, synthetic construct
  source 1..6932: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttatcatttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagctagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcagagccgccgccgccgctgccgcctccgccagatccgcctccgccgcttccgcctccctaacatcccaggccgctcatggagccgattcggtccagcttgaggccgaagcagcccttggacaagcccttcttgttggctcctttgtatttgcgcgcgttggggtgctcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 172 - DNA, 6929 bp, synthetic construct
  source 1..6929: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagcccttcttgttggctcctttgtatttgcgcgcgttggggtgctaagagccgccgccgccgctgccgcctccgccagatccgcctccgccgcttccgcctccgcctttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatctagagggtgtccttgggttttggggggaagaggaagactgacggtccccccaggagttcaggtgctgggcacggtgggcatgtgtgagttttgtcgcactgcacgcccttaaggatggccaccaggaacagccagctcaggccgaactcctaggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 173 - DNA, 6947 bp, synthetic construct
  source 1..6947: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagcccttcttgttggctcctttgtatttgcgcgcgttggggtgctcagagccgcctccgccgcttccgcctccgccagatccgcctccgccgcttccgcctccgcccttgcccaggctcaggctcaggctcttctgggtgtagtggttgtgcagggcctcgtgcatcacgctgcagctgaacacgttgccctcctgccatctgctcttgtccacggtcagtctgctgtacaggaagaagctgccgtcgctgtccagcacggggggggtggtcttgtagttgttctcgggctggccgttgctctcccactccacggcgatgtcgctggggtagaagcccttcaccaggcaggtcaggctcacctggttcttggtcatctcctcctggctggggggcagggtgtacacctggggctctctgggctggcccttggccttgctgatggtcttctcgatgctgctgggcaggcccttgttgctcaccttgcacttgtactccttgccgttcagccagtcctggtgcagcacggtcagcacgctcaccactctgtaggtgctgttgaactgctcctctctgggcttggtcttggcgttgtgcacctccacgccgtccacgtaccagttgaactgcacctcggggtcctcctggctcacgtccaccaccacgcaggtcacctcgggggttctgctgatcatcagggtgtccttgggcttgggggggaacaggaacacgctggggccgcccaggaactcgggggcggggcagggggggcagggggggccgtacttgctctccactctcttgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 174 - DNA, 6871 bp, synthetic construct
  source 1..6871: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagccagagccgcctccgccgcttccgcctccgccagatccgcctccgccgcttccgcctccgcccttgcccaggctcaggctcaggctcttctgggtgtagtggttgtgcagggcctcgtgcatcacgctgcagctgaacacgttgccctcctgccatctgctcttgtccacggtcagtctgctgtacaggaagaagctgccgtcgctgtccagcacggggggggtggtcttgtagttgttctcgggctggccgttgctctcccactccacggcgatgtcgctggggtagaagcccttcaccaggcaggtcaggctcacctggttcttggtcatctcctcctggctggggggcagggtgtacacctggggctctctgggctggcccttggccttgctgatggtcttctcgatgctgctgggcaggcccttgttgctcaccttgcacttgtactccttgccgttcagccagtcctggtgcagcacggtcagcacgctcaccactctgtaggtgctgttgaactgctcctctctgggcttggtcttggcgttgtgcacctccacgccgtccacgtaccagttgaactgcacctcggggtcctcctggctcacgtccaccaccacgcaggtcacctcgggggttctgctgatcatcagggtgtccttgggcttgggggggaacaggaacacgctggggccgcccaggaactcgggggcggggcagggggggcagggggggccgtacttgctctccactctcttgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccagcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 175 - DNA, 6911 bp, synthetic construct
  source 1..6911: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagctacttgcccaggctcaggctcaggctcttctgggtgtagtggttgtgcagggcctcgtgcatcacgctgcagctgaacacgttgccctcctgccatctgctcttgtccacggtcagtctgctgtacaggaagaagctgccgtcgctgtccagcacggggggggtggtcttgtagttgttctcgggctggccgttgctctcccactccacggcgatgtcgctggggtagaagcccttcaccaggcaggtcaggctcacctggttcttggtcatctcctcctggctggggggcagggtgtacacctggggctctctgggctggcccttggccttgctgatggtcttctcgatgctgctgggcaggcccttgttgctcaccttgcacttgtactccttgccgttcagccagtcctggtgcagcacggtcagcacgctcaccactctgtaggtgctgttgaactgctcctctctgggcttggtcttggcgttgtgcacctccacgccgtccacgtaccagttgaactgcacctcggggtcctcctggctcacgtccaccaccacgcaggtcacctcgggggttctgctgatcatcagggtgtccttgggcttgggggggaacaggaacacgctggggccgcccaggaactcgggggcggggcagggggggcagggggggccgtacttgctctccactctcttgtcagagccgcctccgccgcttccgcctccgccagatccgcctccgccgcttccgcctccgccacatcccaggccgctcatggagccgattcggtccagcttgaggccgaagcagcccttggacaagccgatgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttaccagttaactctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 176 - DNA, 7014 bp, synthetic construct
  source 1..7014: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagcccttcttgttggctcctttgtatttgcgcgcgttggggtgctcagagccgcctccgccgcttccgcctccgccagatccgcctccgccgcttccgcctccgcccttgcccaggctcaggctcaggctcttctgggtgtagtggttgtgcagggcctcgtgcatcacgctgcagctgaacacgttgccctcctgccatctgctcttgtccacggtcagtctgctgtacaggaagaagctgccgtcgctgtccagcacggggggggtggtcttgtagttgttctcgggctggccgttgctctcccactccacggcgatgtcgctggggtagaagcccttcaccaggcaggtcaggctcacctggttcttggtcatctcctcctggctggggggcagggtgtacacctggggctctctgggctggcccttggccttgctgatggtcttctcgatgctgctgggcaggcccttgttgctcaccttgcacttgtactccttgccgttcagccagtcctggtgcagcacggtcagcacgctcaccactctgtaggtgctgttgaactgctcctctctgggcttggtcttggcgttgtgcacctccacgccgtccacgtaccagttgaactgcacctcggggtcctcctggctcacgtccaccaccacgcaggtcacctcgggggttctgctgatcatcagggtgtccttgggcttgggggggaacaggaacacgctggggccgcccaggaactcgggggcggggcagggggggcagggggggccgtacttgctctccactctcttgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgagccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 177 - DNA, 6999 bp, synthetic construct
  source 1..6999: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttatcatttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagctagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcagagccgccgccgccgctgccgcctccgccagatccgcctccgccgcttccgcctccctaacatcccaggccgctcatggagccgattcggtccagcttgaggccgaagcagcccttggacaagcccttcttgttggctcctttgtatttgcgcgcgttggggtgctcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgagccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 178 - DNA, 9429 bp, synthetic construct
  source 1..9429: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgaacagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgagcaccccaacgcgcgcaaatacaaaggagccaacaagaagggcttgtccaagggctgcttcggcctcaagctggaccgaatcggctccatgagcggcctgggatgttagggaggcggaagcggcggaggcggatctggcggaggcggcagcggcggcggcggctctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactagctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 179 - DNA, 9444 bp, synthetic construct
  source 1..9444: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgaacagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 180 - DNA, 9426 bp, synthetic construct
  source 1..9426: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgaacagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaatctggccatacacttgagtgacaatgacatccactttgcctttctctccacaggtgtccactcccaggtccaagtttaaacgccgccacctaggagttcggcctgagctggctgttcctggtggccatccttaagggcgtgcagtgcgacaaaactcacacatgcccaccgtgcccagcacctgaactcctggggggaccgtcagtcttcctcttccccccaaaacccaaggacaccctctagatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggaggcggaagcggcggaggcggatctggcggaggcggcagcggcggcggcggctcttagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 181 - DNA, 9418 bp, synthetic construct
  source 1..9418: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgcctccgccctcagacgagtcggatctccctttgggccgcctccccgcctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 182 - DNA, 8842 bp, synthetic construct
  source 1..8842: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgaggtgcagctggtggagagcggcggcggcctggtgcagcccggcggcagcctgagactgagctgcgccgccagcggctacgacttcacccactacggcatgaactgggtgagacaggcccccggcaagggcctggagtgggtgggctggatcaacacctacaccggcgagcccacctacgccgccgacttcaagagaagattcaccttcagcctggacaccagcaagagcaccgcctacctgcagatgaacagcctgagagccgaggacaccgccgtgtactactgcgccaagtacccctactactacggcaccagccactggtacttcgacgtgtggggccagggcaccctggtgaccgtgggcggaggcggaagcggcggaggcggatctggcggaggcggcagcggcggcggcggctctgacatccagctgacccagagccccagcagcctgagcgccagcgtgggcgacagagtgaccatcacctgcagcgccagccaggacatcagcaactacctgaactggtaccagcagaagcccggcaaggcccccaaggtgctgatctacttcaccagcagcctgcacagcggcgtgcccagcagattcagcggcagcggcagcggcaccgacttcaccctgaccatcagcagcctgcagcccgaggacttcgccacctactactgccagcagtacagcaccgtgccctggaccttcggccagggcaccaaggtggagatcaagagaaccgtggccgccggcggaggcggaagcggcggaggcggatctggcggaggcggcagcggcggcggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgcctccgccctcagacgagtcggatctccctttgggccgcctccccgcctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 183 - protein, 95 aa, synthetic construct
MEFGLSWLFLVAILKGVQCEHPNARKYKGANKKGLSKGCFGLKLDRIGSMSGLGCVQQRKDKTHTCPPCPAPELLGGPSVFLFPPKPKDTLMISR

SEQ ID NO: 184 - protein, 287 aa, synthetic construct
MEFGLSWLFLVAILKGVQCEHPNARKYKGANKKGLSKGCFGLKLDRIGSMSGLGCVQQRKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 5 aa, synthetic construct
VGGRK

SEQ ID NO: 186 - protein, 16 aa, synthetic construct
GGGSGGGSGGGSGGGS

SEQ ID NO: 187 - protein, 56 aa, synthetic construct
EHPNARKYKGANKKGLSKGCFGLKLDRIGSMSGLGCGGGGSGGGGSGGGGSGGGGS

SEQ ID NO: 188 - protein, 56 aa, synthetic construct
GGGGSGGGGSGGGGSGGGGSEHPNARKYKGANKKGLSKGCFGLKLDRIGSMSGLGC

SEQ ID NO: 189 - protein, 42 aa, synthetic construct
GGGGSGGGGSGGGGSGGGGSGLSKGCFGLKLDRIGSMSGLGC

SEQ ID NO: 190 - protein, 44 aa, synthetic construct
DIGLSKGCFGLKLDRIGSMSGLGCGGGGSGGGGSGGGGSGGGGS

(19) United States
(12) Patent Application Publication
(10) Pub. No.: US 2025/0099622 A1
(43) Pub. Date: Mar. 27, 2025

(54) COMPOSITIONS AND METHODS FOR EXPRESSING THERAPEUTICS

(71) Applicant: Avirmax Biopharma Inc., Hayward, CA (US)

(72) Inventors: Shengjiang LIU, Lafayette, CA (US); Haifeng CHEN, Hayward, CA (US)

(21) Appl. No.: 18/830,493

(22) Filed: Sep. 10, 2024

Related U.S. Application Data
(63) Continuation of application No. PCT/US2023/064136, filed on Mar. 10, 2023.
(60) Provisional application No. 63/441,643, filed on Jan. 27, 2023.
(60) Provisional application No. 63/440,858, filed on Jan. 24, 2023.
(60) Provisional application No. 63/319,233, filed on Mar. 11, 2022.

(51) Int. Cl. A61K 48/00; A61P 27/02; C07K 14/58; C12N 15/86

(52) U.S. Cl. CPC A61K 48/0058; A61P 27/02; C07K 14/58; C12N 15/86; C07K 2319/30; C07K 2319/50; C12N 2750/14143

(57) ABSTRACT

Described herein are compositions and method for expressing therapeutics.

### CROSS-REFERENCE

This application is a continuation of International Patent Application No. PCT/US2023/064136, filed Mar. 10, 2023, which claims the benefit of U.S. Provisional Application Ser. No. 63/441,643 filed on Jan. 27, 2023; U.S. Provisional Application Ser. No. 63/440,858 filed on Jan. 24, 2023; and U.S. Provisional Application Ser. No. 63/319,233 filed on Mar. 11, 2022, the entirety of which are hereby incorporated by reference herein.

### SEQUENCE LISTING

The instant application contains a Sequence Listing which has been submitted electronically in XML format and is hereby incorporated by reference in its entirety. Said XML copy, created on Dec. 16, 2024, is named 59521-708.301_SL.xml and is 308,429 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Natriuretic peptide precursor C is a prohormone composed of 126 amino acids. Natriuretic peptide precursor C can be further cleaved to yield a receptor binding active version known as C-type natriuretic peptide (CNP), where the CNP can be any one of the three forms: CNP53 (CNP composed of 53 amino acids); CNP36 (CNP composed of 36 amino acids); or CNP22 (CNP composed of 22 amino acids). Upon the CNP binding to its cognate receptor, natriuretic peptide clearance receptor B (NPR-B) and NPR-C, the membrane associated guanylyl cyclase (GC) is activated and catalyzes enzymatic conversion of GTP into the cyclic guanylyl monophosphate (cGMP), the functional effector or the secondary messenger. Because of its ability to activate cGMP, CNP has been utilized as therapeutics.

### SUMMARY

However, cleaved CNP such as CNP53, CNP36, or CNP22 has a half-life of only a few minutes, which limits its use for treating a disease or condition. As such, there remains a need to engineer CNP to increase its stability. There also remains a need to engineer CNP for treating a disease or condition.

Described herein, in some aspects, is an engineered polynucleotide comprising a viral vector, said viral vector comprises an expression cassette, wherein the expression cassette encodes an engineered polypeptide comprising a natriuretic peptide covalently connected to an antibody or fragment thereof. In some embodiments, the natriuretic peptide comprises an amino acid sequence that is at least 80% identical to any one of SEQ ID NOs: 1-5. In some embodiments, the natriuretic peptide comprises an amino acid sequence that is at least 95% identical to any one of SEQ ID NOs: 1-5. In some embodiments, the natriuretic peptide comprises an amino acid sequence of any one of SEQ ID NOs: 1-5. In some embodiments, the natriuretic peptide comprises an amino acid sequence that is at least 80% identical to SEQ ID NO: 4. In some embodiments, the natriuretic peptide comprises an amino acid sequence that is at least 95% identical to SEQ ID NO: 4. In some embodiments, the natriuretic peptide comprises an amino acid sequence of SEQ ID NO: 4. In some embodiments, the antibody or fragment thereof comprises a fragment crystallizable (Fc) region. In some embodiments, the antibody or fragment thereof comprises an amino acid sequence that is at least 80% identical to any one of SEQ ID NOs: 6-8. In some embodiments, the antibody or fragment thereof comprises an amino acid sequence that is at least 95% identical to any one of SEQ ID NOs: 6-8. In some embodiments, the antibody or fragment thereof comprises an amino acid sequence of any one of SEQ ID NOs: 6-8. In some embodiments, the natriuretic peptide is covalently connected to N terminus of the antibody or fragment thereof. In some embodiments, the natriuretic peptide is covalently connected to C terminus of the antibody or fragment thereof. In some embodiments, the natriuretic peptide is covalently connected to the antibody or fragment thereof by a peptide linker. In some embodiments, the peptide linker comprises an amino acid sequence comprising (GGGGS)n, wherein the n is an integer between 0-20 (SEQ ID NO: 141). In some embodiments, the n is an integer of four. In some embodiments, the engineered polypeptide comprises a protease cleavage site. In some embodiments, the proteases cleavage site comprises a Furin protease site. In some embodiments, the engineered polypeptide comprises an amino acid sequence that is at least 80% identical to any one of SEQ ID NOs: 131-140. In some embodiments, the engineered polypeptide comprises an amino acid sequence that is at least 95% identical to any one of SEQ ID NOs: 131-140. In some embodiments, the engineered polypeptide comprises an amino acid sequence of any one of SEQ ID NOs: 131-140. In some embodiments, the natriuretic peptide comprises a C-type natriuretic peptide (CNP) or fragment thereof. In some embodiments, the CNP or fragment thereof comprises 22 contiguous bases of SEQ ID NO: 1. In some embodiments, the CNP or fragment thereof comprises the 22 contiguous bases at C terminus of SEQ ID NO: 1. In some embodiments, the CNP or fragment thereof comprises 36 contiguous bases of SEQ ID NO: 1. In some embodiments, the CNP or fragment thereof comprises the 36 contiguous bases at C terminus of SEQ ID NO: 1. In some embodiments, the CNP or fragment thereof comprises 53 contiguous bases of SEQ ID NO: 1. In some embodiments, the CNP or fragment thereof comprises the 53 contiguous bases at C terminus of SEQ ID NO: 1. In some embodiments, the viral vector is an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV serotype comprises the AAV2. In some embodiments, the AAV vector encodes a modified AAV capsid. In some embodiments, the AAV vector comprises a second expression cassette. In some embodiments, the second expression cassette encodes a therapeutic. In some embodiments, the therapeutic comprises a hormone. In some embodiments, the therapeutic comprises an agonist of a natriuretic peptide receptor (NPR). In some embodiments, the therapeutic comprises an agonist of a cyclic GMP (cGMP) signaling pathway. In some embodiments, the therapeutic comprises an VEGF inhibitor.

Described herein, in some aspects, is an engineered polypeptide comprising an antibody or a fragment thereof operatively coupled to a natriuretic peptide, wherein the antibody or fragment thereof comprises an amino acid sequence that is at least 80% identical to any one of SEQ ID NOs: 6-8. In some embodiments, the natriuretic peptide comprises an amino acid sequence that is at least 80% identical to any one of SEQ ID NOs: 1-5. In some embodiments, the natriuretic peptide comprises an amino acid sequence that is at least 95% identical to any one of SEQ ID NOs: 1-5. In some embodiments, the natriuretic peptide comprises an amino acid sequence of any one of SEQ ID NOs: 1-5. In some embodiments, the natriuretic peptide comprises an amino acid sequence that is at least 80% identical to SEQ ID NO: 4. In some embodiments, the natriuretic peptide comprises an amino acid sequence that is at least 95% identical to SEQ ID NO: 4. In some embodiments, the natriuretic peptide comprises an amino acid sequence of SEQ ID NO: 4. In some embodiments, the antibody or fragment thereof comprises an amino acid sequence that is at least 95% identical to any one of SEQ ID NOs: 6-8. In some embodiments, the antibody or fragment thereof comprises an amino acid sequence of any one of SEQ ID NOs: 6-8. In some embodiments, the natriuretic peptide is covalently connected to N terminus of the antibody or fragment thereof. In some embodiments, the natriuretic peptide is covalently connected to C terminus of the antibody or fragment thereof. In some embodiments, the natriuretic peptide is covalently connected to the antibody or fragment thereof by a peptide linker. In some embodiments, the peptide linker comprises an amino acid sequence comprising (GGGGS)n, wherein the n is an integer between 0-20 (SEQ ID NO: 141). In some embodiments, the n is an integer of four. In some embodiments, the engineered polypeptide comprises a protease cleavage site. In some embodiments, the proteases cleavage site comprises a Furin protease site. In some embodiments, the engineered polypeptide comprises an amino acid sequence that is at least 80% identical to any one of SEQ ID NOs: 131-140. In some embodiments, the engineered polypeptide comprises an amino acid sequence that is at least 95% identical to any one of SEQ ID NOs: 131-140. In some embodiments, the engineered polypeptide comprises an amino acid sequence of any one of SEQ ID NOs: 131-140. In some embodiments, the natriuretic peptide comprises a C-type natriuretic peptide (CNP) or fragment thereof. In some embodiments, the CNP or fragment thereof comprises 22 contiguous bases of SEQ ID NO: 1. In some embodiments, the CNP or fragment thereof comprises the 22 contiguous bases at C terminus of SEQ ID NO: 1. In some embodiments, the CNP or fragment thereof comprises 36 contiguous bases of SEQ ID NO: 1. In some embodiments, the CNP or fragment thereof comprises the 36 contiguous bases at C terminus of SEQ ID NO: 1. In some embodiments, the CNP or fragment thereof comprises 53 contiguous bases of SEQ ID NO: 1. In some embodiments, the CNP or fragment thereof comprises the 53 contiguous bases at C terminus of SEQ ID NO: 1.

Described herein, in some aspects, is a cell comprising the engineered polynucleotide described herein. In some embodiments, at least a fragment of the engineered polynucleotide is integrated into a genome of the cell. Also described herein, in some aspects, is a cell comprising the engineered polypeptide described herein.

Described herein, in some aspects, is a viral particle comprising the engineered polynucleotide described herein. In some embodiments, the viral particle comprises an AAV capsid. In some embodiments, the AAV capsid comprises a modified AAV capsid. In some embodiments, the modified AAV capsid comprises a modified AAV2 capsid.

Described herein, in some aspects, is a pharmaceutical composition comprising the engineered polynucleotide of described herein, the engineered polypeptide described herein, the cell described herein (e.g., a cell transduced with engineered polynucleotide described herein), or the viral particle described herein. In some embodiments, pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition is formulated for administering intravitreally, subretinally, or suprachoroidally. In some embodiments, the pharmaceutical composition is for treating an ocular disease or condition. In some embodiments, the pharmaceutical composition increases natriuretic peptide receptor-B signaling, guanylyl cyclase signaling, cyclic guanosine monophosphate (cGMP) signaling, or a combination thereof in a subject in need thereof.

Described herein, in some aspects, is a method of treating a disease or condition in a subject in need thereof, the method comprising administering to the subject a therapeutically effective amount of the engineered polynucleotide of described herein, the engineered polypeptide described herein, the cell described herein (e.g., a cell transduced with engineered polynucleotide described herein), the viral particle described herein, the pharmaceutical composition described herein, or a combination thereof. Described herein, in some aspects, is a method of treating a disease or condition in a subject in need thereof, the method comprising administering to the subject a therapeutically effective amount of the engineered polynucleotide of described herein, the engineered polypeptide described herein, the cell described herein (e.g., a cell transduced with engineered polynucleotide described herein), the viral particle described herein, the pharmaceutical composition described herein, or a combination thereof, wherein once of the administering is curative of the disease or condition. Described herein, herein, in some aspects, is a method of treating a disease or condition in a subject in need thereof, the method comprising administering to the subject a therapeutically effective amount of the engineered polynucleotide of described herein, the engineered polypeptide described herein, the cell described herein (e.g., a cell transduced with engineered polynucleotide described herein), the viral particle described herein, the pharmaceutical composition described herein, or a combination thereof, wherein the administering does not comprise daily administration. Described herein, in some aspects, is a method of treating a disease or condition in a subject in need thereof, the method comprising administering to the subject a therapeutically effective amount of the engineered polynucleotide of described herein, the engineered polypeptide described herein, the cell described herein (e.g., a cell transduced with engineered polynucleotide described herein), the viral particle described herein, the pharmaceutical composition described herein, or a combination thereof, wherein the administering comprises a weekly administration, a bi-weekly administration, a monthly administration, a bi-month administration, a semiannual administration, an annual administration, or a combination thereof. In some embodiments, the disease or condition comprises an ocular disease. In some embodiments, the ocular disease comprises ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof. In some embodiments, the engineered polynucleotide of described herein, the engineered polypeptide described herein, the cell described herein (e.g., a cell transduced with engineered polynucleotide described herein), the viral particle described herein, the pharmaceutical composition described herein, or a combination thereof increases natriuretic peptide receptor-B signaling, guanylyl cyclase signaling, cyclic guanosine monophosphate (cGMP) signaling, or a combination thereof in the subject, thereby treating the disease or condition. In some embodiments, the engineered polynucleotide of described herein, the engineered polypeptide described herein, the cell described herein (e.g., a cell transduced with engineered polynucleotide described herein), the viral particle described herein, the pharmaceutical composition described herein, or a combination thereof increases a half-life of an agonist for natriuretic peptide receptor-B signaling, guanylyl cyclase signaling, cyclic guanosine monophosphate (cGMP) signaling, or a combination thereof in the subject, thereby treating the disease or condition. In some embodiments, the half-life is increased by at least two-fold, at least five-fold, at least ten-fold, at least twenty-fold, at least fifty-fold, at least one hundred-fold, or more fold compared to half-life of an endogenous agonist for the natriuretic peptide receptor-B signaling, the guanylyl cyclase signaling, the cyclic guanosine monophosphate (cGMP) signaling, or a combination thereof in the subject.

Described herein, in some aspects, is a method of treating a disease or condition in a subject, comprising administering an engineered polynucleotide to the subject, wherein the engineered polynucleotide comprises a viral vector comprising an expression cassette for expressing an engineered polypeptide comprising an antibody or a fragment thereof operatively coupled to a natriuretic peptide in a cell of the subject, and wherein the cell expresses the engineered polypeptide, thereby treating the disease or condition. In some embodiments, the natriuretic peptide comprises an amino acid sequence that is at least 80% identical to any one of SEQ ID NOs: 1-5. In some embodiments, the natriuretic peptide comprises an amino acid sequence that is at least 95% identical to any one of SEQ ID NOs: 1-5. In some embodiments, the natriuretic peptide comprises an amino acid sequence of any one of SEQ ID NOs: 1-5. In some embodiments, the natriuretic peptide comprises an amino acid sequence that is at least 80% identical to SEQ ID NO: 4. In some embodiments, the natriuretic peptide comprises an amino acid sequence that is at least 95% identical to SEQ ID NO: 4. In some embodiments, the natriuretic peptide comprises an amino acid sequence of SEQ ID NO: 4. In some embodiments, the antibody or fragment thereof comprises a fragment crystallizable (Fc) region. In some embodiments, the antibody or fragment thereof comprises an amino acid sequence that is at least 80% identical to any one of SEQ ID NOs: 6-8. In some embodiments, the antibody or fragment thereof comprises an amino acid sequence that is at least 95% identical to any one of SEQ ID NOs: 6-8. In some embodiments, the antibody or fragment thereof comprises an amino acid sequence of any one of SEQ ID NOs: 6-8. In some embodiments, the natriuretic peptide is covalently connected to N terminus of the antibody or fragment thereof. In some embodiments, the natriuretic peptide is covalently connected to C terminus of the antibody or fragment thereof. In some embodiments, the natriuretic peptide is covalently connected to the antibody or fragment thereof by a peptide linker. In some embodiments, the peptide linker comprises an amino acid sequence comprising (GGGGS)n, wherein the n is an integer between 0-20 (SEQ ID NO: 141). In some embodiments, the n is an integer of four. In some embodiments, the engineered polypeptide comprises a protease cleavage site. In some embodiments, the proteases cleavage site comprises a Furin protease site. In some embodiments, the engineered polypeptide comprises an amino acid sequence that is at least 80% identical to any one of SEQ ID NOs: 131-140. In some embodiments, the engineered polypeptide comprises an amino acid sequence that is at least 95% identical to any one of SEQ ID NOs: 131-140. In some embodiments, the engineered polypeptide comprises an amino acid sequence of any one of SEQ ID NOs: 131-140. In some embodiments, the natriuretic peptide comprises a C-type natriuretic peptide (CNP) or fragment thereof. In some embodiments, the CNP or fragment thereof comprises 22 contiguous bases of SEQ ID NO: 1. In some embodiments, the CNP or fragment thereof comprises the 22 contiguous bases at C terminus of SEQ ID NO: 1. In some embodiments, the CNP or fragment thereof comprises 36 contiguous bases of SEQ ID NO: 1. In some embodiments, the CNP or fragment thereof comprises the 36 contiguous bases at C terminus of SEQ ID NO: 1. In some embodiments, the CNP or fragment thereof comprises 53 contiguous bases of SEQ ID NO: 1. In some embodiments, the CNP or fragment thereof comprises the 53 contiguous bases at C terminus of SEQ ID NO: 1. In some embodiments, the engineered polynucleotide comprises an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV serotype comprises the AAV2. In some embodiments, the AAV vector encodes a modified AAV capsid. In some embodiments, the engineered polynucleotide is encapsulated in a viral particle. In some embodiments, the viral particle comprises an AAV capsid. In some embodiments, the AAV capsid is a modified AAV capsid. In some embodiments, the viral vector comprises a second expression cassette. In some embodiments, the second expression cassette encodes a therapeutic. In some embodiments, the therapeutic comprises a hormone. In some embodiments, the therapeutic comprises an agonist of a natriuretic peptide receptor (NPR). In some embodiments, the therapeutic comprises an agonist of a cyclic GMP (cGMP) signaling pathway. In some embodiments, the therapeutic comprises an VEGF inhibitor. the engineered polynucleotide is administered to the subject intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof. In some embodiments, the disease or condition comprises an ocular disease. In some embodiments, the ocular disease comprises ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

Described herein, in some aspects, is a method of treating glaucoma in a subject, the method comprising administering an AAV2 vector to the subject, wherein the AAV2 vector encodes a natriuretic peptide covalently connected to an antibody or fragment thereof. In some embodiments, the natriuretic peptide the natriuretic peptide comprises an amino acid sequence that is at least 80% identical to SEQ ID NO: 4. In some embodiments, the natriuretic peptide comprises an amino acid sequence that is at least 95% identical to SEQ ID NO: 4. In some embodiments, the natriuretic peptide comprises an amino acid sequence that is of SEQ ID NO: 4. In some embodiments, the antibody or fragment thereof comprises a fragment crystallizable (Fc) region. In some embodiments, the antibody or fragment thereof comprises an amino acid sequence that is at least 80% identical to any one of SEQ ID NOs: 6-8. In some embodiments, the antibody or fragment thereof comprises an amino acid sequence that is at least 95% identical to any one of SEQ ID NOs: 6-8. In some embodiments, the antibody or fragment thereof comprises an amino acid sequence of any one of SEQ ID NOs: 6-8. In some embodiments, the natriuretic peptide is covalently connected to N terminus of the antibody or fragment thereof. In some embodiments, the natriuretic peptide is covalently connected to C terminus of the antibody or fragment thereof. In some embodiments, the natriuretic peptide is covalently connected to the antibody or fragment thereof by a peptide linker. In some embodiments, the peptide linker comprises an amino acid sequence comprising (GGGGS)n, wherein the n is an integer between 0-20 (SEQ ID NO: 141). In some embodiments, the n is an integer of four. In some embodiments, a protease cleavage site is flanked by the natriuretic peptide and the antibody or fragment thereof. In some embodiments, the proteases cleavage site comprises a Furin protease site. In some embodiments, the natriuretic peptide comprises a C-type natriuretic peptide (CNP) or fragment thereof. In some embodiments, the CNP or fragment thereof comprises 22 contiguous bases of SEQ ID NO: 1. In some embodiments, the CNP or fragment thereof comprises the 22 contiguous bases at C terminus of SEQ ID NO: 1. In some embodiments, the CNP or fragment thereof comprises 36 contiguous bases of SEQ ID NO: 1. In some embodiments, the CNP or fragment thereof comprises the 36 contiguous bases at C terminus of SEQ ID NO: 1. In some embodiments, the CNP or fragment thereof comprises 53 contiguous bases of SEQ ID NO: 1. In some embodiments, the CNP or fragment thereof comprises the 53 contiguous bases at C terminus of SEQ ID NO: 1. In some embodiments, the AAV2 vector encodes an modified AAV capsid. In some embodiments, the AAV2 vector comprises a second expression cassette. In some embodiments, the second expression cassette encodes a therapeutic. In some embodiments, the therapeutic comprises a hormone. In some embodiments, the therapeutic comprises an agonist of a natriuretic peptide receptor (NPR). In some embodiments, the therapeutic comprises an agonist of a cyclic GMP (cGMP) signaling pathway. In some embodiments, the therapeutic comprises an VEGF inhibitor. In some embodiments, the AAV2 vector is encapsulated in an AAV viral particle prior to the administering. In some embodiments, the AAV2 vector is administered intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the AAV2 vector is administered intravitreally, subretinally, or suprachoroidally. In some embodiments, the administering of the AAV2 vector increases natriuretic peptide receptor-B signaling, guanylyl cyclase signaling, cyclic guanosine monophosphate (cGMP) signaling, or a combination thereof in the subject. In some embodiments, once of the administering of the AAV2 vector is curative of the glaucoma. In some embodiments, the administering of the AAV2 vector does not comprise daily administration. In some embodiments, the administering of the AAV2 vector comprises a weekly administration, a bi-weekly administration, a monthly administration, a bi-month administration, a semiannual administration, an annual administration, or a combination thereof. In some embodiments, the administering of the AAV2 vector increases a half-life of an agonist for natriuretic peptide receptor-B signaling, guanylyl cyclase signaling, cyclic guanosine monophosphate (cGMP) signaling, or a combination thereof in the subject. In some embodiments, the half-life is increased by at least two-fold, at least five-fold, at least ten-fold, at least twenty-fold, at least fifty-fold, at least one hundred-fold, or more fold compared to half-life of an endogenous agonist for the natriuretic peptide receptor-B signaling, the guanylyl cyclase signaling, the cyclic guanosine monophosphate (cGMP) signaling, or a combination thereof in the subject. In some embodiments, the glaucoma comprises neovascular glaucoma (NG), glaucoma, traumatic glaucoma, or a combination thereof.

Described herein, in some aspects, is an engineered polynucleotide comprising an AAV vector comprising one or more expression cassettes, wherein the one or more expression cassettes encode a peptide. Also described herein, in some aspects, is an engineered polynucleotide comprising an AAV vector comprising one or more expression cassettes, wherein the one or more expression cassettes encode an engineered polypeptide comprising: an antibody or fragment thereof operatively coupled to a peptide. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV serotype comprises the AAV2. In some embodiments, the peptide comprises a CNP. In some embodiments, the CNP comprises at least 22 amino acid residues. In some embodiments, the CNP comprises at least 36 amino acid residues. In some embodiments, the CNP comprises at least 53 amino acid residues. In some embodiments, the CNP comprises an amino acid sequence that is at least 80% identical to SEQ ID NOs: 1-5. In some embodiments, the peptide is covalently connected to N terminus of the antibody or fragment thereof. In some embodiments, the peptide is covalently connected to C terminus of the antibody or fragment thereof. In some embodiments, the peptide is operatively coupled to the antibody or fragment thereof by a peptide linker. In some embodiments, the peptide linker comprises an amino acid sequence comprising (GGGGS)ₙ, wherein the n is an integer between 0-10 (SEQ ID NO: 141). In some embodiments, the AAV vector encodes an engineered AAV capsid.

Described herein, in some aspects, is an engineered polypeptide comprising an antibody or a fragment thereof operatively coupled to a peptide, wherein the antibody or fragment thereof comprises an amino acid sequence that is at least 80% identical to any one of SEQ ID NOs: 6-8. In some embodiments, wherein the peptide comprises a CNP. In some embodiments, the CNP comprises at least 22 amino acid residues. In some embodiments, the CNP comprises at least 36 amino acid residues. In some embodiments, the CNP comprises at least 53 amino acid residues. In some embodiments, the CNP comprises a amino acid sequence that is at least 80% identical to SEQ ID NOs: 1-5. In some embodiments, the peptide is covalently connected to N terminus of the antibody or fragment thereof. In some embodiments, the peptide is covalently connected to C terminus of the antibody or fragment thereof. In some embodiments, the peptide is operatively coupled to the antibody or fragment thereof by a peptide linker. In some embodiments, the peptide linker comprises an amino acid sequence comprising (GGGGS)n, wherein the n is an integer between 0-10 (SEQ ID NO: 141).

Described herein, in some aspects, is an engineered polynucleotide encoding an engineered polypeptide described herein. In some embodiments, the engineered polynucleotide is a vector. In some embodiments, the vector is a viral vector. In some embodiments, the viral vector comprises an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV serotype comprises the AAV2. In some embodiments, the AAV vector encodes an engineered AAV capsid. In some embodiments, the viral vector comprises one or more expression cassettes. In some embodiments, the one or more expression cassettes encode a contiguous polypeptide, wherein the contiguous polypeptide comprises an engineered polypeptide described herein. In some embodiments, the contiguous polypeptide comprises a protease cleavable sequence. In some embodiments, the contiguous polypeptide comprises a Furin cleavable sequence. In some embodiments, the contiguous polypeptide comprises a self-cleaving polypeptide sequence. In some embodiments, the one or more expression cassettes express at least one additional therapeutic. In some embodiments, the at least one additional therapeutic comprises a hormone. In some embodiments, the at least one additional therapeutic comprises an agonist of a natriuretic peptide receptor (NPR). In some embodiments, the at least one additional therapeutic comprises an agonist of a cyclic GMP (cGMP) signaling pathway. In some embodiments, the at least one additional therapeutic comprises an VEGF inhibitor. In some embodiments, the VEGF inhibitor binds to and inhibits VEGF-A, VEGF-B, VEGF-C, VEGF-D, or a combination thereof. In some embodiments, the VEGF inhibitor comprises an antibody. In some embodiments, the VEGF inhibitor comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. In some embodiments, the VEGF inhibitor comprises a non-antibody VEGF inhibitor. In some embodiments, the non-antibody VEGF inhibitor is a VEGF receptor 1 (VEGFR1), a VEGF receptor 2 (VEGFR2), a VEGF receptor 3 (VEGFR3), a fragment thereof, or a combination thereof. In some embodiments, the non-antibody VEGF inhibitor comprises a soluble VEGFR1, a soluble VEGFR2, a soluble VEGFR3, a soluble fragment thereof, or a combination thereof. In some embodiments, the non-antibody VEGF inhibitor comprises a VEGF-Trap or a modified version thereof.

Described herein, in some aspects, is a cell comprising an engineered polynucleotide described herein.

Described herein, in some aspects, is a cell comprising an engineered polypeptide described herein.

Described herein, in some aspects, is a pharmaceutical composition comprising an engineered polynucleotide described herein, an engineered polypeptide described herein, or a cell described herein. In some embodiments, pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition is formulated for administering intravitreally, subretinally, or suprachoroidally. In some embodiments, the pharmaceutical composition is for treating an ocular disease or condition. In some embodiments, the pharmaceutical composition increases natriuretic peptide receptor-B signaling, guanylyl cyclase signaling, cyclic guanosine monophosphate (cGMP) signaling, or a combination thereof in a subject in need thereof.

Described herein, in some aspects, is a method of treating a disease or condition in a subject in need thereof, the method comprising administering to the subject a therapeutically effective amount of an engineered polynucleotide described herein, an engineered polypeptide described herein, a cell described herein, or a pharmaceutical composition described herein. In some embodiments, the disease or condition comprises an ocular disease. In some embodiments, the ocular disease comprises ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

Described herein, in some aspects, is a method of treating a disease or condition in a subject in need thereof, the method comprising administering to the subject a therapeutically effective amount of an engineered polynucleotide described herein, an engineered polypeptide described herein, a cell described herein, or a pharmaceutical composition described herein, where once of the administering is curative of the disease or condition. In some embodiments, the disease or condition comprises an ocular disease. In some embodiments, the ocular disease comprises ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

Described herein, in some aspects, is a method of treating a disease or condition in a subject in need thereof, the method comprising administering to the subject a therapeutically effective amount of an engineered polynucleotide described herein, an engineered polypeptide described herein, a cell described herein, or a pharmaceutical composition described herein, where the administering does not comprise daily administration. In some embodiments, the disease or condition comprises an ocular disease. In some embodiments, the ocular disease comprises ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

### BRIEF DESCRIPTION OF THE DRAWINGS

This patent application contains at least one drawing executed in color. Copies of this patent or patent application with color drawing(s) will be provided by the Office upon request and payment of the necessary fee.

FIGS. 1A-F illustrate exemplary constructs for encoding and expressing a natriuretic peptide (e.g., a CNP) described herein. FIG. 1A discloses SEQ ID NO: 183 and “VGGRK” as SEQ ID NO: 185 and “4×GGGS” as SEQ ID NO: 186. FIG. 1B discloses SEQ ID NOS 184, 9 and 9, respectively, in order of appearance. FIG. 1C discloses “VGGRK” as SEQ ID NO: 185. FIG. 1D discloses “4×GGGS” as SEQ ID NO: 186. FIG. 1E discloses “VGGRK” as SEQ ID NO: 185. FIG. 1F discloses “4×GGGS” as SEQ ID NO: 186.

FIG. 2A illustrates an exemplary AAV vector encoding a natriuretic peptide described herein. FIG. 2A discloses “4×GGGS” as SEQ ID NO: 186.

FIG. 2B illustrates exemplary arrangements of CNP fused with a fusion partner described herein. Left panel depicts unfused CNP. Right panel depicts CNP fused with a fusion partner to form a CNP fusion protein (FP).

FIGS. 3A-C illustrate exemplary arrangements of a natriuretic peptide operatively coupled (e.g., covalently attached) to an antibody or fragment thereof (e.g., a fragment crystallizable region or Fc of the antibody). FIG. 3A discloses SEQ ID NOS 187, 187, 188 and 188, respectively, in order of appearance. FIG. 3B discloses SEQ ID NOS 187, 187, 188 and 188, respectively, in order of appearance. FIG. 3C discloses SEQ ID NOS 187, 187, 188, 188, 189, 189, 190 and 190, respectively, in order of appearance.

FIG. 4A illustrates exemplary anti-human CNP antibody titration standard curves.

FIG. 4B illustrates exemplary comparisons of detections of antibody dilutions.

FIG. 4C illustrates exemplary standard curves for abundance of CNP-Fc encoded by AMI061, AMI087, or AMI088.

FIG. 5 illustrates exemplary AAV vector expression profile in HEK293, HCH, or SkMC cells transduced with an AAV vector (AMI061, AMI087, or AMI088) described herein.

FIGS. 6A-B illustrate exemplary SDS-PAGE and peptide sequencing confirming CNP or CNP-Fc expression by the AAV vector transduced cells.

FIG. 7A illustrates that the AAV CNP-Fc36 encoded and expressed from the AAV vector was functional in vitro (as determined by cyclic guanosine monophosphate, cGMP, production stimulated by the expressed CNP36). AAV vector encoded CNP or Fc1-CNP stimulated cyclic GMP production. The highest level of cGMP was produced by CNP-Fc36 encoded by AAV vector AMI088.

FIG. 7B illustrates affinity binding between a CNP fusion described herein and NPR-B protein as measured by BiaCore assay.

FIG. 7C illustrates single-cycle kinetics (SCK) assay of binding between CNP and NPR-B.

FIG. 7D illustrates Biacore assay for AMI263 (Fc4-CNP36) against NPR-B.

FIG. 7E illustrates Biacore assay for CNP-Fc encoded by AMI088 against NPR-B.

FIG. 7F illustrates Biacore assay for ANP against NPR-B.

FIG. 7G illustrates Biacore assay for BNP against NPR-B.

FIG. 7H illustrates Biacore assay for CNP-Fc encoded by AMI263 against NPR-B.

FIG. 7I illustrates Biacore assay for CNP-Fc encoded by AMI087 against NPR-B.

FIGS. 8A-B illustrate that the AAV CNP-Fc36 encoded and expressed from the AAV vector was functional in vitro (as determined by cGMP production stimulated by the expressed CNP36) in time-course measurements.

FIG. 9A illustrates body weight measurements of mice before (day 0) or seven days after administration of AAV vector for in vivo functional study. On the day of intravitreal (IVT) injection, the body weight of all animals was 20-22 g, and all animals generally maintained body weight over the course of the study.

FIG. 9B illustrates representative images from the central retina from the hematoxylin and eosin (H&E) staining.

FIG. 9C illustrates retinal ganglion cell (RGC) count of the H&E staining. The mean RGC count of the right eyes (ODs; experimental eyes) was lower than the mean RGC count of the left eyes (OSs; control eyes), and there were no differences in the mean RGC count in the ODs and OSs between Groups 1 and 2.

FIG. 10A illustrates Flatmount analysis for RGC cell count of Groups 1 and 2 of the mice of the in vivo functional study. Groups 1 and 2 were stained with 4′, 6-diamidino-2-phenylindole (DAPI) and neuron-specific class III beta-tubulin antibody (TUJ-1), and images were captured at 20×. RGCs were counted in four quadrants, and cells/mm² were averaged across each group.

FIG. 10B illustrates retinal ganglion cell (RGC) count from Flatmount analysis (Groups 1 and 2) of FIG. 10A. When the RGC count was quantified across groups, the control group (Group 1 OS; PBS, no NMDA) had an average count of 11,000±700 cells/mm². The NMDA control group (Group 1 OD; PBS+NMDA) had a decreased RGC count of 8,000±1,000 cells/mm². The Group 2 ODs had an RGC count slightly increased over the Group 1 ODs, at 9,000±500 cells/mm².

FIG. 10C illustrates Flatmount analysis for RGC cell count of groups 3-8 of the mice of the in vivo functional study. Groups 3-8 were stained for DAPI, and four images were captured for each retina at 20× approximately 600 μm from the optic nerve head. RGCs were counted in a central 248.1×325.7 μm region and cells/mm² were averaged over each group.

FIG. 10D illustrates retinal ganglion cell (RGC) count from Flatmount analysis (Groups 3-8) of FIG. 10C. When the RGC count was calculated, the control group (Group 7 OS; PBS, no NMDA) had an average count of 12,000±700 cells/mm². The NMDA control group (Group 7 OD; PBS+NMDA) had a decreased RGC count of 9,000±300 cells/mm². The Group 8 ODs had an RGC count similar to the Group 7 OSs, at 12,000±300 cells/mm². Group 3 ODs had an RGC count similar to the Group 7 ODs, at 9,000±1,000 cells/mm². Group 4 ODs had an RGC count of 10,000±1,400 cells/mm². Group 5 ODs had an RGC count of 10,000±600 cells/mm². Group 6 ODs had an RGC count of 10,000±1,500 cells/mm².

FIGS. 11A-B illustrate statistical analyses on the Groups 3-8 (one-way ANOVA followed by multiple comparisons using the Dunnett statistical hypothesis comparison). Treatment of PBS-injected eyes with NMDA caused a statistically significant decrease in RGC counts (P<0.0001). Increasing the dose of MK-801 to 100 μM rescued the Group 8 RGC counts to the Group 7 OS control level and the difference between NMDA-treated (Group 7 OD) and NMDA+MK-801-treated (Group 8) eyes was statistically significant (P<0.0001). When Groups 4-6 were compared to the Group 3 NMDA/sham vector controls, both the AMI 182 (Group 5) and AMI 088 (Group 6) reached statistical significance (FIG. 11A; P=0.0332 for Group 5 versus Group 3; P=0.0021 for Group 6 versus Group 3). When the RGC count of the AAV-treated groups were compared to the NMDA/PBS controls (Group 7 OD), only the AMI 088 construct maintained statistical significance (P=0.002; FIG. 11B).

FIG. 12A illustrates immunohistochemistry images obtained from retina Flatmount. Groups 3-6 were stained for CNP36 in addition to DAPI to evaluate the expression location and levels of the AAV vectors and images (4× and 20×) were captured.

FIG. 12B illustrates phase 1 immunohistochemistry (Groups 1 and 2). Qualitatively, there did not appear to be a difference in number of TUJ-1+ and cone arrestin+ cells between groups. All groups had clear cone arrestin staining with some TUJ-1+ cells observed.

FIGS. 13A-B illustrates quantification of CNP-Fc or CNP. CNP36-Fc quantification in ocular and serum samples: animals from Groups 3 (AMI189) and 6 (AMI088) were analyzed using the CNP36-Fc ELISA. Group 3 (sham vector) animals served as a negative control (FIG. 13A). Group 6 animals were injected with AMI088, which was an AAV2 vector with N-terminal CNP36 fused to the C-terminus of human IgG1-Fc fragment (CNP36-Fc). The data were analyzed in GraphPad Prism software using one-way ANOVA followed by Dunnett's multiple comparison (****=<0.0001). A statistically significant difference was observed between Groups 3 and 6. In the samples where the Fc fusion was not present, CNP36 levels were low due to short half-life of CNP36. Ocular samples from Groups 3, 4 and 5 were analyzed using the commercial ELISA kit described above (FIG. 13B, left graph). Although Groups 4 and 5 showed a slightly higher level of CNP36 compared to Group 3 (sham vector; negative control), the observed differences were not significant. The low levels observed on the ELISA indicated that only a small fraction of the total expressed CNP36 was quantified as most of the protein could have been proteolyzed prior to quantification. Since the test articles for Groups 4 and 5 showed efficacy, CNP36 was expressed but was not protected from proteolysis. Neither CNP36-Fc nor CNP36 were expressed in the serum samples. Right graph of FIG. 13B illustrates an exemplary in vivo expression of CNP fusion detected by ELISA.

FIG. 14 illustrates RGC protection by AAV vectors encoding CNP. Mouse eye was injected via IVT 1 μl of AAV construct at 4E+8 vg/eye, 28 days prior to NMDA injection. Images indicated NMDA induced RGC #reduction, which was rescued by MK-801. Sham vector showed reduced RGCs in the NMDA only treatment group, but the AMI182 and AMI088 groups showed higher RGC counts than the groups of sham vector and NMDA treatments.

FIG. 15 illustrates an exemplary SDS-PAGE showing the pattern associated with expression of VP1, VP2, or VP3. All the AAV samples were: produced in Sf-9 cells; purified by double CsCl2 ultracentrifugation, buffer exchanged; and sterile filtered. Approximately 1e11 vg/lane was loaded on the gel.

FIGS. 16A-C illustrates effect of Fc4-CNP36 (FP) on intraocular pressure (IOP) change in rat partial optic nerve transection (pONT) model. FIG. 16A illustrates animal IOP monitored during the course of the study. FIG. 16B illustrates animal IOP change after Fc4-CNP36 administration intravitreally (IVT). FIG. 16C illustrates effect of Fc4-CNP36 concentration on IOP change post administration intravitreally. Vehicle: 10 mM phosphate, pH7.3, 180 mM NaCl, 0.001% Pluronic F68; FP (L)=2 μg/eye IVT; FP (M)=20 μg/eye IVT; and FP (H)=80 μg/eye IVT.

FIGS. 17A-B illustrates effects of effect of Fc4-CNP36 on RGC protection in rat pONT model. FIG. 17A illustrates Fc4-CNP36 concentration detection of apoptosing retinal cells (DARC) reduction. FIG. 17B illustrates Fc4-CNP36 concentration on RGC count. Vehicle: 10 mM phosphate, pH7.3, 180 mM NaCl, 0.001% Pluronic F68; FP (L)=2 μg/eye IVT; FP (M)=20 μg/eye IVT; and FP (H)=80 μg/eye IVT.

FIG. 18 illustrates protein concentration at the terminal samples of the rat pONT model. Only OS (left eye) was injected, and OD (right eye) was not.

FIG. 19A-B illustrate effect of AAV2.N54-Fc4-CNP36 and AAV2.N54-CPNP36 on retina DARC reduction in rat pONT model. DARC values had been subtracted from the baseline. Terminal DARC counts were plotted. FIG. 19A: AMI273 was the vector of AAV2.N54-Fc4-CNP36 which expressed Fc4-CNP36 protein. FIG. 19B: AMI302 was the vector of AAV2.N54-CNP36 which expressed CNP36 peptide.

FIG. 20A-B illustrate effect of AAV2.N54-Fc4-CNP36 (AAV-FP) and AAV2.N54-CPNP36 (AAV-P) on RGC protection in Rat pONT model. FIG. 20A: AMI273 was the vector of AAV2.N54-Fc4-CNP36 which expressed Fc4-CNP36 protein. FIG. 20B: AMI302 was the vector of AAV2.N54-CNP36 which expressed CNP36 peptide.

FIG. 21 illustrates RGC counts treated eyes of animals dosed with Fc4-CNP36 protein (FP) and AAV vectors in rat pONT model.

FIG. 22 illustrates Fc4-CNP36 concentration in ocular and serum samples after administration of AAV vectors (AAV-FP) IVT. The GOI of Fc4-CNP36 was expressed in the AMI273 dosed eyes but no expression in un-dosed eyes.

FIG. 23 illustrates CNP36 concentration in ocular and serum samples after administration of AAV vectors (AAV-P) IVT.

FIG. 24 illustrates comparison of DARC count and RGC regional density.

FIG. 25A-B illustrate determination of EC₅₀ for CNP-Fc described herein. FIG. 25A illustrates a standard curve of cGMP. FIG. 25B illustrates data fitting and calculations of EC₅₀ of various CNP fusion (e.g., CNP-Fc) and comparable natriuretic peptide described herein.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

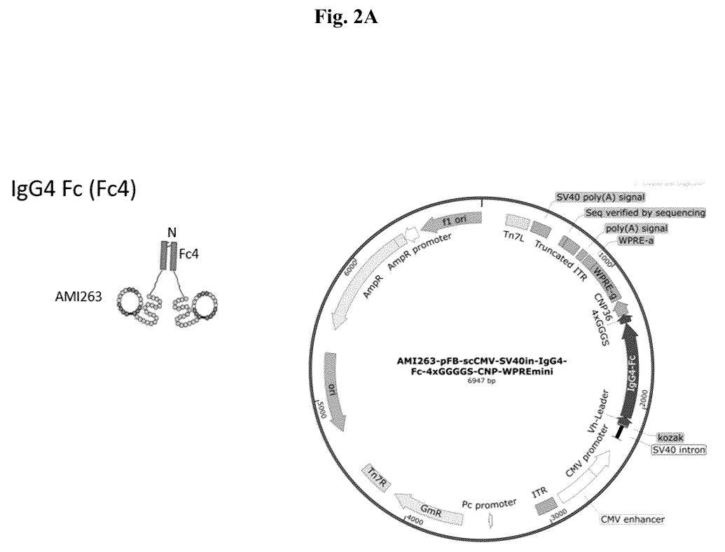

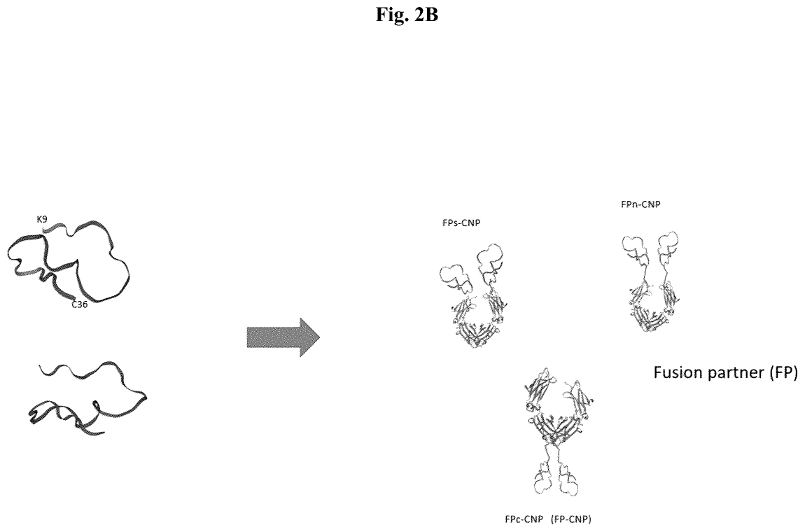

Described herein, in some aspects, is an engineered polynucleotide comprising an AAV vector comprising one or more expression cassettes for encoding a peptide. Also described herein is an engineered polynucleotide comprising an AAV vector comprising one or more expression cassettes for encoding an engineered polypeptide comprising: an antibody or fragment thereof operatively coupled to a peptide. For example, the engineered polypeptide comprises an antibody or fragment thereof operatively coupled to a CNP described herein. FIG. 2A illustrates an exemplary AAV vector for encoding a peptide (e.g., a CNP) or a fusion protein (e.g., a CNP-Fc fusion protein). FIG. 2B illustrates exemplary arrangements of CNP fused with a fusion partner described herein. In some embodiments, the peptide encoded by the engineered polynucleotide is a natriuretic peptide. In some embodiments, the peptide is CNP. In some embodiments, the CNP is a full length CNP. In some embodiments, the CNP is cleaved. In some embodiments, the CNP comprises at least 22 amino acid residues, at least 36 amino acid residues, or at least 53 amino acid residues. In some embodiments, the CNP is CNP-22 (SEQ ID NO: 5). In some embodiments, the CNP is CNP-36 (SEQ ID NO: 4). In some embodiments, the CNP is CNP-53 (SEQ ID NO: 3). In some embodiments, the CNP comprises an amino acid sequence that is at least 50%, at least 60%, at least 70%, at least 80%, at least 90%, at least 95%, or at least 99% identical to any one of SEQ ID NOs: 1-5. In some embodiments, the CNP comprises an amino acid sequence that is 100% identical to any one of SEQ ID NOs: 1-5.

In some embodiments, the peptide is operatively coupled to an antibody or fragment thereof (e.g., an Fc fragment). In some embodiments, the antibody or fragment thereof comprises a signaling peptide (SP). For example, the engineered polypeptide can comprise an amino acid that is at least 50%, at least 60%, at least 70%, at least 80%, at least 90%, at least 95%, or at least 99% identical to SEQ ID NO: 9 or SEQ ID NO: 10. In some embodiments, the antibody or fragment thereof comprises a variable region (e.g., a immunoglobulin heavy-chain-variable or a Vh). For example, the engineered polypeptide can comprise an amino acid that is at least 50%, at least 60%, at least 70%, at least 80%, at least 90%, at least 95%, or at least 99% identical to SEQ ID NO: 11 or SEQ ID NO: 12.

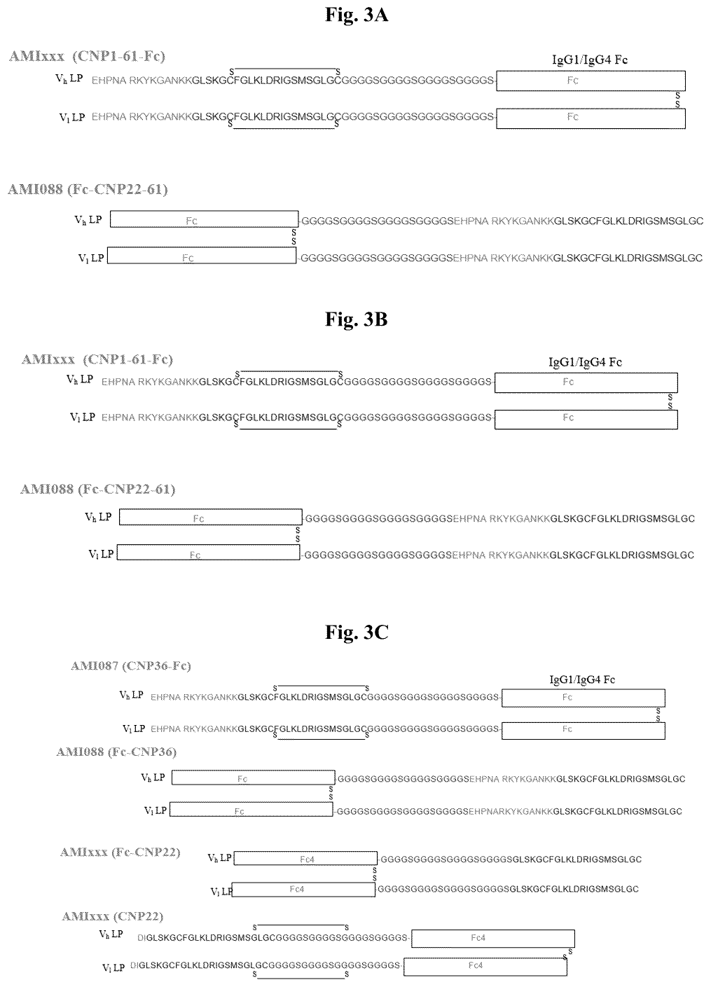

In some embodiments, the peptide is covalently connected to N terminus of the antibody or fragment thereof. In some embodiments, the peptide is covalently connected to C terminus of the antibody or fragment thereof. In some aspects, the operative coupling of the peptide to the antibody or fragment thereof increases the stability of the peptide. In some aspects, the operative coupling of the peptide to the antibody or fragment thereof increases half-life of the peptide. In some aspects, the operative coupling of the peptide to the antibody or fragment thereof increases half-life of the peptide in vivo. In some aspects, the operative coupling of the peptide to the antibody or fragment thereof increases half-life of the peptide in circulation. In some aspects, the operative coupling of the peptide to the antibody or fragment thereof increases half-life of the peptide in a cell. In some embodiments, the antibody or fragment thereof comprises an amino acid sequence that is at least 50%, at least 60%, at least 70%, at least 80%, at least 90%, at least 95%, or at least 99% identical to any one of SEQ ID NOs: 6-8. In some embodiments, the antibody or fragment thereof comprises an amino acid sequence that is 100% identical to any one of SEQ ID NOs: 6-8. In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 131-140. In some embodiments, peptide operatively coupled to the antibody or fragment thereof comprises an amino acid sequence that is 100% identical to any one of SEQ ID NOs: 131-140. In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 9-12. In some embodiments, peptide operatively coupled to the antibody or fragment thereof comprises an amino acid sequence that is 100% identical to any one of SEQ ID NOs: 9-12. FIGS. 3A-C illustrate exemplary depictions of a CNP-Fc fusion protein described herein.

In some embodiments, the engineered polynucleotide described herein comprises a vector. In some embodiments, the vector is a viral vector. In some embodiments, the vector is an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV vector comprises an AAV serotype comprising AAV2. In some embodiments, the AAV vector encodes an engineered AAV capsid. In some embodiments, the viral vector or the AAV vector comprises one or more expression cassettes. In some embodiments, the one or more expression cassettes can encode a CNP or a CNP operatively coupled to an antibody or fragment thereof (e.g., a CNP fusion protein) and at least one additional therapeutic. For example, the at least one additional therapeutic can be a hormone or an agonist for stimulating a natriuretic peptide receptor (NPR) or activating a cyclic GMP (cGMP) signaling pathway. In some embodiments, the at least one additional therapeutic comprises an VEGF inhibitor. For example, the at least one additional therapeutic can be an antibody targeting VEGF or an shRNA targeting VEGF transcript. In some embodiments, the at least one additional therapeutic comprises a cytokine inhibitor (e.g., a tumor necrosis factor inhibitor).

In some embodiments, the engineered polynucleotide described herein is encapsulated or as part of a viral particle. In some embodiments, the viral particle is encoded at least partially from the engineered polynucleotide. In some embodiments, the viral particle is not encoded by the engineered polynucleotide. In some embodiments, the viral particle is an AAV particle. In some embodiments, the viral particle comprises an AAV capsid. In some embodiments, the AAV capsid comprises an modified AAV capsid. In some embodiments, the modified AAV capsid comprises a modified AAV2 capsid. In some embodiments, the viral particle can be administered to a subject for treating a disease or condition. In some embodiments, the viral particle can be formulated into a pharmaceutical composition described herein.

Described herein, in some aspects, is a method for treating a disease or condition in a subject in need thereof, the method comprising administering to the subject a therapeutically effective amount of the engineered polynucleotide described herein, an engineered polypeptide encoded by the engineered polynucleotide described herein, a cell transduced with engineered polynucleotide described herein, or a pharmaceutical composition described herein. In some embodiments, the engineered polynucleotide encodes the engineered polypeptide described herein. In some embodiments, the method cures the disease or condition or significantly decrease the severity associated the disease or condition. In some embodiments, the method confers protection against the disease or condition. In some embodiments, the method cures the disease or condition or significantly decreases the severity associated with the disease to condition after a single administration. In some embodiments, the method cures disease or condition or significantly decreases the severity associated with the disease to condition without the need for daily administration. In some aspects, the disease or condition comprises an ocular disease comprising ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

### Engineered Polynucleotide

Described herein, in some aspects, is an engineered polynucleotide encoding a peptide or a fusion protein. In some embodiments, the engineered polynucleotide encodes a natriuretic peptide such a C-type natriuretic peptide (CNP) or fragment thereof. In some embodiments, the CNP or fragment thereof comprises 22 contiguous bases of SEQ ID NO: 1. In some embodiments, the CNP or fragment thereof comprises the 22 contiguous bases at C terminus of SEQ ID NO: 1. In some embodiments, the CNP or fragment thereof comprises 36 contiguous bases of SEQ ID NO: 1. In some embodiments, the CNP or fragment thereof comprises the 36 contiguous bases at C terminus of SEQ ID NO: 1. In some embodiments, the CNP or fragment thereof comprises 53 contiguous bases of SEQ ID NO: 1. In some embodiments, the CNP or fragment thereof comprises the 53 contiguous bases at C terminus of SEQ ID NO: 1.

In some embodiments, the engineered polynucleotide encodes a CNP or a CNP fusion protein (e.g., a CNP-Fc fusion protein described herein). In some embodiments, the engineered polynucleotide comprises a viral vector such as an AAV vector comprising one or more expression cassettes for encoding a CNP or a CNP fusion protein. In some embodiments, the engineered polynucleotide comprises a vector. In some embodiments, the vector is a viral vector. In some embodiments, the engineered polynucleotide comprises an AAV vector. In some embodiments, the engineered polynucleotide comprises an AAV vector encoding an engineered AAV capsid. In some embodiments, the AAV vector comprises one or more expression cassettes for encoding an engineered polypeptide comprising: a peptide or a fusion protein comprising an antibody or fragment thereof operatively coupled to a peptide. In some embodiments, the one or more expression cassettes encode an engineered polypeptide comprising a CNP. In some embodiments, the one or more expression cassettes encode an engineered polypeptide comprising an antibody or fragment thereof operatively coupled to a CNP. In some embodiments, the CNP comprises at least 22 amino acid residues. In some embodiments, the CNP comprises at least 36 amino acid residues. In some embodiments, the CNP comprises at least 53 amino acid residues. In some embodiments, the CNP comprises an amino acid sequence that is at least 50%, at least 60%, at least 70%, at least 80%, at least 90%, at least 95%, or at least 99% identical to any one of SEQ ID NOs: 1-5. In some embodiments, the CNP comprises an amino acid sequence that is 100% identical to any one of SEQ ID NOs: 1-5. In some embodiments, the engineered polynucleotide encodes a natriuretic peptide comprising an amino acid sequence that is at least 80% identical to SEQ ID NO: 4. In some embodiments, the engineered polynucleotide encodes a natriuretic peptide comprising an amino acid sequence that is at least 95% identical to SEQ ID NO: 4. In some embodiments, the engineered polynucleotide encodes a natriuretic peptide comprising an amino acid sequence of SEQ ID NO: 4.

In some embodiments, the CNP encoded by the engineered polynucleotide is operatively coupled to an antibody or fragment thereof. In some embodiments, the antibody or fragment thereof comprises a fragment crystallizable (Fc) region. In some embodiments, the CNP encoded by the engineered polynucleotide is operatively coupled to N terminus of the antibody or fragment thereof. In some embodiments, the CNP encoded by the engineered polynucleotide is operatively coupled to C terminus of the antibody or fragment thereof. In some embodiments, the CNP encoded by the engineered polynucleotide is covalently connected to an antibody or fragment thereof. In some embodiments, the CNP encoded by the engineered polynucleotide is covalently connected to N terminus of the antibody or fragment thereof. In some embodiments, the CNP encoded by the engineered polynucleotide is covalently connected to C terminus of the antibody or fragment thereof. In some embodiments, the CNP is covalently connected to the antibody or fragment thereof by a peptide linker.

In some embodiments, the antibody or fragment thereof comprises an amino acid sequence that is at least 50%, at least 60%, at least 70%, at least 80%, at least 90%, at least 95%, or at least 99% identical to any one of SEQ ID NOs: 6-8. In some embodiments, the antibody or fragment thereof comprises an amino acid sequence that is 100% identical to any one of SEQ ID NOs: 6-8. In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 131-140. In some embodiments, peptide operatively coupled to the antibody or fragment thereof comprises an amino acid sequence that is 100% identical to any one of SEQ ID NOs: 131-140. In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 9-12. In some embodiments, peptide operatively coupled to the antibody or fragment thereof comprises an amino acid sequence that is 100% identical to any one of SEQ ID NOs: 9-12.

In some embodiments, the engineered polynucleotide is a vector. In some embodiments, the engineered polynucleotide is a viral vector comprising an AAV vector. In some embodiments, the engineered polynucleotide is an AAV vector comprising an AAV serotype comprising AAV1, AAV2, AAV3, AAV4, AAV5, AAV6, AAV7, AAV8, AAV9, AAV10, AAV11, AAV12, or any combination thereof. In some embodiments, the engineered polynucleotide is an AAV vector comprising the AAV2 serotype. In some embodiments, the AAV vector encodes a modified AAV capsid.

In some embodiments, the engineered polynucleotide encodes a contiguous polypeptide, where the contiguous polypeptide comprises an engineered polypeptide described herein. In some embodiments, the contiguous polypeptide comprises a protease cleavable sequence. In some embodiments, the contiguous polypeptide comprises a Furin cleavable sequence. In some embodiments, the contiguous polypeptide comprises a self-cleaving polypeptide sequence.

In some embodiments, the engineered polynucleotide comprises one or more expression cassettes, where the one or more expression cassettes encode a CNP or CNP fusion protein and at least one additional therapeutic. In some embodiments, the at least one additional therapeutic comprises a hormone. In some embodiments, the at least one additional therapeutic comprises an agonist of a natriuretic peptide receptor (NPR). In some embodiments, the at least one additional therapeutic comprises an agonist of a cyclic GMP (cGMP) signaling pathway. In some embodiments, the at least one additional therapeutic comprises a cytokine inhibitor. In some embodiments, the at least one additional therapeutic comprises an VEGF inhibitor, where the VEGF inhibitor binds to and inhibits VEGF-A, VEGF-B, VEGF-C, VEGF-D, or a combination thereof. In some embodiments, the VEGF inhibitor is an antibody. In some embodiments, the VEGF inhibitor is not an antibody.

In some cases, the engineered polynucleotide comprises additional features. Additional features can comprise sequences such as tags, signal peptides, intronic sequences, promoters, stuffer sequences, and the like.

In some cases, the engineered polynucleotide encodes a signal peptide. A signal peptide is sometimes referred to as signal sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signal peptides. A signal peptide can be of any length but typically from 15-30 amino acids long. A signal peptide can be from about: 10-15, 10-20, 10-30, 15-20, 15-25, 15-30, 20-30, or 25-30 amino acids long. Various signal peptides can be utilized and include but are not limited to: human antibody heavy chain (Vh), human antibody light chain (VI), and aflibercept.

In some cases, the engineered polynucleotide comprises an intronic sequence. An intron is any nucleotide sequence within a sequence that can be removed by RNA splicing during maturation of the final RNA product. In other words, introns are non-coding regions of an RNA transcript, or the DNA encoding it, that are eliminated by splicing before translation. While introns do not encode protein products, they are players in gene expression regulation. Some introns themselves encode functional RNAs through further processing after splicing to generate noncoding RNA molecules. Alternative splicing is widely used to generate multiple proteins from a single gene. Furthermore, some introns play essential roles in a wide range of gene expression regulatory functions such as nonsense-mediated decay and mRNA export. In an embodiment, an intronic sequence is included in a nucleic acid of the disclosure and can be selected from: hCMV intron A, adenovirus tripartite leader sequence intron, SV40 intron, hamster EF-1 alpha gene intron 1, intervening sequence intron, human growth hormone intron, and/or human beta globin intron. Any number of intronic sequences are contemplated. In an embodiment, the intronic sequence is SV40. In some cases, at least 1, at least 2, at least 3, at least 4, at least 5, at least 6, at least 7, at least 8, at least 9, or up to 10 intronic sequences can be included in a nucleic acid.

In an embodiment, the engineered polynucleotide comprises an additional feature including a promoter. Promoters are sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. Various promoters are contemplated and can be employed in the engineered polynucleotides of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GAL1 promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

Any of the provided the engineered polynucleotide can comprise viral vector sequences. A viral vector can be, without limitation, a lentivirus, a retrovirus, or an adeno-associated virus. A viral vector can be an adeno-associated viral (AAV) vector. In some cases, a viral vector is an adeno-associated viral vector. Many serotypes of AAV vectors are contemplated and include but are not limited to: AAV1, AAV2, AAV3, AAV4, AAV5, AAV6, AAV7, AAV8, AAV9, AAV10, AAV11, and/or AAV12. Based on these initial serotypes, AAV capsid of each serotype can be engineered to make them better suited for biological functions, tissue or cell selection. In some embodiments, an AAV vector is AAV2 and variants AAV2.N53 and AAV2.N54. Chimeric AAV vectors are also contemplated that may contain at least 2 AAV serotypes. In some cases, at least 3, at least 4, at least 5, at least 6, at least 7, or up to 8 different serotypes are combined in a chimeric AAV vector. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

In some cases, an AAV vector can be modified. For example, an AAV vector can comprise a modification such as an insertion, deletion, chemical alteration, or synthetic modification. In some cases, a single nucleotide is inserted into an AAV vector. In other cases, multiple nucleotides are inserted into a vector.

### Codon Optimization

In an embodiment, the engineered polynucleotide described herein comprises a modification that confers enhanced expression of a biologic such as the CNP or the CNP fusion protein described herein. For example, some biologics are derived from natural gene sequences and contain unmodified sequences that are not optimized for introduction and expression in target cells. In an embodiment, an isolated, engineered polynucleotide is codon optimized. Codon optimization can be specific for cell type-specific codon usage. Different organisms and cell types exhibit bias towards use of certain codons over others for the same amino acid. Some species are known to avoid certain codons almost entirely. Similarly, certain cell types are biased toward use of certain codons over others for the same amino acid. In an embodiment, a method of optimizing a codon of an engineered polynucleotide can comprise reassigning codon usage based on the frequencies of each codon's usage in a target cell. In some cases, a target cell can be of a certain tissue or organ. In some cases, a modification is performed to increase guanine and/or cytosine content.

In an embodiment, an engineered polynucleotide sequence can be modified to replace at least one codon with another codon coding for an identical amino acid. In some cases, a codon is modified within a coding region of a sequence. In some cases, a codon is modified within a non-coding region of a sequence. In some cases, a codon is modified within about 100, about 50, about 25, about 15, or about 5 bases from a termination codon. E-CAI can be utilized to estimate a value of a codon adaptation index.

Various modifications are contemplated herein. In some cases, codons can be interchanged. For example, a sequence can be modified to replace AGA with AGG. In other cases, CCC is replaced with CCT. In other cases, AGC is replaced with TCC. In other cases, CCC is replaced with CCG. Any of the non-limiting replacements provided in Table 1 can be applied to modify a nucleic acid. Any number of codons can be interchanged in a nucleic acid. In some cases, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 34, at least 36, at least 38, at least 40, at least 42, at least 44, at least 46, at least 48, or up to 50 codons can be replaced. In an embodiment, an engineered polynucleotide comprises 3 codon modifications. In an embodiment, an engineered polynucleotide comprises 16 codon modifications. In an embodiment, an engineered polynucleotide comprises 3-5, 5-10, 5-15, 10-15, 10-20, 15-20, 1-20, 12-20, 12-25, 15-30, or 15-25 codon modifications. In an embodiment, an engineered polynucleotide comprises two codon modifications that are: AGA to AGG and at least one of: CCT to CCC, AGC to TCC, or CCC to CCG. In an embodiment, a engineered polynucleotide comprises three codon modifications that are: AGA to AGG and at least two of: CCT to CCC, AGC to TCC, or CCC to CCG. In an embodiment, an engineered polynucleotide comprises four codon modifications that are: AGA to AGG, CCT to CCC, AGC to TCC, and CCC to CCG. Additional modifications can comprise any of the codon modifications provided in Table 1 in combination with any of the above codons and/or any additional modifications possible from Table 1. In an embodiment, a nucleic acid is modified such that AGA is replaced with AGG and CCT is replaced with CCC. In an embodiment, a nucleic acid is modified such that AGA is replaced with AGG and AGC is replaced with TCC. In an embodiment, a nucleic acid is modified such that AGA is replaced with AGG and CCC is replaced with CCG.

TABLE 1
Non-limiting, exemplary codons that can be interchanged for modification of
nucleic acids. Thymine can be replaced with uracil in the below codons

| AA | Codons | AA | Codons |
|---|---|---|---|
| Ala | GCT, GCC, GCA, GCG | Leu | TTA, TTG, CTT, CTC, CTA, CTG |
| Arg | CGT, CGC, CGA, CGG, AGA, AGG | Lys | AAA, AAG |
| Asn | AAT, AAC | Met | ATG |
| Asp | GAT, GAC | Phe | TTT, TTC |
| Cys | TGT, TGC | Pro | CCT, CCC, CCA, CCU |
| Gln | CAA, CAG | Ser | TCT, TCC, TCA, TCG, AGT, AGC |
| Glu | GAA, GAG | Thr | ACT, ACC, ACA, ACG |
| Gly | GGT, GGC, GGA, GGG | Trp | TGG |
| His | CAT, CAC | Tyr | TAT, TAC |
| Ile | ATT, ATC, ATA | Val | GTT, GTC, GTA, GTG |
| Start | ATG | Stop | TAA, TGA, TAG |

In some embodiments, a engineered polynucleotide sequence can comprise a viral vector sequence. In some embodiments, a viral vector sequence can be a scAAV vector sequence. In some embodiments, a AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, a viral vector sequence can comprise sequences of at least 2 AAV serotypes. In some embodiments, at least two serotypes can be selected from AAV1, AAV2, AAV3, AAV4, AAV5, AAV6, AAV8, AAV9, AAV11, and AAV12.

In some cases, a modification can also comprise a chemical modification. Modified nucleic acids can comprise modifications of their backbones, sugars, or nucleobases, and even novel bases or base pairs. Modified nucleic acids can have improved chemical and/or biological stability. Decoration with diverse chemical substituents (e.g., hydrophobic groups) can also yield improved properties and functionalities such as new structural motifs and enhanced target binding.

Exemplary chemical modification includes but are not limited to: 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; PNA, and peptide nucleic acid.

### Dual Expression

Described herein, in some aspects, is an engineered polynucleotide comprising one or more expression cassettes for expressing a peptide (e.g., CNP), a fusion protein (e.g., CNP fusion protein), or a therapeutic. In some embodiments, the one or more expression cassettes encode a contiguous polypeptide. In some embodiments, the contiguous polypeptide comprises a protease peptide sequence. In some embodiments, the protease peptide sequence is cleavable by a protease expressed endogenously in a cell. Non-limiting example of the protease can include a serine endoprotease, an aspartic endoprotease, a cysteine thiol endoprotease, a metalloendoprotease, or a glutamic acid and threonine endoprotease. In some embodiments, the protease peptide sequence is cleavable by a serine endoprotease. In some embodiments, the protease peptide sequence is cleavable by Furin. In some embodiments, the contiguous polypeptide comprises a protease cleavable sequence. In some embodiments, the protease cleavable sequence can be cleaved by any one of the proteases described herein. In some embodiments, the protease cleavable sequence can be cleaved by Furin. In some embodiments, the contiguous polypeptide comprises a self-cleaving polypeptide sequence. In some embodiments, the self-cleaving polypeptide sequence comprises a 2A self-cleaving peptide sequence. Non-limiting examples of the 2A self-cleaving peptide sequence can include T2A, P2A, E2A, F2A, or a combination thereof. In some embodiments, the self-cleaving polypeptide sequence comprises a F2A peptide sequence. In some embodiments, the contiguous polypeptide comprises a protease cleavable sequence and a self-cleaving polypeptide sequence. For example, the contiguous polypeptide described herein can comprise a Furin-F2A fusion polypeptide sequence. In some embodiments, the engineered polynucleotide comprises a viral vector such as an AAV vector.

In some embodiments, the engineered polynucleotide comprises one or more promoters or IRES. In some embodiments, the expression cassette comprises one or more promoters or internal ribosome entry sites (IRES). In some embodiments, the expression cassette is under expression control of a promoter. In some embodiments, the expression cassette is under expression control of a promoter. In some embodiments, expression cassette can further exert expression control via at least one IRES.

In some embodiments, the engineered polynucleotide comprises at least two, at least three, at least four, at least five, or more expression cassettes. In some embodiments, the engineered polynucleotide comprises two expression cassettes. In some embodiments, the CNP or the CNP fusion protein (e.g., a CNP-Fc fusion protein) and at least one additional therapeutic are each expressed from an expression cassette.

In some embodiments, the at least one additional therapeutic is an inhibitor targeting a cytokine (e.g., a tumor necrosis factor). In some embodiments, the at least one additional therapeutic can be an antibody targeting the cytokine or an inhibitory nucleic acid targeting the transcript of the cytokine. For example, the at least one additional therapeutic can be an inhibitory RNA such as short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA) that targets the transcript of the cytokine for degradation, thus decreasing the expression of the cytokine in a cell.

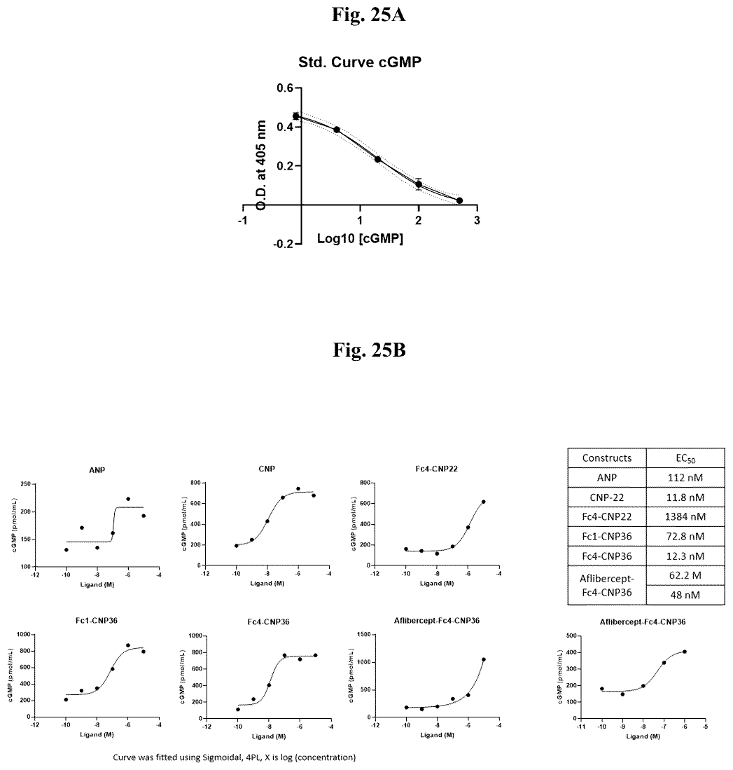

In some embodiments, the at least one additional therapeutic is a VEGF inhibitor. For example, FIG. 25B illustrates functional analysis of dually expressed a CNP fusion (Fc4-CNP36) and an VEGF inhibitor (Aflibercept). In some embodiments, the VEGF inhibitor comprises an inhibitory RNA for targeting and degrading the VEGF transcript. In some embodiments, the VEGF inhibitor comprises an antibody or a fragment thereof. In some embodiments, the VEGF antibody binds to VEGF to decrease neovascularization signaling comprising the VEGF signaling transduction pathway. In some embodiments, the VEGF antibody binds to VEGF-A, VEGF-B, VEGF-C, VEGF-D, or a combination thereof. In some embodiments, the VEGF antibody binds to one or more isoforms of VEGF-A, including VEGF121, VEGF145, VEGF148, VEGF162, VEGF165, VEGF165b, VEGF183, VEGF189, or VEGF206. In some embodiments, the antibody comprises monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. Non-limiting examples of VEGF antibodies include ranibizumab or bevacizumab. In some embodiments, the VEGF antibody comprises a polypeptide sequence that is at least 700%, at least 7500, at least 8000, at least 85%, at least 9000, at least 95%, at least 99%, or more identical to any one of SEQ ID NOs: 21-27 (Table 2), or a combination thereof, or a fragment thereof.

TABLE 2
Non-limiting examples of polypeptide sequences of VEGF antibodies

| SEQ ID | |
|---|---|
| NO | Polypeptide sequence of VEGF antibody |
| SEQ ID | EVQLVESGGGLVQPGGSLRLSCAASGYDFTHYGMNWVRQAPGKGLEWVG |
| NO: 21 | WINTYTGEPTYAADFKRRFTFSLDTSKSTAYLQMNSLRAEDTAVYYCAKYP |
| | YYYGTSHWYFDVWGQGTLVTVSS |
| SEQ ID | DIQLTQSPSSLSASVGDRVTITCSASQDISNYLNWYQQKPGKAPKVLIYFTSS |
| NO: 22 | LHSGVPSRFSGSGSGTDFTLTISSLQPEDFATYYCQQYSTVPWTFGQGTKVEI |
| | KRTVAA |
| SEQ ID | EVQLVESGGGLVQPGGSLRLSCAASGYDFTHYGMNWVRQAPGKGLEWVG |
| NO: 23 | WINTYTGEPTYAADFKRRFTFSLDTSKSTAYLQMNSLRAEDTAVYYCAKYP |
| | YYYGTSHWYFDVWGQGTLVTVSSGGGGSGGGGSGGGGSGGGGSDIQLTQ |
| | SPSSLSASVGDRVTITCSASQDISNYLNWYQQKPGKAPKVLIYFTSSLHSGVP |
| | SRFSGSGSGTDFTLTISSLQPEDFATYYCQQYSTVPWTFGQGTKVEIKRTVA |
| | A |
| SEQ ID | EVQLVESGGGLVQPGGSLRLSCAASGYDFTHYGMNWVRQAPGKGLEWVG |
| NO: 24 | WINTYTGEPTYAADFKRRFTFSLDTSKSTAYLQMNSLRAEDTAVYYCAKYP |
| | YYYGTSHWYFDVWGQGTLVTVSSASTKGPSVFPLAPSSKSTSGGTAALGCL |
| | VKDYFPEPVTVSWNSGALTSGVHTFPAVLQSSGLYSLSSVVTVPSSSLGTQT |
| | YICNVNHKPSNTKVDKKVEPKSCDKTHL |
| SEQ ID | DIQLTQSPSSLSASVGDRVTITCSASQDISNYLNWYQQKPGKAPKVLIYFTSS |
| NO: 25 | LHSGVPSRFSGSGSGTDFTLTISSLQPEDFATYYCQQYSTVPWTFGQGTKVEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC |
| SEQ ID | DIQLTQSPSSLSASVGDRVTITCSASQDISNYLNWYQQKPGKAPKVLIYFTSS |
| NO: 26 | LHSGVPSRFSGSGSGTDFTLTISSLQPEDFATYYCQQYSTVPWTFGQGTKVEI |
| | KRTVAAGGGGSGGGGSGGGGSGGGGSEVQLVESGGGLVQPGGSLRLSCA |
| | ASGYDFTHYGMNWVRQAPGKGLEWVGWINTYTGEPTYAADFKRRFTFSL |
| | DTSKSTAYLQMNSLRAEDTAVYYCAKYPYYYGTSHWYFDVWGQGTLVTV |
| | SS |
| SEQ ID | MEIVMTQSPSTLSASVGDRVIITCQASEIIHSWLAWYQQKPGKAPKLLIYLA |
| NO: 27 | STLASGVPSRFSGSGSGAEFTLTISSLQPDDFATYYCQNVYLASTNGANFGQ |
| | GTKLTVLGGGGGSGGGGSGGGGSGGGGSEVQLVESGGGLVQPGGSLRLSC |
| | TASGFSLTDYYYMTWV RQAPGKGLEWVGFIDPDDDP YYATWAKGRF |
| | TISRDNSKNT LYLQMNSLRA EDTAVYYCAG |
| | GDHNSGWGLDIWGQGTLVTVSS |

In some embodiments, the VEGF inhibitor is not an antibody. For example, the VEGF inhibitor described herein can comprise a VEGF receptor, a combination of VEGF receptors, or a fragment thereof for binding to VEGF for inhibiting or decreasing VEGF signaling transduction pathway. VEGF receptor can include a VEGF receptor 1 (FLT1), a VEGF receptor 2 (KDR/FLK1), a VEGF receptor 3 (FLT4), a fragment thereof, or a combination thereof. In some embodiments, the VEGF receptor can be a soluble VEGF receptor. For example, the soluble VEGF receptor can comprise a soluble VEGFR1, a soluble VEGFR2, a soluble VEGFR3, a soluble fragment thereof, or a combination thereof. In some embodiments, the non-antibody VEGF inhibitor comprises at least one of FLT 1, KDR/FLK 1, FLT4, a fragment thereof, or a combination thereof. In some embodiments, the non-antibody VEGF inhibitor comprises at least one of soluble FLT1, soluble KDR/FLK1, soluble FLT4, a fragment thereof, or a combination thereof. In some embodiments, the non-antibody inhibitor VEGF comprises a VEGF-Trap. In some embodiments, the non-antibody VEGF inhibitor comprises a polypeptide sequence that is at least 70%, at least 7500, at least 8000, is at least 85%, at least 90%, at least 95%, at least 99%, or more identical to any one of SEQ ID NOs: 31-34 (Table 3).

TABLE 3
Non-limiting examples of non-antibody VEGF inhibitors

| SEQ ID | |
|---|---|
| NO | Non-antibody VEGF inhibitor |
| SEQ ID | LPAQVAFTPYAPEPGSTCRLREYYDQTAQMCCSKCSPGQHAKVFCTKTSD |
| NO: 31 | TVCDSCEDSTYTQLWNWVPECLSCGSRCSSDQVETQACTREQNRICTCRP |
| | GWYCALSKQEGCRLCAPLRKCRPGFGVARPGTETSDVVCKPCAPGTFSNT |
| | TSSTDICRPHQICNVVAIPGNASMDAVCTSTSPTRSMAPGAVHLPQPVSTRS |
| | QHTQPTPEPSTAPSTSFLLPMGPSPPAEGSTGDEPKSCDKTHTCPPCPAPELL |
| | GGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKT |
| | ISKAKGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALHNHY |
| | TQKSLSLSPGK |
| SEQ ID | DTGRPFVEMYSEIPEIIHMTEGRELVIPCRVTSPNITVTLKKFPLDTLIPDGK |
| NO: 32 | RIIWDSRKGFIISNATYKEIGLLTCEATVNGHLYKTNYLTHRQTNTIIDVVLS |
| | PSHGIELSVGEKLVLNCTARTELNVGIDENWEYPSSKHQHKKLVNRDLKTQ |
| | SGSEMKKFLSTLTIDGVTRSDQGLYTCAASSGLMTKKNSTFVRVHEK |
| SEQ ID | DTGRPFVEMYSEIPEIIHMTEGRELVIPCRVTSPNITVTLKKFPLDTLIPDGK |
| NO: 33 | RIIWDSRKGFIISNATYKEIGLLTCEATVNGHLYKTNYLTHRQTNTIIDVVLS |
| | PSHGIELSVGEKLVLNCTARTELNVGIDFNWEYPSSKHQHKKLVNRDLKTQ |
| | SGSEMKKFLSTLTIDGVTRSDQGLYTCAASSGLMTKKNSTFVRVHEKDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSC |
| | SVMHEALHNHYTQKSLSLSPGK |
| SEQ ID | SDTGRPFVEMYSEIPEIIHMTEGRELVIPCRVTSPNITVTLKKFPLDTLIPDGK |
| NO: 34 | RIIWDSRKGFIISNATYKEIGLLTCEATVNGHLYKTNYLTHRQTNTIIDVVLS |
| | PSHGIELSVGEKLVLNCTARTELNVGIDENWEYPSSKHQHKKLVNRDLKTQ |
| | SGSEMKKFLSTLTIDGVTRSDQGLYTCAASSGLMTKKNSTFVRVHEKDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSC |
| | SVMHEALHNHYTQKSLSLSPG |

### Modified Capsid

Provided herein are modified adeno-associated virus (AAV) capsid-containing compositions and methods of using the same. A modified AAV capsid can comprise exogenous sequences as compared to an otherwise comparable unmodified AAV capsid. Exogenous sequences can refer to exogenous polypeptide sequences. AAV capsids can be modified to confer upon them, and any compositions and/or methods in which they are utilized, improved functionality thereby resulting in better therapeutics, particularly for ocular use.

The AAV wild-type (WT) genome contains at least three genes: rep, cap, and X. The X gene is located at the 3′ end of the genome (nucleotides 3929-4393 in AAV2) and seems to code for a protein with supportive function in genome replication. Significantly more information is available for rep and cap. The rep gene is located in the first half of the AAV WT genome and codes for a family of non-structural proteins (Rep proteins) required for viral transcription control and replication as well as packaging of viral genomes into the newly produced, pre-assembled capsids. The second half of the AAV genome contains the cap gene, which codes for the viral proteins (VPs) VP1, VP2, and VP3, and the assembly-activating protein (AAP). Transcription of all VPs, which are the capsid monomers, is controlled by a single promoter (p40 in case of AAV2) resulting in a single mRNA. Splicing (VP1) and an unusual translational start codon (VP2) are responsible for an approximately 10 times lower presence of VP1 and VP2 compared with VP3. When encoded by a single gene, AAV VPs share most of their amino acids. Specifically, the entire VP3 sequence is also contained within VP2 and VP1 (“common VP3 region”), and also VP2 and VP1 share approximately 65 amino acids (“common VP1/VP2 region”). Only VP1 contains a unique sequence at its N terminus (approximately 138 amino acids, VP1 unique). AAP was identified in 2010 as a 23 kDa protein encoded in an alternative cap ORF. It is used for stabilizing and transporting newly produced VP proteins from the cytoplasm into the cell nucleus. While AAV serotypes 1-3, 6-9, and rh10 failed to produce capsids in the absence of AAP, a low but detectable capsid production was reported for AAV4 and AAV5.

In an aspect, an AAV can comprise a modification. A modification can be of a rep, cap, and/or X coding polypeptide sequence of an AAV. In some cases, the modification can be of a cap polypeptide. A cap polypeptide can be modified in any one of the VP domains, for example VP1, VP2, and/or VP3. In some cases, VP1 is modified. In some cases, VP2 is modified. In some cases, VP3 is modified. In some aspects, two or all of the VP domains can be modified. In some cases, VP1 and VP2 are modified. In some cases, VP1 and VP3 are modified. Additionally, VP2 and VP3 can be modified or VP1, VP2, and VP3 are modified. Other combinations are contemplated, such as modifications in Rep and Cap, Cap and X, Rep and X, and/or Rep, Cap, and X. Any combination of domains can be modified such as any one of the aforementioned VP modifications in conjunction with a Rep and/or X modification. In some cases, Rep and VP1 and/or VP2 are modified. In some aspects, a subject Rep is modified. A rep modification can comprise a modification as provided herein and can be in at least one of Rep 78, Rep 68, Rep 52 or Rep 40. In some cases, a Rep is of a different AAV serotype than a subject capsid.

In some cases, a modification is of an AAV capsid. Capsids of AAV serotypes are assembled from 60 VP monomers with approximately 50 copies of VP3, 5 copies of VP2, and 5 copies of VP1. Topological prominent capsid surface structures are pores or “channel-like-structures” at each fivefold, depressions at each twofold, and three protrusions surrounding each threefold axis of symmetry. The pores allow exchange between the capsid interior and the outside. The depressions, more precisely the floor at each twofold axis, are the thinnest part of the viral capsid. The protrusions around the threefold axis harbor five of the nine so-called variable regions (VRs). Specifically, VR-IV, -V, and -VIII form loops (loop 1-4) at the top of the protrusions, while VR-VI and -VII are found at their base. VRs differ between serotypes and are responsible for serotype-specific variations in antibody and receptor binding. Because of their exposed positions and their function in receptor binding, VRs forming loops of the protrusions are ideal positions for capsid modifications aiming to re-direct or expand AAV tropism (cell surface targeting). While a re-directed tropism (vector re-targeting) combines ablation of natural receptor binding, for example by site-directed mutagenesis, with insertion of a ligand that mediates transduction through a novel non-natural AAV receptor, AAV vectors with tropism expansion gain the ability to transduce cells through an extra receptor while maintaining their natural receptor binding abilities.

In some aspects, a modification of an AAV capsid, can refer to an insertion of an exogenous polypeptide sequence. In other aspects, a modification can refer to a deletion in a polypeptide sequence. A modification can also refer to a modification of at least one amino acid residue, canonical or non-canonical, in a polypeptide sequence.

An insertion can comprise inserting at least 1 exogenous amino acid residue into a sequence coding an AAV capsid. The amino acid can refer to a canonical amino acid or a non-canonical amino acid. Any number of amino acid residues can be inserted. In some cases, an insertion site can be in the GH loop, or loop IV, of the AAV capsid protein, e.g., in a solvent-accessible portion of the GH loop, or loop IV, of the AAV capsid protein.

In some cases, a modification comprises insertion of an exogenous polypeptide sequence that comprises a sequence of Formula 1: X0-X1-X2-X1-X3-X1-X1-X4. In some cases, X0 is Valine (V), Isoleucine (I), Leucine (L), Phenylalanine (F), Tryptophan (W), Tyrosine (Y) or Methionine (M). In some cases, X1 is Alanine (A), Asparagine (N), Glutamine (Q), Serine (S), Threonine (T), Glutamic Acid (E), Aspartic Acid (D), Lysine (K), Arginine (R), or Histidine (H). In some cases, X2 is V, I, L, or M, where X3 is E, S, or Q. In some cases, X4 is K, R, E, or A. In some cases, Formula 1 further comprises X5. X5 can be Proline (P) or R.

In some cases, Formula 1 comprises: L-A-L-G-X3-X1-X1-X4 (SEQ ID NO: 42), L-K-L-G-X3-X1-X1-X4 (SEQ ID NO: 43), or V-K-L-G-X3-X1-X1-X4 (SEQ ID NO: 44). In some cases, Formula 1 comprises V-K-L-G-X3-X1-X1-X4 (SEQ ID NO: 45). In some cases, an exogenous polypeptide is V-K-L-G-X3-X1-T-X4 (SEQ ID NO: 46) and/or V-K-L-G-X3-X1-X1-K (SEQ ID NO: 47). In some cases, an exogenous polypeptide comprises L-A-L-G-X3-X1-X1-X4 (SEQ ID NO: 48). In some cases, an exogenous polypeptide comprises L-A-L-G-X3-X1-T-X4 (SEQ ID NO: 49) and/or L-A-L-G-X3-X1-S-X4 (SEQ ID NO: 50). In some cases, an exogenous polypeptide comprises: L-A-L-G-X3-X1-T-R (SEQ ID NO: 51), L-A-L-G-X3-X1-T-K (SEQ ID NO: 52), L-A-L-G-X3-X1-T-E (SEQ ID NO: 53), and/or L-A-L-G-X3-X1-T-A (SEQ ID NO: 54). In some cases, an exogenous polypeptide comprises L-A-L-G-X3-X1-S-K (SEQ ID NO: 56). In some cases, an exogenous polypeptide comprises L-K-L-G-X3-X1-X1-X4 (SEQ ID NO: 57). In some cases, an exogenous polypeptide comprises: L-K-L-G-X3-X1-T-X4 (SEQ ID NO: 58). In some cases, an exogenous polypeptide comprises: L-K-L-G-X3-X1-T-K (SEQ ID NO: 59).

In some cases, an exogenous polypeptide comprises a sequence of Formula 1. In some cases, a sequence of Formula I comprises a polypeptide sequence having at least 60%, 62%, 64%, 66%, 68%, 70%, 72%, 74%, 76%, 78%, 80%, 82%, 84%, 86%, 88%, 90%, 92%, 94%, 96%, 98%, or up to about 100% identity with a sequence of Table 4. In some cases, an exogenous polypeptide is one of Table 4 with 0-2 modifications to a residue.

In some cases, at least 2 of the exogenous polypeptides, such as those described by Formula 1, are inserted into a capsid sequence of an AAV provided herein. The at least 2 exogenous polypeptides can be inserted into the same location or at different locations. In an aspect, any one of the exogenous polypeptide sequences provided in Table 4 can be inserted into an unmodified AAV capsid sequence, such as those wildtype sequences provided in Table 5, to generate a modified AAV capsid.

TABLE 4
Exemplary exogenous polypeptide sequences that can be inserted into AAV
capsids (exemplary insertion sites are shown for AAV2 but comparable
locations of other AAV serotypes are also contemplated)

| SEQ ID | | Modified Capsid Region | Exogenous |
|---|---|---|---|
| NO | Clone No. | of AAV2 | Polypeptide Sequence |
| 61 | V466 | 453 | LALGETTRPA |
| 62 | V467 | 587 | LALGETTKPA |
| 63 | V468 | 452 | ALGETTKP |
| 64 | V471 | 452 | ALGETTKP |
| 65 | V471 | 587 | LALGETTKPA |
| 66 | AMI051 | 587 | LKLGQTTKPK |
| 67 | AMI052 | 587 | LALGQTTKPK |
| 68 | AMI053 | 587 | LKLGQTTKPA |
| 69 | AMI054 | 587 | LALGQTTKPA |
| 70 | AMI097 | 453 | LALGQTTKPA |
| 71 | AMI098 | 587 | LALGQTTEPA |
| 72 | AMI099 | 588 | LALGQTTKPA |
| 73 | AMI100 | 585 | LALGQTTKPA |
| 74 | AMI101 | 587 | VALGQTTKPA |
| 75 | AMI102 | 586 | LALGESTARG |
| 76 | AMI103 | 586 | LALGETSKRA |
| 77 | AMI104 | 586 | LALGQSTKPA |
| 78 | AMI105 | 452 | LALGQTTKPA |
| 79 | AMI106 | 453 | LALGQTTKPA |
| 80 | AMI106 | 587 | LALGQTTKPA |
| 81 | AMI107 | 587 | LALGQTTKPALALGQTTKPA |
| 82 | AMI110 | 587 | VKLGQTTKPA |

| SEQ ID | | |
|---|---|---|
| NO | Clone | Modified AAV2 Capsid Sequence |
| 91 | V466 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGETTRPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 92 | V467 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGETTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 93 | V468 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNRQAA |
| | | TADVNTQGVLPGMVWQDRDVYLQGPIWAKIPHTDGHFHP |
| | | SPLMGGFGLKHPPPQILIKNTPVPANPSTTFSAAKFASFITQY |
| | | STGQVSVEIEWELQKENSKRWNPEIQYTSNYNKSVNVDFT |
| | | VDTNGVYSEPRPIGTRYLTRNL |
| 94 | V471ª | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSALGETTKPGTTTQSRLQFSQAGASDIR |
| | | DQSRNWLPGPCYRQQRVSKTSADNNNSEYSWTGATKYHL |
| | | NGRDSLVNPGPAMASHKDDEEKFFPQSGVLIFGKQGSEKT |
| | | NVDIEKVMITDEEEIRTTNPVATEQYGSVSTNLQRGNLALG |
| | | ETTKPARQAATADVNTQGVLPGMVWQDRDVYLQGPIWA |
| | | KIPHTDGHFHPSPLMGGFGLKHPPPQILIKNTPVPANPSTTFS |
| | | AAKFASFITQYSTGQVSVEIEWELQKENSKRWNPEIQYTSN |
| | | YNKSVNVDFTVDTNGVYSEPRPIGTRYLTRNL |
| 95 | AMI051 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 96 | AMI052 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPKR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 97 | AMI053 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLKLGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 98 | AMI054 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 99 | AMI097 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 100 | AMI098 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTEPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 101 | AMI099 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNRLALGQTTKPA |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 102 | AMI100 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRLALGQTTKPAGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 103 | AMI101 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVALGQTTKPAR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 104 | AMI102 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGESTARGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 105 | AMI103 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGETSKRANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 106 | AMI104 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGLALGQSTKPANR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 107 | AMI105 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSLALGQTTKPAGTTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNR |
| | | QAATADVNTQGVLPGMVWQDRDVYLQGPIWAKIPHTDG |
| | | HFHPSPLMGGFGLKHPPPQILIKNTPVPANPSTTFSAAKFAS |
| | | FITQYSTGQVSVEIEWELQKENSKRWNPEIQYTSNYNKSVN |
| | | VDFTVDTNGVYSEPRPIGTRYLTRNL |
| 108 | AMI106 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGLALGQTTKPATTTQSRLQFSQAGAS |
| | | DIRDQSRNWLPGPCYRQQRVSKTSADNNNSEYSWTGATK |
| | | YHLNGRDSLVNPGPAMASHKDDEEKFFPQSGVLIFGKQGS |
| | | EKTNVDIEKVMITDEEEIRTTNPVATEQYGSVSTNLQRGNL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 109 | AMI107 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNLALGQTTKPAL |
| | | ALGQTTKPARQAATADVNTQGVLPGMVWQDRDVYLQGPI |
| | | WAKIPHTDGHFHPSPLMGGFGLKHPPPQILIKNTPVPANPST |
| | | TFSAAKFASFITQYSTGQVSVEIEWELQKENSKRWNPEIQY |
| | | TSNYNKSVNVDFTVDTNGVYSEPRPIGTRYLTRNL |
| 110 | AMI110 | MAADGYLPDWLEDTLSEGIRQWWKLKPGPPPPKPAERHK |
| | | DDSRGLVLPGYKYLGPFNGLDKGEPVNEADAAALEHDKA |
| | | YDRQLDSGDNPYLKYNHADAEFQERLKEDTSFGGNLGRA |
| | | VFQAKKRVLEPLGLVEEPVKTAPGKKRPVEHSPVEPDSSSG |
| | | TGKAGQQPARKRLNFGQTGDADSVPDPQPLGQPPAAPSGL |
| | | GTNTMATGSGAPMADNNEGADGVGNSSGNWHCDSTWM |
| | | GDRVITTSTRTWALPTYNNHLYKQISSQSGASNDNHYFGY |
| | | STPWGYFDFNRFHCHFSPRDWQRLINNNWGFRPKRLNFKL |
| | | FNIQVKEVTQNDGTTTIANNLTSTVQVFTDSEYQLPYVLGS |
| | | AHQGCLPPFPADVFMVPQYGYLTLNNGSQAVGRSSFYCLE |
| | | YFPSQMLRTGNNFTFSYTFEDVPFHSSYAHSQSLDRLMNPL |
| | | IDQYLYYLSRTNTPSGTTTQSRLQFSQAGASDIRDQSRNWL |
| | | PGPCYRQQRVSKTSADNNNSEYSWTGATKYHLNGRDSLV |
| | | NPGPAMASHKDDEEKFFPQSGVLIFGKQGSEKTNVDIEKV |
| | | MITDEEEIRTTNPVATEQYGSVSTNLQRGNVAKGQTTKPA |
| | | RQAATADVNTQGVLPGMVWQDRDVYLQGPIWAKIPHTD |
| | | GHFHPSPLMGGFGLKHPPPQILIKNTPVPANPSTTFSAAKFA |
| | | SFITQYSTGQVSVEIEWELQKENSKRWNPEIQYTSNYNKSV |
| | | NVDFTVDTNGVYSEPRPIGTRYLTRNL |

TABLE 5
Exemplary wild type AAV capsid polypeptide sequences

| SEQ | AAV | |
|---|---|---|
| ID NO | Serotype | WT Polypeptide Sequence |
| 121 | AAV2 | MAADGYLPDWLEDTLSEGIRQWWKLKPGPPPPKPAERHKDDSR |
| | | GLVLPGYKYLGPFNGLDKGEPVNEADAAALEHDKAYDRQLDS |
| | | GDNPYLKYNHADAEFQERLKEDTSFGGNLGRAVFQAKKRVLEP |
| | | LGLVEEPVKTAPGKKRPVEHSPVEPDSSSGTGKAGQQPARKRLN |
| | | FGQTGDADSVPDPQPLGQPPAAPSGLGTNTMATGSGAPMADNN |
| | | EGADGVGNSSGNWHCDSTWMGDRVITTSTRTWALPTYNNHLY |
| | | KQISSQSGASNDNHYFGYSTPWGYFDFNRFHCHFSPRDWQRLIN |
| | | NNWGFRPKRLNFKLFNIQVKEVTQNDGTTTIANNLTSTVQVFTD |
| | | SEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRL |
| | | MNPLIDQYLYYLSRTNTPSGTTTQSRLQFSQAGASDIRDQSRNW |
| | | LPGPCYRQQRVSKTSADNNNSEYSWTGATKYHLNGRDSLVNPG |
| | | PAMASHKDDEEKFFPQSGVLIFGKQGSEKTNVDIEKVMITDEEEI |
| | | RTTNPVATEQYGSVSTNLQRGNRQAATADVNTQGVLPGMVWQ |
| | | DRDVYLQGPIWAKIPHTDGHFHPSPLMGGFGLKHPPPQILIKNTP |
| | | VPANPSTTFSAAKFASFITQYSTGQVSVEIEWELQKENSKRWNPE |
| | | IQYTSNYNKSVNVDFTVDTNGVYSEPRPIGTRYLTRNL |
| 122 | AAV4 | MTDGYLPDWLEDNLSEGVREWWALQPGAPKPKANQQHQDNA |
| | | RGLVLPGYKYLGPGNGLDKGEPVNAADAAALEHDKAYDQQLK |
| | | AGDNPYLKYNHADAEFQQRLQGDTSFGGNLGRAVFQAKKRVL |
| | | EPLGLVEQAGETAPGKKRPLIESPQQPDSSTGIGKKGKQPAKKK |
| | | LVFEDETGAGDGPPEGSTSGAMSDDSEMRAAAGGAAVEGGQG |
| | | ADGVGNASGDWHCDSTWSEGHVTTTSTRTWVLPTYNNHLYKR |
| | | LGESLQSNTYNGFSTPWGYFDFNRFHCHFSPRDWQRLINNNWG |
| | | MRPKAMRVKIFNIQVKEVTTSNGETTVANNLTSTVQIFADSSYE |
| | | LPYVMDAGQEGSLPPFPNDVFMVPQYGYCGLVTGNTSQQQTDR |
| | | NAFYCLEYFPSQMLRTGNNFEITYSFEKVPFHSMYAHSQSLDRL |
| | | MNPLIDQYLWGLQSTTTGTTLNAGTATTNFTKLRPTNFSNFKKN |
| | | WLPGPSIKQQGFSKTANQNYKIPATGSDSLIKYETHSTLDGRWS |
| | | ALTPGPPMATAGPADSKFSNSQLIFAGPKQNGNTATVPGTLIFTS |
| | | EEELAATNATDTDMWGNLPGGDQSNSNLPTVDRLTALGAVPG |
| | | MVWQNRDIYYQGPIWAKIPHTDGHFHPSPLIGGFGLKHPPPQIFI |
| | | KNTPVPANPATTFSSTPVNSFITQYSTGQVSVQIDWEIQKERSKR |
| | | WNPEVQFTSNYGQQNSLLWAPDAAGKYTEPRAIGTRYLTHHL |
| 123 | AAV5 | MSFVDHPPDWLEEVGEGLREFLGLEAGPPKPKPNQQHQDQARG |
| | | LVLPGYNYLGPGNGLDRGEPVNRADEVAREHDISYNEQLEAGD |
| | | NPYLKYNHADAEFQEKLADDTSFGGNLGKAVFQAKKRVLEPFG |
| | | LVEEGAKTAPTGKRIDDHFPKRKKARTEEDSKPSTSSDAEAGPS |
| | | GSQQLQIPAQPASSLGADTMSAGGGGPLGDNNQGADGVGNAS |
| | | GDWHCDSTWMGDRVVTKSTRTWVLPSYNNHQYREIKSGSVDG |
| | | SNANAYFGYSTPWGYFDFNRFHSHWSPRDWQRLINNYWGFRP |
| | | RSLRVKIFNIQVKEVTVQDSTTTIANNLTSTVQVFTDDDYQLPYV |
| | | VGNGTEGCLPAFPPQVFTLPQYGYATLNRDNTENPTERSSFFCLE |
| | | YFPSKMLRTGNNFEFTYNFEEVPFHSSFAPSQNLFKLANPLVDQ |
| | | YLYRFVSTNNTGGVQFNKNLAGRYANTYKNWFPGPMGRTQG |
| | | WNLGSGVNRASVSAFATTNRMELEGASYQVPPQPNGMTNNLQ |
| | | GSNTYALENTMIFNSQPANPGTTATYLEGNMLITSESETQPVNR |
| | | VAYNVGGQMATNNQSSTTAPATGTYNLQEIVPGSVWMERDVY |
| | | LQGPIWAKIPETGAHFHPSPAMGGFGLKHPPPMMLIKNTPVPGNI |
| | | TSFSDVPVSSFITQYSTGQVTVEMEWELKKENSKRWNPEIQYTN |
| | | NYNDPQFVDFAPDSTGEYRTTRPIGTRYLTRPL |
| 124 | AAV6 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPFGLVEEGAKTAPGKKRPVEQSPQEPDSSSGIGKTGQQPAKK |
| | | RLNFGQTGDSESVPDPQPLGEPPATPAAVGPTTMASGGGAPMA |
| | | DNNEGADGVGNASGNWHCDSTWLGDRVITTSTRTWALPTYNN |
| | | HLYKQISSASTGASNDNHYFGYSTPWGYFDFNRFHCHFSPRDW |
| | | QRLINNNWGFRPKRLNFKLFNIQVKEVTTNDGVTTIANNLTSTV |
| | | QVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQ |
| | | SLDRLMNPLIDQYLYYLNRTQNQSGSAQNKDLLFSRGSPAGMS |
| | | VQPKNWLPGPCYRQQRVSKTKTDNNNSNFTWTGASKYNLNGR |
| | | ESIINPGTAMASHKDDKDKFFPMSGVMIFGKESAGASNTALDNV |
| | | MITDEEEIKATNPVATERFGTVAVNLQSSSTDPATGDVHVMGAL |
| | | PGMVWQDRDVYLQGPIWAKIPHTDGHFHPSPLMGGFGLKHPPP |
| | | QILIKNTPVPANPPAEFSATKFASFITQYSTGQVSVEIEWELQKEN |
| | | SKRWNPEVQYTSNYAKSANVDFTVDNNGLYTEPRPIGTRYLTR |
| | | PL |
| 125 | AAV11 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPLGLVEEGAKTAPGKKRPLESPQEPDSSSGIGKKGKQPARKR |
| | | LNFEEDTGAGDGPPEGSDTSAMSSDIEMRAAPGGNAVDAGQGS |
| | | DGVGNASGDWHCDSTWSEGKVTTTSTRTWVLPTYNNHLYLRL |
| | | GTTSSSNTYNGFSTPWGYFDFNRFHCHFSPRDWQRLINNNWGL |
| | | RPKAMRVKIFNIQVKEVTTSNGETTVANNLTSTVQIFADSSYELP |
| | | YVMDAGQEGSLPPFPNDVFMVPQYGYCGIVTGENQNQTDRNAF |
| | | YCLEYFPSQMLRTGNNFEMAYNFEKVPFHSMYAHSQSLDRLMN |
| | | PLLDQYLWHLQSTTSGETLNQGNAATTFGKIRSGDFAFYRKNW |
| | | LPGPCVKQQRFSKTASQNYKIPASGGNALLKYDTHYTLNNRWS |
| | | NIAPGPPMATAGPSDGDFSNAQLIFPGPSVTGNTTTSANNLLFTS |
| | | EEEIAATNPRDTDMFGQIADNNQNATTAPITGNVTAMGVLPGM |
| | | VWQNRDIYYQGPIWAKIPHADGHFHPSPLIGGFGLKHPPPQIFIK |
| | | NTPVPANPATTFTAARVDSFITQYSTGQVAVQIEWEIEKERSKR |
| | | WNPEVQFTSNYGNQSSMLWAPDTTGKYTEPRVIGSRYLTNHL |
| 126 | AAV12 | MAADGYLPDWLEDNLSEGIREWWALKPGAPQPKANQQHQDN |
| | | GRGLVLPGYKYLGPFNGLDKGEPVNEADAAALEHDKAYDKQL |
| | | EQGDNPYLKYNHADAEFQQRLATDTSFGGNLGRAVFQAKKRIL |
| | | EPLGLVEEGVKTAPGKKRPLEKTPNRPTNPDSGKAPAKKKQKD |
| | | GEPADSARRTLDFEDSGAGDGPPEGSSSGEMSHDAEMRAAPGG |
| | | NAVEAGQGADGVGNASGDWHCDSTWSEGRVTTTSTRTWVLPT |
| | | YNNHLYLRIGTTANSNTYNGFSTPWGYFDFNRFHCHFSPRDWQ |
| | | RLINNNWGLRPKSMRVKIFNIQVKEVTTSNGETTVANNLTSTVQ |
| | | IFADSTYELPYVMDAGQEGSFPPFPNDVFMVPQYGYCGVVTGK |
| | | NQNQTDRNAFYCLEYFPSQMLRTGNNFEVSYQFEKVPFHSMYA |
| | | HSQSLDRMMNPLLDQYLWHLQSTTTGNSLNQGTATTTYGKITT |
| | | GDFAYYRKNWLPGACIKQQKFSKNANQNYKIPASGGDALLKY |
| | | DTHTTLNGRWSNMAPGPPMATAGAGDSDFSNSQLIFAGPNPSG |
| | | NTTTSSNNLLFTSEEEIATTNPRDTDMFGQIADNNQNATTAPHIA |
| | | NLDAMGIVPGMVWQNRDIYYQGPIWAKVPHTDGHFHPSPLMG |
| | | GFGLKHPPPQIFIKNTPVPANPNTTFSAARINSFLTQYSTGQVAV |
| | | QIDWEIQKEHSKRWNPEVQFTSNYGTQNSMLWAPDNAGNYHE |
| | | LRAIGSRFLTHHL |

Similarly, a deletion can comprise deleting at least 1 amino acid residue in a sequence that codes for an AAV capsid. Any number of amino acids can be deleted. In some cases, at least, or at most: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or up to about 50 exogenous amino acid residues can be inserted and/or deleted in a polypeptide sequence that codes for an AAV capsid. In some cases, at least or at most: 1-5, 5-10, 10-15, 15-20, or combinations thereof of exogenous amino acid residues can be inserted and/or deleted in a polypeptide sequence that codes for an AAV capsid. In some cases, from about or up to about: 5 amino acids to about 11 amino acids are inserted in an insertion site in the GH loop or loop IV of the capsid protein relative to a corresponding unmodified AAV capsid protein. For example, the insertion site can be between amino acids 587 and 588 of AAV2, or the corresponding positions of the capsid subunit of another AAV serotype. It should be noted that the insertion site 587-588 is based on an AAV2 capsid protein. From about 5 amino acids to about 11 amino acids can be inserted in a corresponding site in an AAV serotype other than AAV2 (e.g., AAV5, AAV6, AAV8, AAV9, etc.).

In some embodiments, the insertion site is a single insertion site between two adjacent amino acids located between amino acids 570-614 of VP1 of any AAV serotype, e.g., the insertion site is between two adjacent amino acids located in amino acids 570-610, amino acids 580-600, amino acids 570-575, amino acids 575-580, amino acids 580-585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600.

In some aspects, an insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 588 and 589 of AAV10.

As another example, the insertion site can be between amino acids 450 and 460 of an AAV capsid protein, as shown in Table 5. For example, the insertion site can be at (e.g., immediately N-terminal to) amino acid 453 of AAV2, at amino acid 454 of AAV1, at amino acid 454 of AAV6, at amino acid 456 of AAV7, at amino acid 456 of AAV8, at amino acid 454 of AAV9, or at amino acid 456 of AAV10.

In some embodiments, a subject capsid protein includes a GH loop comprising an amino acid sequence having at least about 85%, at least about 90%, at least about 95%, at least about 98%, at least about 99%, or 100%, amino acid sequence identity to an amino acid sequence set forth in Table 5. Those skilled in the art would know, based on a comparison of the amino acid sequences of capsid proteins of various AAV serotypes, where an insertion site “corresponding to amino acids 587-588 of AAV2” would be in a capsid protein of any given AAV serotype.

In some cases, an exogenous polypeptide can have from 0 to 4 spacer amino acids (Y₁-Y₄) at the amino terminus and/or at the carboxyl terminus of any one of the exemplary polypeptides of Table 4 or Formula 1. Suitable spacer amino acids include, but are not limited to, leucine, alanine, glycine, and/or serine.

A modification of an AAV capsid can comprise a modification of at least one amino acid residue in a polypeptide sequence. In some cases, a modification can be made at any AAV capsid position, as described herein, and can include any number of modifications. In some cases, a modification can comprise a mutation. A mutation can comprise: a point mutation, missense mutation, nonsense mutation, deletion, duplication, frameshift, and/or repeat expansion.

In an aspect, an amino acid can be a non-polar, aliphatic residue such as glycine, alanine, valine, leucine, isoleucine, or proline. In an aspect, an amino acid residue is aromatic and is phenylalanine, tyrosine, or tryptophan. In an aspect, an amino acid residue is polar, non-charged and is serine, threonine, cysteine, methionine, asparagine, or glutamine. In an aspect, an amino acid is positively charged and is lysine, arginine, or histidine. In an aspect, an amino acid is negatively charged and is aspartate or glutamate.

In some cases, a mutation is a point mutation. A point mutation comprises a change from a charged amino acid residue to a polar or non-polar amino acid residue. In some cases, the charged amino acid is positively charged. In some cases, the charged amino acid is negatively charged.

A point mutation can be a conservative mutation. Non-limiting examples of conservative mutations comprise: a nonpolar aliphatic amino acid to a nonpolar aliphatic amino acid, a polar amino acid to a polar amino acid, a positively charged amino acid to a positively charged amino acid, a negatively charged amino acid to a negatively charged amino acid, and an aromatic amino acid to an aromatic amino acid. For example, 20 naturally occurring amino acids can share similar characteristics. Aliphatic amino acids can be: glycine, alanine, valine, leucine, or isoleucine. Hydroxyl or sulfur/selenium-containing amino acids can be: Serine, cysteine, selenocysteine, threonine, or methionine. A cyclic amino acid can be proline. An aromatic amino acid can be phenylalanine, tyrosine, or tryptophan. A basic amino acid can be histidine, lysine, and arginine. An acidic amino acid can be aspartate, glutamate, asparagine, or glutamine. A conservative mutation can be, serine to glycine, serine to alanine, serine to serine, serine to threonine, serine to proline. A conservative mutation can be arginine to asparagine, arginine to lysine, arginine to glutamine, arginine to arginine, arginine to histidine. A conservative mutation can be Leucine to phenylalanine, leucine to isoleucine, leucine to valine, leucine to leucine, leucine to methionine. A conservative mutation can be proline to glycine, proline to alanine, proline to serine, proline to threonine, proline to proline. A conservative mutation can be threonine to glycine, threonine to alanine, threonine to serine, threonine to threonine, threonine to proline. A conservative mutation can be alanine to glycine, alanine to threonine, alanine to proline, alanine to alanine, alanine to serine. A conservative mutation can be valine to methionine, valine to phenylalanine, valine to isoleucine, valine to leucine, valine to valine. A conservative mutation can be glycine to alanine, glycine to threonine, glycine to proline, glycine to serine, glycine to glycine. A conservative mutation can be Isoleucine to phenylalanine, isoleucine to isoleucine, isoleucine to valine, isoleucine to leucine, isoleucine to methionine. A conservative mutation can be phenylalanine to tryptophan, phenylalanine to phenylalanine, phenylalanine to tyrosine. A conservative mutation can be tyrosine to tryptophan, tyrosine to phenylalanine, tyrosine to tyrosine. A conservative mutation can be cysteine to serine, cysteine to threonine, cysteine to cysteine. A conservative mutation can be histidine to asparagine, histidine to lysine, histidine to glutamine, histidine to arginine, histidine to histidine. A conservative mutation can be glutamine to glutamic acid, glutamine to asparagine, glutamine to aspartic acid, glutamine to glutamine. A conservative mutation can be asparagine to glutamic acid, asparagine to asparagine, asparagine to aspartic acid, asparagine to glutamine. A conservative mutation can be lysine to asparagine, lysine to lysine, lysine to glutamine, lysine to arginine, lysine to histidine. A conservative mutation can be aspartic acid to glutamic acid, aspartic acid to asparagine, aspartic acid to aspartic acid, aspartic acid to glutamine. A conservative mutation can be glutamine to glutamine, glutamine to asparagine, glutamine to aspartic acid, glutamine to glutamine. A conservative mutation can be methionine to phenylalanine, methionine to isoleucine, methionine to valine, methionine to leucine, methionine to methionine. A conservative mutation can be tryptophan to tryptophan, tryptophan to phenylalanine, tryptophan to tyrosine.

Non-limiting examples of additional amino acid mutations can be: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

Any one of the aforementioned modifications, insertions, deletions, and/or mutations, can be made at any residue in an AAV sequence. The sequence may be a capsid sequence. In other cases, the sequence may not be a capsid sequence but rather a Rep and/or X sequence. The sequence may be in a VP1, VP2, and/or VP3 as previously described. In some cases, the sequence modification is of a loop of a capsid sequence, such as loop 3 and/or loop 4. In some cases, the modification is of a residue of a sequence in Table 5.

In some cases, a modification, such as insertion, deletion, and/or mutation is of a residue of a capsid polypeptide sequence in Table 5. In some cases, a modification is from 1-100, 100-200, 200-300, 300-400, 400-500, 500-600, 600-700, 700-800, or combinations thereof. In some cases, a modification is in a residue at position 200-300, 300-400, 400-500, 500-600 or combinations thereof. In some cases, a modification is in a residue at position 300-500 or combinations thereof. In an aspect, an insertion site is in the GH loop, or loop IV, of the AAV capsid protein, e.g., in a solvent-accessible portion of the GH loop, or loop IV, of the AAV capsid protein. For example, the insertion site is within amino acids 570-611 of AAV2, within amino acids 571-612 of AAV1, within amino acids 560-601 of AAV5, within amino acids 571 to 612 of AAV6, within amino acids 572 to 613 of AAV7, within amino acids 573 to 614 of AAV8, within amino acids 571 to 612 of AAV9, or within amino acids 573 to 614 of AAV10.

For example, the insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 589 and 590 of AAV10. In some cases, a modification is at position 452, 453, 466, 467, 468, 471, 585, 586, 587, and/or 588 of AAV2. In some cases, a modification is at position 452 or 453 of AAV2. In some cases, a modification is at position 587 or 588 of AAV2. In some cases, a modification is an insertion at position 452, 453, 466, 467, 468, 471, 585, 586, 587, and/or 588 of any one of SEQ ID NOs: 121-126. In some cases, a modification is an insertion at position 452, 453, 466, 467, 468, 471, 585, 586, 587, and/or 588 of SEQ ID NO: 121. In some cases, a modification is a mutation and the mutation is R585A or R588A of any one of SEQ ID NOs: 121-126. In some cases, a modification is a mutation and the mutation is R585A or R588A of SEQ ID NO: 121.

In some embodiments, a subject modified AAV capsid does not include any other amino acid modifications mutations, substitutions, insertions, or deletions, other than an insertion of from about 5 amino acids to about 11 amino acids in a loop (loop 3 and/or 4) relative to a corresponding unmodified AAV capsid protein. In other embodiments, a subject variant AAV capsid includes from 1 to about 25 amino acid insertions, deletions, or substitutions, compared to an unmodified AAV capsid protein, in addition to an insertion of from about 5 amino acids to about 11 amino acids in the loop 3 and/or loop 4 relative to an unmodified AAV capsid protein. In an embodiment, a subject AAV virion capsid does not include any other amino acid substitutions, insertions, or deletions, other than an insertion of from about 7 amino acids to about 10 amino acids in a GH loop or loop IV relative to a corresponding parental AAV capsid protein. In other embodiments, a subject AAV virion capsid includes from 1 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein. For example, in some embodiments, a subject AAV virion capsid includes from 1 to about 5, from about 5 to about 10, from about 10 to about 15, from about 15 to about 20, or from about 20 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein.

In some cases, a chimeric AAV capsid is provided herein. A chimeric capsid comprises a polypeptide sequence from at least 2 AAV serotypes. A chimeric capsid can comprise a mix of sequences selected from serotypes AAV1, AAV2, AAV3, AAV4, AAV5, AAV6, AAV7, AAV8, AAV9, AAV10, AAV11, and/or AAV12. In some cases, the chimeric serotypes are different between VP1, VP2, and/or VP3. In some cases, a chimeric capsid comprises sequences from at least 2 serotypes selected from: AAV4 and AAV6, AAV5 and AAV6, AAV11 and AAV6, AAV12 and AAV6, and any combination thereof. In some cases, a first AAV serotype can be AAV4 and a second serotype can be AAV6. In some cases, a first AAV serotype and a second AAV serotype of a chimeric AAV vector can be AAV11 and AAV6. In some cases, a first AAV serotype and a second AAV serotype of a chimeric AAV vector can be AAV12 and AAV6. In some cases, a chimeric capsid comprises sequences from: AAV2 and AAV5 or AAV2 and AAV6. In some cases, a chimeric capsid comprises sequences from: AAV2 and AAV5, AAV2 and AAV6, AAV2 and AAV8, AAV2 and AAV9, AAV2 and AAV1, and AAV2 and AAV12.

The modifications to an AAV provided herein can confer enhanced activity to the modified AAV as compared to an otherwise unmodified or wildtype AAV. Modifications provided herein can improve cell transduction, tropism, and/or reduce immunogenicity associated with the capsid.

In some cases, a modification provided herein enhances cellular transduction. Cellular transduction can refer to the ability of an AAV to infect a cell (in vivo or in vitro) and/or deliver a transgene into the cell.

In some cases, a modification provided herein enhances tropism. Enhanced tropism refers to gaining the ability to transduce cells through an extra receptor, as compared to an otherwise unmodified AAV. In some aspects, enhanced tropism can improve infectivity of an ocular cell, thereby improving gene therapy by way utilization of the modified AAV. In some cases, a modification provided herein can improve tropism to an ocular cell selected from: bipolar, retinal ganglion, horizontal, amacrine, epithelial, retinal pigment, photoreceptor, or any combination thereof. In some cases, a modification improves tropism to a retinal cell.

Also provided herein are AAV vectors. AAV vectors comprise: inverted terminal repeats (ITRs), Rep, Cap, AAP, and X sequences. Typically, the AAV viral genome is flanked by the ITRs, which serve as packaging signal and origin of replication. The rep gene encodes a family of multifunctional proteins (Rep proteins) responsible for controlling viral transcription, replication, packaging, and integration in AAVS1. For AAV2, four Rep proteins are described. Expression of Rep78 and Rep68 is controlled by the AAV2-specific p5 promoter, while p19 controls expression of the smaller Rep proteins (Rep52 and Rep40). Rep68 and Rep40 are splice variants of Rep78 and Rep52, respectively. Numbers indicate the molecular weight. Expression of AAP and the viral capsid proteins VP1 (90 kDa), VP2 (72 kDa), and VP3 (60 kDa), all encoded in the cap gene, is controlled by the p40 promoter. The X gene is located at the 3′ end of the genome within a region shared with the cap gene and possesses its own promoter (p81). While the X protein seems to enhance viral replication, AAP is essential for capsid assembly. The three different VPs contribute in a 1 (VP1):1 (VP2):10 (VP3) ratio to the icosahedral AAV2 capsid.

A modified capsid protein disclosed herein can be isolated, e.g., purified. In some embodiments, a modified capsid disclosed herein is included in an AAV vector or an AAV virion (for example recombinant AAV virion, rAAV, or an AAV viral particle). In other embodiments, such modified AAV vectors and/or AAV variant virions are used in an in vivo or ex vivo method of treating ocular disease in a primate retina, for example human retina.

Provided herein are also vectors that comprise modified AAV capsids. Any one of the previously described modifications can be encompassed in a vector provided herein. In some cases, an AAV vector comprises a modified capsid that comprises an exogenous sequence in at least two loops of a VP domain as compared to an otherwise comparable AAV capsid sequence that lacks the exogenous sequence. In some aspects, vectors provided herein can further comprise a transgene sequence.

### Engineered Polypeptide

Described herein are engineered polypeptide comprising a peptide operatively coupled to an antibody or fragment thereof. In some embodiments, the engineered polypeptide is encoded from an engineered polynucleotide described herein. In some embodiments, the engineered polypeptide comprises a natriuretic peptide. In some embodiments, the engineered polypeptide comprises a CNP. In some embodiments, the CNP comprises at least 20 amino acid residues, at least 22 amino acid residues, at least 25 amino acid residues, at least 30 amino acid residues, at least 35 amino acid residues, at least 36 amino acid residues, at least 40 amino acid residues, at least 45 amino acid residues, at least 50 amino acid residues, at least 53 amino acid residues, at least 55 amino acid residues, at least 60 amino acid residues, at least 61 amino acid residues, at least 65 amino acid residues, at least 70 amino acid residues, at least 75 amino acid residues, at least 80 amino acid residues, at least 85 amino acid residues, at least 90 amino acid residues, at least 95 amino acid residues, at least 100 amino acid residues, at least 105 amino acid residues, at least 110 amino acid residues, at least 115 amino acid residues, at least 120 amino acid residues, at least 125 amino acid residues, or at least 126 amino acid residues. In some embodiments, the CNP comprises 20 amino acid residues, 23 amino acid residues, 25 amino acid residues, 30 amino acid residues, 35 amino acid residues, 36 amino acid residues, 40 amino acid residues, 45 amino acid residues, 50 amino acid residues, 53 amino acid residues, 55 amino acid residues, 60 amino acid residues, 61 amino acid residues, 65 amino acid residues, 70 amino acid residues, 75 amino acid residues, 80 amino acid residues, 85 amino acid residues, 90 amino acid residues, 95 amino acid residues, 100 amino acid residues, 105 amino acid residues, 110 amino acid residues, 115 amino acid residues, 120 amino acid residues, 125 amino acid residues, or 126 amino acid residues. In some embodiments, the CNP comprises 22 amino acid residues. In some embodiments, the CNP comprises 36 amino acid residues. In some embodiments, the CNP comprises 53 amino acid residues. In some embodiments, the CNP comprises 61 amino acid residues. In some embodiments, the CNP comprises 126 amino acid residues. In some embodiments, the CNP comprises an amino acid sequence that is at least 50%, at least 60%, at least 70%, at least 80%, at least 90%, at least 95%, or at least 99% identical to any one of SEQ ID NOs: 1-5. In some embodiments, the CNP comprises an amino acid sequence that is 100% identical to SEQ ID NOs: 1-5. In some embodiments, the CNP comprises an amino acid sequence that is at least 50%, at least 60%, at least 70%, at least 80%, at least 90%, at least 95%, or at least 99% identical to SEQ ID NO: 1. In some embodiments, the CNP comprises an amino acid sequence that is 100% identical to SEQ ID NO: 1. In some embodiments, the CNP comprises an amino acid sequence that is at least 50%, at least 60%, at least 70%, at least 80%, at least 90%, at least 95%, or at least 99% identical to SEQ ID NO: 2. In some embodiments, the CNP comprises an amino acid sequence that is 100% identical to SEQ ID NO: 2. In some embodiments, the CNP comprises an amino acid sequence that is at least 50%, at least 60%, at least 70%, at least 80%, at least 90%, at least 95%, or at least 99% identical to SEQ ID NO: 3. In some embodiments, the CNP comprises an amino acid sequence that is 100% identical to SEQ ID NO: 3. In some embodiments, the CNP comprises an amino acid sequence that is at least 50%, at least 60%, at least 70%, at least 80%, at least 90%, at least 95%, or at least 99% identical to SEQ ID NO: 4. In some embodiments, the CNP comprises an amino acid sequence that is 100% identical to SEQ ID NO: 4. In some embodiments, the CNP comprises an amino acid sequence that is at least 50%, at least 60%, at least 70%, at least 80%, at least 90%, at least 95%, or at least 99% identical to SEQ ID NO: 5. In some embodiments, the CNP comprises an amino acid sequence that is 100% identical to SEQ ID NO: 5.

In some embodiments, the engineered polypeptide comprises an antibody or fragment thereof operatively coupled to the peptide or the CNP. In some embodiments, the antibody or fragment thereof comprises an Fc region of the antibody. In some embodiments, the antibody or fragment thereof comprises an amino acid sequence that is at least 50%, at least 60%, at least 70%, at least 80%, at least 90%, at least 95%, at least 99% identical to any one of SEQ ID NOs: 6-8. In some embodiments, the antibody or fragment thereof comprises an amino acid sequence that is at least 50%, at least 60%, at least 70%, at least 80%, at least 90%, at least 95%, at least 99% identical to SEQ ID NO: 6. In some embodiments, the antibody or fragment thereof comprises an amino acid sequence that is 100% identical to SEQ ID NO: 6. In some embodiments, the antibody or fragment thereof comprises an amino acid sequence that is at least 50%, at least 60%, at least 70%, at least 80%, at least 90%, at least 95%, at least 99% identical to SEQ ID NO: 7. In some embodiments, the antibody or fragment thereof comprises an amino acid sequence that is 100% identical to SEQ ID NO: 7. In some embodiments, the antibody or fragment thereof comprises an amino acid sequence that is at least 50%, at least 60%, at least 70%, at least 80%, at least 90%, at least 95%, at least 99% identical to SEQ ID NO: 8. In some embodiments, the antibody or fragment thereof comprises an amino acid sequence that is 100% identical to SEQ ID NO: 8.

In some embodiments, the engineered polypeptide comprises a peptide covalently connected to N terminus of the antibody or fragment thereof. In some embodiments, the engineered polypeptide comprises a peptide covalently connected to C terminus of the antibody or fragment thereof. In some embodiments, the engineered polypeptide comprises a peptide covalently connected or operatively coupled to the antibody or fragment thereof by a peptide linker. In some embodiments, the peptide linker comprises at least one glycine followed by a serine. In some embodiments, the peptide linker comprises a subunit of at least one glycine followed by a serine. For example, the subunit can be denoted as (GS)ₙ, where the n is an integer between 0-20 (SEQ ID NO: 143). In such scenario n equals to two would equate to a peptide linker having an amino acid sequence of (GS)₂ or GSGS (SEQ ID NO: 144). In some embodiments, the subunit can include one glycine followed by a serine (e.g., GS), two glycine followed by a serine (e.g., GGS), three glycine followed by a serine (e.g., GGGS (SEQ ID NO: 145)), four glycine followed by a serine (e.g., GGGGS (SEQ ID NO: 146)), five glycine followed by a serine (e.g., GGGGGS (SEQ ID NO: 147)), or six glycine followed by a serine (e.g., GGGGGGS (SEQ ID NO: 148)). In some embodiments, the peptide linker comprises a subunit of at least one glycine followed by a serine, at least two glycine followed by a serine, at least three glycine followed by a serine, at least four glycine followed by a serine, or at least six glycine followed by a serine. In some embodiments, the ₙ is an integer of one, two, three, four, five, six, seven, eight, nine, ten, 11, 12, 13, 14, 15, 16, 16, 17, 18, 19, or 20. In some embodiments, the n is an integer of more than 20. In some embodiments, the n is an integer of four. In some embodiments, the n is an integer of five.

In some embodiments, the engineered polypeptide comprises a peptide covalently connected to the antibody or fragment thereof by a peptide linker, where the peptide linker comprises an amino acid sequence comprising (GGGGS)ₙ, where the n is an integer between 0-20 (SEQ ID NO: 141). In some embodiments, the engineered polypeptide comprises a peptide covalently connected to the antibody or fragment thereof by a peptide linker, where the peptide linker comprises an amino acid sequence comprising (GGGGS)ₙ, where the n is an integer between 0-5 (SEQ ID NO: 149). In some embodiments, the engineered polypeptide comprises a peptide covalently connected to the antibody or fragment thereof by a peptide linker, where the peptide linker comprises an amino acid sequence comprising (GGGGS)ₙ, where the n is an integer of four (SEQ ID NO: 150). In some embodiments, the engineered polypeptide comprises a peptide covalently connected to the antibody or fragment thereof by a peptide linker, where the peptide linker comprises an amino acid sequence comprising (GGGGS)ₙ, where the n is an integer of five (SEQ ID NO: 151).

In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 131-140. In some embodiments, peptide operatively coupled to the antibody or fragment thereof comprises an amino acid sequence that is 100% identical to any one of SEQ ID NOs: 131-140. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 131. In some embodiments, peptide operatively coupled to the antibody or fragment thereof comprises an amino acid sequence that is 100% identical to SEQ ID NO: 131. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 132. In some embodiments, peptide operatively coupled to the antibody or fragment thereof comprises an amino acid sequence that is 100% identical to SEQ ID NO: 132. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 133. In some embodiments, peptide operatively coupled to the antibody or fragment thereof comprises an amino acid sequence that is 100% identical to SEQ ID NO: 133. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 134. In some embodiments, peptide operatively coupled to the antibody or fragment thereof comprises an amino acid sequence that is 100% identical to SEQ ID NO: 134. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 135. In some embodiments, peptide operatively coupled to the antibody or fragment thereof comprises an amino acid sequence that is 100% identical to SEQ ID NO: 135. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 136. In some embodiments, peptide operatively coupled to the antibody or fragment thereof comprises an amino acid sequence that is 100% identical to SEQ ID NO: 136. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 137. In some embodiments, peptide operatively coupled to the antibody or fragment thereof comprises an amino acid sequence that is 100% identical to SEQ ID NO: 137. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 138. In some embodiments, peptide operatively coupled to the antibody or fragment thereof comprises an amino acid sequence that is 100% identical to SEQ ID NO: 138. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 139. In some embodiments, peptide operatively coupled to the antibody or fragment thereof comprises an amino acid sequence that is 100% identical to SEQ ID NO: 139. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 140. In some embodiments, peptide operatively coupled to the antibody or fragment thereof comprises an amino acid sequence that is 100% identical to SEQ ID NO: 140.

In some embodiments, peptide operatively coupled to the antibody or fragment thereof comprises an amino acid sequence that is at least 50%, at least 60%, at least 70%, at least 80%, at least 90%, at least 95%, or at least 99% identical to any one of SEQ ID NOs: 9-12. In some embodiments, peptide operatively coupled to the antibody or fragment thereof comprises an amino acid sequence that is 100% identical to any one of SEQ ID NOs: 9-12. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 9. In some embodiments, peptide operatively coupled to the antibody or fragment thereof comprises an amino acid sequence that is 100% identical to SEQ ID NO: 9. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 10. In some embodiments, peptide operatively coupled to the antibody or fragment thereof comprises an amino acid sequence that is 100% identical to SEQ ID NO: 10. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 11. In some embodiments, peptide operatively coupled to the antibody or fragment thereof comprises an amino acid sequence that is 100% identical to SEQ ID NO: 11. In some embodiments, peptide operatively coupled to the antibody or fragment thereof comprises an amino acid sequence that is at least 50%, at least 60%, at least 70%, at least 80%, at least 90%, at least 95%, or at least 99% identical to SEQ ID NO: 12. In some embodiments, peptide operatively coupled to the antibody or fragment thereof comprises an amino acid sequence that is 100% identical to SEQ ID NO: 12.

In some embodiments, the engineered polypeptide comprising the CNP operatively coupled to the antibody or fragment thereof increases half-life of the coupled CNP compared to an uncoupled CNP. In some embodiments, the CNP operatively coupled to the antibody or fragment thereof increases the half-life by at least 0.1 fold, at least 0.2 fold, at least 0.3 fold, at least 0.4 fold, at least 0.5 fold, at least 0.6 fold, at least 0.7 fold, at least 0.8 fold, at least 0.9 fold, at least 1.0 fold, at least 2.0 fold, at least 5.0 fold, at least 10.0 fold, at least 20.0 fold, at least 50.0 fold, or at least 100.0 fold compared to a half-life of an uncoupled CNP. In some embodiments, the CNP operatively coupled to the antibody or fragment thereof increases the half-life by at least 1 minute, at least 2 minutes, at least 3 minutes, at least 5 minutes, at least 10 minutes, at least 30 minutes, at least 60 minutes, at least 120 minutes, at least 3 hours, at least 4 hours, at least 5 hours, at least 6 hours, at least 12 hours, at least 24 hours, at least 2 days, at least 3 days at least 4 days, at least 5 days at least 10 days, at least 15 days at least 20 days, or at least 30 days compared to a half-life of an uncoupled CNP. In some embodiments, the CNP operatively coupled to the antibody or fragment thereof increases the in vivo half-life by at least 1 minute, at least 2 minutes, at least 3 minutes, at least 5 minutes, at least 10 minutes, at least 30 minutes, at least 60 minutes, at least 120 minutes, at least 3 hours, at least 4 hours, at least 5 hours, at least 6 hours, at least 12 hours, at least 24 hours, at least 2 days, at least 3 days at least 4 days, at least 5 days at least 10 days, at least 15 days at least 20 days, or at least 30 days compared to an in vivo half-life of an uncoupled CNP.

In some embodiments, the engineered polypeptide comprising the CNP operatively coupled to the antibody or fragment thereof increases protection against degradation of the coupled CNP compared to an uncoupled CNP. In some embodiments, the CNP operatively coupled to the antibody or fragment thereof increases the protection against degradation by at least 0.1 fold, at least 0.2 fold, at least 0.3 fold, at least 0.4 fold, at least 0.5 fold, at least 0.6 fold, at least 0.7 fold, at least 0.8 fold, at least 0.9 fold, at least 1.0 fold, at least 2.0 fold, at least 5.0 fold, at least 10.0 fold, at least 20.0 fold, at least 50.0 fold, or at least 100.0 fold compared to degradation of an uncoupled CNP.

In some embodiments, the engineered polypeptide can be administered to a subject to treat a disease or condition. In some embodiments, the engineered polypeptide can be formulated into a pharmaceutical composition to be administered to a subject to treat a disease or condition. In some embodiments, the engineered polypeptide comprising the CNP and the antibody or fragment thereof can be administered to a subject to treat a disease or condition. In some embodiments, the engineered polypeptide comprising the CNP and the antibody or fragment thereof can be formulated into a pharmaceutical composition to be administered to a subject to treat a disease or condition.

In some embodiments, the engineered polypeptide can increase activity or signal cascade associated with a natriuretic peptide receptor (NPR). In some embodiments, the engineered polypeptide can increase activity or signal cascade associated with a cyclic GMP (cGMP) signaling pathway. In some embodiments, the engineered polypeptide comprising the CNP and the antibody or fragment thereof can increase activity or signal cascade associated with a natriuretic peptide receptor (NPR). In some embodiments, the engineered polypeptide comprising the CNP and the antibody or fragment thereof can increase activity or signal cascade associated with a cyclic GMP (cGMP) signaling pathway.

In some embodiments, the engineered polypeptide can be administered to a subject to treat a disease or condition by increasing increases activity or signal cascade associated with a natriuretic peptide receptor (NPR). In some embodiments, the engineered polypeptide can be administered to a subject to treat a disease or condition by increasing increases activity or signal cascade associated with a cGMP signaling pathway. In some embodiments, the engineered polypeptide comprising the CNP and the antibody or fragment thereof can be administered to a subject to treat a disease or condition by increasing increases activity or signal cascade associated with a natriuretic peptide receptor (NPR). In some embodiments, the engineered polypeptide comprising the CNP and the antibody or fragment thereof can be administered to a subject to treat a disease or condition by increasing increases activity or signal cascade associated with a cGMP signaling pathway.

### Pharmaceutical Composition

Described herein are pharmaceutical compositions comprising an engineered polynucleotide, an AAV vector comprising the engineered polynucleotide, an engineered polypeptide, a cell transduced by an AAV vector comprising an engineered polynucleotide, a viral particle comprising the engineered polynucleotide, or a combination thereof. In some embodiments, the pharmaceutical composition further comprises as pharmaceutically acceptable: carrier, excipient, or diluent. In some embodiments, the pharmaceutical composition comprises two or more active agents as disclosed herein. In some embodiments, the pharmaceutical composition comprising the engineered polynucleotide, the AAV vector comprising the engineered polynucleotide, or the AAV vector comprising the engineered polynucleotide treats a disease or condition described herein. In some embodiments, the disease or condition comprises an ocular disease. In some embodiments, the disease or condition comprises ocular ischemic syndrome, proliferative retinopathies, neovascular glaucoma (NG), uveitis, neovascular uveitis, achromatopsia, age-related macular degeneration (nAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

For in vivo delivery, the engineered polynucleotide, the AAV vector comprising the engineered polynucleotide, the engineered polypeptide, the cell transduced by an AAV vector comprising the engineered polynucleotide, or a combination thereof can be formulated into pharmaceutical compositions and can generally be administered intravitreally or parenterally (e.g., administered via an intramuscular, subcutaneous, intratumoral, transdermal, intrathecal, etc., route of administration). In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof to a subject in need thereof.

In some aspects, a pharmaceutical composition can be used to treat a subject such as a human or mammal, in need thereof. In some cases, a subject can be diagnosed with a disease, e.g., ocular disease. In some aspects, subject pharmaceutical compositions are co-administered with secondary therapies. A secondary therapy can comprise any therapy for ocular use. In some cases, a secondary therapy comprises nutritional therapy, vitamins, laser treatment, such as laser photocoagulation, photodynamic therapy, Visudyne, anti-VEGF therapy, eye-wear, eye drops, numbing agents, Orthoptic vision therapy, Behavioral/perceptual vision therapy, and the like. In some aspects, any of the previously described biologics can be considered a secondary therapy.

In some embodiments, an effective amount of the pharmaceutical composition results in a decrease in the rate of loss of retinal function, anatomical integrity, or retinal health, e.g., a 2-fold, 3-fold, 4-fold, or 5-fold or more decrease in the rate of loss and hence progression of disease, for example, a 10-fold decrease or more in the rate of loss and hence progression of disease.

In some embodiments, an effective amount of the pharmaceutical composition decreases neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in a cell that is not treated with the pharmaceutical composition. In some embodiments, an effective amount of the pharmaceutical composition decreases neovascularization in a subject in need thereof at least 10%, at least 20%, at least 30%, at least 40%, at least 50%, at least 60%, at least 70%, at least 80%, at least 90%, at least 100%, at least 200%, at least 500%, or more compared to neovascularization in the subject if the subject is not treated with the pharmaceutical composition. In some embodiments, an effective amount of the pharmaceutical composition decreases blood vessel leakage in a subject in need thereof at least 10%, at least 20%, at least 30%, at least 40%, at least 50%, at least 60%, at least 70%, at least 80%, at least 90%, at least 100%, at least 200%, at least 500%, or more compared to blood vessel leakage in the subject if the subject is not treated with the pharmaceutical composition. In some embodiments, an effective amount of the pharmaceutical composition decreases inflammation in a subject in need thereof at least 10%, at least 20%, at least 30%, at least 40%, at least 50%, at least 60%, at least 70%, at least 80%, at least 90%, at least 100%, at least 200%, at least 500%, or more compared to inflammation in the subject if the subject is not treated with the pharmaceutical composition.

In some embodiments, the effective amount of the subject rAAV virion results in a gain in visual function, retinal function, an improvement in retinal anatomy or health, and/or an improvement in ocular motility and/or improvement in neurological function, e.g. a 2-fold, 3-fold, 4-fold or 5-fold improvement or more in retinal function, retinal anatomy or health, and/or improvement in ocular motility, e.g. a 10-fold improvement or more in retinal function, retinal anatomy or health, and/or improvement in ocular motility. As will be readily appreciated by the ordinarily skilled artisan, the dose required to achieve the desired treatment effect will typically be in the range of 1×10⁸ to about 1×10¹⁵ recombinant virions, typically referred to by the ordinarily skilled artisan as 1×10⁸ to about 1×10¹⁵ “vector genomes”.

In some aspects, compositions provided herein, such as pharmaceutical compositions are administered to a subject in need thereof. In some cases, an administration comprises delivering a dosage of an AAV of about vector 0.5×10⁹ vg, 1.0×109 vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, 1.5×10¹⁵ vg. For example, for in vivo injection, e.g., injection directly into the eye, a therapeutically effective dose can be on the order of from about 10⁶ to about 10¹⁵ of subject AAV virions, e.g., from about 10⁸ to 10¹² engineered AAV virions. For in vitro transduction, an effective amount of engineered AAV virions to be delivered to cells will be on the order of from about 10⁸ to about 10¹³ of the engineered AAV virions. Other effective dosages can be readily established by one of ordinary skill in the art through routine trials establishing dose response curves.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In practicing the methods of treatment or use provided herein, therapeutically effective amounts of the pharmaceutical composition described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical composition described herein is self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Kit

Disclosed herein, in some embodiments, are kits for using comprising an engineered polynucleotide, an AAV comprising the engineered polynucleotide, an engineered polypeptide, a cell transduced by an AAV vector comprising an engineered polynucleotide, a viral particle comprising the engineered polynucleotide, a pharmaceutical composition, or a combination thereof described herein. In some embodiments, the kit disclosed herein may be used to treat a disease or condition in a subject. In some embodiments, the kit comprises an assemblage of materials or components apart from comprising the engineered polynucleotide, the AAV comprising the engineered polynucleotide, the engineered polypeptide, the cell transduced by an AAV vector comprising an engineered polynucleotide, or the pharmaceutical composition.

In some embodiments, the kit described herein comprises components for selecting for a homogenous population of AAV containing the engineered polynucleotide described herein. In some embodiments, the kit comprises the components for assaying the number of units of a biomolecule (e.g., the AAV) synthesized, and/or released or expressed on the surface by a host cell. In some embodiments, the kit comprises components for performing assays such as enzyme-linked immunosorbent assay (ELISA). The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the engineered polynucleotide, the AAV vector comprising the engineered polynucleotide, the engineered polypeptide, the AAV comprising the engineered polynucleotide, the cell transduced with the AAV vector, the pharmaceutical composition, or a combination thereof to a subject in need thereof. In some embodiments, the kit comprises instructions for further engineering a cell to express a biomolecule (e.g., the engineered polynucleotide, the AAV vector comprising the engineered polynucleotide, the engineered polypeptide, the AAV comprising the engineered polynucleotide, or the cell transduced with the AAV vector). In some embodiments, the kit comprises instructions for thawing or otherwise restoring biological activity of the engineered polynucleotide, the AAV vector comprising the engineered polynucleotide, AAV comprising the engineered polynucleotide, which may have been cryopreserved or lyophilized during storage or transportation. In some embodiments, the kit comprises instructions for measuring the viability of the restored the engineered polynucleotide, the AAV vector comprising the engineered polynucleotide, AAV comprising the engineered polynucleotide to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

### Method of Delivery

The engineered polynucleotide can be readily introduced into a host cell, e.g., a mammalian, bacterial, yeast, or insect cell by any method in the art. For example, the engineered polynucleotide can be transferred into a host cell by physical, chemical, or biological means. In some embodiments, the engineered polynucleotide can be delivered to a host cell by encapsulating the engineered polynucleotide in a viral particle such as an AAV particle. In some embodiments, the engineered polynucleotide can be delivered into the cell via physical methods such as calcium phosphate precipitation, lipofection, particle bombardment, microinjection, gene gun, electroporation, and the like.

Physical methods for introducing the engineered polynucleotide encoding into the cell can include calcium phosphate precipitation, lipofection, particle bombardment, microinjection, gene gun, electroporation, and the like. One method for the introduction of the engineered polynucleotide a host cell is calcium phosphate transfection.

Chemical means for introducing the engineered polynucleotide encoding the non-naturally into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles.

In the case where a non-viral delivery system is utilized, an example delivery vehicle is a liposome. The use of lipid formulations is contemplated for the introduction of the engineered polynucleotide or vector encoding the engineered polynucleotide into a cell (in vitro, ex vivo, or in vivo). In another aspect, the vector can be associated with a lipid. The vector associated with a lipid can be encapsulated in the aqueous interior of a liposome, interspersed within the lipid bilayer of a liposome, attached to a liposome via a linking molecule that is associated with both the liposome and the engineered polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the engineered polynucleotide can be delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV comprises a serotype, including AAV1, AAV2, AAV4, AAV5, AAV6, AAV7, AAV8, AAV9, AAV10, AAV11, AAV12, or a combination thereof. Based on these initial serotypes, AAV capsid of each serotype can be engineered to make them better suited for biological functions, tissue or cell selection. In some embodiments, an AAV is AAV2 and variants AAV2.N53 and AAV2.N54 which are used in the examples of the present disclosure. Chimeric AAVs are also contemplated that may contain at least 2 AAV serotypes. In some cases, at least 3, at least 4, at least 5, at least 6, at least 7, or up to 8 different serotypes are combined in a chimeric AAV. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

### Method of Modifying Cell

In an aspect, provided herein are also methods of modifying cells to thereby generate engineered cells. Cells can refer to primary cells, recombinant cells, or cell lines. In some cases, a cell is a packaging cell. A packaging cell can be any one of: HEK 293 cells, HeLa cells, and Vero cells to name a few. An engineered cell can be a primary cell. In some cases, an engineered cell can be an ocular cell. Suitable ocular cells include but are not limited to a: photoreceptor, ganglion cell, RPE cell, amacrine cell, horizontal cell, muller cell, and the like.

In some cases, a cell is a packaging cell utilized to generate viral particles. To generate AAV virions or viral particles, an AAV vector is introduced into a suitable host cell using known techniques, such as by transfection. In some cases, transfection techniques are used, e.g., CaPO4 transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins). Suitable transfection methods include calcium phosphate co-precipitation, direct micro-injection, electroporation, liposome mediated gene transfer, and nucleic acid delivery using high-velocity microprojectiles, which are known in the art.

To engineer a cell, a plurality of cells may be contacted with an isolated engineered polynucleotide. Contacting can comprise any length of time and may include from about 5 min to about 5 days. Contacting can last from about 5, about 10, about 15, about 20, about 25, about 30, about 35, about 40, about 45, about 50, about 55, or about 60 minutes. In some cases, the contacting can last from 1 hour, 3 hours, 5 hours, 10 hours, 15 hours, 20 hours, 1 day, 2 days, 3 days, 4 days or up to about 5 days.

In some cases, supernatant of the packaging cell line is treated by PEG precipitation for concentrating the virus. In other cases, a centrifugation step can be used to concentrate a virus. For example, a column can be used to concentration a virus during a centrifugation. In some embodiments, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some embodiments, the recombinant AAV is isolated from the PEG-precipitated supernatant by low-speed centrifugation followed by CsCl gradient. The low-speed centrifugation can be to can be about 4000 rpm, about 4500 rpm, about 5000 rpm, or about 6000 rpm for about 20 minutes, about 30 minutes, about 40 minutes, about 50 minutes or about 60 minutes. In some cases, recombinant AAV is isolated from the PEG-precipitated supernatant by centrifugation at about 5000 rpm for about 30 minutes followed by CsCl gradient. In some cases, CsCl purification can be replaced with IDX gradient ultracentrifugation. Supernatant can be collected at about 12 hours, about 24 hours, about 36 hours, about 48 hours, about 72 hours, about 96 hours, about 120 hours, or a time between any of these two time points after a transfection. Supernatant can also be purified, concentrated, or a combination thereof. For example, a concentration or viral titer can be determined by qPCR or silver stain.

In an aspect, provided is also a plurality of AAV particles (containing the engineered polynucleotide described herein) isolated from an engineered cell. A viral titer can be from about 10² vp/mL, about 10³ vp/mL, about 10⁴ vp/mL, about 10⁵ vp/mL, about 10⁶ vp/mL, about 10⁷ vp/mL, about 10⁸ vp/mL, or up to about 10⁹ vp/mL. A viral titer can be from about 10² GC/mL, about 10³ GC/mL, about 10⁴ GC/mL, about 10⁵ GC/mL, about 10⁶ GC/mL, about 10⁷ GC/mL, about 10⁸ GC/mL, or up to about 10⁹ GC/mL. In some cases, a viral titer can be from about 10² TU/mL, about 10³ TU/mL, about 10⁴ TU/mL, about 10⁵ TU/mL, about 10⁶ TU/mL, about 10⁷ TU/mL, about 10⁸ TU/mL, or up to about 10⁹ TU/mL. An optimal viral titer can vary depending on cell type to be transduced. A range of virus can be from about 1000 MOI to about 2000 MOI, from about 1500 MOI to about 2500 MOI, from about 2000 MOI to about 3000 MOI, from about 3000 MOI to about 4000 MOI, from about 4000 MOI to about 5000 MOI, from about 5000 MOI to about 6000 MOI, from about 6000 MOI to about 7000 MOI, from about 7000 MOI to about 8000 MOI, from about 8000 MOI to about 9000 MOI, from about 9000 MOI to about 10,000 MOI. For example, to infect 1 million cells using a MOI of 10,000, one will need 10,000×1,000,000=10¹⁰ GC.

In some cases, a plurality of AAV particles can be formulated into unit dose form. Various formulations are contemplated for adult or pediatric delivery and include but are not limited to: 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, or up to 1.5×10¹⁵ vg. Compositions of viral particles can be cryopreserved or otherwise stored in suitable containers.

Provided compositions and methods herein can be sufficient to enhance delivery and/or expression of subject biologic by at least about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or up to 100% more than an otherwise comparable unmodified nucleic acid. In some cases, the otherwise comparable unmodified nucleic acid is one that encodes VEGF-Trap. In some cases, modifications can be sufficient to enhance delivery and/or expression of subject biologics by at least about 1-fold, about 6-fold, about 11-fold, about 16-fold, about 21-fold, about 26-fold, about 31-fold, about 36-fold, about 41-fold, about 46-fold, about 51-fold, about 56-fold, about 61-fold, about 66-fold, about 71-fold, about 76-fold, about 81-fold, about 86-fold, about 91-fold, about 96-fold, about 101-fold, about 106-fold, about 111-fold, about 116-fold, about 121-fold, about 126-fold, about 131-fold, about 136-fold, about 141-fold, about 146-fold, about 151-fold, about 156-fold, about 161-fold, about 166-fold, about 171-fold, about 176-fold, about 181-fold, about 186-fold, about 191-fold, about 196-fold, about 201-fold, about 206-fold, about 211-fold, about 216-fold, about 221-fold, about 226-fold, about 231-fold, about 236-fold, about 241-fold, about 246-fold, about 251-fold, about 256-fold, about 261-fold, about 266-fold, about 271-fold, about 276-fold, about 281-fold, about 286-fold, about 291-fold, about 296-fold, about 301-fold, about 306-fold, about 311-fold, about 316-fold, about 321-fold, about 326-fold, about 331-fold, about 336-fold, about 341-fold, about 346-fold, or about 350-fold more than an otherwise comparable unmodified nucleic acid. In an embodiment, increased expression comprises at least a 5-fold, at least a 10-fold, at least a 20-fold, at least a 50-fold, at least a 100-fold, at least a 200-fold, or at least a 500-fold increase as determined by in in vitro assay. Suitable in vitro assays include ELISA, western blot, Luminex, microscopy, imaging, and/or flow cytometry.

A subject AAV virion can exhibit at least 1-fold, at least 6-fold, at least 10-fold, at least 15-fold, at least 20-fold, at least 25-fold, at least 50-fold, or more than 50-fold, increased infectivity of a retinal cell, compared to the infectivity of the retinal cell (photoreceptor, ganglion cell, RPE cell, amacrine cell, horizontal cell, muller cell, and the like) by an AAV virion comprising an otherwise comparable WT AAV capsid protein.

### Method of Treatment

Provided herein are methods of treating a disease or condition described here. In some aspects, the method confers protection against the disease or condition. A method of treatment can comprise introducing to a subject in need an engineered polynucleotide, an AAV vector comprising the engineered polynucleotide, an AAV comprising the engineered polynucleotide, a cell transduced with an AAV vector, a viral particle comprising the engineered polynucleotide, a pharmaceutical composition, or a combination thereof. Also provided is a method of treating disease or condition that comprises administering a pharmaceutical composition to a subject in need thereof. A pharmaceutical composition can comprise a sequence that encodes a biologic that comprises the engineered polynucleotide, the AAV vector comprising the engineered polynucleotide, the AAV vector comprising the engineered polynucleotide, a viral particle comprising the engineered polynucleotide, or a combination thereof. In some embodiments, administration is by any suitable mode of administration, including systemic administration (e.g., intravenous, intravitreal, subretinal, or etc.). In some embodiments, the subject is human.

In some embodiments, the method comprises treating a disease or condition in a subject in need thereof by administering to the subject a therapeutically effective amount of an engineered polynucleotide, an engineered polypeptide, a cell transduced with an engineered polynucleotide, or pharmaceutical composition described herein. In some embodiments, the method treats a disease or condition, where once of the administering of an engineered polynucleotide, an engineered polypeptide, a cell transduced with an engineered polynucleotide, or pharmaceutical composition described herein is curative of the disease or condition. In some embodiments, the method treats a disease or condition, where the administering of an engineered polynucleotide, an engineered polypeptide, a cell transduced with an engineered polynucleotide, or pharmaceutical composition described herein does not comprise daily administration. In some embodiments, the disease or condition comprises an ocular disease. Non-limiting example of the ocular disease can include ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof. In some embodiments, the disease or condition is neovascular glaucoma (NG). In some embodiments, the disease or condition is glaucoma. In some embodiments, the disease or condition is traumatic glaucoma.

In some embodiments, administering a therapeutically effective amount of an engineered polynucleotide, an engineered polypeptide, a cell transduced with an engineered polynucleotide, or pharmaceutical composition described herein to a subject protects the subjection from the disease or condition. For example, administering a therapeutically effective amount of an engineered polynucleotide, an engineered polypeptide, a cell transduced with an engineered polynucleotide, or pharmaceutical composition can protect the subject from developing disease or condition stemmed from injury. As shown in Example 4, the engineered polypeptide promoted protection of retina ganglion cells in eyes after transection injury. In some embodiments, administering a therapeutically effective amount of an engineered polynucleotide, an engineered polypeptide, a cell transduced with an engineered polynucleotide, or pharmaceutical composition protects or promotes survival of cells in a subject. In some embodiments, administering a therapeutically effective amount of an engineered polynucleotide, an engineered polypeptide, a cell transduced with an engineered polynucleotide, or pharmaceutical composition protects or promotes survival of ocular cells in a subject. In some embodiments, administering a therapeutically effective amount of an engineered polynucleotide, an engineered polypeptide, a cell transduced with an engineered polynucleotide or, pharmaceutical composition protects or promotes survival of retinal ganglion cells in a subject. In some embodiments, administering a therapeutically effective amount of an engineered polynucleotide, an engineered polypeptide, a cell transduced with an engineered polynucleotide or, pharmaceutical composition decreases intraocular pressure in a subject.

In some embodiments, the engineered polynucleotide, the AAV vector comprising the engineered polynucleotide, the AAV comprising the engineered polynucleotide, the cell transduced with the AAV vector, or the pharmaceutical composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time. In some embodiments, the administration described herein comprises a single administration. In some embodiments, the administration described herein does not include daily administration.

In some embodiments, the method comprises administering the engineered polynucleotide, the AAV vector comprising the engineered polynucleotide, the AAV comprising the engineered polynucleotide, the cell transduced with the AAV vector, or the pharmaceutical composition in a therapeutically-effective amount by various forms and routes including, for example, oral, or topical administration. In some embodiments, a composition may be administered by intravitreal, subretinal, suprachoroidal, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the method comprises administering the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof by intravenous infusion. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered by slow continuous infusion over a long period, such as more than 24 hours. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered as an intravenous injection or a short infusion. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered via vitreous route. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered in a local manner, for example, via injection of the agent directly into an organ, optionally in a depot or sustained release formulation or implant.

In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered in conjunction with other therapies, for example, an antiviral therapy, a chemotherapy, an antibiotic, a cell therapy, a cytokine therapy, or an anti-inflammatory agent. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered to a subject before the onset of the symptoms. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent may be administered after (e.g., as soon as is practicable after) the onset of a disease or condition is detected or suspected. A therapeutic agent may be administered after (e.g., as soon as is practicable after) a potential exposure to a coronavirus, for example, after a subject (e.g., the subject for immunization or the subject for treatment) has contact with an infected subject or learns they had contact with an infected subject that may be contagious.

Actual dosage levels of an agent of the disclosure (e.g., the engineered polynucleotide or a pharmaceutical composition) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present disclosure employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

In some cases, an increased level of a biologic in a subject is at least a 5-fold, a 10-fold, a 20-fold, a 50-fold, a 100-fold, a 200-fold, or a 500-fold increased, as determined by a diagnostic assay.

Suitable diagnostic assays can include ocular diagnostic assays. Ocular diagnostic assays can include ophthalmic testing such as refraction testing, ocular scans, Ocular coherence tomography, Farnworth-Munsell 100 Hue Test, Computerized Optic Disc Imaging and Nerve Fiber Layer Analysis (GDX, HRT, OCT), Corneal Topography, Electroretinography (ERG), electro-oculography (EOG), visual evoked potentials (VEP), visual evoked response (VER), Fluorescein Angiography, Ocular Coherence Tomography (OCT), retinal photography, fundus photography, Specular Microscopy, Goldmann, Humphrey, FDT, Octopus, Biometry/IOL calculation, A-Scan, B-Scan, and combinations thereof.

In some cases, a retinal test can be utilized. Nonlimiting methods for assessing retinal function and changes thereof include assessing visual acuity (e.g. best-corrected visual acuity [BCVA], ambulation, navigation, object detection and discrimination), assessing visual field (e.g. static and kinetic visual field perimetry), performing a clinical examination (e.g. slit lamp examination of the anterior and posterior segments of the eye), assessing electrophysiological responsiveness to all wavelengths of light and dark (e.g. all forms of electroretinography (ERG) [full-field, multifocal and pattern], all forms of visual evoked potential (VEP), electrooculography (EOG), color vision, dark adaptation and/or contrast sensitivity). Nonlimiting methods for assessing anatomy and retinal health and changes thereof include Optical Coherence Tomography (OCT), fundus photography, adaptive optics scanning laser ophthalmoscopy (AO-SLO), fluorescence and/or autofluorescence; measuring ocular motility and eye movements (e.g. nystagmus, fixation preference, and stability), measuring reported outcomes (patient-reported changes in visual and non-visually-guided behaviors and activities, patient-reported outcomes [PRO], questionnaire-based assessments of quality-of-life, daily activities and measures of neurological function (e.g. functional Magnetic Resonance Imaging (MRI)).

In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof 20%, at least 30%, at least 40%, at least 50%, at least 60%, at least 70%, at least 80%, at least 90%, at least 100%, at least 200%, at least 500%, or more compared to a comparable cell that is not contacted with the engineered polynucleotide, the AAV vector comprising the engineered polynucleotide, the AAV comprising the engineered polynucleotide, or the pharmaceutical composition.

In some embodiments, the method of treatment described herein can treat an ocular disease. Relevant ocular diseases and conditions can include but are not limited to: blindness, Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In an embodiment, the ocular disease or condition is AMD. AMD can be wet AMD or dry AMD.

In some cases, an administration of a pharmaceutical composition is sufficient to reduce at least a symptom of a disease or condition, treat the disease or condition, and/or eliminate the disease or condition. In some cases, improvements of diseases or conditions can be ascertained by any of the provided diagnostic assays. In other cases, an improvement can be obtained via an interview with the treated subject. For example, a subject may be able to communicate to an attending physician that their vision is improved as compared to their vision prior to administration of a subject pharmaceutical. In other cases, an in vivo animal model may be used to ascertain reduction of a disease or condition after treatment. Suitable animal models include mouse models, primate models, rat models, canine models, and the like.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “AAV,” “AAV construct,” or “recombinant AAV” or “AAV” refer to adeno-associated virus of any of the known serotypes, including AAV-1, AAV-2, AAV-3, AAV-4, AAV-5, AAV-6, AAV-7, AAV-8, AAV-9, AAV-10, AAV-11, AAV-12, AAV-13, or scAAV, rh10, chimeric or hybrid AAV, or any combination, derivative, or variant thereof. AAV is a small non-enveloped single-stranded DNA virus. They are non-pathogenic parvoviruses and can require helper viruses, such as adenovirus, herpes simplex virus, vaccinia virus, and CMV, for replication. Wild-type AAV is common in the general population and is not associated with any known pathologies. A hybrid AAV is an AAV comprising a capsid protein of one AAV serotype and genomic material from another AAV serotype. A chimeric AAV comprises genetic and/or protein sequences derived from two or more AAV serotypes and can include mutations made to the genetic sequences of those two or more AAV serotypes. An exemplary chimeric AAV can comprise a chimeric AAV capsid, for example, a capsid protein with one or more regions of amino acids derived from two or more AAV serotypes. An AAV variant is an AAV comprising one or more amino acid mutations in its genome or proteins as compared to its parental AAV, e.g., one or more amino acid mutations in its capsid protein as compared to its parental AAV. AAV, as used herein, includes avian AAV, bovine AAV, canine AAV, equine AAV, primate AAV, non-primate AAV, and ovine AAV, where primate AAV refers to AAV that infect non-primates, and where non-primate AAV refers to AAV that infect non-primate animals, such as avian AAV that infects avian animals. In some cases, the wild-type AAV contains rep and cap genes, where the rep gene is required for viral replication and the cap gene is required for the synthesis of capsid proteins. As used herein, the terms “recombinant AAV” and “rAAV” are interchangeable.

The terms “recombinant AAV vector” or “AAV vector” or “AAV vector” refer to a vector derived from any of the AAV serotypes mentioned above. In some cases, an AAV vector can comprise one or more of the AAV wild-type genes deleted in whole or part, such as the rep and/or cap genes, but contains functional elements that are required for packaging and use of AAV virus for gene therapy. For example, functional inverted terminal repeats or ITR sequences that flank an open reading frame or exogenous sequences cloned in are known to be important for replication and packaging of an AAV virion, but the ITR sequences can be modified from the wild-type nucleotide sequences, including insertions, deletions, or substitutions of nucleotides, so that the AAV is suitable for use for the embodiments described herein, such as a gene therapy or gene delivery system. In some aspects, a self-complementary vector (sc) can be used, such as a self-complementary AAV vector, which can bypass the requirement for viral second-strand DNA synthesis and can lead to higher rate of expression of a transgene protein. In some aspects, AAV vectors can be generated to allow selection of an optimal serotype, promoter, and transgene. In some cases, the vector can be targeted vector or a modified vector that selectively binds or infects immune cells.

The terms “AAV virion” or “AAV virion” refer to a virus particle comprising a capsid comprising at least one AAV capsid protein that encapsidates an AAV vector as described herein, where the vector can further comprise a heterologous polynucleotide sequence or a transgene in some embodiments. A virion can be an engineered virion.

The term “subject,” “host,” “individual,” and “patient” are as used interchangeably herein to refer to animals, typically mammalian animals. Any suitable mammal can be administered a composition as described herein (such as an engineered guide RNA) or treated by a method as described herein. A subject can be a vertebrate or an invertebrate. A subject can be a laboratory animal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

The term “protein”, “peptide”, and “polypeptide” are used interchangeably and in their broadest sense to refer to a compound of two or more subunit amino acids, amino acid analogs or peptidomimetics. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

Embodiment 1. An engineered polynucleotide comprising an AAV vector comprising one or more expression cassettes, wherein the one or more expression cassettes encode a peptide.

Embodiment 2. An engineered polynucleotide comprising an AAV vector comprising one or more expression cassettes, wherein the one or more expression cassettes encode an engineered polypeptide comprising: an antibody or fragment thereof operatively coupled to a peptide.

Embodiment 3. The engineered polynucleotide of Embodiment 1 or 2, wherein the AAV vector comprises an AAV serotype comprising AAV1, AAV2, AAV3, AAV4, AAV5, AAV6, AAV7, AAV8, AAV9, AAV10, AAV11, AAV12, or any combination thereof.

Embodiment 4. The engineered polynucleotide of Embodiment 3, wherein the AAV serotype comprises the AAV2.

Embodiment 5. The engineered polynucleotide of any one of Embodiments 1-4, wherein the peptide comprises a CNP.

Embodiment 6. The engineered polynucleotide of Embodiment 5, wherein the CNP comprises at least 22 amino acid residues.

Embodiment 7. The engineered polynucleotide of Embodiment 5, wherein the CNP comprises at least 36 amino acid residues.

Embodiment 8. The engineered polynucleotide of Embodiment 5, wherein the CNP comprises at least 53 amino acid residues.

Embodiment 9. The engineered polynucleotide of any one of Embodiments 6-8, wherein the CNP comprises a amino acid sequence that is at least 80% identical to SEQ ID NOs: 1-5.

Embodiment 10. The engineered polynucleotide of Embodiment 2, wherein the peptide is covalently connected to N terminus of the antibody or fragment thereof.

Embodiment 11. The engineered polynucleotide of Embodiment 2, wherein the peptide is covalently connected to C terminus of the antibody or fragment thereof.

Embodiment 12. The engineered polynucleotide of Embodiment 2, wherein the peptide is operatively coupled to the antibody or fragment thereof by a peptide linker.

Embodiment 13. The engineered polynucleotide of Embodiment 12, wherein the peptide linker comprises an amino acid sequence comprising (GGGGS)ₙ, wherein the ₙ is an integer between 0-10 (SEQ ID NO: 142).

Embodiment 14. The engineered polynucleotide of any one of Embodiments 1-4, wherein the AAV vector encodes an engineered AAV capsid.

Embodiment 15. An engineered polypeptide comprising an antibody, or a fragment thereof operatively coupled to a peptide, wherein the antibody or fragment thereof comprises an amino acid sequence that is at least 80% identical to any one of SEQ ID NOs: 6-8.

Embodiment 16. The engineered polypeptide of Embodiment 15, wherein the peptide comprises a CNP.

Embodiment 17. The engineered polypeptide of Embodiment 16, wherein the CNP comprises at least 22 amino acid residues.

Embodiment 18. The engineered polypeptide of Embodiment 16, wherein the CNP comprises at least 36 amino acid residues.

Embodiment 19. The engineered polypeptide of Embodiment 16, wherein the CNP comprises at least 53 amino acid residues.

Embodiment 20. The engineered polypeptide of any one of Embodiments 17-19, wherein the CNP comprises a amino acid sequence that is at least 80% identical to SEQ ID NOs: 1-5.

Embodiment 21. The engineered polypeptide of any one of Embodiments 15-20, wherein the peptide is covalently connected to N terminus of the antibody or fragment thereof.

Embodiment 22. The engineered polypeptide of any one of Embodiments 15-20, wherein the peptide is covalently connected to C terminus of the antibody or fragment thereof.

Embodiment 23. The engineered polypeptide of any one of Embodiments 15-22, wherein the peptide is operatively coupled to the antibody or fragment thereof by a peptide linker.

Embodiment 24. The engineered polypeptide of Embodiment 23, wherein the peptide linker comprises an amino acid sequence comprising (GGGGS)n, wherein the n is an integer between 0-10 (SEQ ID NO: 142).

Embodiment 25. An engineered polynucleotide encoding the engineered polypeptide of any one of Embodiments 15-24.

Embodiment 26. The engineered polynucleotide of Embodiment 25, wherein the engineered polynucleotide is a vector.

Embodiment 27. The engineered polynucleotide of Embodiment 26, wherein the vector is a viral vector.

Embodiment 28. The engineered polynucleotide of Embodiment 27, wherein the viral vector comprises an AAV vector.

Embodiment 29. The engineered polynucleotide of Embodiment 28, wherein the AAV vector comprises an AAV serotype comprising AAV1, AAV2, AAV3, AAV4, AAV5, AAV6, AAV7, AAV8, AAV9, AAV10, AAV11, AAV12, or any combination thereof.

Embodiment 30. The engineered polynucleotide of Embodiment 29, wherein the AAV serotype comprises the AAV2.

Embodiment 31. The engineered polynucleotide of any one of Embodiments 28-30, wherein the AAV vector encodes an engineered AAV capsid.

Embodiment 32. The engineered polynucleotide of any one of Embodiments 27-31, wherein the viral vector comprises one or more expression cassettes.

Embodiment 33. The engineered polynucleotide of Embodiment 2 or Embodiment 32, wherein the one or more expression cassettes encode a contiguous polypeptide, wherein the contiguous polypeptide comprises the engineered polypeptide of any one of Embodiments 2-33.

Embodiment 34. The engineered polynucleotide of Embodiment 33, wherein the contiguous polypeptide comprises a protease cleavable sequence.

Embodiment 35. The engineered polynucleotide of Embodiment 33, wherein the contiguous polypeptide comprises a Furin cleavable sequence.

Embodiment 36. The engineered polynucleotide of Embodiment 33, wherein the contiguous polypeptide comprises a self-cleaving polypeptide sequence.

Embodiment 37. The engineered polynucleotide of Embodiment 1, 2, or 32, wherein the one or more expression cassettes express at least one additional therapeutic.

Embodiment 38. The engineered polynucleotide of Embodiment 37, wherein the at least one additional therapeutic comprises a hormone.

Embodiment 39. The engineered polynucleotide of Embodiment 38, wherein the at least one additional therapeutic comprises an agonist of a natriuretic peptide receptor (NPR).

Embodiment 40. The engineered polynucleotide of Embodiment 38, wherein the at least one additional therapeutic comprises an agonist of a cyclic GMP (cGMP) signaling pathway.

Embodiment 41. The engineered polynucleotide of Embodiment 37, wherein the at least one additional therapeutic comprises an VEGF inhibitor.

Embodiment 42. The engineered polynucleotide of Embodiment 41, wherein the VEGF inhibitor binds to and inhibits VEGF-A, VEGF-B, VEGF-C, VEGF-D, or a combination thereof.

Embodiment 43. The engineered polynucleotide of Embodiment 41 or Embodiment 42, wherein the VEGF inhibitor comprises an antibody.

Embodiment 44. The engineered polynucleotide of Embodiment 43, wherein the VEGF inhibitor comprises a monovalent Fab′, a divalent Fab2, a F(ab)′3 fragments, a single-chain variable fragment (scFv), a bis-scFv, (scFv)2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof.

Embodiment 45. The engineered polynucleotide of Embodiment 41 or Embodiment 42, wherein the VEGF inhibitor comprises a non-antibody VEGF inhibitor.

Embodiment 46. The engineered polynucleotide Embodiment 45, wherein the non-antibody VEGF inhibitor is a VEGF receptor 1 (VEGFR1), a VEGF receptor 2 (VEGFR2), a VEGF receptor 3 (VEGFR3), a fragment thereof, or a combination thereof.

Embodiment 47. The engineered polynucleotide Embodiment 45, wherein the non-antibody VEGF inhibitor comprises a soluble VEGFR1, a soluble VEGFR2, a soluble VEGFR3, a soluble fragment thereof, or a combination thereof.

Embodiment 48. The engineered polynucleotide Embodiment 45, wherein the non-antibody VEGF inhibitor comprises a VEGF-Trap or a modified version thereof.

Embodiment 49. A cell comprising the engineered polynucleotide of any one of Embodiments 1-14 or 25-48.

Embodiment 50. A cell comprising the engineered polypeptide of any one of Embodiments 15-24.

Embodiment 51. A pharmaceutical composition comprising the engineered polynucleotide of any one Embodiments 1-14 or 25-48, the engineered polypeptide of any one of Embodiments 15-24, or the cell of Embodiment 49 or Embodiment 50.

Embodiment 52. The pharmaceutical composition of Embodiment 51, wherein pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Embodiment 53. The pharmaceutical composition of Embodiment 52, wherein the pharmaceutical composition is formulated for administering intravitreally, subretinally, or suprachoroidally.

Embodiment 54. The pharmaceutical composition of Embodiment 52, wherein the pharmaceutical composition is for treating an ocular disease or condition.

Embodiment 55. The pharmaceutical composition of Embodiment 52, wherein the pharmaceutical composition increases natriuretic peptide receptor-B signaling, guanylyl cyclase signaling, cyclic guanosine monophosphate (cGMP) signaling, or a combination thereof in a subject in need thereof.

Embodiment 56. A method of treating a disease or condition in a subject in need thereof, the method comprising administering to the subject a therapeutically effective amount of the engineered polynucleotide of any one Embodiments 1-14 or 25-48, the engineered polypeptide of any one of Embodiments 15-24, the cell of Embodiment 47 or Embodiment 48, or the pharmaceutical composition of Embodiments 49-52.

Embodiment 57. A method of treating a disease or condition in a subject in need thereof, the method comprising administering to the subject a therapeutically effective amount of the engineered polynucleotide of any one Embodiments 1-13 or 24-46, the engineered polypeptide of any one of Embodiments 14-23, the cell of Embodiment 49 or Embodiment 50, or the pharmaceutical composition of Embodiments 49-52, wherein once of the administering is curative of the disease or condition.

Embodiment 58. A method of treating a disease or condition in a subject in need thereof, the method comprising administering to the subject a therapeutically effective amount of the engineered polynucleotide of any one Embodiments 1-13 or 24-46, the engineered polypeptide of any one of Embodiments 14-23, the cell of Embodiment 47 or Embodiment 48, or the pharmaceutical composition of Embodiments 51-55, wherein the administering does not comprise daily administration.

Embodiment 59. The method of any one of Embodiment 56-58, wherein the disease or condition comprises an ocular disease.

Embodiment 60. The method of Embodiment 59, wherein the ocular disease comprises ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

### EXAMPLES

The following illustrative examples are representative of embodiments of the stimulation, systems, and methods described herein and are not meant to be limiting in any way.

### Example 1. Design and Experiment for Expressing CNP and CNP Fusion Protein

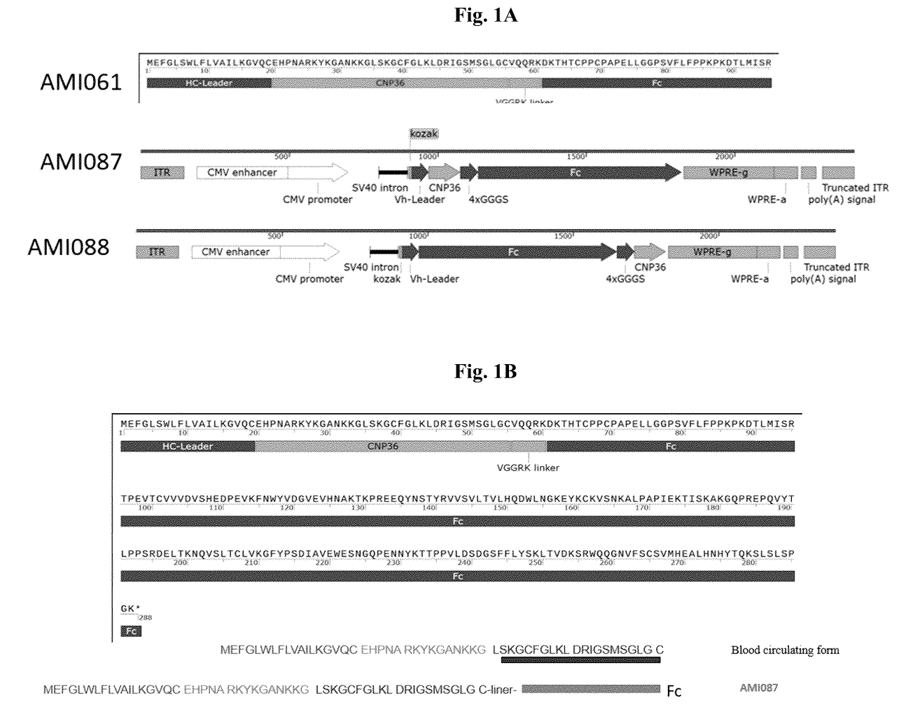

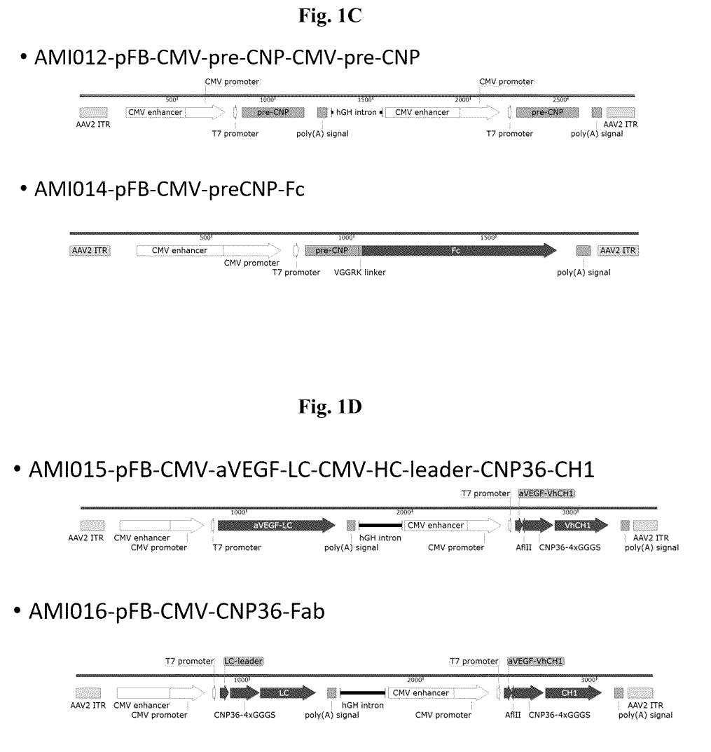

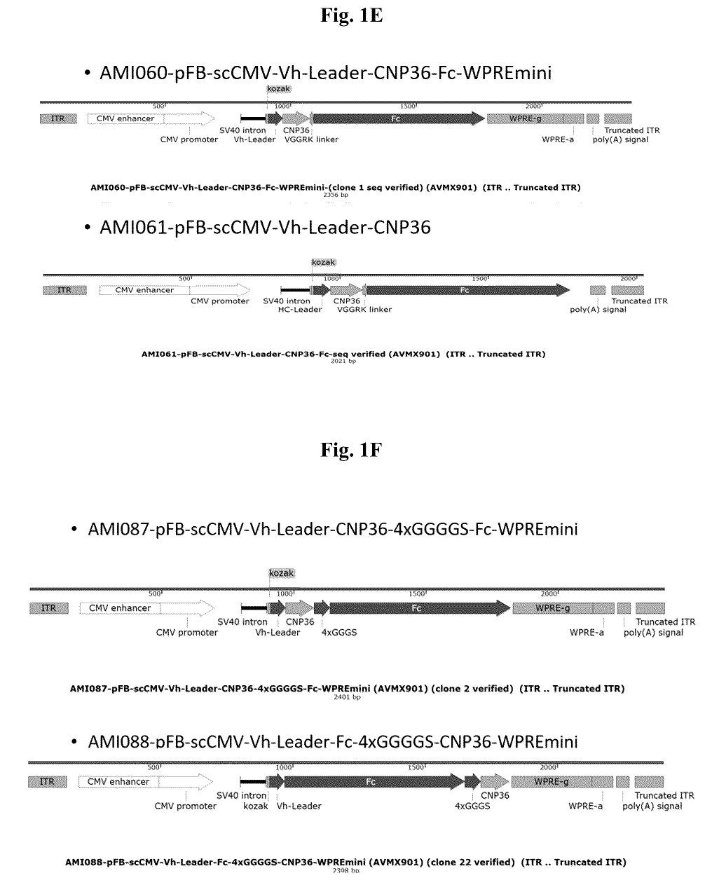

A series of CNP and CNP fusion protein (CNP fused to human antibody heavy chain secretion signal peptide at the 5′-end, IgG₁ Fc fragment, or IgG₄ Fc fragment at N-terminal or C-terminal of the CNP as illustrated in FIG. 3) were designed and tested. The designed CNP22 or CNP36 and CNP fusion proteins were reverse translated into DNA sequence with Homo sapiens codon output. The CNP and CNP fusion protein DNA sequence was further modified manually to adjust the GC content for synthesis as three overlapping DNA fragments to generate an AAV construct described herein (e.g., any one of the AAV construct in FIGS. 1-2).

For example, to create AMI061-pFB-scCMV-Vh-Leader-CNP36-Fc, AMI059 was first cut with StuI and SphI to remove the Aflibercept coding sequence and poly A signal. Then the codon-optimized Vh-Leader-CNP36-Fc fragment was PCR-amplified with primers A056, A057, A025 and AMI014 as template. Finally, the PCR fragment was assembled into the StuI and SphI sites of AMI059 to create AMI061 with the NEBuilder HiFi DNA Assembly kit. To create AMI087-pFB-scCMV-Vh-Leader-CNP36-4×GGGGS-Fc-WPREmini, AMI061 was first cut with SnaBI and SphI to remove partial CMV promoter and CNP36-4×GGGS-Fc fragment. Then the partial CMV promoter and CNP36-4×GGGGS fragment were amplified with primers A051 and A166 and AMI061 as template. The Fc fragment was amplified with primers A167 and A025 and AMI061 as template. These two fragments were joined together by PCR with primers A051 and A025 and assembled into the SnaBI and SphI sites of AMI061 with the NEBuilder HiFi DNA Assembly kit to create AMI087. To create AMI088-pFB-scCMV-Vh-Leader-Fc-4×GGGGS-CNP36-WPREmini, AMI060 AMI059 (was first cut with AflII and XhoI to remove the CNP-VGGRK-Fc fragment. Then the Fc fragment was amplified with primers A180 and A181 and the 4×GGGGS-CNP36 fragment with primers A182 and A183 and AMI060 as template. These two PCR fragments were joined together with primers A180 and A183 and finally assembled into the AflII and XhoI sites of AMI060 with the NEBuilder HiFi DNA Assembly kit to create AMI088. To create AMI182-pFB-scCMV-Vh-Leader-CNP36-2×STOP-4×GGGGS-Fc-WPREmini, the 4×GGGGS-Fc fragment was first PCR amplified with primers A618 and A619 and AMI087 as template. The PCR fragment was then assembled into the XcmI and EcoNI sites of AMI087 with the NEBuilder HiFi DNA Assembly kit to create AMI182. To create AMI183-pFB-scCMV-3×STOP-Vh-Leader-CNP36-4×GGGGS-Fc-WPREmini, AMI087 was first cut with StuI and EcoNI to remove the CNP and partial Fc coding sequence. Then the CNP and partial Fc fragment with 3 incorporated stop codons were amplified using primers A620, A621, A622, and A619, and AMI087 as template. Finally, the CNP and partial Fc fragment with 3 stop codons was assembled into the StuI and EcoNI sites of AMI087 to create AMI183 using the NEBuilder HiFi DNA Assembly kit. The identity of the AAV construct was confirmed by PCR amplification and sequencing analysis by utilizing the primers listed in Table 6.

TABLE 6
DNA primers used for PCR amplifications and DNA sequencing analyses

| | | SEQ | |
|---|---|---|---|
| | Primer | ID | |
| Item | ID | NO: | DNA Sequence |
| 1 | A025 | 152 | 5′-TGGGGTTGATCTCTCCCCAGCATGCCACACAAAAAACCAA-3′ |
| 2 | A051 | 153 | 5′-GGGACTTTCCTACTTGGCAGTACA-3′ |
| 3 | A056 | 154 | 5′-GTTGCCTTTACTTCTAGGCCTGCCGCCACCATGGAGTTCGGCCTGAGCTGGCTGTTCCT-3′ |
| 4 | A057 | 155 | 5′- |
| | | | GAGCTGGCTGTTCCTGGTGGCCATCCTTAAGGGCGTGCAGTGCGAGCACCCCAACGCGC-3′ |
| 5 | A166 | 156 | 5′-CCGCCTCCGCCAGATCCGCCTCCGCCGCTTCCGCCTCCGCCACATCCCAGGCCGCTCAT-3′ |
| 6 | A167 | 157 | 5′- |
| | | | GAGGCGGATCTGGCGGAGGCGGCAGCGGCGGCGGCGGCTCTGACAAAACTCACACATGC-3′ |
| 7 | A180 | 158 | 5′-TGTTCCTGGTGGCCATCCTTAAGGGCGTGCAGTGCGACAAAACTCACACATGCCC-3′ |
| 8 | A181 | 159 | 5′- |
| | | | CCGCCTCCGCCAGATCCGCCTCCGCCGCTTCCGCCTCCGCCTTTACCCGGAGACAGGGA-3′ |
| 9 | A182 | 160 | 5′- |
| | | | GGCGGATCTGGCGGAGGCGGCAGCGGCGGCGGCGGCTCTGAGCACCCCAACGCGCGCAA-3′ |
| 10 | A183 | 161 | 5′-TTGTAATCCAGAGGTTGATTCTCGAGTTAACATCCCAGGCCGCTCATGG-3′ |
| 11 | A618 | 162 | 5′-GAATCGGCTCCATGAGCGGCCTGGGATGTTAGGGAGGCGGAAGCGGCGG-3′ |
| 12 | A619 | 163 | 5′-TACTCCTTGCCATTCAGCTAGTCCTGGTGCAGGACGGTGAGGACGCTGA-3′ |
| 13 | A620 | 164 | 5′-GTTGCCTTTACTTCTAGGCCTGCCGCCACCTAGGAGTTCGGCCTGAGCT-3′ |
| 14 | A621 | 165 | 5′-CTAGGAGTTCGGCCTGAGCTGGCTGTTCCTGGTGGCCATCCTTAAGGGC-3′ |
| 15 | A622 | 166 | 5′-TGGCCATCCTTAAGGGCGTGCAGTGCTAGCACCCCAACGCGCGCAAAT-3′ |

### Generation of Recombinant Baculoviruses for AAV Vector Production

Recombinant baculoviruses (rBVs) were generated using the Bac-to-Bac Baculovirus Expression System according to the manufacturer's instruction. Briefly, the pFB shuttle plasmids containing the target genes were each diluted into 1 ng/μL in TE buffer, and 2 ng of each DNA was mixed with 20 μL of Δcath-DH10Bac competent bacteria containing a bacmid DNA molecule with the cathepsin gene deleted and incubated on ice for 30 min followed by heat-shock at 42° C. for 30 seconds. After incubating on ice for 2 minutes, the bacteria were cultured at 37° C. for 4 hours to recover and then plated on agar plates containing 50 μg/mL of kanamycin, 7 μg/mL of gentamycin, 10 μg/mL of tetracycline, 40 μg/mL of IPTG, and 100 μg/mL of X-gal. After 48 hours of incubation at 37° C., 2 white colonies containing the recombinant bacmid DNAs were picked and miniprep bacmid DNAs purified under sterile condition. About 5 μg of each bacmid DNA and 10 μL of GeneJet Reagent (SignaGen Laboratories, Fredrick, MID) were respectively diluted in 100 μL ESFAF media and then mixed together for about 30 min to form the transfection mixture. Sf9 cells were plated in a 6-well plate at 1.5e+6 cells/well in 2 mL ESFAF media at 28° C. for about 30 minutes. After removing the old media from the Sf9 cells, each transfection mixture was diluted in 800 μL ESFAF media and then added to the Sf9 cells. After incubation at 28° C. overnight, each well was added with additional 1 mL ESFAF media. After a total incubation time of 4 days, media containing the rBVs were collected and amplified at 1:200 ratio to generate sufficient quantity of rBVs ready for use in the AAV production process.

### AAV Production and Purification

The rBVs carrying the AAV2 Rep and mutant capsid genes and the target expression cassettes respectively were used to co-infect Sf9-V432AG cells for AAV production. Briefly, 10 moi of rBV-Cap-Rep and 5 moi of rBV-target cassettes were used to co-infect the Sf9 cell line at density of ˜-5e+6 cells/mL with 50% fresh ESFAF media for 3 days at 28° C. with shaking speed of 180 revolution per minute (rpm) in a shaker incubator. At the end of infection, cell pellets were collected by centrifugation at 3,000 rpm for 10 min. The cells were lysed in Sf9 lysis buffer containing 50 mM Tris-HCl, pH 8.0, 2 mM MgCl₂, 1% Sarkosyl, 1% Triton X-100, and 125 units/mL Benzonase with vigorous vortex followed by shaking at 350 rpm, 37° C. for 1 hour. At the end of shaking, salt concentration was increased to 500 mM by vortexing and the lysates were cleared by centrifugation at 8,000 rpm for 20 minutes at 4° C. The cleared lysates were transferred to ultraclear centrifuge tubes for SW28 swing bucket rotor which contain 5 mL of 1.50 g/cc and 10 mL of 1.30 g/cc cesium chloride solutions. After centrifugation at 28,000 rpm, 15° C. for ˜18 hours, the AAV bands were collected with syringes and transferred to ultraclear centrifuge tubes for the 70 ti centrifuge rotor. The centrifuge tubes were filled with 1.38 g/cc cesium chloride solution and heat-sealed. The AAV samples were subjected to a second round of ultracentrifugation at 65,000 rpm, 15° C. for ˜18 hours and AAV bands were collected with syringes. The purified AAV samples were buffer-exchanged into PBS buffer containing 0.001% Pluronic F-68 and filter-sterilized with 0.22 μm syringe filters. The sterilized AAV samples were stored at 4° C. within a month and then transferred to −80° C. for long term storage. AAV titer was determined with real-time PCR method.

### Transient Expression of the Constructs in Mammalian Cell Culture System

Human HEK293 cells were cultured in DMEM medium with 10% FBS in a CO₂ incubator at 37° C. For maintenance passage, cells were split 1:10 twice a week. For transfection, cells were seeded on 10-cm cell culture dish at 2×10⁶ cells/dish in 10 mL media overnight. 14 μg of plasmid DNA and 22 μL of Lipofectamine 3000 were each diluted in 0.5 mL of Opti-medium and mixed together. After incubation at room temperature for 5 minutes, the mixture was added to the cells dropwise and incubated at 37° C. in the CO₂ incubator for 48 hours. Medium was harvested for further experiments.

HEK293 cells were seeded onto 10 cm tissue culture dishes at a density 2×10⁶ one day prior to transient transfection. Each transfection of CNP22 or CNP36 or CNP fusion variant plasmid was performed using 14 μg/dish DNA with Lipofectamine 3000 reagent following the manufacturer's protocol. Cell culture supernatants were collected and analyzed for protein expression by western blot, at 48-hour post-transfection. All transfections were performed in triplicate in at least three independent experiments.

CNP fusion (either (CNP-Fc or CNP-Fc) variant proteins were determined by the SDS-PAGE and western blot analysis. HEK293 cell media (supernatants) was collected 48 hours or 72 hours after vector transduction. A total volume of 30 μL of cell supernatants was mixed with 10 ul of 4× loading buffer and loaded onto the NuPAGE 10% Tris-Glycine gels for electrophoresis. Proteins were subsequently transferred onto PVDF membranes. Membranes were treated with casein blocker in PBS for at least one hour at room temperature and probed with the goat anti-human IgG₁ Fc antibody biotin conjugate followed by incubation with streptavidin conjugated with horseradish peroxidase.

### Purification of CNP and CNP Fusion Proteins

All functional protein sequences are converted into DNA sequence and cloned into plasmids for expression and cloned into baculovirus vector for recombinant AAV packaging. The expressible plasmids were amplified, and DNA preparation were made and used for transfection transiently to HEK293 cells. Cell culture harvests were used for purification of CNP36-Fc proteins by protein A affinity column chromatography. These fusion proteins were purified to homogeneity and characterized for purity by SDS-PAGE.

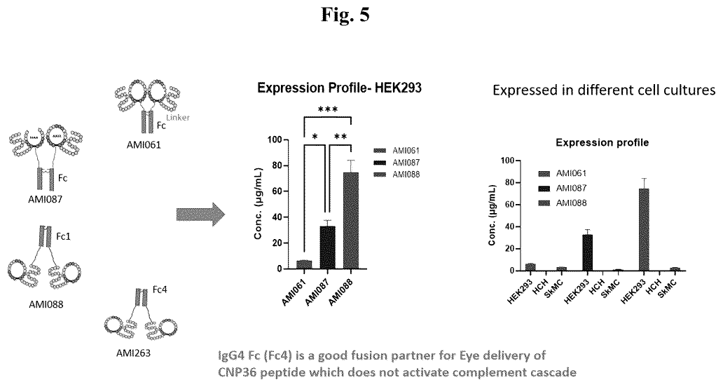

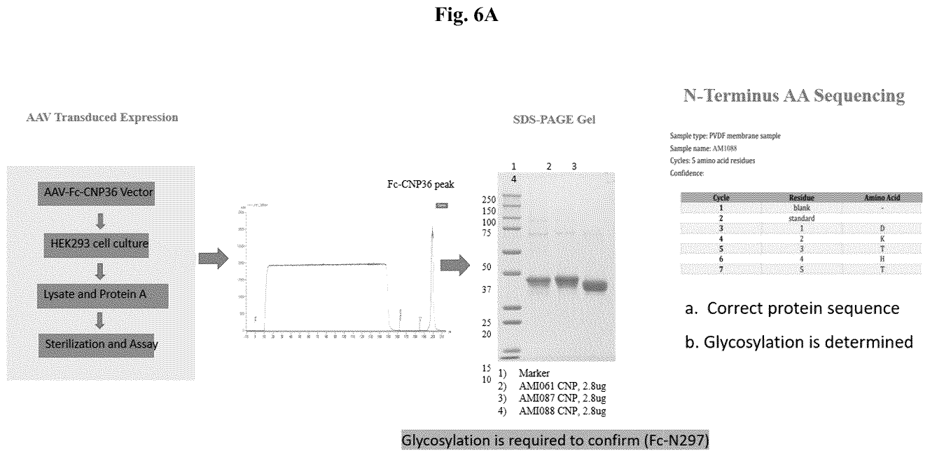

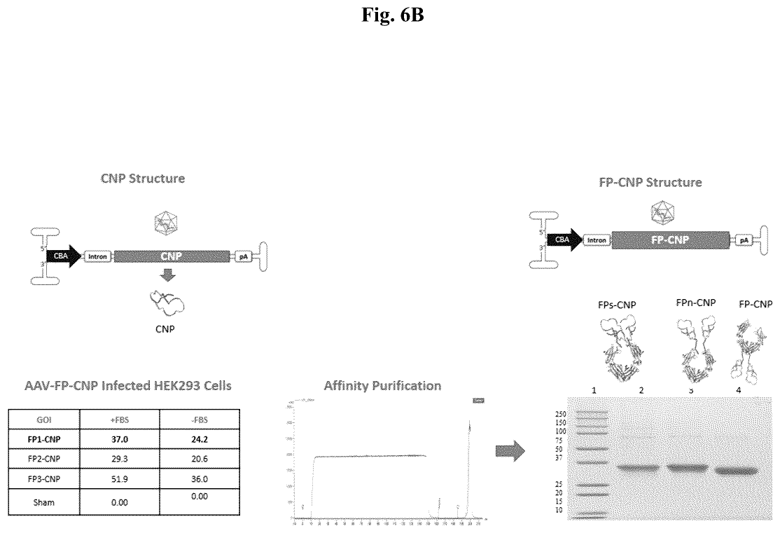

CNP, CNP-Fc, or CNP-Fc proteins expressed were purified from HEK293 cell culture harvests by protein A affinity column chromatography. The harvested serum-free media were filtered with 0.2 μm filter to remove particulates and loaded on to the protein A column (1-mL size) at a flow rate of 1.5˜2.0 mL/min. The column was washed with wash buffer (20 mM Tris-HCl, pH 7.3, 150 mM NaCl, 5 mM EDTA), eluted with elution buffer (0.1 M glycine, pH 2.5), and neutralized with 1/10 of the neutralization buffer (1.0 M Tris-HCl, pH 10) to pH 6.8-7.4. The neutralized protein was buffer exchanged to 1×PBS and filter sterilized with 0.2 μm syringe filter pre-wet with PBS and stored at −80° C. A. The column chromatogram showed a sharp peak of eluate CNP fusion protein off the column when pH reached 3-4.0 (FIG. 6A). Similar observations of FP-CNP expression were made based on analysis of chromatogram and SDS-PAGE (FIG. 6B). Protein concentration of each preparation was determined by the BCA protein assay (FIG. 5). Table 7 shows the correct size of either CNP-Fc or CNP-Fc expressed by the transduced HEK293 cells. The N-terminus sequence of CNP-Fc protein (AMI088) was determined by Edman degradation (FIG. 6A). The purified protein preparation was used for in vitro biofunction assays. The protein AMI263 was also used in the optic nerve crush (ONC) evaluation for its role in protection of retinal ganglion cells (RGC) from the detriment damage (Table 8).

TABLE 7
Summary of CNP-Fc Proteins Purified from HEK 293 Cell
Culture Supernatant

| | | | Theoretical | |
|---|---|---|---|---|
| Protein | | Monomeric | Isoelectric | |
| code | Protein | MW | point (pI) | AAV Code |
| AMI061 | CNP36-VGGRK- | 28227.18 | 8.54 | AAV2.N54-061 |
| | Fc1 | | | |
| AMI087 | CNP36-(G4S)4-Fc1 | 30613.57 | 8.78 | AAV2.N54-087 |
| AMI088 | Fc1-(G4S)4-CNP36 | 30613.57 | 8.78 | AAV2.N54-088 |
| AMI263 | Fc4--(G4S)4-CNP36 | 31338.31 | 8.52 | AAV2.N54-263 |
| AMI269 | Fc4--(G4S)4-CNP22 | 29715.46 | 7.09 | AAV2.N54-269 |

Fc1 = IgG1 Fc;
Fc4 = IgG4 Fc

TABLE 8
CNP-Fc Proteins Usage

| | | Protein | |
|---|---|---|---|
| Protein | | concentration | |
| code | Protein | (mg/mL) | Usage |
| AMI061 | CNP36-VGGRK- | 1.8 | In vitro function |
| | Fc1 | | |
| AMI087 | CNP36-(G4S)4-Fc1 | 8.2 | In vitro function |
| AMI088 | Fc1-(G4S)4-CNP36 | 7.0 and 8.3 | In vitro & in vivo function |
| AMI263 | Fc4--(G4S)4-CNP36 | 40 | In vitro & in vivo function |
| AMI269 | Fc4--(G4S)4-CNP22 | | In vitro function |

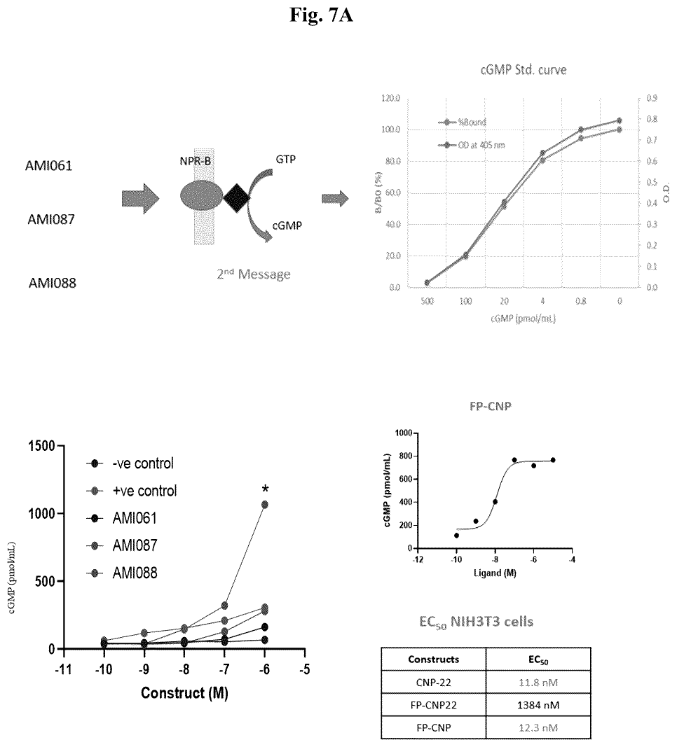

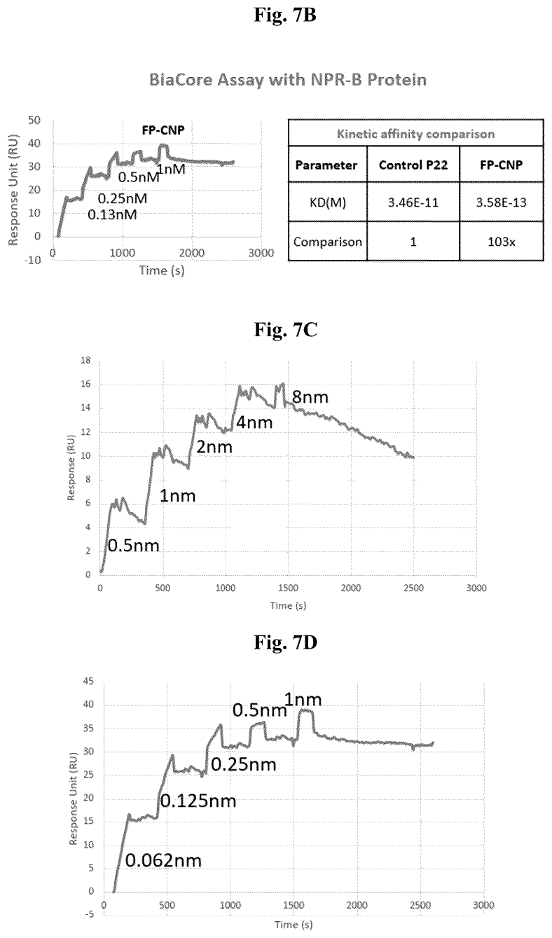

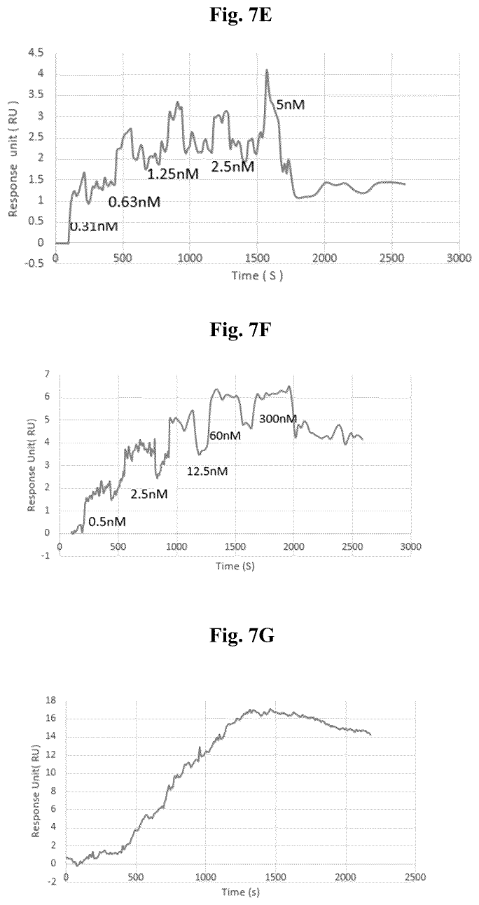

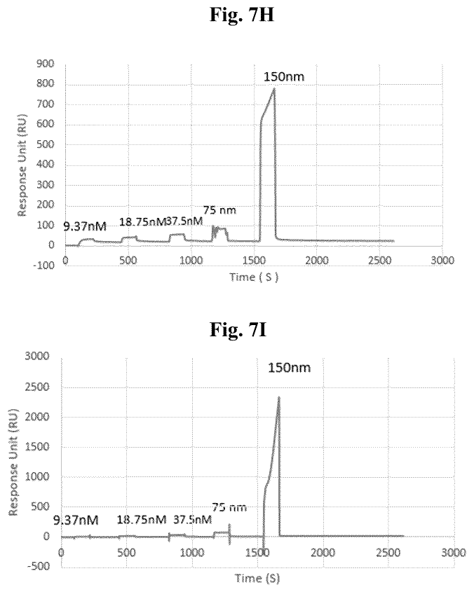

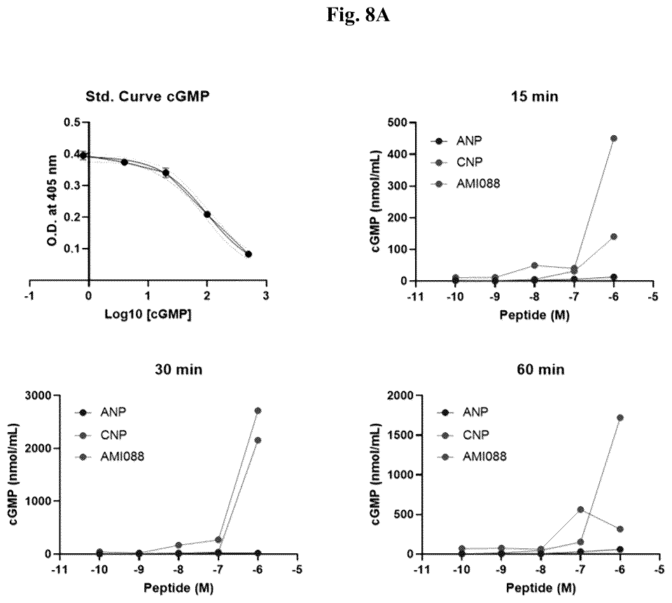

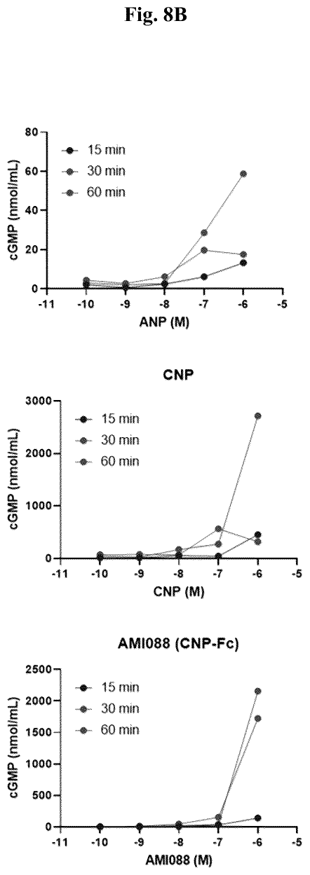

The biological function of these purified protein was assayed for their stimulation of production of cGMP, using NPR-B receptor positive cell line, NIH/3T3 and NPR-B negative cell line HEK293 cells. The determination of cGMP was assay by ELISA using commercial CNP as standard curves. FIG. 7A and FIGS. 8A-B (time course) illustrate the simulation of cGMP produced by CNP-Fc36. FIG. 7A illustrates cGMP release stemmed from a CNP fusion described herein binding to NPR-B. FIG. 7B illustrates kinetic affinity binding between a CNP fusion described herein and NPR-B protein as measured by BiaCore assay. Kinetic affinity binding analysis was conducted at 25° C. in HBS running buffer (20 mM NaH2PO4-Na2HPO4·H2O, 150 mM NaCl, pH 7.4), supplemented with 0.005% (v/v) surfactant P20, using a Biacore 3000 optical biosensor equipped with a research-grade CM5 sensor chip. Surface preparation. NBR-B was immobilized at different levels in three flow cells via standard amine coupling, leaving Fc 1 unmodified to serve as a reference. All surface plasmon resonance (SPR) analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 6600 RU was used for measurements with natriuretic peptides Sequential injections of CNP (0.5-8 nM) were performed at a flow rate of 30 l/min (300 s for each), followed by a dissociation time of 900 s. Binding site saturation was observed, and the surface was regenerated by 2 injections of 0.5M NaCl (60 s each). Kinetic parameters were calculated assuming a simple 1:1 (Langmuir) binding. FIG. 7C illustrates single-cycle kinetics (SCK) assay of binding between CNP and NPR-B (KD=36.3 Pm; ka=1.31e7; and kd=4.74E-4). FIG. 7D illustrates Biacore assay for AMI263 (Fc4-CNP36) against NPR-B (KD=0.36 pM; ka=1.38E6; and kd=4.92E-6). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI263 (FC4-CNP) (0.125-2 nM) were performed at a flow rate of 30 l/min (300 s for each), followed by a dissociation time of 900 s. Binding site saturation was observed. Kinetic parameters were calculated assuming a simple 1:1 (Langmuir) binding. FIG. 7E illustrates Biacore assay for AMI088 against NPR-B (KD=34.1 pM; ka=1.37E7; and kb=4.47E-4). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI088 (FC1-CNP) (0.31-5 nM) were performed at a flow rate of 30 l/min (300 s for each), followed by a dissociation time of 900 s. Binding site saturation was observed. Kinetic parameters were calculated assuming a simple 1:1 (Langmuir) binding. FIG. 7F illustrates Biacore assay for ANP against NPR-B (KD=52.3 nM; ka=2.36E3; and kd=1.24E-4). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI263 (FC4-CNP) (0.5-300 nM) were performed at a flow rate of 30 l/min (300 s for each), followed by a dissociation time of 900 s. Binding site saturation was observed. Kinetic parameters were calculated assuming a simple 1:1 (Langmuir) binding. FIG. 7G illustrates Biacore assay for BNP against NPR-B (KD=664 pM; ka=9.4E5; and kb=6.24E-4). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI263 (FC4-CNP) (0.5-300 nM) were performed at a flow rate of 30 l/min (300 s for each), followed by a dissociation time of 900 s. Binding site saturation was observed. Kinetic parameters were calculated assuming a simple 1:1 (Langmuir) binding. FIG. 7H illustrates Biacore assay for AMI263 against NPR-B (KD=13.1 nM; ka=1.14E4; and kd=1.49E-3). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI263 (FC4-CNP) (9.37-150 nM) were performed at a flow rate of 30 l/min (300 s for each), followed by a dissociation time of 900 s. Binding site saturation was observed. Kinetic parameters were calculated assuming a simple 1:1 (Langmuir) binding. FIG. 7I illustrates Biacore assay for AMI087 against NPR-B (KD=41.9 nM; ka=3.65E3; and kd=1.53E-3). All SPR analysis was performed on a BIAcore 3000 system using series CM5 sensor chips. Data processing and analysis were performed using BIAevaluation software. All sensorgrams were double referenced by subtracting the response on a reference flow cell and a blank sample. Human NPR-B (R&D system) was covalently attached to a CM5 chip via amine coupling. A surface density of 3100 RU was used for measurements with natriuretic peptides Sequential injections of AMI087 (FC4-CNP) (9.37-150 nM) were performed at a flow rate of 30 μl/min (300 s for each), followed by a dissociation time of 900 s. Binding site saturation was observed. Kinetic parameters were calculated assuming a simple 1:1 (Langmuir) binding. Table 32 illustrates summary of affinity between NPR-B and CNP or CNP fusion described herein. Table 33 illustrates corrected concentration of proteins for Biacore assay.

TABLE 32
Summary of affinity between NPR-B and CNP or CNP fusion

| Molecule | ka | kd | KD (M |
|---|---|---|---|
| CNP-22 | 1.37E+07 | 4.74E−04 | 3.46E−11 |
| ANP | 2.36E+03 | 1.24E−03 | 5.25E−07 |
| BNP | 9.40E+05 | 6.24E−04 | 6.64E−10 |
| AMI088 (Fc1-4xGGGS-CNP36) | 4.09E+07 | 2.96E−05 | 6.95E−13 |
| AMI087 (CNP36-4XGGGS-FC1) | 3.65E+03 | 1.53E−03 | 4.19E−07 |
| AMI061 (CNP36-Fc1 shorter linker) | 1.14E+04 | 1.49E−03 | 1.31E−07 |
| AMI263 (Fc4-4GGGS-CNP36) | 1.38E+08 | 4.92E−5 | 3.58E−13 |

TABLE 33
Corrected concentration of proteins for Biacore assay

| | | | | | | ul added to | | Corrected |
|---|---|---|---|---|---|---|---|---|
| | | | Concen- | | | make 500 | | concen- |
| | | MW KD | tration | | | ul of 100 | dimer | tration |
| | | (Monomer) | (mg/ml) | umol/ml | nmol/ml | nMol/ml | MW(KD) | (nMol/ml) |
| CNP-22: | CNP-22 | 2.197 | 1 | 0.4551661 | 455.1661356 | 109.85 | | 100 |
| 1 mg/mL | | | | | | | | |
| ANP: | ANP-28 | 3.08 | 1 | 0.3246753 | 324.6753247 | 154 | | 100 |
| 1 mg/mL | | | | | | | | |
| BNP: | BNP-32 | 3.464 | 1 | 0.2886836 | 288.6836028 | 173.2 | | 100 |
| 088 | 88 | 30.613 | 8.3 | 0.2711266 | 271.1266455 | 184.4156627 | 60.213 | 50.8411805 |
| (Fc1-CNP36): | | | | | | | | |
| 8.3 mg/mL | | | | | | | | |
| 087 | 87 | 30.617 | 8.2 | 0.2678251 | 267.8250645 | 186.6890244 | 60.221 | 50.8410687 |
| (CNP36-F1): | | | | | | | | |
| 8.2 mg/mL | | | | | | | | |
| 061 | 61 | 28.839 | 1.8 | 0.0624155 | 62.41547904 | 801.0833333 | 56.665 | 50.8938498 |
| (CNP36-Fc1 | | | | | | | | |
| short | | | | | | | | |
| linker): | | | | | | | | |
| 1.8 mg/mL | | | | | | | | |
| 263 | 263 | 31.338 | 15.7 | 0.5009892 | 500.9892144 | 99.80254777 | 61.663 | 50.8214002 |
| (Fc4-CNP36): | | | | | | | | |
| 15.7 mg/ml | | | | | | | | |

Stimulation of Cyclic Guanynyl Monophosphate (cGM)) Production by CNP

Purified CNP fusion proteins were evaluated for the biological function in a cell-based assays with natrieuretic peptide type C receptor B (NPR-B) positive cell line, NIH/3T3 and the negative cell line of HEK293 cells. The test were performed also in the presence of natrieuretic peptide A (ANP) and CNP22 controls (FIGS. 7A-B and FIGS. 8A-B).

### Production and Delivery of Adeno Associated Viral (AAV) Vector

Several AAV2.N54-CNP and AAV2.N54-CNP-FC constructs were produced for delivering various forms of CNP22, CNP36, or CNP fusion (CNP fused to either N-terminus or C-terminus of Fc fragment). AAV2.N54-CNP and AAV2.N54-CNP-FC construct was able to transduce airway epithelia cells by sinus, nose, and/or lung delivery methods. Other serotypes of AAV can also be used dependent on the target tissues or cells to be delivered. For example, AAV6 has a tendency to preferentially transduce lung cells.

The Sf9 derived insect cell line, V432A cells were cultured in storage bottles at 28° C. in ESF AF medium supplemented with 100 units/ml penicillin and 100 μg/ml streptomycin. The cells were split 1:4 once the cell density reached 7×10⁶ cells/ml for maintenance. Recombinant baculovirus (rBVs) were generated according to manufacturer's protocol. Briefly, the constructs were used to transduce DH10Bac, and recombinant bacmid DNAs were isolated. The bacmid DNAs were transduced into V432A cells to generate rBVs. The rBVs were quantified with qPCR. Table 9 lists exemplary AAV constructs encoding CNP or CNP fusion proteins. Table 10 lists the amino acid sequence of the CNP and CNP fusion protein used in the experiments of Examples 1-3. Table 11 lists the nucleic acid sequence of AAV construct encoding the CNP and CNP fusion protein used in the experiments of Examples 1-3.

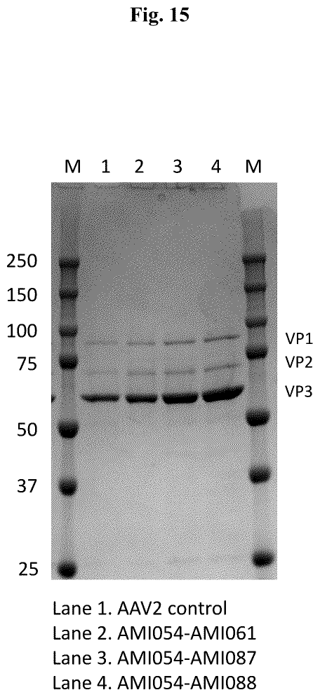

The purity of AAV vectors was determined by SimplyBlue Staining assay. Briefly, 26 μl AAV samples were mixed with 10 μL of 4× loading buffer plus 4 μL 10× reducing reagent and incubated at 95° C. for 2 min. About 1E+11 vg of each AAV sample was loaded on each lane of a 10% SDS-PAGE gel and ran at 100 volts until the dye reached the bottom of the gel. The gel was stained according to the manufacturer's protocol. A SDS-PAGE gel pattern was obtained with expected VP1, VP2 and VP3 component levels (FIG. 15).

The AAV2.N54-CNP or AAV2.N54-CNP-FC construct listed Table 2 were further evaluated for production of each construct protein using suspension HEK293 cell cultures. V432A cells were cultured to 7×10⁶ cells/ml and diluted 1:1 with fresh ESF AF media. About 200 virus per cell of rBV containing the designated rep-cap genes and 100 virus per cell of rBV containing the DNA sequences encoding CNP-Fc or CNP-Fc proteins was added separately to infect the V432A cells for 3 days at 28° C. in shaker incubator. The infected V432A cells were harvested by centrifugation at 3,000 rpm for 10 minutes. Cell pellets were lysed in SF9 lysis buffer (50 mM Tris-HCl, pH7.8, 50 mM NaCl, 2 mM MgCl2, 1% Sarkosyl, 1% Triton X-100, and 140 units/ml Benzonase®. Genomic DNA was digested by incubation at 37° C. for one hour. At the end of incubation, sodium chloride was added to adjust the salt concentration of the lysate to about 1 M to further dissociate the AAV vectors from cell matrix. Cell debris was removed by centrifugation at 8,000 rpm for 30 minutes. The cleared lysates were loaded onto CsCl step-gradient and subjected to ultracentrifugation at 28,000 rpm for 20 hours in swing bucket rotors. The viral band was drawn through a syringe with an 18-gauge needle and loaded onto a second CsCl gradient and subjected to linear-ultracentrifugation at 65,000 rpm for 20 hours. Then, the viral band was drawn and passed through two PD-10 desalting columns to remove the CsCl and detergents and at the same time exchanged to Buffer B (1×PBS, 0.1 M Sodium Citrate, and 0.001% pluronic F-68). Quantitative real-time PCR (qPCR) was performed to determine the AAV vector genome copy numbers with ITR primers and probe (Table 12).

TABLE 9
Summary of AAV constructs encoding various forms of CNP
and CNP fusion proteins

| Construct | Vector | | | In | In |
|---|---|---|---|---|---|
| ID | type | AAV | Protein | vitro | vivo |
| AMI061 | CMV- | AAV2.N54- | CNP36- | cGMP | No |
| | scAAV | 061 | VGGRK-Fc1ª | | |
| AMI087 | CMV- | AAV2.N54- | CNP36-(G4S)4-Fc1 | cGMP | No |
| | sCAAV | 087 | | | |
| AMI088 | CMV- | AAV2.N54- | Fc1-(G4S)4-CNP36 | cGMP | NMDA |
| | sCAAV | 088 | | | |
| AMI169 | CMV- | AAV2.N54- | CNP36 Furin 2A- | | NMDA |
| | scAAV | 169 | Anti-VEGF-A-ScFV | | |
| AMI182 | CMV- | AAV2.N54- | CNP36 | cGMP | NMDA |
| | sCAAV | 182 | | | |
| AMI189 | CMV- | AAV2.N54- | Sham AMI263 | NA | NMDA |
| | scAAV | 189 | | | |
| AMI263 | CMV- | AAV2.N54- | Fc4-(G4S)4-CNP36 | cGMP | NOC |
| | sCAAV | 263 | | | |
| AMI269 | CMV- | AAV2.N54- | Fc4-(G4S)4-CNP22 | cGMP | NMDA |
| | sCAAV | 269 | | | |
| AMI270 | CMV- | AAV2.N54- | CNP22-(G4S)4-Fc4^{b} | cGMP | No |
| | sCAAV | 270 | | | |
| AMI273 | CBA- | AAV2.N54- | Fc4-(G4S)4-CNP36 | NA | NOC |
| | scAAV | 273 | | | |
| AMI302 | CBA- | AAV2.N54- | CNP36 | NA | NOC |
| | scAAV | 302 | | | |
| AMI303 | CBA- | AAV2.N54- | CNP36 | NA | NOC |
| | ssAAV | 303 | | | |
| AMI304 | CBA- | AAV2.N54- | Fc4-(G4S)4-CNP36 | NA | NOC |
| | ssAAV | 304 | | | |
| AMI305 | CBA- | AAV2.N54- | Sham CBA-AMI305 | NA | NOC |
| | ssAAV | 305 | | | |
| AMI315 | CBA- | AAV2.N54- | Anti-VEGF-A- | cGMP | NMDA |
| | ssAAV | 315 | ScFV-CNP36 | | |

sc: self-complementary,
ss: single stranded;
ªIgG1 Fc,
^{b}IgG4 Fc

TABLE 10
Amino acid sequence of CNP and CNP Fusion Protein

| Clone | | |
|---|---|---|
| ID | Protein | Protein Sequence |
| AMI061 | CNP36- | GLSKGCFGLKLDRIGSMSGLGCVGGRKDKTHTCPPCPAP |
| (Seq ID | VGGRK-Fc1 | ELLGGPSVFLFPPKPKDTLMISRTPEVTCVVV |
| NO: | | DVSHEDPEVKFNWYVDGVEVHNAKTKPREE |
| 131) | | QYNSTYRVVSVLTVLHQDWLNGKEYKCKVS |
| | | NKALPAPIEKTISKAKGQPREPQVYTLPPSRD |
| | | ELTKNQVSLTCLVKGFYPSDIAVEWESNGQPE |
| | | NNYKTTPPVLDSDGSFFLYSKLTVDKSRWQQ |
| | | GNVFSCSVMHEALHNHYTQKSLSLSPGK |
| AMI087 | CNP36- | EHPNARKYKGANKKGLSKGCFGLKLDRIGSMSGLGCGGGGSG |
| (Seq ID | (G4S)4-Fc1 | GGGSGGGGSGGGGSDKTHTCPPCPAPELLGGPSVF |
| NO: | | LFPPKPKDTLMISRTPEVTCVVVDVSHEDPEV |
| 132) | | KFNWYVDGVEVHNAKTKPREEQYNSTYRVV |
| | | SVLTVLHQDWLNGKEYKCKVSNKALPAPIEK |
| | | TISKAKGQPREPQVYTLPPSRDELTKNQVSLT |
| | | CLVKGFYPSDIAVEWESNGQPENNYKTTPPV |
| | | LDSDGSFFLYSKLTVDKSRWQQGNVFSCSV |
| | | MHEALHNHYTQKSLSLSPGK |
| AMI088 | Fc1-(G4S)4- | DKTHTCPPCPAPELLGGPSVFLFPPKPKDTLM |
| (Seq ID | CNP36 | ISRTPEVTCVVVDVSHEDPEVKFNWYVDGVE |
| NO: | | VHNAKTKPREEQYNSTYRVVSVLTVLHQDWL |
| 133) | | NGKEYKCKVSNKALPAPIEKTISKAKGQPREP |
| | | QVYTLPPSRDELTKNQVSLTCLVKGFYPSDIA |
| | | VEWESNGQPENNYKTTPPVLDSDGSFFLYSK |
| | | LTVDKSRWQQGNVFSCSVMHE |
| | | ALHNHYTQKSLSLSPGKGGGGSGGGGSGGGGSGGGGSEHPNA |
| | | RKYKGANKKGLSKGCFGLKLDRIGSMSGLGC |
| AMI169 | Anti-VEGF- | EHPNARKYKGANKKGLSKGCFGLKLDRIGSMSGLGCRRKRAP |
| (Seq ID | A-ScFV- | VKQTLNFDLLKLAGDVESNPGPMEFGLSWLFLVAILKGVQCEV |
| NO: | CNP36 | QLVESGGGLVQPGGSLRLSCAASGYDFTHYGMNWVRQAPGK |
| 134) | | GLEWVGWINTYTGEPTYAADFKRRFTFSLDTSKSTAYLQMNSL |
| | | RAEDTAVYYCAKYPYYYGTSHWYFDVWGQGTLVTVGGGGSG |
| | | GGGSGGGGSGGGGSDIQLTQSPSSLSASVGDRVTITCSASQDISN |
| | | YLNWYQQKPGKAPKVLIYFTSSLHSGVPSRFSGSGSGTDFTLTIS |
| | | SLQPEDFATYYCQQYSTVPWTFGQGTKVEIKRTVAA |
| AMI182 | CNP36 | EHPNARKYKGANKKGLSKGCFGLKLDRIGSM |
| (Seq ID | | SGLGC |
| NO: 4) | | |
| AMI189 | Sham | None encoded |
| | AMI263 | |
| AMI263 | Fc4-(G4S)4- | DKRVESKYGPPCPPCPAPEFLGGPSVFLFPPKPKDTLMISRTPEV |
| (Seq ID | CNP36 | TCVVVDVSQEDPEVQFNWYVDGVEVHNAKTKPREEQFNSTYR |
| NO: | | VVSVLTVLHQDWLNGKEYKCKVSNKGLPSSIEKTISKAKGQPR |
| 135) | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMHE |
| | | ALHNHYTQKSLSLSLGKGGGGSGGGGSGGGGSGGGGSEHPNA |
| | | RKYKGANKKGLSKGCFGLKLDRIGSMSGLGC |
| AMI269 | Fc4-(G4S)4- | DKRVESKYGPPCPPCPAPEFLGGPSVFLFPPKPKDTLMISRTPEV |
| (Seq ID | CNP22 | TCVVVDVSQEDPEVQFNWYVDGVEVHNAKTKPREEQFNSTYR |
| NO: | | VVSVLTVLHQDWLNGKEYKCKVSNKGLPSSIEKTISKAKGQPR |
| 136) | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMHE |
| | | ALHNHYTQKSLSLSLGKGGGGSGGGGSGGGGSGGGGSGLSKG |
| | | CFGLKLDRIGSMSGLGC |
| AMI270 | CNP22- | GLSKGCFGLKLDRIGSMSGLGCGGGGSGGGGSGGGGSGGGGS |
| (Seq ID | (G4S)4-Fc4 | DKRVESKYGPPCPPCPAPEFLGGPSVFLFPPKPKDTLMISRTPEV |
| NO: | | TCVVVDVSQEDPEVQFNWYVDGVEVHNAKTKPREEQFNSTYR |
| 137) | | VVSVLTVLHQDWLNGKEYKCKVSNKGLPSSIEKTISKAKGQPR |
| | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMHE |
| | | ALHNHYTQKSLSLSLGK |
| AMI273 | Fc4-(G4S)4- | DKRVESKYGPPCPPCPAPEFLGGPSVFLFPPKPKDTLMISRTPEV |
| (Seq ID | CNP36 | TCVVVDVSQEDPEVQFNWYVDGVEVHNAKTKPREEQFNSTYR |
| NO: | | VVSVLTVLHQDWLNGKEYKCKVSNKGLPSSIEKTISKAKGQPR |
| 138) | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMHE |
| | | ALHNHYTQKSLSLSLGKGGGGSGGGGSGGGGSGGGGSEHPNA |
| | | RKYKGANKKGLSKGCFGLKLDRIGSMSGLGC |
| AMI302 | CNP36 | EHPNARKYKGANKKGLSKGCFGLKLDRIGSM |
| (Seq ID | | SGLGC |
| NO: 4) | | |
| AMI303 | CNP36 | EHPNARKYKGANKKGLSKGCFGLKLDRIGSM |
| (Seq ID | | SGLGC |
| NO: 4) | | |
| AMI304 | Fc4-(G4S)4- | DKRVESKYGPPCPPCPAPEFLGGPSVFLFPPKPKDTLMISRTPEV |
| (Seq ID | CNP36 | TCVVVDVSQEDPEVQFNWYVDGVEVHNAKTKPREEQFNSTYR |
| NO: | | VVSVLTVLHQDWLNGKEYKCKVSNKGLPSSIEKTISKAKGQPR |
| 138) | | EPQVYTLPPSQEEMTKNQVSLTCLVKGFYPSDIAVEWESNGQP |
| | | ENNYKTTPPVLDSDGSFFLYSRLTVDKSRWQEGNVFSCSVMHE |
| | | ALHNHYTQKSLSLSLGKGGGGSGGGGSGGGGSGGGGSEHPNA |
| | | RKYKGANKKGLSKGCFGLKLDRIGSMSGLGC |
| AMI305 | Sham CBA- | None encoded |
| | AMI305 | |
| AMI312 | Flt1-KDR- | SDTGRPFVEMYSEIPEIIHMTEGRELVIPCRVTSPNITVTLKKFPL |
| (Seq ID | CNP-Fc36 | DTLIPDGKRIIWDSRKGFIISNATYKEIGLLTCEATVNGHLYKTN |
| NO: | (VEGF- | YLTHRQTNTIIDVVLSPSHGIELSVGEKLVLNCTARTELNVGIDF |
| 139) | binding | NWEYPSSKHQHKKLVNRDLKTQSGSEMKKFLSTLTIDGVTRSD |
| | domains) | QGLYTCAASSGLMTKKNSTFVRVHEKDKRVESKYGPPCPPCPA |
| | | PEFLGGPSVFLFPPKPKDTLMISRTPEVTCVVVDVSQEDPEVQF |
| | | NWYVDGVEVHNAKTKPREEQFNSTYRVVSVLTVLHQDWLNG |
| | | KEYKCKVSNKGLPSSIEKTISKAKGQPREPQVYTLPPSQEEMTK |
| | | NQVSLTCLVKGFYPSDIAVEWESNGQPENNYKTTPPVLDSDGS |
| | | FFLYSRLTVDKSRWQEGNVFSCSVMHEALHNHYTQKSLSLSLG |
| | | K |
| | | GGGGSGGGGSGGGGSGGGGSEHPNARKYKGANKKGLSKGCF |
| | | GLKLDRIGSMSGLGC |
| AMI315 | Anti-VEGF- | EVQLVESGGGLVQPGGSLRLSCAASGYDFTHYGMNWVRQAPG |
| (Seq ID | A-ScFv- | KGLEWVGWINTYTGEPTYAADFKRRFTFSLDTSKSTAYLQMNS |
| NO: | CNP36 | LRAEDTAVYYCAKYPYYYGTSHWYFDVWGQGTLVTVSS |
| 140) | | GGGGSGGGGSGGGGSGGGGSDIQLTQSPSSLSASVGDRVTITCS |
| | | ASQDISNYLNWYQQKPGKAPKVLIYFTSSLHSGVPSRFSGSGSG |
| | | TDFTLTISSLQPEDFATYYCQQYSTVPWTFGQGTKVEIKRTVAA |
| | | GGGGSGGGGSGGGGSGGGGSEHPNARKYKGANKKGLSKGCF |
| | | GLKLDRIGSMSGLGC |

TABLE 11
Nucleic acid sequence of AAV constructs

| Clone no. | SEQ ID NO: | Sequence |
|---|---|---|
| AMI061 | 167 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTGCTAGTGCGGCCATCG |
| | | TGATGGCAGGTTGGGCGTCGCTTGGTCGGTCATTTCGAAC |
| | | CCCAGAGTCCCGCTCATTTACCCGGAGACAGGGAGAGGCT |
| | | CTTCTGCGTGTAGTGGTTGTGCAGAGCCTCATGCATCACG |
| | | GAGCATGAGAAGACGTTCCCCTGCTGCCACCTGCTCTTGT |
| | | CCACGGTGAGCTTGCTGTAGAGGAAGAAGGAGCCGTCGG |
| | | AGTCCAGCACGGGAGGCGTGGTCTTGTAGTTGTTCTCCGG |
| | | CTGCCCATTGCTCTCCCACTCCACGGCGATGTCGCTGGGA |
| | | TAGAAGCCTTTGACCAGGCAGGTCAGGCTGACCTGGTTCT |
| | | TGGTCAGCTCATCCCGGGATGGGGGCAGGGTGTACACCTG |
| | | TGGTTCTCGGGGCTGCCCTTTGGCTTTGGAGATGGTTTTCT |
| | | CGATGGGGGCTGGGAGGGCTTTGTTGGAGACCTTGCACTT |
| | | GTACTCCTTGCCATTCAGCCAGTCCTGGTGCAGGACGGTG |
| | | AGGACGCTGACCACACGGTACGTGCTGTTGTACTGCTCCT |
| | | CCCGCGGCTTTGTCTTGGCATTATGCACCTCCACGCCGTCC |
| | | ACGTACCAGTTGAACTTGACCTCAGGGTCTTCGTGGCTCA |
| | | CGTCCACCACCACGCATGTGACCTCAGGGGTCCGGGAGAT |
| | | CATGAGGGTGTCCTTGGGTTTTGGGGGGAAGAGGAAGACT |
| | | GACGGTCCCCCCAGGAGTTCAGGTGCTGGGCACGGTGGGC |
| | | ATGTGTGAGTTTTGTCCTTTCTCTGCTGCACACATCCCAGG |
| | | CCGCTCATGGAGCCGATTCGGTCCAGCTTGAGGCCGAAGC |
| | | AGCCCTTGGACAAGCCCTTCTTGTTGGCTCCTTTGTATTTG |
| | | CGCGCGTTGGGGTGCTCGCACTGCACGCCCTTAAGGATGG |
| | | CCACCAGGAACAGCCAGCTCAGGCCGAACTCCATGGTGG |
| | | CGGCAGGCCTAGAAGTAAAGGCAACATCCACTGAGGAGC |
| | | AGTTCTTTGATTTGCACCACCACCGGATCCGGGACCTGAA |
| | | ATAAAAGACAAAAAGACTAAACTTACCAGTTAACTCTAG |
| | | AGTCCGGAGGCTGGATCGGTCCCGGTGTCTTCTATGGAGG |
| | | TCAAAACAGCGTGGATGGCGTCTCCAGGCGATCTGACGGT |
| | | TCACTAAACGAGCTCTGCTTATATAGACCTCCCACCGTAC |
| | | ACGCCTACCGCCCATTTGCGTCAATGGGGCGGAGTTGTTA |
| | | CGACATTTTGGAAAGTCCCGTTGATTTTGGTGCCAAAACA |
| | | AACTCCCATTGACGTCAATGGGGTGGAGACTTGGAAATCC |
| | | CCGTGAGTCAAACCGCTATCCACGCCCATTGATGTACTGC |
| | | CAAAACCGCATCACCATGGTAATAGCGATGACTAATACGT |
| | | AGATGTACTGCCAAGTAGGAAAGTCCCATAAGGTCATGTA |
| | | CTGGGCATAATGCCAGGCGGGCCATTTACCGTCATTGACG |
| | | TCAATAGGGGGCGTACTTGGCATATGATACACTTGATGTA |
| | | CTGCCAAGTGGGCAGTTTACCGTAAATACTCCACCCATTG |
| | | ACGTCAATGGAAAGTCCCTATTGGCGTTACTATGGGAACA |
| | | TACGTCATTATTGACGTCAATGGGCGGGGGTCGTTGGGCG |
| | | GTCAGCCAGGCGGGCCATTTACCGTAAGTTATGTAACGGG |
| | | TACCGAATTCAGATCTGCTTCCTAGGAACCCCTAGTGATG |
| | | GAGTTGGCCACTCCCTCTCTGCGCGCTCGCTCGCTCACTGA |
| | | GGCCGGGCGACCAAAGGTCGCCCGACGCCCGGGCTTTGCC |
| | | CGGGCGGCCTCAGTGAGCGAGCGAGCGCGCCAGGATCCG |
| | | AGCTTGTCGAGAAGTACTAGAGGATCATCAAGCTGTAGCC |
| | | AACCACTAGAACTATAGCTAGAGTCCTGGGCGAACAAAC |
| | | GATGCTCGCCTTCCAGAAAACCGAGGATGCGAACCACTTC |
| | | ATCCGGGGTCAGCACCACCGGCAAGCGCCGCGACGGCCG |
| | | AGGTCTTCCGATCTCCTGAAGCCAGGGCAGATCCGTGCAC |
| | | AGCACCTTGCCGTAGAAGAACAGCAAGGCCGCCAATGCC |
| | | TGACGATGCGTGGAGACCGAAACCTTGCGCTCGTTCGCCA |
| | | GCCAGGACAGAAATGCCTCGACTTCGCTGCTGCCCAAGGT |
| | | TGCCGGGTGACGCACACCGTGGAAACGGATGAAGGCACG |
| | | AACCCAGTTGACATAAGCCTGTTCGGTTCGTAAACTGTAA |
| | | TGCAAGTAGCGTATGCGCTCACGCAACTGGTCCAGAACCT |
| | | TGACCGAACGCAGCGGTGGTAACGGCGCAGTGGCGGTTTT |
| | | CATGGCTTGTTATGACTGTTTTTTTGTACAGTCTATGCCTC |
| | | GGGCATCCAAGCAGCAAGCGCGTTACGCCGTGGGTCGAT |
| | | GTTTGATGTTATGGAGCAGCAACGATGTTACGCAGCAGCA |
| | | ACGATGTTACGCAGCAGGGCAGTCGCCCTAAAACAAAGTT |
| | | AGGTGGCTCAAGTATGGGCATCATTCGCACATGTAGGCTC |
| | | GGCCCTGACCAAGTCAAATCCATGCGGGCTGCTCTTGATC |
| | | TTTTCGGTCGTGAGTTCGGAGACGTAGCCACCTACTCCCA |
| | | ACATCAGCCGGACTCCGATTACCTCGGGAACTTGCTCCGT |
| | | AGTAAGACATTCATCGCGCTTGCTGCCTTCGACCAAGAAG |
| | | CGGTTGTTGGCGCTCTCGCGGCTTACGTTCTGCCCAGGTTT |
| | | GAGCAGCCGCGTAGTGAGATCTATATCTATGATCTCGCAG |
| | | TCTCCGGCGAGCACCGGAGGCAGGGCATTGCCACCGCGCT |
| | | CATCAATCTCCTCAAGCATGAGGCCAACGCGCTTGGTGCT |
| | | TATGTGATCTACGTGCAAGCAGATTACGGTGACGATCCCG |
| | | CAGTGGCTCTCTATACAAAGTTGGGCATACGGGAAGAAGT |
| | | GATGCACTTTGATATCGACCCAAGTACCGCCACCTAACAA |
| | | TTCGTTCAAGCCGAGATCGGCTTCCCGGCCGCGGAGTTGT |
| | | TCGGTAAATTGTCACAACGCCGCGAATATAGTCTTTACCA |
| | | TGCCCTTGGCCACGCCCCTCTTTAATACGACGGGCAATTT |
| | | GCACTTCAGAAAATGAAGAGTTTGCTTTAGCCATAACAAA |
| | | AGTCCAGTATGCTTTTTCACAGCATAACTGGACTGATTTCA |
| | | GTTTACAACTATTCTGTCTAGTTTAAGACTTTATTGTCATA |
| | | GTTTAGATCTATTTTGTTCAGTTTAAGACTTTATTGTCCGC |
| | | CCACACCCGCTTACGCAGGGCATCCATTTATTACTCAACC |
| | | GTAACCGATTTTGCCAGGTTACGCGGCTGGTCTGCGGTGT |
| | | GAAATACCGCACAGATGCGTAAGGAGAAAATACCGCATC |
| | | AGGCGCTCTTCCGCTTCCTCGCTCACTGACTCGCTGCGCTC |
| | | GGTCGTTCGGCTGCGGCGAGCGGTATCAGCTCACTCAAAG |
| | | GCGGTAATACGGTTATCCACAGAATCAGGGGATAACGCA |
| | | GGAAAGAACATGTGAGCAAAAGGCCAGCAAAAGGCCAGG |
| | | AACCGTAAAAAGGCCGCGTTGCTGGCGTTTTTCCATAGGC |
| | | TCCGCCCCCCTGACGAGCATCACAAAAATCGACGCTCAAG |
| | | TCAGAGGTGGCGAAACCCGACAGGACTATAAAGATACCA |
| | | GGCGTTTCCCCCTGGAAGCTCCCTCGTGCGCTCTCCTGTTC |
| | | CGACCCTGCCGCTTACCGGATACCTGTCCGCCTTTCTCCCT |
| | | TCGGGAAGCGTGGCGCTTTCTCAATGCTCACGCTGTAGGT |
| | | ATCTCAGTTCGGTGTAGGTCGTTCGCTCCAAGCTGGGCTG |
| | | TGTGCACGAACCCCCCGTTCAGCCCGACCGCTGCGCCTTA |
| | | TCCGGTAACTATCGTCTTGAGTCCAACCCGGTAAGACACG |
| | | ACTTATCGCCACTGGCAGCAGCCACTGGTAACAGGATTAG |
| | | CAGAGCGAGGTATGTAGGCGGTGCTACAGAGTTCTTGAAG |
| | | TGGTGGCCTAACTACGGCTACACTAGAAGGACAGTATTTG |
| | | GTATCTGCGCTCTGCTGAAGCCAGTTACCTTCGGAAAAAG |
| | | AGTTGGTAGCTCTTGATCCGGCAAACAAACCACCGCTGGT |
| | | AGCGGTGGTTTTTTTGTTTGCAAGCAGCAGATTACGCGCA |
| | | GAAAAAAAGGATCTCAAGAAGATCCTTTGATCTTTTCTAC |
| | | GGGGTCTGACGCTCAGTGGAACGAAAACTCACGTTAAGG |
| | | GATTTTGGTCATGAGATTATCAAAAAGGATCTTCACCTAG |
| | | ATCCTTTTAAATTAAAAATGAAGTTTTAAATCAATCTAAA |
| | | GTATATATGAGTAAACTTGGTCTGACAGTTACCAATGCTT |
| | | AATCAGTGAGGCACCTATCTCAGCGATCTGTCTATTTCGTT |
| | | CATCCATAGTTGCCTGACTCCCCGTCGTGTAGATAACTAC |
| | | GATACGGGAGGGCTTACCATCTGGCCCCAGTGCTGCAATG |
| | | ATACCGCGAGACCCACGCTCACCGGCTCCAGATTTATCAG |
| | | CAATAAACCAGCCAGCCGGAAGGGCCGAGCGCAGAAGTG |
| | | GTCCTGCAACTTTATCCGCCTCCATCCAGTCTATTAATTGT |
| | | TGCCGGGAAGCTAGAGTAAGTAGTTCGCCAGTTAATAGTT |
| | | TGCGCAACGTTGTTGCCATTGCTACAGGCATCGTGGTGTC |
| | | ACGCTCGTCGTTTGGTATGGCTTCATTCAGCTCCGGTTCCC |
| | | AACGATCAAGGCGAGTTACATGATCCCCCATGTTGTGCAA |
| | | AAAAGCGGTTAGCTCCTTCGGTCCTCCGATCGTTGTCAGA |
| | | AGTAAGTTGGCCGCAGTGTTATCACTCATGGTTATGGCAG |
| | | CACTGCATAATTCTCTTACTGTCATGCCATCCGTAAGATGC |
| | | TTTTCTGTGACTGGTGAGTACTCAACCAAGTCATTCTGAG |
| | | AATAGTGTATGCGGCGACCGAGTTGCTCTTGCCCGGCGTC |
| | | AATACGGGATAATACCGCGCCACATAGCAGAACTTTAAA |
| | | AGTGCTCATCATTGGAAAACGTTCTTCGGGGCGAAAACTC |
| | | TCAAGGATCTTACCGCTGTTGAGATCCAGTTCGATGTAAC |
| | | CCACTCGTGCACCCAACTGATCTTCAGCATCTTTTACTTTC |
| | | ACCAGCGTTTCTGGGTGAGCAAAAACAGGAAGGCAAAAT |
| | | GCCGCAAAAAAGGGAATAAGGGCGACACGGAAATGTTGA |
| | | ATACTCATACTCTTCCTTTTTCAATATTATTGAAGCATTTA |
| | | TCAGGGTTATTGTCTCATGAGCGGATACATATTTGAATGT |
| | | ATTTAGAAAAATAAACAAATAGGGGTTCCGCGCACATTTC |
| | | CCCGAAAAGTGCCACCTGAAATTGTAAACGTTAATATTTT |
| | | GTTAAAATTCGCGTTAAATTTTTGTTAAATCAGCTCATTTT |
| | | TTAACCAATAGGCCGAAATCGGCAAAATCCCTTATAAATC |
| | | AAAAGAATAGACCGAGATAGGGTTGAGTGTTGTTCCAGTT |
| | | TGGAACAAGAGTCCACTATTAAAGAACGTGGACTCCAAC |
| | | GTCAAAGGGCGAAAAACCGTCTATCAGGGCGATGGCCCA |
| | | CTACGTGAACCATCACCCTAATCAAGTTTTTTGGGGTCGA |
| | | GGTGCCGTAAAGCACTAAATCGGAACCCTAAAGGGAGCC |
| | | CCCGATTTAGAGCTTGACGGGGAAAGCCGGCGAACGTGG |
| | | CGAGAAAGGAAGGGAAGAAAGCGAAAGGAGCGGGCGCT |
| | | AGGGCGCTGGCAAGTGTAGCGGTCACGCTGCGCGTAACC |
| | | ACCACACCCGCCGCGCTTAATGCGCCGCTACAGGGCGCGT |
| | | C |
| AMI087 | 168 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTATCATTTAC |
| | | CCGGAGACAGGGAGAGGCTCTTCTGCGTGTAGTGGTTGTG |
| | | CAGAGCCTCATGCATCACGGAGCATGAGAAGACGTTCCCC |
| | | TGCTGCCACCTGCTCTTGTCCACGGTGAGCTTGCTGTAGA |
| | | GGAAGAAGGAGCCGTCGGAGTCCAGCACGGGAGGCGTGG |
| | | TCTTGTAGTTGTTCTCCGGCTGCCCATTGCTCTCCCACTCC |
| | | ACGGCGATGTCGCTGGGATAGAAGCCTTTGACCAGGCAG |
| | | GTCAGGCTGACCTGGTTCTTGGTCAGCTCATCCCGGGATG |
| | | GGGGCAGGGTGTACACCTGTGGTTCTCGGGGCTGCCCTTT |
| | | GGCTTTGGAGATGGTTTTCTCGATGGGGGCTGGGAGGGCT |
| | | TTGTTGGAGACCTTGCACTTGTACTCCTTGCCATTCAGCCA |
| | | GTCCTGGTGCAGGACGGTGAGGACGCTGACCACACGGTA |
| | | CGTGCTGTTGTACTGCTCCTCCCGCGGCTTTGTCTTGGCAT |
| | | TATGCACCTCCACGCCGTCCACGTACCAGTTGAACTTGAC |
| | | CTCAGGGTCTTCGTGGCTCACGTCCACCACCACGCATGTG |
| | | ACCTCAGGGGTCCGGGAGATCATGAGGGTGTCCTTGGGTT |
| | | TTGGGGGGAAGAGGAAGACTGACGGTCCCCCCAGGAGTT |
| | | CAGGTGCTGGGCACGGTGGGCATGTGTGAGTTTTGTCAGA |
| | | GCCGCCGCCGCCGCTGCCGCCTCCGCCAGATCCGCCTCCG |
| | | CCGCTTCCGCCTCCGCCACATCCCAGGCCGCTCATGGAGC |
| | | CGATTCGGTCCAGCTTGAGGCCGAAGCAGCCCTTGGACAA |
| | | GCCCTTCTTGTTGGCTCCTTTGTATTTGCGCGCGTTGGGGT |
| | | GCTCGCACTGCACGCCCTTAAGGATGGCCACCAGGAACAG |
| | | CCAGCTCAGGCCGAACTCCATGGTGGCGGCAGGCCTAGA |
| | | AGTAAAGGCAACATCCACTGAGGAGCAGTTCTTTGATTTG |
| | | CACCACCACCGGATCCGGGACCTGAAATAAAAGACAAAA |
| | | AGACTAAACTTACCAGTTAACTCTAGAGTCCGGAGGCTGG |
| | | ATCGGTCCCGGTGTCTTCTATGGAGGTCAAAACAGCGTGG |
| | | ATGGCGTCTCCAGGCGATCTGACGGTTCACTAAACGAGCT |
| | | CTGCTTATATAGACCTCCCACCGTACACGCCTACCGCCCA |
| | | TTTGCGTCAATGGGGCGGAGTTGTTACGACATTTTGGAAA |
| | | GTCCCGTTGATTTTGGTGCCAAAACAAACTCCCATTGACG |
| | | TCAATGGGGTGGAGACTTGGAAATCCCCGTGAGTCAAACC |
| | | GCTATCCACGCCCATTGATGTACTGCCAAAACCGCATCAC |
| | | CATGGTAATAGCGATGACTAATACGTAGATGTACTGCCAA |
| | | GTAGGAAAGTCCCATAAGGTCATGTACTGGGCATAATGCC |
| | | AGGCGGGCCATTTACCGTCATTGACGTCAATAGGGGGCGT |
| | | ACTTGGCATATGATACACTTGATGTACTGCCAAGTGGGCA |
| | | GTTTACCGTAAATACTCCACCCATTGACGTCAATGGAAAG |
| | | TCCCTATTGGCGTTACTATGGGAACATACGTCATTATTGAC |
| | | GTCAATGGGCGGGGGTCGTTGGGCGGTCAGCCAGGCGGG |
| | | CCATTTACCGTAAGTTATGTAACGGGTACCGAATTCAGAT |
| | | CAGCTTCCTAGGAACCCCTAGTGATGGAGTTGGCCACTCC |
| | | CTCTCTGCGCGCTCGCTCGCTCACTGAGGCCGGGCGACCA |
| | | AAGGTCGCCCGACGCCCGGGCTTTGCCCGGGCGGCCTCAG |
| | | TGAGCGAGCGAGCGCGCCAGGATCCGAGCTTGTCGAGAA |
| | | GTACTAGAGGATCATCAAGCTGTAGCCAACCACTAGAACT |
| | | ATAGCTAGAGTCCTGGGCGAACAAACGATGCTCGCCTTCC |
| | | AGAAAACCGAGGATGCGAACCACTTCATCCGGGGTCAGC |
| | | ACCACCGGCAAGCGCCGCGACGGCCGAGGTCTTCCGATCT |
| | | CCTGAAGCCAGGGCAGATCCGTGCACAGCACCTTGCCGTA |
| | | GAAGAACAGCAAGGCCGCCAATGCCTGACGATGCGTGGA |
| | | GACCGAAACCTTGCGCTCGTTCGCCAGCCAGGACAGAAAT |
| | | GCCTCGACTTCGCTGCTGCCCAAGGTTGCCGGGTGACGCA |
| | | CACCGTGGAAACGGATGAAGGCACGAACCCAGTTGACAT |
| | | AAGCCTGTTCGGTTCGTAAACTGTAATGCAAGTAGCGTAT |
| | | GCGCTCACGCAACTGGTCCAGAACCTTGACCGAACGCAGC |
| | | GGTGGTAACGGCGCAGTGGCGGTTTTCATGGCTTGTTATG |
| | | ACTGTTTTTTTGTACAGTCTATGCCTCGGGCATCCAAGCAG |
| | | CAAGCGCGTTACGCCGTGGGTCGATGTTTGATGTTATGGA |
| | | GCAGCAACGATGTTACGCAGCAGCAACGATGTTACGCAG |
| | | CAGGGCAGTCGCCCTAAAACAAAGTTAGGTGGCTCAAGT |
| | | ATGGGCATCATTCGCACATGTAGGCTCGGCCCTGACCAAG |
| | | TCAAATCCATGCGGGCTGCTCTTGATCTTTTCGGTCGTGAG |
| | | TTCGGAGACGTAGCCACCTACTCCCAACATCAGCCGGACT |
| | | CCGATTACCTCGGGAACTTGCTCCGTAGTAAGACATTCAT |
| | | CGCGCTTGCTGCCTTCGACCAAGAAGCGGTTGTTGGCGCT |
| | | CTCGCGGCTTACGTTCTGCCCAGGTTTGAGCAGCCGCGTA |
| | | GTGAGATCTATATCTATGATCTCGCAGTCTCCGGCGAGCA |
| | | CCGGAGGCAGGGCATTGCCACCGCGCTCATCAATCTCCTC |
| | | AAGCATGAGGCCAACGCGCTTGGTGCTTATGTGATCTACG |
| | | TGCAAGCAGATTACGGTGACGATCCCGCAGTGGCTCTCTA |
| | | TACAAAGTTGGGCATACGGGAAGAAGTGATGCACTTTGAT |
| | | ATCGACCCAAGTACCGCCACCTAACAATTCGTTCAAGCCG |
| | | AGATCGGCTTCCCGGCCGCGGAGTTGTTCGGTAAATTGTC |
| | | ACAACGCCGCGAATATAGTCTTTACCATGCCCTTGGCCAC |
| | | GCCCCTCTTTAATACGACGGGCAATTTGCACTTCAGAAAA |
| | | TGAAGAGTTTGCTTTAGCCATAACAAAAGTCCAGTATGCT |
| | | TTTTCACAGCATAACTGGACTGATTTCAGTTTACAACTATT |
| | | CTGTCTAGTTTAAGACTTTATTGTCATAGTTTAGATCTATT |
| | | TTGTTCAGTTTAAGACTTTATTGTCCGCCCACACCCGCTTA |
| | | CGCAGGGCATCCATTTATTACTCAACCGTAACCGATTTTG |
| | | CCAGGTTACGCGGCTGGTCTGCGGTGTGAAATACCGCACA |
| | | GATGCGTAAGGAGAAAATACCGCATCAGGCGCTCTTCCGC |
| | | TTCCTCGCTCACTGACTCGCTGCGCTCGGTCGTTCGGCTGC |
| | | GGCGAGCGGTATCAGCTCACTCAAAGGCGGTAATACGGTT |
| | | ATCCACAGAATCAGGGGATAACGCAGGAAAGAACATGTG |
| | | AGCAAAAGGCCAGCAAAAGGCCAGGAACCGTAAAAAGGC |
| | | CGCGTTGCTGGCGTTTTTCCATAGGCTCCGCCCCCCTGACG |
| | | AGCATCACAAAAATCGACGCTCAAGTCAGAGGTGGCGAA |
| | | ACCCGACAGGACTATAAAGATACCAGGCGTTTCCCCCTGG |
| | | AAGCTCCCTCGTGCGCTCTCCTGTTCCGACCCTGCCGCTTA |
| | | CCGGATACCTGTCCGCCTTTCTCCCTTCGGGAAGCGTGGC |
| | | GCTTTCTCAATGCTCACGCTGTAGGTATCTCAGTTCGGTGT |
| | | AGGTCGTTCGCTCCAAGCTGGGCTGTGTGCACGAACCCCC |
| | | CGTTCAGCCCGACCGCTGCGCCTTATCCGGTAACTATCGT |
| | | CTTGAGTCCAACCCGGTAAGACACGACTTATCGCCACTGG |
| | | CAGCAGCCACTGGTAACAGGATTAGCAGAGCGAGGTATG |
| | | TAGGCGGTGCTACAGAGTTCTTGAAGTGGTGGCCTAACTA |
| | | CGGCTACACTAGAAGGACAGTATTTGGTATCTGCGCTCTG |
| | | CTGAAGCCAGTTACCTTCGGAAAAAGAGTTGGTAGCTCTT |
| | | GATCCGGCAAACAAACCACCGCTGGTAGCGGTGGTTTTTT |
| | | TGTTTGCAAGCAGCAGATTACGCGCAGAAAAAAAGGATC |
| | | TCAAGAAGATCCTTTGATCTTTTCTACGGGGTCTGACGCTC |
| | | AGTGGAACGAAAACTCACGTTAAGGGATTTTGGTCATGAG |
| | | ATTATCAAAAAGGATCTTCACCTAGATCCTTTTAAATTAA |
| | | AAATGAAGTTTTAAATCAATCTAAAGTATATATGAGTAAA |
| | | CTTGGTCTGACAGTTACCAATGCTTAATCAGTGAGGCACC |
| | | TATCTCAGCGATCTGTCTATTTCGTTCATCCATAGTTGCCT |
| | | GACTCCCCGTCGTGTAGATAACTACGATACGGGAGGGCTT |
| | | ACCATCTGGCCCCAGTGCTGCAATGATACCGCGAGACCCA |
| | | CGCTCACCGGCTCCAGATTTATCAGCAATAAACCAGCCAG |
| | | CCGGAAGGGCCGAGCGCAGAAGTGGTCCTGCAACTTTATC |
| | | CGCCTCCATCCAGTCTATTAATTGTTGCCGGGAAGCTAGA |
| | | GTAAGTAGTTCGCCAGTTAATAGTTTGCGCAACGTTGTTG |
| | | CCATTGCTACAGGCATCGTGGTGTCACGCTCGTCGTTTGGT |
| | | ATGGCTTCATTCAGCTCCGGTTCCCAACGATCAAGGCGAG |
| | | TTACATGATCCCCCATGTTGTGCAAAAAAGCGGTTAGCTC |
| | | CTTCGGTCCTCCGATCGTTGTCAGAAGTAAGTTGGCCGCA |
| | | GTGTTATCACTCATGGTTATGGCAGCACTGCATAATTCTCT |
| | | TACTGTCATGCCATCCGTAAGATGCTTTTCTGTGACTGGTG |
| | | AGTACTCAACCAAGTCATTCTGAGAATAGTGTATGCGGCG |
| | | ACCGAGTTGCTCTTGCCCGGCGTCAATACGGGATAATACC |
| | | GCGCCACATAGCAGAACTTTAAAAGTGCTCATCATTGGAA |
| | | AACGTTCTTCGGGGCGAAAACTCTCAAGGATCTTACCGCT |
| | | GTTGAGATCCAGTTCGATGTAACCCACTCGTGCACCCAAC |
| | | TGATCTTCAGCATCTTTTACTTTCACCAGCGTTTCTGGGTG |
| | | AGCAAAAACAGGAAGGCAAAATGCCGCAAAAAAGGGAA |
| | | TAAGGGCGACACGGAAATGTTGAATACTCATACTCTTCCT |
| | | TTTTCAATATTATTGAAGCATTTATCAGGGTTATTGTCTCA |
| | | TGAGCGGATACATATTTGAATGTATTTAGAAAAATAAACA |
| | | AATAGGGGTTCCGCGCACATTTCCCCGAAAAGTGCCACCT |
| | | GAAATTGTAAACGTTAATATTTTGTTAAAATTCGCGTTAA |
| | | ATTTTTGTTAAATCAGCTCATTTTTTAACCAATAGGCCGAA |
| | | ATCGGCAAAATCCCTTATAAATCAAAAGAATAGACCGAG |
| | | ATAGGGTTGAGTGTTGTTCCAGTTTGGAACAAGAGTCCAC |
| | | TATTAAAGAACGTGGACTCCAACGTCAAAGGGCGAAAAA |
| | | CCGTCTATCAGGGCGATGGCCCACTACGTGAACCATCACC |
| | | CTAATCAAGTTTTTTGGGGTCGAGGTGCCGTAAAGCACTA |
| | | AATCGGAACCCTAAAGGGAGCCCCCGATTTAGAGCTTGAC |
| | | GGGGAAAGCCGGCGAACGTGGCGAGAAAGGAAGGGAAG |
| | | AAAGCGAAAGGAGCGGGCGCTAGGGCGCTGGCAAGTGTA |
| | | GCGGTCACGCTGCGCGTAACCACCACACCCGCCGCGCTTA |
| | | ATGCGCCGCTACAGGGCGCGTC |
| AMI088 | 169 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTAACATCCC |
| | | AGGCCGCTCATGGAGCCGATTCGGTCCAGCTTGAGGCCGA |
| | | AGCAGCCCTTGGACAAGCCCTTCTTGTTGGCTCCTTTGTAT |
| | | TTGCGCGCGTTGGGGTGCTCAGAGCCGCCGCCGCCGCTGC |
| | | CGCCTCCGCCAGATCCGCCTCCGCCGCTTCCGCCTCCGCCT |
| | | TTACCCGGAGACAGGGAGAGGCTCTTCTGCGTGTAGTGGT |
| | | TGTGCAGAGCCTCATGCATCACGGAGCATGAGAAGACGTT |
| | | CCCCTGCTGCCACCTGCTCTTGTCCACGGTGAGCTTGCTGT |
| | | AGAGGAAGAAGGAGCCGTCGGAGTCCAGCACGGGAGGCG |
| | | TGGTCTTGTAGTTGTTCTCCGGCTGCCCATTGCTCTCCCAC |
| | | TCCACGGCGATGTCGCTGGGATAGAAGCCTTTGACCAGGC |
| | | AGGTCAGGCTGACCTGGTTCTTGGTCAGCTCATCCCGGGA |
| | | TGGGGGCAGGGTGTACACCTGTGGTTCTCGGGGCTGCCCT |
| | | TTGGCTTTGGAGATGGTTTTCTCGATGGGGGCTGGGAGGG |
| | | CTTTGTTGGAGACCTTGCACTTGTACTCCTTGCCATTCAGC |
| | | CAGTCCTGGTGCAGGACGGTGAGGACGCTGACCACACGG |
| | | TACGTGCTGTTGTACTGCTCCTCCCGCGGCTTTGTCTTGGC |
| | | ATTATGCACCTCCACGCCGTCCACGTACCAGTTGAACTTG |
| | | ACCTCAGGGTCTTCGTGGCTCACGTCCACCACCACGCATG |
| | | TGACCTCAGGGGTCCGGGAGATCATGAGGGTGTCCTTGGG |
| | | TTTTGGGGGGAAGAGGAAGACTGACGGTCCCCCCAGGAG |
| | | TTCAGGTGCTGGGCACGGTGGGCATGTGTGAGTTTTGTCG |
| | | CACTGCACGCCCTTAAGGATGGCCACCAGGAACAGCCAG |
| | | CTCAGGCCGAACTCCATGGTGGCGGCAGGCCTAGAAGTA |
| | | AAGGCAACATCCACTGAGGAGCAGTTCTTTGATTTGCACC |
| | | ACCACCGGATCCGGGACCTGAAATAAAAGACAAAAAGAC |
| | | TAAACTTACCAGTTAACTCTAGAGTCCGGAGGCTGGATCG |
| | | GTCCCGGTGTCTTCTATGGAGGTCAAAACAGCGTGGATGG |
| | | CGTCTCCAGGCGATCTGACGGTTCACTAAACGAGCTCTGC |
| | | TTATATAGACCTCCCACCGTACACGCCTACCGCCCATTTGC |
| | | GTCAATGGGGCGGAGTTGTTACGACATTTTGGAAAGTCCC |
| | | GTTGATTTTGGTGCCAAAACAAACTCCCATTGACGTCAAT |
| | | GGGGTGGAGACTTGGAAATCCCCGTGAGTCAAACCGCTAT |
| | | CCACGCCCATTGATGTACTGCCAAAACCGCATCACCATGG |
| | | TAATAGCGATGACTAATACGTAGATGTACTGCCAAGTAGG |
| | | AAAGTCCCATAAGGTCATGTACTGGGCATAATGCCAGGCG |
| | | GGCCATTTACCGTCATTGACGTCAATAGGGGGCGTACTTG |
| | | GCATATGATACACTTGATGTACTGCCAAGTGGGCAGTTTA |
| | | CCGTAAATACTCCACCCATTGACGTCAATGGAAAGTCCCT |
| | | ATTGGCGTTACTATGGGAACATACGTCATTATTGACGTCA |
| | | ATGGGCGGGGGTCGTTGGGCGGTCAGCCAGGCGGGCCAT |
| | | TTACCGTAAGTTATGTAACGGGTACCGAATTCAGATCAGC |
| | | TTCCTAGGAACCCCTAGTGATGGAGTTGGCCACTCCCTCT |
| | | CTGCGCGCTCGCTCGCTCACTGAGGCCGGGCGACCAAAGG |
| | | TCGCCCGACGCCCGGGCTTTGCCCGGGCGGCCTCAGTGAG |
| | | CGAGCGAGCGCGCCAGGATCCGAGCTTGTCGAGAAGTAC |
| | | TAGAGGATCATCAAGCTGTAGCCAACCACTAGAACTATAG |
| | | CTAGAGTCCTGGGCGAACAAACGATGCTCGCCTTCCAGAA |
| | | AACCGAGGATGCGAACCACTTCATCCGGGGTCAGCACCAC |
| | | CGGCAAGCGCCGCGACGGCCGAGGTCTTCCGATCTCCTGA |
| | | AGCCAGGGCAGATCCGTGCACAGCACCTTGCCGTAGAAG |
| | | AACAGCAAGGCCGCCAATGCCTGACGATGCGTGGAGACC |
| | | GAAACCTTGCGCTCGTTCGCCAGCCAGGACAGAAATGCCT |
| | | CGACTTCGCTGCTGCCCAAGGTTGCCGGGTGACGCACACC |
| | | GTGGAAACGGATGAAGGCACGAACCCAGTTGACATAAGC |
| | | CTGTTCGGTTCGTAAACTGTAATGCAAGTAGCGTATGCGC |
| | | TCACGCAACTGGTCCAGAACCTTGACCGAACGCAGCGGTG |
| | | GTAACGGCGCAGTGGCGGTTTTCATGGCTTGTTATGACTG |
| | | TTTTTTTGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGCAACGATGTTACGCAGCAG |
| | | GGCAGTCGCCCTAAAACAAAGTTAGGTGGCTCAAGTATGG |
| | | GCATCATTCGCACATGTAGGCTCGGCCCTGACCAAGTCAA |
| | | ATCCATGCGGGCTGCTCTTGATCTTTTCGGTCGTGAGTTCG |
| | | GAGACGTAGCCACCTACTCCCAACATCAGCCGGACTCCGA |
| | | TTACCTCGGGAACTTGCTCCGTAGTAAGACATTCATCGCG |
| | | CTTGCTGCCTTCGACCAAGAAGCGGTTGTTGGCGCTCTCG |
| | | CGGCTTACGTTCTGCCCAGGTTTGAGCAGCCGCGTAGTGA |
| | | GATCTATATCTATGATCTCGCAGTCTCCGGCGAGCACCGG |
| | | AGGCAGGGCATTGCCACCGCGCTCATCAATCTCCTCAAGC |
| | | ATGAGGCCAACGCGCTTGGTGCTTATGTGATCTACGTGCA |
| | | AGCAGATTACGGTGACGATCCCGCAGTGGCTCTCTATACA |
| | | AAGTTGGGCATACGGGAAGAAGTGATGCACTTTGATATCG |
| | | ACCCAAGTACCGCCACCTAACAATTCGTTCAAGCCGAGAT |
| | | CGGCTTCCCGGCCGCGGAGTTGTTCGGTAAATTGTCACAA |
| | | CGCCGCGAATATAGTCTTTACCATGCCCTTGGCCACGCCC |
| | | CTCTTTAATACGACGGGCAATTTGCACTTCAGAAAATGAA |
| | | GAGTTTGCTTTAGCCATAACAAAAGTCCAGTATGCTTTTTC |
| | | ACAGCATAACTGGACTGATTTCAGTTTACAACTATTCTGTC |
| | | TAGTTTAAGACTTTATTGTCATAGTTTAGATCTATTTTGTT |
| | | CAGTTTAAGACTTTATTGTCCGCCCACACCCGCTTACGCA |
| | | GGGCATCCATTTATTACTCAACCGTAACCGATTTTGCCAG |
| | | GTTACGCGGCTGGTCTGCGGTGTGAAATACCGCACAGATG |
| | | CGTAAGGAGAAAATACCGCATCAGGCGCTCTTCCGCTTCC |
| | | TCGCTCACTGACTCGCTGCGCTCGGTCGTTCGGCTGCGGC |
| | | GAGCGGTATCAGCTCACTCAAAGGCGGTAATACGGTTATC |
| | | CACAGAATCAGGGGATAACGCAGGAAAGAACATGTGAGC |
| | | AAAAGGCCAGCAAAAGGCCAGGAACCGTAAAAAGGCCGC |
| | | GTTGCTGGCGTTTTTCCATAGGCTCCGCCCCCCTGACGAGC |
| | | ATCACAAAAATCGACGCTCAAGTCAGAGGTGGCGAAACC |
| | | CGACAGGACTATAAAGATACCAGGCGTTTCCCCCTGGAAG |
| | | CTCCCTCGTGCGCTCTCCTGTTCCGACCCTGCCGCTTACCG |
| | | GATACCTGTCCGCCTTTCTCCCTTCGGGAAGCGTGGCGCTT |
| | | TCTCAATGCTCACGCTGTAGGTATCTCAGTTCGGTGTAGGT |
| | | CGTTCGCTCCAAGCTGGGCTGTGTGCACGAACCCCCCGTT |
| | | CAGCCCGACCGCTGCGCCTTATCCGGTAACTATCGTCTTG |
| | | AGTCCAACCCGGTAAGACACGACTTATCGCCACTGGCAGC |
| | | AGCCACTGGTAACAGGATTAGCAGAGCGAGGTATGTAGG |
| | | CGGTGCTACAGAGTTCTTGAAGTGGTGGCCTAACTACGGC |
| | | TACACTAGAAGGACAGTATTTGGTATCTGCGCTCTGCTGA |
| | | AGCCAGTTACCTTCGGAAAAAGAGTTGGTAGCTCTTGATC |
| | | CGGCAAACAAACCACCGCTGGTAGCGGTGGTTTTTTTGTT |
| | | TGCAAGCAGCAGATTACGCGCAGAAAAAAAGGATCTCAA |
| | | GAAGATCCTTTGATCTTTTCTACGGGGTCTGACGCTCAGTG |
| | | GAACGAAAACTCACGTTAAGGGATTTTGGTCATGAGATTA |
| | | TCAAAAAGGATCTTCACCTAGATCCTTTTAAATTAAAAAT |
| | | GAAGTTTTAAATCAATCTAAAGTATATATGAGTAAACTTG |
| | | GTCTGACAGTTACCAATGCTTAATCAGTGAGGCACCTATC |
| | | TCAGCGATCTGTCTATTTCGTTCATCCATAGTTGCCTGACT |
| | | CCCCGTCGTGTAGATAACTACGATACGGGAGGGCTTACCA |
| | | TCTGGCCCCAGTGCTGCAATGATACCGCGAGACCCACGCT |
| | | CACCGGCTCCAGATTTATCAGCAATAAACCAGCCAGCCGG |
| | | AAGGGCCGAGCGCAGAAGTGGTCCTGCAACTTTATCCGCC |
| | | TCCATCCAGTCTATTAATTGTTGCCGGGAAGCTAGAGTAA |
| | | GTAGTTCGCCAGTTAATAGTTTGCGCAACGTTGTTGCCATT |
| | | GCTACAGGCATCGTGGTGTCACGCTCGTCGTTTGGTATGG |
| | | CTTCATTCAGCTCCGGTTCCCAACGATCAAGGCGAGTTAC |
| | | ATGATCCCCCATGTTGTGCAAAAAAGCGGTTAGCTCCTTC |
| | | GGTCCTCCGATCGTTGTCAGAAGTAAGTTGGCCGCAGTGT |
| | | TATCACTCATGGTTATGGCAGCACTGCATAATTCTCTTACT |
| | | GTCATGCCATCCGTAAGATGCTTTTCTGTGACTGGTGAGT |
| | | ACTCAACCAAGTCATTCTGAGAATAGTGTATGCGGCGACC |
| | | GAGTTGCTCTTGCCCGGCGTCAATACGGGATAATACCGCG |
| | | CCACATAGCAGAACTTTAAAAGTGCTCATCATTGGAAAAC |
| | | GTTCTTCGGGGCGAAAACTCTCAAGGATCTTACCGCTGTT |
| | | GAGATCCAGTTCGATGTAACCCACTCGTGCACCCAACTGA |
| | | TCTTCAGCATCTTTTACTTTCACCAGCGTTTCTGGGTGAGC |
| | | AAAAACAGGAAGGCAAAATGCCGCAAAAAAGGGAATAA |
| | | GGGCGACACGGAAATGTTGAATACTCATACTCTTCCTTTTT |
| | | CAATATTATTGAAGCATTTATCAGGGTTATTGTCTCATGAG |
| | | CGGATACATATTTGAATGTATTTAGAAAAATAAACAAATA |
| | | GGGGTTCCGCGCACATTTCCCCGAAAAGTGCCACCTGAAA |
| | | TTGTAAACGTTAATATTTTGTTAAAATTCGCGTTAAATTTT |
| | | TGTTAAATCAGCTCATTTTTTAACCAATAGGCCGAAATCG |
| | | GCAAAATCCCTTATAAATCAAAAGAATAGACCGAGATAG |
| | | GGTTGAGTGTTGTTCCAGTTTGGAACAAGAGTCCACTATT |
| | | AAAGAACGTGGACTCCAACGTCAAAGGGCGAAAAACCGT |
| | | CTATCAGGGCGATGGCCCACTACGTGAACCATCACCCTAA |
| | | TCAAGTTTTTTGGGGTCGAGGTGCCGTAAAGCACTAAATC |
| | | GGAACCCTAAAGGGAGCCCCCGATTTAGAGCTTGACGGG |
| | | GAAAGCCGGCGAACGTGGCGAGAAAGGAAGGGAAGAAA |
| | | GCGAAAGGAGCGGGCGCTAGGGCGCTGGCAAGTGTAGCG |
| | | GTCACGCTGCGCGTAACCACCACACCCGCCGCGCTTAATG |
| | | CGCCGCTACAGGGCGCGTC |
| AMI169 | 170 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAATCAGGCGGCC |
| | | ACGGTTCTCTTGATCTCCACCTTGGTGCCCTGGCCGAAGGT |
| | | CCAGGGCACGGTGCTGTACTGCTGGCAGTAGTAGGTGGCG |
| | | AAGTCCTCGGGCTGCAGGCTGCTGATGGTCAGGGTGAAGT |
| | | CGGTGCCGCTGCCGCTGCCGCTGAATCTGCTGGGCACGCC |
| | | GCTGTGCAGGCTGCTGGTGAAGTAGATCAGCACCTTGGGG |
| | | GCCTTGCCGGGCTTCTGCTGGTACCAGTTCAGGTAGTTGCT |
| | | GATGTCCTGGCTGGCGCTGCAGGTGATGGTCACTCTGTCG |
| | | CCCACGCTGGCGCTCAGGCTGCTGGGGCTCTGGGTCAGCT |
| | | GGATGTCAGAGCCGCCGCCGCCGCTGCCGCCTCCGCCAGA |
| | | TCCGCCTCCGCCGCTTCCGCCTCCGCCCACGGTCACCAGG |
| | | GTGCCCTGGCCCCACACGTCGAAGTACCAGTGGCTGGTGC |
| | | CGTAGTAGTAGGGGTACTTGGCGCAGTAGTACACGGCGGT |
| | | GTCCTCGGCTCTCAGGCTGTTCATCTGCAGGTAGGCGGTG |
| | | CTCTTGCTGGTGTCCAGGCTGAAGGTGAATCTTCTCTTGAA |
| | | GTCGGCGGCGTAGGTGGGCTCGCCGGTGTAGGTGTTGATC |
| | | CAGCCCACCCACTCCAGGCCCTTGCCGGGGGCCTGTCTCA |
| | | CCCAGTTCATGCCGTAGTGGGTGAAGTCGTAGCCGCTGGC |
| | | GGCGCAGCTCAGTCTCAGGCTGCCGCCGGGCTGCACCAGG |
| | | CCGCCGCCGCTCTCCACCAGCTGCACCTCGCACTGCACGC |
| | | CCTTAAGGATGGCCACCAGGAACAGCCAGCTCAGGCCGA |
| | | ACTCCATGGGGCCGGGGTTGCTCTCCACGTCGCCGGCCAG |
| | | CTTCAGCAGGTCGAAGTTCAGGGTCTGCTTCACGGGGGCT |
| | | CTCTTTCTTCTACATCCCAGGCCGCTCATGGAGCCGATTCG |
| | | GTCCAGCTTGAGGCCGAAGCAGCCCTTGGACAAGCCCTTC |
| | | TTGTTGGCTCCTTTGTATTTGCGCGCGTTGGGGTGCTCGCA |
| | | CTGCACGCCCTTAAGGATGGCCACCAGGAACAGCCAGCTC |
| | | AGGCCGAACTCCATGGTGGCGGCAGGCCTAGAAGTAAAG |
| | | GCAACATCCACTGAGGAGCAGTTCTTTGATTTGCACCACC |
| | | ACCGGATCCGGGACCTGAAATAAAAGACAAAAAGACTAA |
| | | ACTTACCAGTTAACTCTAGAGTCCGGAGGCTGGATCGGTC |
| | | CCGGTGTCTTCTATGGAGGTCAAAACAGCGTGGATGGCGT |
| | | CTCCAGGCGATCTGACGGTTCACTAAACGAGCTCTGCTTA |
| | | TATAGACCTCCCACCGTACACGCCTACCGCCCATTTGCGT |
| | | CAATGGGGCGGAGTTGTTACGACATTTTGGAAAGTCCCGT |
| | | TGATTTTGGTGCCAAAACAAACTCCCATTGACGTCAATGG |
| | | GGTGGAGACTTGGAAATCCCCGTGAGTCAAACCGCTATCC |
| | | ACGCCCATTGATGTACTGCCAAAACCGCATCACCATGGTA |
| | | ATAGCGATGACTAATACGTAGATGTACTGCCAAGTAGGAA |
| | | AGTCCCATAAGGTCATGTACTGGGCATAATGCCAGGCGGG |
| | | CCATTTACCGTCATTGACGTCAATAGGGGGCGTACTTGGC |
| | | ATATGATACACTTGATGTACTGCCAAGTGGGCAGTTTACC |
| | | GTAAATACTCCACCCATTGACGTCAATGGAAAGTCCCTAT |
| | | TGGCGTTACTATGGGAACATACGTCATTATTGACGTCAAT |
| | | GGGCGGGGGTCGTTGGGCGGTCAGCCAGGCGGGCCATTT |
| | | ACCGTAAGTTATGTAACGGGTACCGAATTCAGATCAGCTT |
| | | CCTAGGAACCCCTAGTGATGGAGTTGGCCACTCCCTCTCT |
| | | GCGCGCTCGCTCGCTCACTGAGGCCGGGCGACCAAAGGTC |
| | | GCCCGACGCCCGGGCTTTGCCCGGGCGGCCTCAGTGAGCG |
| | | AGCGAGCGCGCCAGGATCCGAGCTTGTCGAGAAGTACTA |
| | | GAGGATCATCAAGCTGTAGCCAACCACTAGAACTATAGCT |
| | | AGAGTCCTGGGCGAACAAACGATGCTCGCCTTCCAGAAA |
| | | ACCGAGGATGCGAACCACTTCATCCGGGGTCAGCACCACC |
| | | GGCAAGCGCCGCGACGGCCGAGGTCTTCCGATCTCCTGAA |
| | | GCCAGGGCAGATCCGTGCACAGCACCTTGCCGTAGAAGA |
| | | ACAGCAAGGCCGCCAATGCCTGACGATGCGTGGAGACCG |
| | | AAACCTTGCGCTCGTTCGCCAGCCAGGACAGAAATGCCTC |
| | | GACTTCGCTGCTGCCCAAGGTTGCCGGGTGACGCACACCG |
| | | TGGAAACGGATGAAGGCACGAACCCAGTTGACATAAGCC |
| | | TGTTCGGTTCGTAAACTGTAATGCAAGTAGCGTATGCGCT |
| | | CACGCAACTGGTCCAGAACCTTGACCGAACGCAGCGGTG |
| | | GTAACGGCGCAGTGGCGGTTTTCATGGCTTGTTATGACTG |
| | | TTTTTTTGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGCAACGATGTTACGCAGCAG |
| | | GGCAGTCGCCCTAAAACAAAGTTAGGTGGCTCAAGTATGG |
| | | GCATCATTCGCACATGTAGGCTCGGCCCTGACCAAGTCAA |
| | | ATCCATGCGGGCTGCTCTTGATCTTTTCGGTCGTGAGTTCG |
| | | GAGACGTAGCCACCTACTCCCAACATCAGCCGGACTCCGA |
| | | TTACCTCGGGAACTTGCTCCGTAGTAAGACATTCATCGCG |
| | | CTTGCTGCCTTCGACCAAGAAGCGGTTGTTGGCGCTCTCG |
| | | CGGCTTACGTTCTGCCCAGGTTTGAGCAGCCGCGTAGTGA |
| | | GATCTATATCTATGATCTCGCAGTCTCCGGCGAGCACCGG |
| | | AGGCAGGGCATTGCCACCGCGCTCATCAATCTCCTCAAGC |
| | | ATGAGGCCAACGCGCTTGGTGCTTATGTGATCTACGTGCA |
| | | AGCAGATTACGGTGACGATCCCGCAGTGGCTCTCTATACA |
| | | AAGTTGGGCATACGGGAAGAAGTGATGCACTTTGATATCG |
| | | ACCCAAGTACCGCCACCTAACAATTCGTTCAAGCCGAGAT |
| | | CGGCTTCCCGGCCGCGGAGTTGTTCGGTAAATTGTCACAA |
| | | CGCCGCGAATATAGTCTTTACCATGCCCTTGGCCACGCCC |
| | | CTCTTTAATACGACGGGCAATTTGCACTTCAGAAAATGAA |
| | | GAGTTTGCTTTAGCCATAACAAAAGTCCAGTATGCTTTTTC |
| | | ACAGCATAACTGGACTGATTTCAGTTTACAACTATTCTGTC |
| | | TAGTTTAAGACTTTATTGTCATAGTTTAGATCTATTTTGTT |
| | | CAGTTTAAGACTTTATTGTCCGCCCACACCCGCTTACGCA |
| | | GGGCATCCATTTATTACTCAACCGTAACCGATTTTGCCAG |
| | | GTTACGCGGCTGGTCTGCGGTGTGAAATACCGCACAGATG |
| | | CGTAAGGAGAAAATACCGCATCAGGCGCTCTTCCGCTTCC |
| | | TCGCTCACTGACTCGCTGCGCTCGGTCGTTCGGCTGCGGC |
| | | GAGCGGTATCAGCTCACTCAAAGGCGGTAATACGGTTATC |
| | | CACAGAATCAGGGGATAACGCAGGAAAGAACATGTGAGC |
| | | AAAAGGCCAGCAAAAGGCCAGGAACCGTAAAAAGGCCGC |
| | | GTTGCTGGCGTTTTTCCATAGGCTCCGCCCCCCTGACGAGC |
| | | ATCACAAAAATCGACGCTCAAGTCAGAGGTGGCGAAACC |
| | | CGACAGGACTATAAAGATACCAGGCGTTTCCCCCTGGAAG |
| | | CTCCCTCGTGCGCTCTCCTGTTCCGACCCTGCCGCTTACCG |
| | | GATACCTGTCCGCCTTTCTCCCTTCGGGAAGCGTGGCGCTT |
| | | TCTCAATGCTCACGCTGTAGGTATCTCAGTTCGGTGTAGGT |
| | | CGTTCGCTCCAAGCTGGGCTGTGTGCACGAACCCCCCGTT |
| | | CAGCCCGACCGCTGCGCCTTATCCGGTAACTATCGTCTTG |
| | | AGTCCAACCCGGTAAGACACGACTTATCGCCACTGGCAGC |
| | | AGCCACTGGTAACAGGATTAGCAGAGCGAGGTATGTAGG |
| | | CGGTGCTACAGAGTTCTTGAAGTGGTGGCCTAACTACGGC |
| | | TACACTAGAAGGACAGTATTTGGTATCTGCGCTCTGCTGA |
| | | AGCCAGTTACCTTCGGAAAAAGAGTTGGTAGCTCTTGATC |
| | | CGGCAAACAAACCACCGCTGGTAGCGGTGGTTTTTTTGTT |
| | | TGCAAGCAGCAGATTACGCGCAGAAAAAAAGGATCTCAA |
| | | GAAGATCCTTTGATCTTTTCTACGGGGTCTGACGCTCAGTG |
| | | GAACGAAAACTCACGTTAAGGGATTTTGGTCATGAGATTA |
| | | TCAAAAAGGATCTTCACCTAGATCCTTTTAAATTAAAAAT |
| | | GAAGTTTTAAATCAATCTAAAGTATATATGAGTAAACTTG |
| | | GTCTGACAGTTACCAATGCTTAATCAGTGAGGCACCTATC |
| | | TCAGCGATCTGTCTATTTCGTTCATCCATAGTTGCCTGACT |
| | | CCCCGTCGTGTAGATAACTACGATACGGGAGGGCTTACCA |
| | | TCTGGCCCCAGTGCTGCAATGATACCGCGAGACCCACGCT |
| | | CACCGGCTCCAGATTTATCAGCAATAAACCAGCCAGCCGG |
| | | AAGGGCCGAGCGCAGAAGTGGTCCTGCAACTTTATCCGCC |
| | | TCCATCCAGTCTATTAATTGTTGCCGGGAAGCTAGAGTAA |
| | | GTAGTTCGCCAGTTAATAGTTTGCGCAACGTTGTTGCCATT |
| | | GCTACAGGCATCGTGGTGTCACGCTCGTCGTTTGGTATGG |
| | | CTTCATTCAGCTCCGGTTCCCAACGATCAAGGCGAGTTAC |
| | | ATGATCCCCCATGTTGTGCAAAAAAGCGGTTAGCTCCTTC |
| | | GGTCCTCCGATCGTTGTCAGAAGTAAGTTGGCCGCAGTGT |
| | | TATCACTCATGGTTATGGCAGCACTGCATAATTCTCTTACT |
| | | GTCATGCCATCCGTAAGATGCTTTTCTGTGACTGGTGAGT |
| | | ACTCAACCAAGTCATTCTGAGAATAGTGTATGCGGCGACC |
| | | GAGTTGCTCTTGCCCGGCGTCAATACGGGATAATACCGCG |
| | | CCACATAGCAGAACTTTAAAAGTGCTCATCATTGGAAAAC |
| | | GTTCTTCGGGGCGAAAACTCTCAAGGATCTTACCGCTGTT |
| | | GAGATCCAGTTCGATGTAACCCACTCGTGCACCCAACTGA |
| | | TCTTCAGCATCTTTTACTTTCACCAGCGTTTCTGGGTGAGC |
| | | AAAAACAGGAAGGCAAAATGCCGCAAAAAAGGGAATAA |
| | | GGGCGACACGGAAATGTTGAATACTCATACTCTTCCTTTTT |
| | | CAATATTATTGAAGCATTTATCAGGGTTATTGTCTCATGAG |
| | | CGGATACATATTTGAATGTATTTAGAAAAATAAACAAATA |
| | | GGGGTTCCGCGCACATTTCCCCGAAAAGTGCCACCTGAAA |
| | | TTGTAAACGTTAATATTTTGTTAAAATTCGCGTTAAATTTT |
| | | TGTTAAATCAGCTCATTTTTTAACCAATAGGCCGAAATCG |
| | | GCAAAATCCCTTATAAATCAAAAGAATAGACCGAGATAG |
| | | GGTTGAGTGTTGTTCCAGTTTGGAACAAGAGTCCACTATT |
| | | AAAGAACGTGGACTCCAACGTCAAAGGGCGAAAAACCGT |
| | | CTATCAGGGCGATGGCCCACTACGTGAACCATCACCCTAA |
| | | TCAAGTTTTTTGGGGTCGAGGTGCCGTAAAGCACTAAATC |
| | | GGAACCCTAAAGGGAGCCCCCGATTTAGAGCTTGACGGG |
| | | GAAAGCCGGCGAACGTGGCGAGAAAGGAAGGGAAGAAA |
| | | GCGAAAGGAGCGGGCGCTAGGGCGCTGGCAAGTGTAGCG |
| | | GTCACGCTGCGCGTAACCACCACACCCGCCGCGCTTAATG |
| | | CGCCGCTACAGGGCGCGTC |
| AMI182 | 171 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTATCATTTAC |
| | | CCGGAGACAGGGAGAGGCTCTTCTGCGTGTAGTGGTTGTG |
| | | CAGAGCCTCATGCATCACGGAGCATGAGAAGACGTTCCCC |
| | | TGCTGCCACCTGCTCTTGTCCACGGTGAGCTTGCTGTAGA |
| | | GGAAGAAGGAGCCGTCGGAGTCCAGCACGGGAGGCGTGG |
| | | TCTTGTAGTTGTTCTCCGGCTGCCCATTGCTCTCCCACTCC |
| | | ACGGCGATGTCGCTGGGATAGAAGCCTTTGACCAGGCAG |
| | | GTCAGGCTGACCTGGTTCTTGGTCAGCTCATCCCGGGATG |
| | | GGGGCAGGGTGTACACCTGTGGTTCTCGGGGCTGCCCTTT |
| | | GGCTTTGGAGATGGTTTTCTCGATGGGGGCTGGGAGGGCT |
| | | TTGTTGGAGACCTTGCACTTGTACTCCTTGCCATTCAGCTA |
| | | GTCCTGGTGCAGGACGGTGAGGACGCTGACCACACGGTA |
| | | CGTGCTGTTGTACTGCTCCTCCCGCGGCTTTGTCTTGGCAT |
| | | TATGCACCTCCACGCCGTCCACGTACCAGTTGAACTTGAC |
| | | CTCAGGGTCTTCGTGGCTCACGTCCACCACCACGCATGTG |
| | | ACCTCAGGGGTCCGGGAGATCATGAGGGTGTCCTTGGGTT |
| | | TTGGGGGGAAGAGGAAGACTGACGGTCCCCCCAGGAGTT |
| | | CAGGTGCTGGGCACGGTGGGCATGTGTGAGTTTTGTCAGA |
| | | GCCGCCGCCGCCGCTGCCGCCTCCGCCAGATCCGCCTCCG |
| | | CCGCTTCCGCCTCCCTAACATCCCAGGCCGCTCATGGAGC |
| | | CGATTCGGTCCAGCTTGAGGCCGAAGCAGCCCTTGGACAA |
| | | GCCCTTCTTGTTGGCTCCTTTGTATTTGCGCGCGTTGGGGT |
| | | GCTCGCACTGCACGCCCTTAAGGATGGCCACCAGGAACAG |
| | | CCAGCTCAGGCCGAACTCCATGGTGGCGGCAGGCCTAGA |
| | | AGTAAAGGCAACATCCACTGAGGAGCAGTTCTTTGATTTG |
| | | CACCACCACCGGATCCGGGACCTGAAATAAAAGACAAAA |
| | | AGACTAAACTTACCAGTTAACTCTAGAGTCCGGAGGCTGG |
| | | ATCGGTCCCGGTGTCTTCTATGGAGGTCAAAACAGCGTGG |
| | | ATGGCGTCTCCAGGCGATCTGACGGTTCACTAAACGAGCT |
| | | CTGCTTATATAGACCTCCCACCGTACACGCCTACCGCCCA |
| | | TTTGCGTCAATGGGGCGGAGTTGTTACGACATTTTGGAAA |
| | | GTCCCGTTGATTTTGGTGCCAAAACAAACTCCCATTGACG |
| | | TCAATGGGGTGGAGACTTGGAAATCCCCGTGAGTCAAACC |
| | | GCTATCCACGCCCATTGATGTACTGCCAAAACCGCATCAC |
| | | CATGGTAATAGCGATGACTAATACGTAGATGTACTGCCAA |
| | | GTAGGAAAGTCCCATAAGGTCATGTACTGGGCATAATGCC |
| | | AGGCGGGCCATTTACCGTCATTGACGTCAATAGGGGGCGT |
| | | ACTTGGCATATGATACACTTGATGTACTGCCAAGTGGGCA |
| | | GTTTACCGTAAATACTCCACCCATTGACGTCAATGGAAAG |
| | | TCCCTATTGGCGTTACTATGGGAACATACGTCATTATTGAC |
| | | GTCAATGGGCGGGGGTCGTTGGGCGGTCAGCCAGGCGGG |
| | | CCATTTACCGTAAGTTATGTAACGGGTACCGAATTCAGAT |
| | | CAGCTTCCTAGGAACCCCTAGTGATGGAGTTGGCCACTCC |
| | | CTCTCTGCGCGCTCGCTCGCTCACTGAGGCCGGGCGACCA |
| | | AAGGTCGCCCGACGCCCGGGCTTTGCCCGGGCGGCCTCAG |
| | | TGAGCGAGCGAGCGCGCCAGGATCCGAGCTTGTCGAGAA |
| | | GTACTAGAGGATCATCAAGCTGTAGCCAACCACTAGAACT |
| | | ATAGCTAGAGTCCTGGGCGAACAAACGATGCTCGCCTTCC |
| | | AGAAAACCGAGGATGCGAACCACTTCATCCGGGGTCAGC |
| | | ACCACCGGCAAGCGCCGCGACGGCCGAGGTCTTCCGATCT |
| | | CCTGAAGCCAGGGCAGATCCGTGCACAGCACCTTGCCGTA |
| | | GAAGAACAGCAAGGCCGCCAATGCCTGACGATGCGTGGA |
| | | GACCGAAACCTTGCGCTCGTTCGCCAGCCAGGACAGAAAT |
| | | GCCTCGACTTCGCTGCTGCCCAAGGTTGCCGGGTGACGCA |
| | | CACCGTGGAAACGGATGAAGGCACGAACCCAGTTGACAT |
| | | AAGCCTGTTCGGTTCGTAAACTGTAATGCAAGTAGCGTAT |
| | | GCGCTCACGCAACTGGTCCAGAACCTTGACCGAACGCAGC |
| | | GGTGGTAACGGCGCAGTGGCGGTTTTCATGGCTTGTTATG |
| | | ACTGTTTTTTTGTACAGTCTATGCCTCGGGCATCCAAGCAG |
| | | CAAGCGCGTTACGCCGTGGGTCGATGTTTGATGTTATGGA |
| | | GCAGCAACGATGTTACGCAGCAGCAACGATGTTACGCAG |
| | | CAGGGCAGTCGCCCTAAAACAAAGTTAGGTGGCTCAAGT |
| | | ATGGGCATCATTCGCACATGTAGGCTCGGCCCTGACCAAG |
| | | TCAAATCCATGCGGGCTGCTCTTGATCTTTTCGGTCGTGAG |
| | | TTCGGAGACGTAGCCACCTACTCCCAACATCAGCCGGACT |
| | | CCGATTACCTCGGGAACTTGCTCCGTAGTAAGACATTCAT |
| | | CGCGCTTGCTGCCTTCGACCAAGAAGCGGTTGTTGGCGCT |
| | | CTCGCGGCTTACGTTCTGCCCAGGTTTGAGCAGCCGCGTA |
| | | GTGAGATCTATATCTATGATCTCGCAGTCTCCGGCGAGCA |
| | | CCGGAGGCAGGGCATTGCCACCGCGCTCATCAATCTCCTC |
| | | AAGCATGAGGCCAACGCGCTTGGTGCTTATGTGATCTACG |
| | | TGCAAGCAGATTACGGTGACGATCCCGCAGTGGCTCTCTA |
| | | TACAAAGTTGGGCATACGGGAAGAAGTGATGCACTTTGAT |
| | | ATCGACCCAAGTACCGCCACCTAACAATTCGTTCAAGCCG |
| | | AGATCGGCTTCCCGGCCGCGGAGTTGTTCGGTAAATTGTC |
| | | ACAACGCCGCGAATATAGTCTTTACCATGCCCTTGGCCAC |
| | | GCCCCTCTTTAATACGACGGGCAATTTGCACTTCAGAAAA |
| | | TGAAGAGTTTGCTTTAGCCATAACAAAAGTCCAGTATGCT |
| | | TTTTCACAGCATAACTGGACTGATTTCAGTTTACAACTATT |
| | | CTGTCTAGTTTAAGACTTTATTGTCATAGTTTAGATCTATT |
| | | TTGTTCAGTTTAAGACTTTATTGTCCGCCCACACCCGCTTA |
| | | CGCAGGGCATCCATTTATTACTCAACCGTAACCGATTTTG |
| | | CCAGGTTACGCGGCTGGTCTGCGGTGTGAAATACCGCACA |
| | | GATGCGTAAGGAGAAAATACCGCATCAGGCGCTCTTCCGC |
| | | TTCCTCGCTCACTGACTCGCTGCGCTCGGTCGTTCGGCTGC |
| | | GGCGAGCGGTATCAGCTCACTCAAAGGCGGTAATACGGTT |
| | | ATCCACAGAATCAGGGGATAACGCAGGAAAGAACATGTG |
| | | AGCAAAAGGCCAGCAAAAGGCCAGGAACCGTAAAAAGGC |
| | | CGCGTTGCTGGCGTTTTTCCATAGGCTCCGCCCCCCTGACG |
| | | AGCATCACAAAAATCGACGCTCAAGTCAGAGGTGGCGAA |
| | | ACCCGACAGGACTATAAAGATACCAGGCGTTTCCCCCTGG |
| | | AAGCTCCCTCGTGCGCTCTCCTGTTCCGACCCTGCCGCTTA |
| | | CCGGATACCTGTCCGCCTTTCTCCCTTCGGGAAGCGTGGC |
| | | GCTTTCTCAATGCTCACGCTGTAGGTATCTCAGTTCGGTGT |
| | | AGGTCGTTCGCTCCAAGCTGGGCTGTGTGCACGAACCCCC |
| | | CGTTCAGCCCGACCGCTGCGCCTTATCCGGTAACTATCGT |
| | | CTTGAGTCCAACCCGGTAAGACACGACTTATCGCCACTGG |
| | | CAGCAGCCACTGGTAACAGGATTAGCAGAGCGAGGTATG |
| | | TAGGCGGTGCTACAGAGTTCTTGAAGTGGTGGCCTAACTA |
| | | CGGCTACACTAGAAGGACAGTATTTGGTATCTGCGCTCTG |
| | | CTGAAGCCAGTTACCTTCGGAAAAAGAGTTGGTAGCTCTT |
| | | GATCCGGCAAACAAACCACCGCTGGTAGCGGTGGTTTTTT |
| | | TGTTTGCAAGCAGCAGATTACGCGCAGAAAAAAAGGATC |
| | | TCAAGAAGATCCTTTGATCTTTTCTACGGGGTCTGACGCTC |
| | | AGTGGAACGAAAACTCACGTTAAGGGATTTTGGTCATGAG |
| | | ATTATCAAAAAGGATCTTCACCTAGATCCTTTTAAATTAA |
| | | AAATGAAGTTTTAAATCAATCTAAAGTATATATGAGTAAA |
| | | CTTGGTCTGACAGTTACCAATGCTTAATCAGTGAGGCACC |
| | | TATCTCAGCGATCTGTCTATTTCGTTCATCCATAGTTGCCT |
| | | GACTCCCCGTCGTGTAGATAACTACGATACGGGAGGGCTT |
| | | ACCATCTGGCCCCAGTGCTGCAATGATACCGCGAGACCCA |
| | | CGCTCACCGGCTCCAGATTTATCAGCAATAAACCAGCCAG |
| | | CCGGAAGGGCCGAGCGCAGAAGTGGTCCTGCAACTTTATC |
| | | CGCCTCCATCCAGTCTATTAATTGTTGCCGGGAAGCTAGA |
| | | GTAAGTAGTTCGCCAGTTAATAGTTTGCGCAACGTTGTTG |
| | | CCATTGCTACAGGCATCGTGGTGTCACGCTCGTCGTTTGGT |
| | | ATGGCTTCATTCAGCTCCGGTTCCCAACGATCAAGGCGAG |
| | | TTACATGATCCCCCATGTTGTGCAAAAAAGCGGTTAGCTC |
| | | CTTCGGTCCTCCGATCGTTGTCAGAAGTAAGTTGGCCGCA |
| | | GTGTTATCACTCATGGTTATGGCAGCACTGCATAATTCTCT |
| | | TACTGTCATGCCATCCGTAAGATGCTTTTCTGTGACTGGTG |
| | | AGTACTCAACCAAGTCATTCTGAGAATAGTGTATGCGGCG |
| | | ACCGAGTTGCTCTTGCCCGGCGTCAATACGGGATAATACC |
| | | GCGCCACATAGCAGAACTTTAAAAGTGCTCATCATTGGAA |
| | | AACGTTCTTCGGGGCGAAAACTCTCAAGGATCTTACCGCT |
| | | GTTGAGATCCAGTTCGATGTAACCCACTCGTGCACCCAAC |
| | | TGATCTTCAGCATCTTTTACTTTCACCAGCGTTTCTGGGTG |
| | | AGCAAAAACAGGAAGGCAAAATGCCGCAAAAAAGGGAA |
| | | TAAGGGCGACACGGAAATGTTGAATACTCATACTCTTCCT |
| | | TTTTCAATATTATTGAAGCATTTATCAGGGTTATTGTCTCA |
| | | TGAGCGGATACATATTTGAATGTATTTAGAAAAATAAACA |
| | | AATAGGGGTTCCGCGCACATTTCCCCGAAAAGTGCCACCT |
| | | GAAATTGTAAACGTTAATATTTTGTTAAAATTCGCGTTAA |
| | | ATTTTTGTTAAATCAGCTCATTTTTTAACCAATAGGCCGAA |
| | | ATCGGCAAAATCCCTTATAAATCAAAAGAATAGACCGAG |
| | | ATAGGGTTGAGTGTTGTTCCAGTTTGGAACAAGAGTCCAC |
| | | TATTAAAGAACGTGGACTCCAACGTCAAAGGGCGAAAAA |
| | | CCGTCTATCAGGGCGATGGCCCACTACGTGAACCATCACC |
| | | CTAATCAAGTTTTTTGGGGTCGAGGTGCCGTAAAGCACTA |
| | | AATCGGAACCCTAAAGGGAGCCCCCGATTTAGAGCTTGAC |
| | | GGGGAAAGCCGGCGAACGTGGCGAGAAAGGAAGGGAAG |
| | | AAAGCGAAAGGAGCGGGCGCTAGGGCGCTGGCAAGTGTA |
| | | GCGGTCACGCTGCGCGTAACCACCACACCCGCCGCGCTTA |
| | | ATGCGCCGCTACAGGGCGCGTC |
| AMI189 | 172 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTAACATCCC |
| | | AGGCCGCTCATGGAGCCGATTCGGTCCAGCTTGAGGCCGA |
| | | AGCAGCCCTTGGACAAGCCCTTCTTGTTGGCTCCTTTGTAT |
| | | TTGCGCGCGTTGGGGTGCTAAGAGCCGCCGCCGCCGCTGC |
| | | CGCCTCCGCCAGATCCGCCTCCGCCGCTTCCGCCTCCGCCT |
| | | TTACCCGGAGACAGGGAGAGGCTCTTCTGCGTGTAGTGGT |
| | | TGTGCAGAGCCTCATGCATCACGGAGCATGAGAAGACGTT |
| | | CCCCTGCTGCCACCTGCTCTTGTCCACGGTGAGCTTGCTGT |
| | | AGAGGAAGAAGGAGCCGTCGGAGTCCAGCACGGGAGGCG |
| | | TGGTCTTGTAGTTGTTCTCCGGCTGCCCATTGCTCTCCCAC |
| | | TCCACGGCGATGTCGCTGGGATAGAAGCCTTTGACCAGGC |
| | | AGGTCAGGCTGACCTGGTTCTTGGTCAGCTCATCCCGGGA |
| | | TGGGGGCAGGGTGTACACCTGTGGTTCTCGGGGCTGCCCT |
| | | TTGGCTTTGGAGATGGTTTTCTCGATGGGGGCTGGGAGGG |
| | | CTTTGTTGGAGACCTTGCACTTGTACTCCTTGCCATTCAGC |
| | | CAGTCCTGGTGCAGGACGGTGAGGACGCTGACCACACGG |
| | | TACGTGCTGTTGTACTGCTCCTCCCGCGGCTTTGTCTTGGC |
| | | ATTATGCACCTCCACGCCGTCCACGTACCAGTTGAACTTG |
| | | ACCTCAGGGTCTTCGTGGCTCACGTCCACCACCACGCATG |
| | | TGACCTCAGGGGTCCGGGAGATCTAGAGGGTGTCCTTGGG |
| | | TTTTGGGGGGAAGAGGAAGACTGACGGTCCCCCCAGGAG |
| | | TTCAGGTGCTGGGCACGGTGGGCATGTGTGAGTTTTGTCG |
| | | CACTGCACGCCCTTAAGGATGGCCACCAGGAACAGCCAG |
| | | CTCAGGCCGAACTCCTAGGTGGCGGCAGGCCTAGAAGTA |
| | | AAGGCAACATCCACTGAGGAGCAGTTCTTTGATTTGCACC |
| | | ACCACCGGATCCGGGACCTGAAATAAAAGACAAAAAGAC |
| | | TAAACTTACCAGTTAACTCTAGAGTCCGGAGGCTGGATCG |
| | | GTCCCGGTGTCTTCTATGGAGGTCAAAACAGCGTGGATGG |
| | | CGTCTCCAGGCGATCTGACGGTTCACTAAACGAGCTCTGC |
| | | TTATATAGACCTCCCACCGTACACGCCTACCGCCCATTTGC |
| | | GTCAATGGGGCGGAGTTGTTACGACATTTTGGAAAGTCCC |
| | | GTTGATTTTGGTGCCAAAACAAACTCCCATTGACGTCAAT |
| | | GGGGTGGAGACTTGGAAATCCCCGTGAGTCAAACCGCTAT |
| | | CCACGCCCATTGATGTACTGCCAAAACCGCATCACCATGG |
| | | TAATAGCGATGACTAATACGTAGATGTACTGCCAAGTAGG |
| | | AAAGTCCCATAAGGTCATGTACTGGGCATAATGCCAGGCG |
| | | GGCCATTTACCGTCATTGACGTCAATAGGGGGCGTACTTG |
| | | GCATATGATACACTTGATGTACTGCCAAGTGGGCAGTTTA |
| | | CCGTAAATACTCCACCCATTGACGTCAATGGAAAGTCCCT |
| | | ATTGGCGTTACTATGGGAACATACGTCATTATTGACGTCA |
| | | ATGGGCGGGGGTCGTTGGGCGGTCAGCCAGGCGGGCCAT |
| | | TTACCGTAAGTTATGTAACGGGTACCGAATTCAGATCAGC |
| | | TTCCTAGGAACCCCTAGTGATGGAGTTGGCCACTCCCTCT |
| | | CTGCGCGCTCGCTCGCTCACTGAGGCCGGGCGACCAAAGG |
| | | TCGCCCGACGCCCGGGCTTTGCCCGGGCGGCCTCAGTGAG |
| | | CGAGCGAGCGCGCCAGGATCCGAGCTTGTCGAGAAGTAC |
| | | TAGAGGATCATCAAGCTGTAGCCAACCACTAGAACTATAG |
| | | CTAGAGTCCTGGGCGAACAAACGATGCTCGCCTTCCAGAA |
| | | AACCGAGGATGCGAACCACTTCATCCGGGGTCAGCACCAC |
| | | CGGCAAGCGCCGCGACGGCCGAGGTCTTCCGATCTCCTGA |
| | | AGCCAGGGCAGATCCGTGCACAGCACCTTGCCGTAGAAG |
| | | AACAGCAAGGCCGCCAATGCCTGACGATGCGTGGAGACC |
| | | GAAACCTTGCGCTCGTTCGCCAGCCAGGACAGAAATGCCT |
| | | CGACTTCGCTGCTGCCCAAGGTTGCCGGGTGACGCACACC |
| | | GTGGAAACGGATGAAGGCACGAACCCAGTTGACATAAGC |
| | | CTGTTCGGTTCGTAAACTGTAATGCAAGTAGCGTATGCGC |
| | | TCACGCAACTGGTCCAGAACCTTGACCGAACGCAGCGGTG |
| | | GTAACGGCGCAGTGGCGGTTTTCATGGCTTGTTATGACTG |
| | | TTTTTTTGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGCAACGATGTTACGCAGCAG |
| | | GGCAGTCGCCCTAAAACAAAGTTAGGTGGCTCAAGTATGG |
| | | GCATCATTCGCACATGTAGGCTCGGCCCTGACCAAGTCAA |
| | | ATCCATGCGGGCTGCTCTTGATCTTTTCGGTCGTGAGTTCG |
| | | GAGACGTAGCCACCTACTCCCAACATCAGCCGGACTCCGA |
| | | TTACCTCGGGAACTTGCTCCGTAGTAAGACATTCATCGCG |
| | | CTTGCTGCCTTCGACCAAGAAGCGGTTGTTGGCGCTCTCG |
| | | CGGCTTACGTTCTGCCCAGGTTTGAGCAGCCGCGTAGTGA |
| | | GATCTATATCTATGATCTCGCAGTCTCCGGCGAGCACCGG |
| | | AGGCAGGGCATTGCCACCGCGCTCATCAATCTCCTCAAGC |
| | | ATGAGGCCAACGCGCTTGGTGCTTATGTGATCTACGTGCA |
| | | AGCAGATTACGGTGACGATCCCGCAGTGGCTCTCTATACA |
| | | AAGTTGGGCATACGGGAAGAAGTGATGCACTTTGATATCG |
| | | ACCCAAGTACCGCCACCTAACAATTCGTTCAAGCCGAGAT |
| | | CGGCTTCCCGGCCGCGGAGTTGTTCGGTAAATTGTCACAA |
| | | CGCCGCGAATATAGTCTTTACCATGCCCTTGGCCACGCCC |
| | | CTCTTTAATACGACGGGCAATTTGCACTTCAGAAAATGAA |
| | | GAGTTTGCTTTAGCCATAACAAAAGTCCAGTATGCTTTTTC |
| | | ACAGCATAACTGGACTGATTTCAGTTTACAACTATTCTGTC |
| | | TAGTTTAAGACTTTATTGTCATAGTTTAGATCTATTTTGTT |
| | | CAGTTTAAGACTTTATTGTCCGCCCACACCCGCTTACGCA |
| | | GGGCATCCATTTATTACTCAACCGTAACCGATTTTGCCAG |
| | | GTTACGCGGCTGGTCTGCGGTGTGAAATACCGCACAGATG |
| | | CGTAAGGAGAAAATACCGCATCAGGCGCTCTTCCGCTTCC |
| | | TCGCTCACTGACTCGCTGCGCTCGGTCGTTCGGCTGCGGC |
| | | GAGCGGTATCAGCTCACTCAAAGGCGGTAATACGGTTATC |
| | | CACAGAATCAGGGGATAACGCAGGAAAGAACATGTGAGC |
| | | AAAAGGCCAGCAAAAGGCCAGGAACCGTAAAAAGGCCGC |
| | | GTTGCTGGCGTTTTTCCATAGGCTCCGCCCCCCTGACGAGC |
| | | ATCACAAAAATCGACGCTCAAGTCAGAGGTGGCGAAACC |
| | | CGACAGGACTATAAAGATACCAGGCGTTTCCCCCTGGAAG |
| | | CTCCCTCGTGCGCTCTCCTGTTCCGACCCTGCCGCTTACCG |
| | | GATACCTGTCCGCCTTTCTCCCTTCGGGAAGCGTGGCGCTT |
| | | TCTCAATGCTCACGCTGTAGGTATCTCAGTTCGGTGTAGGT |
| | | CGTTCGCTCCAAGCTGGGCTGTGTGCACGAACCCCCCGTT |
| | | CAGCCCGACCGCTGCGCCTTATCCGGTAACTATCGTCTTG |
| | | AGTCCAACCCGGTAAGACACGACTTATCGCCACTGGCAGC |
| | | AGCCACTGGTAACAGGATTAGCAGAGCGAGGTATGTAGG |
| | | CGGTGCTACAGAGTTCTTGAAGTGGTGGCCTAACTACGGC |
| | | TACACTAGAAGGACAGTATTTGGTATCTGCGCTCTGCTGA |
| | | AGCCAGTTACCTTCGGAAAAAGAGTTGGTAGCTCTTGATC |
| | | CGGCAAACAAACCACCGCTGGTAGCGGTGGTTTTTTTGTT |
| | | TGCAAGCAGCAGATTACGCGCAGAAAAAAAGGATCTCAA |
| | | GAAGATCCTTTGATCTTTTCTACGGGGTCTGACGCTCAGTG |
| | | GAACGAAAACTCACGTTAAGGGATTTTGGTCATGAGATTA |
| | | TCAAAAAGGATCTTCACCTAGATCCTTTTAAATTAAAAAT |
| | | GAAGTTTTAAATCAATCTAAAGTATATATGAGTAAACTTG |
| | | GTCTGACAGTTACCAATGCTTAATCAGTGAGGCACCTATC |
| | | TCAGCGATCTGTCTATTTCGTTCATCCATAGTTGCCTGACT |
| | | CCCCGTCGTGTAGATAACTACGATACGGGAGGGCTTACCA |
| | | TCTGGCCCCAGTGCTGCAATGATACCGCGAGACCCACGCT |
| | | CACCGGCTCCAGATTTATCAGCAATAAACCAGCCAGCCGG |
| | | AAGGGCCGAGCGCAGAAGTGGTCCTGCAACTTTATCCGCC |
| | | TCCATCCAGTCTATTAATTGTTGCCGGGAAGCTAGAGTAA |
| | | GTAGTTCGCCAGTTAATAGTTTGCGCAACGTTGTTGCCATT |
| | | GCTACAGGCATCGTGGTGTCACGCTCGTCGTTTGGTATGG |
| | | CTTCATTCAGCTCCGGTTCCCAACGATCAAGGCGAGTTAC |
| | | ATGATCCCCCATGTTGTGCAAAAAAGCGGTTAGCTCCTTC |
| | | GGTCCTCCGATCGTTGTCAGAAGTAAGTTGGCCGCAGTGT |
| | | TATCACTCATGGTTATGGCAGCACTGCATAATTCTCTTACT |
| | | GTCATGCCATCCGTAAGATGCTTTTCTGTGACTGGTGAGT |
| | | ACTCAACCAAGTCATTCTGAGAATAGTGTATGCGGCGACC |
| | | GAGTTGCTCTTGCCCGGCGTCAATACGGGATAATACCGCG |
| | | CCACATAGCAGAACTTTAAAAGTGCTCATCATTGGAAAAC |
| | | GTTCTTCGGGGCGAAAACTCTCAAGGATCTTACCGCTGTT |
| | | GAGATCCAGTTCGATGTAACCCACTCGTGCACCCAACTGA |
| | | TCTTCAGCATCTTTTACTTTCACCAGCGTTTCTGGGTGAGC |
| | | AAAAACAGGAAGGCAAAATGCCGCAAAAAAGGGAATAA |
| | | GGGCGACACGGAAATGTTGAATACTCATACTCTTCCTTTTT |
| | | CAATATTATTGAAGCATTTATCAGGGTTATTGTCTCATGAG |
| | | CGGATACATATTTGAATGTATTTAGAAAAATAAACAAATA |
| | | GGGGTTCCGCGCACATTTCCCCGAAAAGTGCCACCTGAAA |
| | | TTGTAAACGTTAATATTTTGTTAAAATTCGCGTTAAATTTT |
| | | TGTTAAATCAGCTCATTTTTTAACCAATAGGCCGAAATCG |
| | | GCAAAATCCCTTATAAATCAAAAGAATAGACCGAGATAG |
| | | GGTTGAGTGTTGTTCCAGTTTGGAACAAGAGTCCACTATT |
| | | AAAGAACGTGGACTCCAACGTCAAAGGGCGAAAAACCGT |
| | | CTATCAGGGCGATGGCCCACTACGTGAACCATCACCCTAA |
| | | TCAAGTTTTTTGGGGTCGAGGTGCCGTAAAGCACTAAATC |
| | | GGAACCCTAAAGGGAGCCCCCGATTTAGAGCTTGACGGG |
| | | GAAAGCCGGCGAACGTGGCGAGAAAGGAAGGGAAGAAA |
| | | GCGAAAGGAGCGGGCGCTAGGGCGCTGGCAAGTGTAGCG |
| | | GTCACGCTGCGCGTAACCACCACACCCGCCGCGCTTAATG |
| | | CGCCGCTACAGGGCGCGTC |
| AMI263 | 173 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTAACATCCC |
| | | AGGCCGCTCATGGAGCCGATTCGGTCCAGCTTGAGGCCGA |
| | | AGCAGCCCTTGGACAAGCCCTTCTTGTTGGCTCCTTTGTAT |
| | | TTGCGCGCGTTGGGGTGCTCAGAGCCGCCTCCGCCGCTTC |
| | | CGCCTCCGCCAGATCCGCCTCCGCCGCTTCCGCCTCCGCCC |
| | | TTGCCCAGGCTCAGGCTCAGGCTCTTCTGGGTGTAGTGGT |
| | | TGTGCAGGGCCTCGTGCATCACGCTGCAGCTGAACACGTT |
| | | GCCCTCCTGCCATCTGCTCTTGTCCACGGTCAGTCTGCTGT |
| | | ACAGGAAGAAGCTGCCGTCGCTGTCCAGCACGGGGGGGG |
| | | TGGTCTTGTAGTTGTTCTCGGGCTGGCCGTTGCTCTCCCAC |
| | | TCCACGGCGATGTCGCTGGGGTAGAAGCCCTTCACCAGGC |
| | | AGGTCAGGCTCACCTGGTTCTTGGTCATCTCCTCCTGGCTG |
| | | GGGGGCAGGGTGTACACCTGGGGCTCTCTGGGCTGGCCCT |
| | | TGGCCTTGCTGATGGTCTTCTCGATGCTGCTGGGCAGGCC |
| | | CTTGTTGCTCACCTTGCACTTGTACTCCTTGCCGTTCAGCC |
| | | AGTCCTGGTGCAGCACGGTCAGCACGCTCACCACTCTGTA |
| | | GGTGCTGTTGAACTGCTCCTCTCTGGGCTTGGTCTTGGCGT |
| | | TGTGCACCTCCACGCCGTCCACGTACCAGTTGAACTGCAC |
| | | CTCGGGGTCCTCCTGGCTCACGTCCACCACCACGCAGGTC |
| | | ACCTCGGGGGTTCTGCTGATCATCAGGGTGTCCTTGGGCT |
| | | TGGGGGGGAACAGGAACACGCTGGGGCCGCCCAGGAACT |
| | | CGGGGGCGGGGCAGGGGGGGCAGGGGGGGCCGTACTTGC |
| | | TCTCCACTCTCTTGTCGCACTGCACGCCCTTAAGGATGGCC |
| | | ACCAGGAACAGCCAGCTCAGGCCGAACTCCATGGTGGCG |
| | | GCAGGCCTAGAAGTAAAGGCAACATCCACTGAGGAGCAG |
| | | TTCTTTGATTTGCACCACCACCGGATCCGGGACCTGAAAT |
| | | AAAAGACAAAAAGACTAAACTTACCAGTTAACTCTAGAG |
| | | TCCGGAGGCTGGATCGGTCCCGGTGTCTTCTATGGAGGTC |
| | | AAAACAGCGTGGATGGCGTCTCCAGGCGATCTGACGGTTC |
| | | ACTAAACGAGCTCTGCTTATATAGACCTCCCACCGTACAC |
| | | GCCTACCGCCCATTTGCGTCAATGGGGCGGAGTTGTTACG |
| | | ACATTTTGGAAAGTCCCGTTGATTTTGGTGCCAAAACAAA |
| | | CTCCCATTGACGTCAATGGGGTGGAGACTTGGAAATCCCC |
| | | GTGAGTCAAACCGCTATCCACGCCCATTGATGTACTGCCA |
| | | AAACCGCATCACCATGGTAATAGCGATGACTAATACGTAG |
| | | ATGTACTGCCAAGTAGGAAAGTCCCATAAGGTCATGTACT |
| | | GGGCATAATGCCAGGCGGGCCATTTACCGTCATTGACGTC |
| | | AATAGGGGGCGTACTTGGCATATGATACACTTGATGTACT |
| | | GCCAAGTGGGCAGTTTACCGTAAATACTCCACCCATTGAC |
| | | GTCAATGGAAAGTCCCTATTGGCGTTACTATGGGAACATA |
| | | CGTCATTATTGACGTCAATGGGCGGGGGTCGTTGGGCGGT |
| | | CAGCCAGGCGGGCCATTTACCGTAAGTTATGTAACGGGTA |
| | | CCGAATTCAGATCAGCTTCCTAGGAACCCCTAGTGATGGA |
| | | GTTGGCCACTCCCTCTCTGCGCGCTCGCTCGCTCACTGAGG |
| | | CCGGGCGACCAAAGGTCGCCCGACGCCCGGGCTTTGCCCG |
| | | GGCGGCCTCAGTGAGCGAGCGAGCGCGCCAGGATCCGAG |
| | | CTTGTCGAGAAGTACTAGAGGATCATCAAGCTGTAGCCAA |
| | | CCACTAGAACTATAGCTAGAGTCCTGGGCGAACAAACGAT |
| | | GCTCGCCTTCCAGAAAACCGAGGATGCGAACCACTTCATC |
| | | CGGGGTCAGCACCACCGGCAAGCGCCGCGACGGCCGAGG |
| | | TCTTCCGATCTCCTGAAGCCAGGGCAGATCCGTGCACAGC |
| | | ACCTTGCCGTAGAAGAACAGCAAGGCCGCCAATGCCTGA |
| | | CGATGCGTGGAGACCGAAACCTTGCGCTCGTTCGCCAGCC |
| | | AGGACAGAAATGCCTCGACTTCGCTGCTGCCCAAGGTTGC |
| | | CGGGTGACGCACACCGTGGAAACGGATGAAGGCACGAAC |
| | | CCAGTTGACATAAGCCTGTTCGGTTCGTAAACTGTAATGC |
| | | AAGTAGCGTATGCGCTCACGCAACTGGTCCAGAACCTTGA |
| | | CCGAACGCAGCGGTGGTAACGGCGCAGTGGCGGTTTTCAT |
| | | GGCTTGTTATGACTGTTTTTTTGTACAGTCTATGCCTCGGG |
| | | CATCCAAGCAGCAAGCGCGTTACGCCGTGGGTCGATGTTT |
| | | GATGTTATGGAGCAGCAACGATGTTACGCAGCAGCAACG |
| | | ATGTTACGCAGCAGGGCAGTCGCCCTAAAACAAAGTTAG |
| | | GTGGCTCAAGTATGGGCATCATTCGCACATGTAGGCTCGG |
| | | CCCTGACCAAGTCAAATCCATGCGGGCTGCTCTTGATCTTT |
| | | TCGGTCGTGAGTTCGGAGACGTAGCCACCTACTCCCAACA |
| | | TCAGCCGGACTCCGATTACCTCGGGAACTTGCTCCGTAGT |
| | | AAGACATTCATCGCGCTTGCTGCCTTCGACCAAGAAGCGG |
| | | TTGTTGGCGCTCTCGCGGCTTACGTTCTGCCCAGGTTTGAG |
| | | CAGCCGCGTAGTGAGATCTATATCTATGATCTCGCAGTCT |
| | | CCGGCGAGCACCGGAGGCAGGGCATTGCCACCGCGCTCA |
| | | TCAATCTCCTCAAGCATGAGGCCAACGCGCTTGGTGCTTA |
| | | TGTGATCTACGTGCAAGCAGATTACGGTGACGATCCCGCA |
| | | GTGGCTCTCTATACAAAGTTGGGCATACGGGAAGAAGTGA |
| | | TGCACTTTGATATCGACCCAAGTACCGCCACCTAACAATT |
| | | CGTTCAAGCCGAGATCGGCTTCCCGGCCGCGGAGTTGTTC |
| | | GGTAAATTGTCACAACGCCGCGAATATAGTCTTTACCATG |
| | | CCCTTGGCCACGCCCCTCTTTAATACGACGGGCAATTTGC |
| | | ACTTCAGAAAATGAAGAGTTTGCTTTAGCCATAACAAAAG |
| | | TCCAGTATGCTTTTTCACAGCATAACTGGACTGATTTCAGT |
| | | TTACAACTATTCTGTCTAGTTTAAGACTTTATTGTCATAGT |
| | | TTAGATCTATTTTGTTCAGTTTAAGACTTTATTGTCCGCCC |
| | | ACACCCGCTTACGCAGGGCATCCATTTATTACTCAACCGT |
| | | AACCGATTTTGCCAGGTTACGCGGCTGGTCTGCGGTGTGA |
| | | AATACCGCACAGATGCGTAAGGAGAAAATACCGCATCAG |
| | | GCGCTCTTCCGCTTCCTCGCTCACTGACTCGCTGCGCTCGG |
| | | TCGTTCGGCTGCGGCGAGCGGTATCAGCTCACTCAAAGGC |
| | | GGTAATACGGTTATCCACAGAATCAGGGGATAACGCAGG |
| | | AAAGAACATGTGAGCAAAAGGCCAGCAAAAGGCCAGGAA |
| | | CCGTAAAAAGGCCGCGTTGCTGGCGTTTTTCCATAGGCTC |
| | | CGCCCCCCTGACGAGCATCACAAAAATCGACGCTCAAGTC |
| | | AGAGGTGGCGAAACCCGACAGGACTATAAAGATACCAGG |
| | | CGTTTCCCCCTGGAAGCTCCCTCGTGCGCTCTCCTGTTCCG |
| | | ACCCTGCCGCTTACCGGATACCTGTCCGCCTTTCTCCCTTC |
| | | GGGAAGCGTGGCGCTTTCTCAATGCTCACGCTGTAGGTAT |
| | | CTCAGTTCGGTGTAGGTCGTTCGCTCCAAGCTGGGCTGTG |
| | | TGCACGAACCCCCCGTTCAGCCCGACCGCTGCGCCTTATC |
| | | CGGTAACTATCGTCTTGAGTCCAACCCGGTAAGACACGAC |
| | | TTATCGCCACTGGCAGCAGCCACTGGTAACAGGATTAGCA |
| | | GAGCGAGGTATGTAGGCGGTGCTACAGAGTTCTTGAAGTG |
| | | GTGGCCTAACTACGGCTACACTAGAAGGACAGTATTTGGT |
| | | ATCTGCGCTCTGCTGAAGCCAGTTACCTTCGGAAAAAGAG |
| | | TTGGTAGCTCTTGATCCGGCAAACAAACCACCGCTGGTAG |
| | | CGGTGGTTTTTTTGTTTGCAAGCAGCAGATTACGCGCAGA |
| | | AAAAAAGGATCTCAAGAAGATCCTTTGATCTTTTCTACGG |
| | | GGTCTGACGCTCAGTGGAACGAAAACTCACGTTAAGGGAT |
| | | TTTGGTCATGAGATTATCAAAAAGGATCTTCACCTAGATC |
| | | CTTTTAAATTAAAAATGAAGTTTTAAATCAATCTAAAGTA |
| | | TATATGAGTAAACTTGGTCTGACAGTTACCAATGCTTAAT |
| | | CAGTGAGGCACCTATCTCAGCGATCTGTCTATTTCGTTCAT |
| | | CCATAGTTGCCTGACTCCCCGTCGTGTAGATAACTACGAT |
| | | ACGGGAGGGCTTACCATCTGGCCCCAGTGCTGCAATGATA |
| | | CCGCGAGACCCACGCTCACCGGCTCCAGATTTATCAGCAA |
| | | TAAACCAGCCAGCCGGAAGGGCCGAGCGCAGAAGTGGTC |
| | | CTGCAACTTTATCCGCCTCCATCCAGTCTATTAATTGTTGC |
| | | CGGGAAGCTAGAGTAAGTAGTTCGCCAGTTAATAGTTTGC |
| | | GCAACGTTGTTGCCATTGCTACAGGCATCGTGGTGTCACG |
| | | CTCGTCGTTTGGTATGGCTTCATTCAGCTCCGGTTCCCAAC |
| | | GATCAAGGCGAGTTACATGATCCCCCATGTTGTGCAAAAA |
| | | AGCGGTTAGCTCCTTCGGTCCTCCGATCGTTGTCAGAAGT |
| | | AAGTTGGCCGCAGTGTTATCACTCATGGTTATGGCAGCAC |
| | | TGCATAATTCTCTTACTGTCATGCCATCCGTAAGATGCTTT |
| | | TCTGTGACTGGTGAGTACTCAACCAAGTCATTCTGAGAAT |
| | | AGTGTATGCGGCGACCGAGTTGCTCTTGCCCGGCGTCAAT |
| | | ACGGGATAATACCGCGCCACATAGCAGAACTTTAAAAGT |
| | | GCTCATCATTGGAAAACGTTCTTCGGGGCGAAAACTCTCA |
| | | AGGATCTTACCGCTGTTGAGATCCAGTTCGATGTAACCCA |
| | | CTCGTGCACCCAACTGATCTTCAGCATCTTTTACTTTCACC |
| | | AGCGTTTCTGGGTGAGCAAAAACAGGAAGGCAAAATGCC |
| | | GCAAAAAAGGGAATAAGGGCGACACGGAAATGTTGAATA |
| | | CTCATACTCTTCCTTTTTCAATATTATTGAAGCATTTATCA |
| | | GGGTTATTGTCTCATGAGCGGATACATATTTGAATGTATTT |
| | | AGAAAAATAAACAAATAGGGGTTCCGCGCACATTTCCCCG |
| | | AAAAGTGCCACCTGAAATTGTAAACGTTAATATTTTGTTA |
| | | AAATTCGCGTTAAATTTTTGTTAAATCAGCTCATTTTTTAA |
| | | CCAATAGGCCGAAATCGGCAAAATCCCTTATAAATCAAAA |
| | | GAATAGACCGAGATAGGGTTGAGTGTTGTTCCAGTTTGGA |
| | | ACAAGAGTCCACTATTAAAGAACGTGGACTCCAACGTCAA |
| | | AGGGCGAAAAACCGTCTATCAGGGCGATGGCCCACTACG |
| | | TGAACCATCACCCTAATCAAGTTTTTTGGGGTCGAGGTGC |
| | | CGTAAAGCACTAAATCGGAACCCTAAAGGGAGCCCCCGA |
| | | TTTAGAGCTTGACGGGGAAAGCCGGCGAACGTGGCGAGA |
| | | AAGGAAGGGAAGAAAGCGAAAGGAGCGGGCGCTAGGGC |
| | | GCTGGCAAGTGTAGCGGTCACGCTGCGCGTAACCACCACA |
| | | CCCGCCGCGCTTAATGCGCCGCTACAGGGCGCGTC |
| AMI269 | 174 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTAACATCCC |
| | | AGGCCGCTCATGGAGCCGATTCGGTCCAGCTTGAGGCCGA |
| | | AGCAGCCCTTGGACAAGCCAGAGCCGCCTCCGCCGCTTCC |
| | | GCCTCCGCCAGATCCGCCTCCGCCGCTTCCGCCTCCGCCCT |
| | | TGCCCAGGCTCAGGCTCAGGCTCTTCTGGGTGTAGTGGTT |
| | | GTGCAGGGCCTCGTGCATCACGCTGCAGCTGAACACGTTG |
| | | CCCTCCTGCCATCTGCTCTTGTCCACGGTCAGTCTGCTGTA |
| | | CAGGAAGAAGCTGCCGTCGCTGTCCAGCACGGGGGGGGT |
| | | GGTCTTGTAGTTGTTCTCGGGCTGGCCGTTGCTCTCCCACT |
| | | CCACGGCGATGTCGCTGGGGTAGAAGCCCTTCACCAGGCA |
| | | GGTCAGGCTCACCTGGTTCTTGGTCATCTCCTCCTGGCTGG |
| | | GGGGCAGGGTGTACACCTGGGGCTCTCTGGGCTGGCCCTT |
| | | GGCCTTGCTGATGGTCTTCTCGATGCTGCTGGGCAGGCCC |
| | | TTGTTGCTCACCTTGCACTTGTACTCCTTGCCGTTCAGCCA |
| | | GTCCTGGTGCAGCACGGTCAGCACGCTCACCACTCTGTAG |
| | | GTGCTGTTGAACTGCTCCTCTCTGGGCTTGGTCTTGGCGTT |
| | | GTGCACCTCCACGCCGTCCACGTACCAGTTGAACTGCACC |
| | | TCGGGGTCCTCCTGGCTCACGTCCACCACCACGCAGGTCA |
| | | CCTCGGGGGTTCTGCTGATCATCAGGGTGTCCTTGGGCTT |
| | | GGGGGGGAACAGGAACACGCTGGGGCCGCCCAGGAACTC |
| | | GGGGGCGGGGCAGGGGGGGCAGGGGGGGCCGTACTTGCT |
| | | CTCCACTCTCTTGTCGCACTGCACGCCCTTAAGGATGGCC |
| | | ACCAGGAACAGCCAGCTCAGGCCGAACTCCATGGTGGCG |
| | | GCAGGCCTAGAAGTAAAGGCAACATCCACTGAGGAGCAG |
| | | TTCTTTGATTTGCACCACCACCGGATCCGGGACCTGAAAT |
| | | AAAAGACAAAAAGACTAAACTTACCAGTTAACTCTAGAG |
| | | TCCGGAGGCTGGATCGGTCCCGGTGTCTTCTATGGAGGTC |
| | | AAAACAGCGTGGATGGCGTCTCCAGGCGATCTGACGGTTC |
| | | ACTAAACGAGCTCTGCTTATATAGACCTCCCACCGTACAC |
| | | GCCTACCGCCCATTTGCGTCAATGGGGCGGAGTTGTTACG |
| | | ACATTTTGGAAAGTCCCGTTGATTTTGGTGCCAAAACAAA |
| | | CTCCCATTGACGTCAATGGGGTGGAGACTTGGAAATCCCC |
| | | GTGAGTCAAACCGCTATCCACGCCCATTGATGTACTGCCA |
| | | AAACCGCATCACCATGGTAATAGCGATGACTAATACGTAG |
| | | ATGTACTGCCAAGTAGGAAAGTCCCATAAGGTCATGTACT |
| | | GGGCATAATGCCAGGCGGGCCATTTACCGTCATTGACGTC |
| | | AATAGGGGGCGTACTTGGCATATGATACACTTGATGTACT |
| | | GCCAAGTGGGCAGTTTACCGTAAATACTCCACCCATTGAC |
| | | GTCAATGGAAAGTCCCTATTGGCGTTACTATGGGAACATA |
| | | CGTCATTATTGACGTCAATGGGCGGGGGTCGTTGGGCGGT |
| | | CAGCCAGGCGGGCCATTTACCGTAAGTTATGTAACGGGTA |
| | | CCGAATTCAGATCAGCTTCCTAGGAACCCCTAGTGATGGA |
| | | GTTGGCCACTCCCTCTCTGCGCGCTCGCTCGCTCACTGAGG |
| | | CCGGGCGACCAAAGGTCGCCCGACGCCCGGGCTTTGCCCG |
| | | GGCGGCCTCAGTGAGCGAGCGAGCGCGCCAGCAAGCTGT |
| | | AGCCAACCACTAGAACTATAGCTAGAGTCCTGGGCGAAC |
| | | AAACGATGCTCGCCTTCCAGAAAACCGAGGATGCGAACC |
| | | ACTTCATCCGGGGTCAGCACCACCGGCAAGCGCCGCGACG |
| | | GCCGAGGTCTTCCGATCTCCTGAAGCCAGGGCAGATCCGT |
| | | GCACAGCACCTTGCCGTAGAAGAACAGCAAGGCCGCCAA |
| | | TGCCTGACGATGCGTGGAGACCGAAACCTTGCGCTCGTTC |
| | | GCCAGCCAGGACAGAAATGCCTCGACTTCGCTGCTGCCCA |
| | | AGGTTGCCGGGTGACGCACACCGTGGAAACGGATGAAGG |
| | | CACGAACCCAGTTGACATAAGCCTGTTCGGTTCGTAAACT |
| | | GTAATGCAAGTAGCGTATGCGCTCACGCAACTGGTCCAGA |
| | | ACCTTGACCGAACGCAGCGGTGGTAACGGCGCAGTGGCG |
| | | GTTTTCATGGCTTGTTATGACTGTTTTTTTGTACAGTCTAT |
| | | GCCTCGGGCATCCAAGCAGCAAGCGCGTTACGCCGTGGGT |
| | | CGATGTTTGATGTTATGGAGCAGCAACGATGTTACGCAGC |
| | | AGCAACGATGTTACGCAGCAGGGCAGTCGCCCTAAAACA |
| | | AAGTTAGGTGGCTCAAGTATGGGCATCATTCGCACATGTA |
| | | GGCTCGGCCCTGACCAAGTCAAATCCATGCGGGCTGCTCT |
| | | TGATCTTTTCGGTCGTGAGTTCGGAGACGTAGCCACCTAC |
| | | TCCCAACATCAGCCGGACTCCGATTACCTCGGGAACTTGC |
| | | TCCGTAGTAAGACATTCATCGCGCTTGCTGCCTTCGACCA |
| | | AGAAGCGGTTGTTGGCGCTCTCGCGGCTTACGTTCTGCCC |
| | | AGGTTTGAGCAGCCGCGTAGTGAGATCTATATCTATGATC |
| | | TCGCAGTCTCCGGCGAGCACCGGAGGCAGGGCATTGCCAC |
| | | CGCGCTCATCAATCTCCTCAAGCATGAGGCCAACGCGCTT |
| | | GGTGCTTATGTGATCTACGTGCAAGCAGATTACGGTGACG |
| | | ATCCCGCAGTGGCTCTCTATACAAAGTTGGGCATACGGGA |
| | | AGAAGTGATGCACTTTGATATCGACCCAAGTACCGCCACC |
| | | TAACAATTCGTTCAAGCCGAGATCGGCTTCCCGGCCGCGG |
| | | AGTTGTTCGGTAAATTGTCACAACGCCGCGAATATAGTCT |
| | | TTACCATGCCCTTGGCCACGCCCCTCTTTAATACGACGGG |
| | | CAATTTGCACTTCAGAAAATGAAGAGTTTGCTTTAGCCAT |
| | | AACAAAAGTCCAGTATGCTTTTTCACAGCATAACTGGACT |
| | | GATTTCAGTTTACAACTATTCTGTCTAGTTTAAGACTTTAT |
| | | TGTCATAGTTTAGATCTATTTTGTTCAGTTTAAGACTTTAT |
| | | TGTCCGCCCACACCCGCTTACGCAGGGCATCCATTTATTA |
| | | CTCAACCGTAACCGATTTTGCCAGGTTACGCGGCTGGTCT |
| | | GCGGTGTGAAATACCGCACAGATGCGTAAGGAGAAAATA |
| | | CCGCATCAGGCGCTCTTCCGCTTCCTCGCTCACTGACTCGC |
| | | TGCGCTCGGTCGTTCGGCTGCGGCGAGCGGTATCAGCTCA |
| | | CTCAAAGGCGGTAATACGGTTATCCACAGAATCAGGGGAT |
| | | AACGCAGGAAAGAACATGTGAGCAAAAGGCCAGCAAAAG |
| | | GCCAGGAACCGTAAAAAGGCCGCGTTGCTGGCGTTTTTCC |
| | | ATAGGCTCCGCCCCCCTGACGAGCATCACAAAAATCGACG |
| | | CTCAAGTCAGAGGTGGCGAAACCCGACAGGACTATAAAG |
| | | ATACCAGGCGTTTCCCCCTGGAAGCTCCCTCGTGCGCTCTC |
| | | CTGTTCCGACCCTGCCGCTTACCGGATACCTGTCCGCCTTT |
| | | CTCCCTTCGGGAAGCGTGGCGCTTTCTCAATGCTCACGCT |
| | | GTAGGTATCTCAGTTCGGTGTAGGTCGTTCGCTCCAAGCT |
| | | GGGCTGTGTGCACGAACCCCCCGTTCAGCCCGACCGCTGC |
| | | GCCTTATCCGGTAACTATCGTCTTGAGTCCAACCCGGTAA |
| | | GACACGACTTATCGCCACTGGCAGCAGCCACTGGTAACAG |
| | | GATTAGCAGAGCGAGGTATGTAGGCGGTGCTACAGAGTTC |
| | | TTGAAGTGGTGGCCTAACTACGGCTACACTAGAAGGACAG |
| | | TATTTGGTATCTGCGCTCTGCTGAAGCCAGTTACCTTCGGA |
| | | AAAAGAGTTGGTAGCTCTTGATCCGGCAAACAAACCACCG |
| | | CTGGTAGCGGTGGTTTTTTTGTTTGCAAGCAGCAGATTAC |
| | | GCGCAGAAAAAAAGGATCTCAAGAAGATCCTTTGATCTTT |
| | | TCTACGGGGTCTGACGCTCAGTGGAACGAAAACTCACGTT |
| | | AAGGGATTTTGGTCATGAGATTATCAAAAAGGATCTTCAC |
| | | CTAGATCCTTTTAAATTAAAAATGAAGTTTTAAATCAATCT |
| | | AAAGTATATATGAGTAAACTTGGTCTGACAGTTACCAATG |
| | | CTTAATCAGTGAGGCACCTATCTCAGCGATCTGTCTATTTC |
| | | GTTCATCCATAGTTGCCTGACTCCCCGTCGTGTAGATAACT |
| | | ACGATACGGGAGGGCTTACCATCTGGCCCCAGTGCTGCAA |
| | | TGATACCGCGAGACCCACGCTCACCGGCTCCAGATTTATC |
| | | AGCAATAAACCAGCCAGCCGGAAGGGCCGAGCGCAGAAG |
| | | TGGTCCTGCAACTTTATCCGCCTCCATCCAGTCTATTAATT |
| | | GTTGCCGGGAAGCTAGAGTAAGTAGTTCGCCAGTTAATAG |
| | | TTTGCGCAACGTTGTTGCCATTGCTACAGGCATCGTGGTGT |
| | | CACGCTCGTCGTTTGGTATGGCTTCATTCAGCTCCGGTTCC |
| | | CAACGATCAAGGCGAGTTACATGATCCCCCATGTTGTGCA |
| | | AAAAAGCGGTTAGCTCCTTCGGTCCTCCGATCGTTGTCAG |
| | | AAGTAAGTTGGCCGCAGTGTTATCACTCATGGTTATGGCA |
| | | GCACTGCATAATTCTCTTACTGTCATGCCATCCGTAAGATG |
| | | CTTTTCTGTGACTGGTGAGTACTCAACCAAGTCATTCTGAG |
| | | AATAGTGTATGCGGCGACCGAGTTGCTCTTGCCCGGCGTC |
| | | AATACGGGATAATACCGCGCCACATAGCAGAACTTTAAA |
| | | AGTGCTCATCATTGGAAAACGTTCTTCGGGGCGAAAACTC |
| | | TCAAGGATCTTACCGCTGTTGAGATCCAGTTCGATGTAAC |
| | | CCACTCGTGCACCCAACTGATCTTCAGCATCTTTTACTTTC |
| | | ACCAGCGTTTCTGGGTGAGCAAAAACAGGAAGGCAAAAT |
| | | GCCGCAAAAAAGGGAATAAGGGCGACACGGAAATGTTGA |
| | | ATACTCATACTCTTCCTTTTTCAATATTATTGAAGCATTTA |
| | | TCAGGGTTATTGTCTCATGAGCGGATACATATTTGAATGT |
| | | ATTTAGAAAAATAAACAAATAGGGGTTCCGCGCACATTTC |
| | | CCCGAAAAGTGCCACCTGAAATTGTAAACGTTAATATTTT |
| | | GTTAAAATTCGCGTTAAATTTTTGTTAAATCAGCTCATTTT |
| | | TTAACCAATAGGCCGAAATCGGCAAAATCCCTTATAAATC |
| | | AAAAGAATAGACCGAGATAGGGTTGAGTGTTGTTCCAGTT |
| | | TGGAACAAGAGTCCACTATTAAAGAACGTGGACTCCAAC |
| | | GTCAAAGGGCGAAAAACCGTCTATCAGGGCGATGGCCCA |
| | | CTACGTGAACCATCACCCTAATCAAGTTTTTTGGGGTCGA |
| | | GGTGCCGTAAAGCACTAAATCGGAACCCTAAAGGGAGCC |
| | | CCCGATTTAGAGCTTGACGGGGAAAGCCGGCGAACGTGG |
| | | CGAGAAAGGAAGGGAAGAAAGCGAAAGGAGCGGGCGCT |
| | | AGGGCGCTGGCAAGTGTAGCGGTCACGCTGCGCGTAACC |
| | | ACCACACCCGCCGCGCTTAATGCGCCGCTACAGGGCGCGT |
| | | C |
| AMI270 | 175 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGCTACTTGCCCA |
| | | GGCTCAGGCTCAGGCTCTTCTGGGTGTAGTGGTTGTGCAG |
| | | GGCCTCGTGCATCACGCTGCAGCTGAACACGTTGCCCTCC |
| | | TGCCATCTGCTCTTGTCCACGGTCAGTCTGCTGTACAGGA |
| | | AGAAGCTGCCGTCGCTGTCCAGCACGGGGGGGGTGGTCTT |
| | | GTAGTTGTTCTCGGGCTGGCCGTTGCTCTCCCACTCCACGG |
| | | CGATGTCGCTGGGGTAGAAGCCCTTCACCAGGCAGGTCAG |
| | | GCTCACCTGGTTCTTGGTCATCTCCTCCTGGCTGGGGGGCA |
| | | GGGTGTACACCTGGGGCTCTCTGGGCTGGCCCTTGGCCTT |
| | | GCTGATGGTCTTCTCGATGCTGCTGGGCAGGCCCTTGTTGC |
| | | TCACCTTGCACTTGTACTCCTTGCCGTTCAGCCAGTCCTGG |
| | | TGCAGCACGGTCAGCACGCTCACCACTCTGTAGGTGCTGT |
| | | TGAACTGCTCCTCTCTGGGCTTGGTCTTGGCGTTGTGCACC |
| | | TCCACGCCGTCCACGTACCAGTTGAACTGCACCTCGGGGT |
| | | CCTCCTGGCTCACGTCCACCACCACGCAGGTCACCTCGGG |
| | | GGTTCTGCTGATCATCAGGGTGTCCTTGGGCTTGGGGGGG |
| | | AACAGGAACACGCTGGGGCCGCCCAGGAACTCGGGGGCG |
| | | GGGCAGGGGGGGCAGGGGGGGCCGTACTTGCTCTCCACT |
| | | CTCTTGTCAGAGCCGCCTCCGCCGCTTCCGCCTCCGCCAG |
| | | ATCCGCCTCCGCCGCTTCCGCCTCCGCCACATCCCAGGCC |
| | | GCTCATGGAGCCGATTCGGTCCAGCTTGAGGCCGAAGCAG |
| | | CCCTTGGACAAGCCGATGTCGCACTGCACGCCCTTAAGGA |
| | | TGGCCACCAGGAACAGCCAGCTCAGGCCGAACTCCATGGT |
| | | GGCGGCAGGCCTAGAAGTAAAGGCAACATCCACTGAGGA |
| | | GCAGTTCTTTGATTTGCACCACCACCGGATCCGGGACCTG |
| | | AAATAAAAGACAAAAAGACTAAACTTACCAGTTAACTCT |
| | | AGAGTCCGGAGGCTGGATCGGTCCCGGTGTCTTCTATGGA |
| | | GGTCAAAACAGCGTGGATGGCGTCTCCAGGCGATCTGACG |
| | | GTTCACTAAACGAGCTCTGCTTATATAGACCTCCCACCGT |
| | | ACACGCCTACCGCCCATTTGCGTCAATGGGGCGGAGTTGT |
| | | TACGACATTTTGGAAAGTCCCGTTGATTTTGGTGCCAAAA |
| | | CAAACTCCCATTGACGTCAATGGGGTGGAGACTTGGAAAT |
| | | CCCCGTGAGTCAAACCGCTATCCACGCCCATTGATGTACT |
| | | GCCAAAACCGCATCACCATGGTAATAGCGATGACTAATAC |
| | | GTAGATGTACTGCCAAGTAGGAAAGTCCCATAAGGTCATG |
| | | TACTGGGCATAATGCCAGGCGGGCCATTTACCGTCATTGA |
| | | CGTCAATAGGGGGCGTACTTGGCATATGATACACTTGATG |
| | | TACTGCCAAGTGGGCAGTTTACCGTAAATACTCCACCCAT |
| | | TGACGTCAATGGAAAGTCCCTATTGGCGTTACTATGGGAA |
| | | CATACGTCATTATTGACGTCAATGGGCGGGGGTCGTTGGG |
| | | CGGTCAGCCAGGCGGGCCATTTACCGTAAGTTATGTAACG |
| | | GGTACCGAATTCAGATCAGCTTCCTAGGAACCCCTAGTGA |
| | | TGGAGTTGGCCACTCCCTCTCTGCGCGCTCGCTCGCTCACT |
| | | GAGGCCGGGCGACCAAAGGTCGCCCGACGCCCGGGCTTT |
| | | GCCCGGGCGGCCTCAGTGAGCGAGCGAGCGCGCCAGGAT |
| | | CCGAGCTTGTCGAGAAGTACTAGAGGATCATCAAGCTGTA |
| | | GCCAACCACTAGAACTATAGCTAGAGTCCTGGGCGAACA |
| | | AACGATGCTCGCCTTCCAGAAAACCGAGGATGCGAACCA |
| | | CTTCATCCGGGGTCAGCACCACCGGCAAGCGCCGCGACGG |
| | | CCGAGGTCTTCCGATCTCCTGAAGCCAGGGCAGATCCGTG |
| | | CACAGCACCTTGCCGTAGAAGAACAGCAAGGCCGCCAAT |
| | | GCCTGACGATGCGTGGAGACCGAAACCTTGCGCTCGTTCG |
| | | CCAGCCAGGACAGAAATGCCTCGACTTCGCTGCTGCCCAA |
| | | GGTTGCCGGGTGACGCACACCGTGGAAACGGATGAAGGC |
| | | ACGAACCCAGTTGACATAAGCCTGTTCGGTTCGTAAACTG |
| | | TAATGCAAGTAGCGTATGCGCTCACGCAACTGGTCCAGAA |
| | | CCTTGACCGAACGCAGCGGTGGTAACGGCGCAGTGGCGG |
| | | TTTTCATGGCTTGTTATGACTGTTTTTTTGTACAGTCTATGC |
| | | CTCGGGCATCCAAGCAGCAAGCGCGTTACGCCGTGGGTCG |
| | | ATGTTTGATGTTATGGAGCAGCAACGATGTTACGCAGCAG |
| | | CAACGATGTTACGCAGCAGGGCAGTCGCCCTAAAACAAA |
| | | GTTAGGTGGCTCAAGTATGGGCATCATTCGCACATGTAGG |
| | | CTCGGCCCTGACCAAGTCAAATCCATGCGGGCTGCTCTTG |
| | | ATCTTTTCGGTCGTGAGTTCGGAGACGTAGCCACCTACTC |
| | | CCAACATCAGCCGGACTCCGATTACCTCGGGAACTTGCTC |
| | | CGTAGTAAGACATTCATCGCGCTTGCTGCCTTCGACCAAG |
| | | AAGCGGTTGTTGGCGCTCTCGCGGCTTACGTTCTGCCCAG |
| | | GTTTGAGCAGCCGCGTAGTGAGATCTATATCTATGATCTC |
| | | GCAGTCTCCGGCGAGCACCGGAGGCAGGGCATTGCCACC |
| | | GCGCTCATCAATCTCCTCAAGCATGAGGCCAACGCGCTTG |
| | | GTGCTTATGTGATCTACGTGCAAGCAGATTACGGTGACGA |
| | | TCCCGCAGTGGCTCTCTATACAAAGTTGGGCATACGGGAA |
| | | GAAGTGATGCACTTTGATATCGACCCAAGTACCGCCACCT |
| | | AACAATTCGTTCAAGCCGAGATCGGCTTCCCGGCCGCGGA |
| | | GTTGTTCGGTAAATTGTCACAACGCCGCGAATATAGTCTT |
| | | TACCATGCCCTTGGCCACGCCCCTCTTTAATACGACGGGC |
| | | AATTTGCACTTCAGAAAATGAAGAGTTTGCTTTAGCCATA |
| | | ACAAAAGTCCAGTATGCTTTTTCACAGCATAACTGGACTG |
| | | ATTTCAGTTTACAACTATTCTGTCTAGTTTAAGACTTTATT |
| | | GTCATAGTTTAGATCTATTTTGTTCAGTTTAAGACTTTATT |
| | | GTCCGCCCACACCCGCTTACGCAGGGCATCCATTTATTAC |
| | | TCAACCGTAACCGATTTTGCCAGGTTACGCGGCTGGTCTG |
| | | CGGTGTGAAATACCGCACAGATGCGTAAGGAGAAAATAC |
| | | CGCATCAGGCGCTCTTCCGCTTCCTCGCTCACTGACTCGCT |
| | | GCGCTCGGTCGTTCGGCTGCGGCGAGCGGTATCAGCTCAC |
| | | TCAAAGGCGGTAATACGGTTATCCACAGAATCAGGGGAT |
| | | AACGCAGGAAAGAACATGTGAGCAAAAGGCCAGCAAAAG |
| | | GCCAGGAACCGTAAAAAGGCCGCGTTGCTGGCGTTTTTCC |
| | | ATAGGCTCCGCCCCCCTGACGAGCATCACAAAAATCGACG |
| | | CTCAAGTCAGAGGTGGCGAAACCCGACAGGACTATAAAG |
| | | ATACCAGGCGTTTCCCCCTGGAAGCTCCCTCGTGCGCTCTC |
| | | CTGTTCCGACCCTGCCGCTTACCGGATACCTGTCCGCCTTT |
| | | CTCCCTTCGGGAAGCGTGGCGCTTTCTCAATGCTCACGCT |
| | | GTAGGTATCTCAGTTCGGTGTAGGTCGTTCGCTCCAAGCT |
| | | GGGCTGTGTGCACGAACCCCCCGTTCAGCCCGACCGCTGC |
| | | GCCTTATCCGGTAACTATCGTCTTGAGTCCAACCCGGTAA |
| | | GACACGACTTATCGCCACTGGCAGCAGCCACTGGTAACAG |
| | | GATTAGCAGAGCGAGGTATGTAGGCGGTGCTACAGAGTTC |
| | | TTGAAGTGGTGGCCTAACTACGGCTACACTAGAAGGACAG |
| | | TATTTGGTATCTGCGCTCTGCTGAAGCCAGTTACCTTCGGA |
| | | AAAAGAGTTGGTAGCTCTTGATCCGGCAAACAAACCACCG |
| | | CTGGTAGCGGTGGTTTTTTTGTTTGCAAGCAGCAGATTAC |
| | | GCGCAGAAAAAAAGGATCTCAAGAAGATCCTTTGATCTTT |
| | | TCTACGGGGTCTGACGCTCAGTGGAACGAAAACTCACGTT |
| | | AAGGGATTTTGGTCATGAGATTATCAAAAAGGATCTTCAC |
| | | CTAGATCCTTTTAAATTAAAAATGAAGTTTTAAATCAATCT |
| | | AAAGTATATATGAGTAAACTTGGTCTGACAGTTACCAATG |
| | | CTTAATCAGTGAGGCACCTATCTCAGCGATCTGTCTATTTC |
| | | GTTCATCCATAGTTGCCTGACTCCCCGTCGTGTAGATAACT |
| | | ACGATACGGGAGGGCTTACCATCTGGCCCCAGTGCTGCAA |
| | | TGATACCGCGAGACCCACGCTCACCGGCTCCAGATTTATC |
| | | AGCAATAAACCAGCCAGCCGGAAGGGCCGAGCGCAGAAG |
| | | TGGTCCTGCAACTTTATCCGCCTCCATCCAGTCTATTAATT |
| | | GTTGCCGGGAAGCTAGAGTAAGTAGTTCGCCAGTTAATAG |
| | | TTTGCGCAACGTTGTTGCCATTGCTACAGGCATCGTGGTGT |
| | | CACGCTCGTCGTTTGGTATGGCTTCATTCAGCTCCGGTTCC |
| | | CAACGATCAAGGCGAGTTACATGATCCCCCATGTTGTGCA |
| | | AAAAAGCGGTTAGCTCCTTCGGTCCTCCGATCGTTGTCAG |
| | | AAGTAAGTTGGCCGCAGTGTTATCACTCATGGTTATGGCA |
| | | GCACTGCATAATTCTCTTACTGTCATGCCATCCGTAAGATG |
| | | CTTTTCTGTGACTGGTGAGTACTCAACCAAGTCATTCTGAG |
| | | AATAGTGTATGCGGCGACCGAGTTGCTCTTGCCCGGCGTC |
| | | AATACGGGATAATACCGCGCCACATAGCAGAACTTTAAA |
| | | AGTGCTCATCATTGGAAAACGTTCTTCGGGGCGAAAACTC |
| | | TCAAGGATCTTACCGCTGTTGAGATCCAGTTCGATGTAAC |
| | | CCACTCGTGCACCCAACTGATCTTCAGCATCTTTTACTTTC |
| | | ACCAGCGTTTCTGGGTGAGCAAAAACAGGAAGGCAAAAT |
| | | GCCGCAAAAAAGGGAATAAGGGCGACACGGAAATGTTGA |
| | | ATACTCATACTCTTCCTTTTTCAATATTATTGAAGCATTTA |
| | | TCAGGGTTATTGTCTCATGAGCGGATACATATTTGAATGT |
| | | ATTTAGAAAAATAAACAAATAGGGGTTCCGCGCACATTTC |
| | | CCCGAAAAGTGCCACCTGAAATTGTAAACGTTAATATTTT |
| | | GTTAAAATTCGCGTTAAATTTTTGTTAAATCAGCTCATTTT |
| | | TTAACCAATAGGCCGAAATCGGCAAAATCCCTTATAAATC |
| | | AAAAGAATAGACCGAGATAGGGTTGAGTGTTGTTCCAGTT |
| | | TGGAACAAGAGTCCACTATTAAAGAACGTGGACTCCAAC |
| | | GTCAAAGGGCGAAAAACCGTCTATCAGGGCGATGGCCCA |
| | | CTACGTGAACCATCACCCTAATCAAGTTTTTTGGGGTCGA |
| | | GGTGCCGTAAAGCACTAAATCGGAACCCTAAAGGGAGCC |
| | | CCCGATTTAGAGCTTGACGGGGAAAGCCGGCGAACGTGG |
| | | CGAGAAAGGAAGGGAAGAAAGCGAAAGGAGCGGGCGCT |
| | | AGGGCGCTGGCAAGTGTAGCGGTCACGCTGCGCGTAACC |
| | | ACCACACCCGCCGCGCTTAATGCGCCGCTACAGGGCGCGT |
| | | C |
| AMI273 | 176 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTAACATCCC |
| | | AGGCCGCTCATGGAGCCGATTCGGTCCAGCTTGAGGCCGA |
| | | AGCAGCCCTTGGACAAGCCCTTCTTGTTGGCTCCTTTGTAT |
| | | TTGCGCGCGTTGGGGTGCTCAGAGCCGCCTCCGCCGCTTC |
| | | CGCCTCCGCCAGATCCGCCTCCGCCGCTTCCGCCTCCGCCC |
| | | TTGCCCAGGCTCAGGCTCAGGCTCTTCTGGGTGTAGTGGT |
| | | TGTGCAGGGCCTCGTGCATCACGCTGCAGCTGAACACGTT |
| | | GCCCTCCTGCCATCTGCTCTTGTCCACGGTCAGTCTGCTGT |
| | | ACAGGAAGAAGCTGCCGTCGCTGTCCAGCACGGGGGGGG |
| | | TGGTCTTGTAGTTGTTCTCGGGCTGGCCGTTGCTCTCCCAC |
| | | TCCACGGCGATGTCGCTGGGGTAGAAGCCCTTCACCAGGC |
| | | AGGTCAGGCTCACCTGGTTCTTGGTCATCTCCTCCTGGCTG |
| | | GGGGGCAGGGTGTACACCTGGGGCTCTCTGGGCTGGCCCT |
| | | TGGCCTTGCTGATGGTCTTCTCGATGCTGCTGGGCAGGCC |
| | | CTTGTTGCTCACCTTGCACTTGTACTCCTTGCCGTTCAGCC |
| | | AGTCCTGGTGCAGCACGGTCAGCACGCTCACCACTCTGTA |
| | | GGTGCTGTTGAACTGCTCCTCTCTGGGCTTGGTCTTGGCGT |
| | | TGTGCACCTCCACGCCGTCCACGTACCAGTTGAACTGCAC |
| | | CTCGGGGTCCTCCTGGCTCACGTCCACCACCACGCAGGTC |
| | | ACCTCGGGGGTTCTGCTGATCATCAGGGTGTCCTTGGGCT |
| | | TGGGGGGGAACAGGAACACGCTGGGGCCGCCCAGGAACT |
| | | CGGGGGCGGGGCAGGGGGGGCAGGGGGGGCCGTACTTGC |
| | | TCTCCACTCTCTTGTCGCACTGCACGCCCTTAAGGATGGCC |
| | | ACCAGGAACAGCCAGCTCAGGCCGAACTCCATGGTGGCG |
| | | GCAGGCCTAGAAGTAAAGGCAACATCCACTGAGGAGCAG |
| | | TTCTTTGATTTGCACCACCACCGGATCCGGGACCTGAAAT |
| | | AAAAGACAAAAAGACTAAACTTACCTGTGGGAGTAACGC |
| | | GGTCAGTCAGAGCCGGGGGGGGCGGCGCGAGGCGGCGGC |
| | | GGAGCGGGGCACGGGGCGAAGGCAGCGTCGCAGCGACTC |
| | | CCGCCCGCCGCGCGCTTCGCTTTTTATAGGGCCGCCGCCG |
| | | CCGCCGCCTCGCCATAAAAGGAAACTTTCGGAGCGCGCCG |
| | | CTCTGATTGGCTGCCGCCGCACCTCTCCGCCTCGCCCCGCC |
| | | CCGCCCCTCGCCCCGCCCCGCCCCGCCTGGCGCGCGCCCC |
| | | CCCCCCCCCCCCGCCCCCATCGCTGCACAAAATAATTAAA |
| | | AAATAAATAAATACAAAATTGGGGGTGGGGAGGGGGGGG |
| | | AGATGGGGAGAGTGAAGCAGAACGTGGGGCTCACCTCGA |
| | | GCCATGGTAATAGCGATGACTAATACGTAGATGTACTGCC |
| | | AAGTAGGAAAGTCCCATAAGGTCATGTACTGGGCATAATG |
| | | CCAGGCGGGCCATTTACCGTCATTGACGTCAATAGGGGGC |
| | | GTACTTGGCATATGATACACTTGATGTACTGCCAAGTGGG |
| | | CAGTTTACCGTAAATACTCCACCCATTGACGTCAATGGAA |
| | | AGTCCCTATTGGCGTTACTATGGGAACATACGTCATTATT |
| | | GACGTCAATGGGCGGGGGTCGTTGGGCGGTCAGCCAGGC |
| | | GGGCCATTTACCGTAAGTTATGTAACGGGTACCGAATTCA |
| | | GATCAGCTTCCTAGGAACCCCTAGTGATGGAGTTGGCCAC |
| | | TCCCTCTCTGCGCGCTCGCTCGCTCACTGAGGCCGGGCGA |
| | | CCAAAGGTCGCCCGACGCCCGGGCTTTGCCCGGGCGGCCT |
| | | CAGTGAGCGAGCGAGCGCGCCAGGATCCGAGCTTGTCGA |
| | | GAAGTACTAGAGGATCATCAAGCTGTAGCCAACCACTAG |
| | | AACTATAGCTAGAGTCCTGGGCGAACAAACGATGCTCGCC |
| | | TTCCAGAAAACCGAGGATGCGAACCACTTCATCCGGGGTC |
| | | AGCACCACCGGCAAGCGCCGCGACGGCCGAGGTCTTCCG |
| | | ATCTCCTGAAGCCAGGGCAGATCCGTGCACAGCACCTTGC |
| | | CGTAGAAGAACAGCAAGGCCGCCAATGCCTGACGATGCG |
| | | TGGAGACCGAAACCTTGCGCTCGTTCGCCAGCCAGGACAG |
| | | AAATGCCTCGACTTCGCTGCTGCCCAAGGTTGCCGGGTGA |
| | | CGCACACCGTGGAAACGGATGAAGGCACGAACCCAGTTG |
| | | ACATAAGCCTGTTCGGTTCGTAAACTGTAATGCAAGTAGC |
| | | GTATGCGCTCACGCAACTGGTCCAGAACCTTGACCGAACG |
| | | CAGCGGTGGTAACGGCGCAGTGGCGGTTTTCATGGCTTGT |
| | | TATGACTGTTTTTTTGTACAGTCTATGCCTCGGGCATCCAA |
| | | GCAGCAAGCGCGTTACGCCGTGGGTCGATGTTTGATGTTA |
| | | TGGAGCAGCAACGATGTTACGCAGCAGCAACGATGTTAC |
| | | GCAGCAGGGCAGTCGCCCTAAAACAAAGTTAGGTGGCTC |
| | | AAGTATGGGCATCATTCGCACATGTAGGCTCGGCCCTGAC |
| | | CAAGTCAAATCCATGCGGGCTGCTCTTGATCTTTTCGGTCG |
| | | TGAGTTCGGAGACGTAGCCACCTACTCCCAACATCAGCCG |
| | | GACTCCGATTACCTCGGGAACTTGCTCCGTAGTAAGACAT |
| | | TCATCGCGCTTGCTGCCTTCGACCAAGAAGCGGTTGTTGG |
| | | CGCTCTCGCGGCTTACGTTCTGCCCAGGTTTGAGCAGCCG |
| | | CGTAGTGAGATCTATATCTATGATCTCGCAGTCTCCGGCG |
| | | AGCACCGGAGGCAGGGCATTGCCACCGCGCTCATCAATCT |
| | | CCTCAAGCATGAGGCCAACGCGCTTGGTGCTTATGTGATC |
| | | TACGTGCAAGCAGATTACGGTGACGATCCCGCAGTGGCTC |
| | | TCTATACAAAGTTGGGCATACGGGAAGAAGTGATGCACTT |
| | | TGATATCGACCCAAGTACCGCCACCTAACAATTCGTTCAA |
| | | GCCGAGATCGGCTTCCCGGCCGCGGAGTTGTTCGGTAAAT |
| | | TGTCACAACGCCGCGAATATAGTCTTTACCATGCCCTTGG |
| | | CCACGCCCCTCTTTAATACGACGGGCAATTTGCACTTCAG |
| | | AAAATGAAGAGTTTGCTTTAGCCATAACAAAAGTCCAGTA |
| | | TGCTTTTTCACAGCATAACTGGACTGATTTCAGTTTACAAC |
| | | TATTCTGTCTAGTTTAAGACTTTATTGTCATAGTTTAGATC |
| | | TATTTTGTTCAGTTTAAGACTTTATTGTCCGCCCACACCCG |
| | | CTTACGCAGGGCATCCATTTATTACTCAACCGTAACCGAT |
| | | TTTGCCAGGTTACGCGGCTGGTCTGCGGTGTGAAATACCG |
| | | CACAGATGCGTAAGGAGAAAATACCGCATCAGGCGCTCTT |
| | | CCGCTTCCTCGCTCACTGACTCGCTGCGCTCGGTCGTTCGG |
| | | CTGCGGCGAGCGGTATCAGCTCACTCAAAGGCGGTAATAC |
| | | GGTTATCCACAGAATCAGGGGATAACGCAGGAAAGAACA |
| | | TGTGAGCAAAAGGCCAGCAAAAGGCCAGGAACCGTAAAA |
| | | AGGCCGCGTTGCTGGCGTTTTTCCATAGGCTCCGCCCCCCT |
| | | GACGAGCATCACAAAAATCGACGCTCAAGTCAGAGGTGG |
| | | CGAAACCCGACAGGACTATAAAGATACCAGGCGTTTCCCC |
| | | CTGGAAGCTCCCTCGTGCGCTCTCCTGTTCCGACCCTGCCG |
| | | CTTACCGGATACCTGTCCGCCTTTCTCCCTTCGGGAAGCGT |
| | | GGCGCTTTCTCAATGCTCACGCTGTAGGTATCTCAGTTCGG |
| | | TGTAGGTCGTTCGCTCCAAGCTGGGCTGTGTGCACGAACC |
| | | CCCCGTTCAGCCCGACCGCTGCGCCTTATCCGGTAACTAT |
| | | CGTCTTGAGTCCAACCCGGTAAGACACGACTTATCGCCAC |
| | | TGGCAGCAGCCACTGGTAACAGGATTAGCAGAGCGAGGT |
| | | ATGTAGGCGGTGCTACAGAGTTCTTGAAGTGGTGGCCTAA |
| | | CTACGGCTACACTAGAAGGACAGTATTTGGTATCTGCGCT |
| | | CTGCTGAAGCCAGTTACCTTCGGAAAAAGAGTTGGTAGCT |
| | | CTTGATCCGGCAAACAAACCACCGCTGGTAGCGGTGGTTT |
| | | TTTTGTTTGCAAGCAGCAGATTACGCGCAGAAAAAAAGGA |
| | | TCTCAAGAAGATCCTTTGATCTTTTCTACGGGGTCTGACGC |
| | | TCAGTGGAACGAAAACTCACGTTAAGGGATTTTGGTCATG |
| | | AGATTATCAAAAAGGATCTTCACCTAGATCCTTTTAAATT |
| | | AAAAATGAAGTTTTAAATCAATCTAAAGTATATATGAGTA |
| | | AACTTGGTCTGACAGTTACCAATGCTTAATCAGTGAGGCA |
| | | CCTATCTCAGCGATCTGTCTATTTCGTTCATCCATAGTTGC |
| | | CTGACTCCCCGTCGTGTAGATAACTACGATACGGGAGGGC |
| | | TTACCATCTGGCCCCAGTGCTGCAATGATACCGCGAGACC |
| | | CACGCTCACCGGCTCCAGATTTATCAGCAATAAACCAGCC |
| | | AGCCGGAAGGGCCGAGCGCAGAAGTGGTCCTGCAACTTT |
| | | ATCCGCCTCCATCCAGTCTATTAATTGTTGCCGGGAAGCT |
| | | AGAGTAAGTAGTTCGCCAGTTAATAGTTTGCGCAACGTTG |
| | | TTGCCATTGCTACAGGCATCGTGGTGTCACGCTCGTCGTTT |
| | | GGTATGGCTTCATTCAGCTCCGGTTCCCAACGATCAAGGC |
| | | GAGTTACATGATCCCCCATGTTGTGCAAAAAAGCGGTTAG |
| | | CTCCTTCGGTCCTCCGATCGTTGTCAGAAGTAAGTTGGCC |
| | | GCAGTGTTATCACTCATGGTTATGGCAGCACTGCATAATT |
| | | CTCTTACTGTCATGCCATCCGTAAGATGCTTTTCTGTGACT |
| | | GGTGAGTACTCAACCAAGTCATTCTGAGAATAGTGTATGC |
| | | GGCGACCGAGTTGCTCTTGCCCGGCGTCAATACGGGATAA |
| | | TACCGCGCCACATAGCAGAACTTTAAAAGTGCTCATCATT |
| | | GGAAAACGTTCTTCGGGGCGAAAACTCTCAAGGATCTTAC |
| | | CGCTGTTGAGATCCAGTTCGATGTAACCCACTCGTGCACC |
| | | CAACTGATCTTCAGCATCTTTTACTTTCACCAGCGTTTCTG |
| | | GGTGAGCAAAAACAGGAAGGCAAAATGCCGCAAAAAAG |
| | | GGAATAAGGGCGACACGGAAATGTTGAATACTCATACTCT |
| | | TCCTTTTTCAATATTATTGAAGCATTTATCAGGGTTATTGT |
| | | CTCATGAGCGGATACATATTTGAATGTATTTAGAAAAATA |
| | | AACAAATAGGGGTTCCGCGCACATTTCCCCGAAAAGTGCC |
| | | ACCTGAAATTGTAAACGTTAATATTTTGTTAAAATTCGCGT |
| | | TAAATTTTTGTTAAATCAGCTCATTTTTTAACCAATAGGCC |
| | | GAAATCGGCAAAATCCCTTATAAATCAAAAGAATAGACC |
| | | GAGATAGGGTTGAGTGTTGTTCCAGTTTGGAACAAGAGTC |
| | | CACTATTAAAGAACGTGGACTCCAACGTCAAAGGGCGAA |
| | | AAACCGTCTATCAGGGCGATGGCCCACTACGTGAACCATC |
| | | ACCCTAATCAAGTTTTTTGGGGTCGAGGTGCCGTAAAGCA |
| | | CTAAATCGGAACCCTAAAGGGAGCCCCCGATTTAGAGCTT |
| | | GACGGGGAAAGCCGGCGAACGTGGCGAGAAAGGAAGGG |
| | | AAGAAAGCGAAAGGAGCGGGCGCTAGGGCGCTGGCAAGT |
| | | GTAGCGGTCACGCTGCGCGTAACCACCACACCCGCCGCGC |
| | | TTAATGCGCCGCTACAGGGCGCGTC |
| AMI302 | 177 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGCTCAAGCAGTGATCAGATCCAGACATGATAAGATAC |
| | | ATTGATGAGTTTGGACAAACCACAACTAGAATGCAGTGAA |
| | | AAAAATGCTTTATTTGTGAAATTTGTGATGCTATTGCTTTA |
| | | TTTGTAACCATTATAAGCTGCAATAAACAAGTTAACAACA |
| | | ACAATTGCATTCATTTTATGTTTCAGGTTCAGGGGGAGGT |
| | | GTGGGAGGTTTTTTAAAGCAAGTAAAACCTCTACAAATGT |
| | | GGTATGGCTGATTATGATCCTCTAGTACTTCTCGACAAGCT |
| | | CGGATCCTGCGCGCTCGCTCGCTCACTGAGGCCGCCCGGG |
| | | CAAAGCCCGGGCGTCGGGCGACCTTTGGTCGCCCGGCCTC |
| | | AGTGAGCGAGCGAGCGCGCAGAGAGGGAGTGGGGTTGAT |
| | | CTCTCCCCAGCATGCCACACAAAAAACCAACACACAGATG |
| | | TAATGAAAATAAAGATATTTTATTTTCGAAGGTACCACCA |
| | | CGGAATTGTCAGTGCCCAACAGCCGAGCCCCTGTCCAGCA |
| | | GCGGGCAAGGCAGGCGGCGATGAGTTCCGCCGTGGCAAT |
| | | AGGGAGGGGGAAAGCGAAAGTCCCGGAAAGGAGCTGAC |
| | | AGGTGGTGGCAATGCCCCAACCAGTGGGGGTTGCGTCAGC |
| | | AAACACAGTGCACACCACGCCACGTTGCCTGACAACGGG |
| | | CCACAACTCCTCATAAAGAGACAGCAACCAGGATTTATAC |
| | | AAGGAGGAGAAAATGAAAGCCATACGGGAAGCAATAGCA |
| | | TGATACAAAGGCATTAAAGCAGCGTATCCACATAGCGTAA |
| | | AAGGAGCAACATAGTTAAGAATACCAGTCAATCTTTCACA |
| | | AATTTTGTAATCCAGAGGTTGATTCTCGAGTTATCATTTAC |
| | | CCGGAGACAGGGAGAGGCTCTTCTGCGTGTAGTGGTTGTG |
| | | CAGAGCCTCATGCATCACGGAGCATGAGAAGACGTTCCCC |
| | | TGCTGCCACCTGCTCTTGTCCACGGTGAGCTTGCTGTAGA |
| | | GGAAGAAGGAGCCGTCGGAGTCCAGCACGGGAGGCGTGG |
| | | TCTTGTAGTTGTTCTCCGGCTGCCCATTGCTCTCCCACTCC |
| | | ACGGCGATGTCGCTGGGATAGAAGCCTTTGACCAGGCAG |
| | | GTCAGGCTGACCTGGTTCTTGGTCAGCTCATCCCGGGATG |
| | | GGGGCAGGGTGTACACCTGTGGTTCTCGGGGCTGCCCTTT |
| | | GGCTTTGGAGATGGTTTTCTCGATGGGGGCTGGGAGGGCT |
| | | TTGTTGGAGACCTTGCACTTGTACTCCTTGCCATTCAGCTA |
| | | GTCCTGGTGCAGGACGGTGAGGACGCTGACCACACGGTA |
| | | CGTGCTGTTGTACTGCTCCTCCCGCGGCTTTGTCTTGGCAT |
| | | TATGCACCTCCACGCCGTCCACGTACCAGTTGAACTTGAC |
| | | CTCAGGGTCTTCGTGGCTCACGTCCACCACCACGCATGTG |
| | | ACCTCAGGGGTCCGGGAGATCATGAGGGTGTCCTTGGGTT |
| | | TTGGGGGGAAGAGGAAGACTGACGGTCCCCCCAGGAGTT |
| | | CAGGTGCTGGGCACGGTGGGCATGTGTGAGTTTTGTCAGA |
| | | GCCGCCGCCGCCGCTGCCGCCTCCGCCAGATCCGCCTCCG |
| | | CCGCTTCCGCCTCCCTAACATCCCAGGCCGCTCATGGAGC |
| | | CGATTCGGTCCAGCTTGAGGCCGAAGCAGCCCTTGGACAA |
| | | GCCCTTCTTGTTGGCTCCTTTGTATTTGCGCGCGTTGGGGT |
| | | GCTCGCACTGCACGCCCTTAAGGATGGCCACCAGGAACAG |
| | | CCAGCTCAGGCCGAACTCCATGGTGGCGGCAGGCCTAGA |
| | | AGTAAAGGCAACATCCACTGAGGAGCAGTTCTTTGATTTG |
| | | CACCACCACCGGATCCGGGACCTGAAATAAAAGACAAAA |
| | | AGACTAAACTTACCTGTGGGAGTAACGCGGTCAGTCAGAG |
| | | CCGGGGCGGGCGGCGCGAGGCGGCGGCGGAGCGGGGCAC |
| | | GGGGCGAAGGCAGCGTCGCAGCGACTCCCGCCCGCCGCG |
| | | CGCTTCGCTTTTTATAGGGCCGCCGCCGCCGCCGCCTCGCC |
| | | ATAAAAGGAAACTTTCGGAGCGCGCCGCTCTGATTGGCTG |
| | | CCGCCGCACCTCTCCGCCTCGCCCCGCCCCGCCCCTCGCCC |
| | | CGCCCCGCCCCGCCTGGCGCGCGCCCCCCCCCCCCCCCCG |
| | | CCCCCATCGCTGCACAAAATAATTAAAAAATAAATAAATA |
| | | CAAAATTGGGGGTGGGGGGGGGGGGAGATGGGGAGAGT |
| | | GAAGCAGAACGTGGGGCTCACCTCGAGCCATGGTAATAG |
| | | CGATGACTAATACGTAGATGTACTGCCAAGTAGGAAAGTC |
| | | CCATAAGGTCATGTACTGGGCATAATGCCAGGCGGGCCAT |
| | | TTACCGTCATTGACGTCAATAGGGGGCGTACTTGGCATAT |
| | | GATACACTTGATGTACTGCCAAGTGGGCAGTTTACCGTAA |
| | | ATACTCCACCCATTGACGTCAATGGAAAGTCCCTATTGGC |
| | | GTTACTATGGGAACATACGTCATTATTGACGTCAATGGGC |
| | | GGGGGTCGTTGGGCGGTCAGCCAGGCGGGCCATTTACCGT |
| | | AAGTTATGTAACGGGTACCGAATTCAGATCAGCTTCCTAG |
| | | GAACCCCTAGTGATGGAGTTGGCCACTCCCTCTCTGCGCG |
| | | CTCGCTCGCTCACTGAGGCCGGGCGACCAAAGGTCGCCCG |
| | | ACGCCCGGGCTTTGCCCGGGCGGCCTCAGTGAGCGAGCGA |
| | | GCGCGCCAGGATCCGAGCTTGTCGAGAAGTACTAGAGGA |
| | | TCATCAAGCTGTAGCCAACCACTAGAACTATAGCTAGAGT |
| | | CCTGGGCGAACAAACGATGCTCGCCTTCCAGAAAACCGA |
| | | GGATGCGAACCACTTCATCCGGGGTCAGCACCACCGGCAA |
| | | GCGCCGCGACGGCCGAGGTCTTCCGATCTCCTGAAGCCAG |
| | | GGCAGATCCGTGCACAGCACCTTGCCGTAGAAGAACAGC |
| | | AAGGCCGCCAATGCCTGACGATGCGTGGAGACCGAAACC |
| | | TTGCGCTCGTTCGCCAGCCAGGACAGAAATGCCTCGACTT |
| | | CGCTGCTGCCCAAGGTTGCCGGGTGACGCACACCGTGGAA |
| | | ACGGATGAAGGCACGAACCCAGTTGACATAAGCCTGTTCG |
| | | GTTCGTAAACTGTAATGCAAGTAGCGTATGCGCTCACGCA |
| | | ACTGGTCCAGAACCTTGACCGAACGCAGCGGTGGTAACG |
| | | GCGCAGTGGCGGTTTTCATGGCTTGTTATGACTGTTTTTTT |
| | | GTACAGTCTATGCCTCGGGCATCCAAGCAGCAAGCGCGTT |
| | | ACGCCGTGGGTCGATGTTTGATGTTATGGAGCAGCAACGA |
| | | TGTTACGCAGCAGCAACGATGTTACGCAGCAGGGCAGTCG |
| | | CCCTAAAACAAAGTTAGGTGGCTCAAGTATGGGCATCATT |
| | | CGCACATGTAGGCTCGGCCCTGACCAAGTCAAATCCATGC |
| | | GGGCTGCTCTTGATCTTTTCGGTCGTGAGTTCGGAGACGT |
| | | AGCCACCTACTCCCAACATCAGCCGGACTCCGATTACCTC |
| | | GGGAACTTGCTCCGTAGTAAGACATTCATCGCGCTTGCTG |
| | | CCTTCGACCAAGAAGCGGTTGTTGGCGCTCTCGCGGCTTA |
| | | CGTTCTGCCCAGGTTTGAGCAGCCGCGTAGTGAGATCTAT |
| | | ATCTATGATCTCGCAGTCTCCGGCGAGCACCGGAGGCAGG |
| | | GCATTGCCACCGCGCTCATCAATCTCCTCAAGCATGAGGC |
| | | CAACGCGCTTGGTGCTTATGTGATCTACGTGCAAGCAGAT |
| | | TACGGTGACGATCCCGCAGTGGCTCTCTATACAAAGTTGG |
| | | GCATACGGGAAGAAGTGATGCACTTTGATATCGACCCAAG |
| | | TACCGCCACCTAACAATTCGTTCAAGCCGAGATCGGCTTC |
| | | CCGGCCGCGGAGTTGTTCGGTAAATTGTCACAACGCCGCG |
| | | AATATAGTCTTTACCATGCCCTTGGCCACGCCCCTCTTTAA |
| | | TACGACGGGCAATTTGCACTTCAGAAAATGAAGAGTTTGC |
| | | TTTAGCCATAACAAAAGTCCAGTATGCTTTTTCACAGCAT |
| | | AACTGGACTGATTTCAGTTTACAACTATTCTGTCTAGTTTA |
| | | AGACTTTATTGTCATAGTTTAGATCTATTTTGTTCAGTTTA |
| | | AGACTTTATTGTCCGCCCACACCCGCTTACGCAGGGCATC |
| | | CATTTATTACTCAACCGTAACCGATTTTGCCAGGTTACGCG |
| | | GCTGGTCTGCGGTGTGAAATACCGCACAGATGCGTAAGGA |
| | | GAAAATACCGCATCAGGCGCTCTTCCGCTTCCTCGCTCAC |
| | | TGACTCGCTGCGCTCGGTCGTTCGGCTGCGGCGAGCGGTA |
| | | TCAGCTCACTCAAAGGCGGTAATACGGTTATCCACAGAAT |
| | | CAGGGGATAACGCAGGAAAGAACATGTGAGCAAAAGGCC |
| | | AGCAAAAGGCCAGGAACCGTAAAAAGGCCGCGTTGCTGG |
| | | CGTTTTTCCATAGGCTCCGCCCCCCTGACGAGCATCACAA |
| | | AAATCGACGCTCAAGTCAGAGGTGGCGAAACCCGACAGG |
| | | ACTATAAAGATACCAGGCGTTTCCCCCTGGAAGCTCCCTC |
| | | GTGCGCTCTCCTGTTCCGACCCTGCCGCTTACCGGATACCT |
| | | GTCCGCCTTTCTCCCTTCGGGAAGCGTGGCGCTTTCTCAAT |
| | | GCTCACGCTGTAGGTATCTCAGTTCGGTGTAGGTCGTTCG |
| | | CTCCAAGCTGGGCTGTGTGCACGAACCCCCCGTTCAGCCC |
| | | GACCGCTGCGCCTTATCCGGTAACTATCGTCTTGAGTCCA |
| | | ACCCGGTAAGACACGACTTATCGCCACTGGCAGCAGCCAC |
| | | TGGTAACAGGATTAGCAGAGCGAGGTATGTAGGCGGTGC |
| | | TACAGAGTTCTTGAAGTGGTGGCCTAACTACGGCTACACT |
| | | AGAAGGACAGTATTTGGTATCTGCGCTCTGCTGAAGCCAG |
| | | TTACCTTCGGAAAAAGAGTTGGTAGCTCTTGATCCGGCAA |
| | | ACAAACCACCGCTGGTAGCGGTGGTTTTTTTGTTTGCAAG |
| | | CAGCAGATTACGCGCAGAAAAAAAGGATCTCAAGAAGAT |
| | | CCTTTGATCTTTTCTACGGGGTCTGACGCTCAGTGGAACG |
| | | AAAACTCACGTTAAGGGATTTTGGTCATGAGATTATCAAA |
| | | AAGGATCTTCACCTAGATCCTTTTAAATTAAAAATGAAGT |
| | | TTTAAATCAATCTAAAGTATATATGAGTAAACTTGGTCTG |
| | | ACAGTTACCAATGCTTAATCAGTGAGGCACCTATCTCAGC |
| | | GATCTGTCTATTTCGTTCATCCATAGTTGCCTGACTCCCCG |
| | | TCGTGTAGATAACTACGATACGGGAGGGCTTACCATCTGG |
| | | CCCCAGTGCTGCAATGATACCGCGAGACCCACGCTCACCG |
| | | GCTCCAGATTTATCAGCAATAAACCAGCCAGCCGGAAGG |
| | | GCCGAGCGCAGAAGTGGTCCTGCAACTTTATCCGCCTCCA |
| | | TCCAGTCTATTAATTGTTGCCGGGAAGCTAGAGTAAGTAG |
| | | TTCGCCAGTTAATAGTTTGCGCAACGTTGTTGCCATTGCTA |
| | | CAGGCATCGTGGTGTCACGCTCGTCGTTTGGTATGGCTTC |
| | | ATTCAGCTCCGGTTCCCAACGATCAAGGCGAGTTACATGA |
| | | TCCCCCATGTTGTGCAAAAAAGCGGTTAGCTCCTTCGGTC |
| | | CTCCGATCGTTGTCAGAAGTAAGTTGGCCGCAGTGTTATC |
| | | ACTCATGGTTATGGCAGCACTGCATAATTCTCTTACTGTCA |
| | | TGCCATCCGTAAGATGCTTTTCTGTGACTGGTGAGTACTCA |
| | | ACCAAGTCATTCTGAGAATAGTGTATGCGGCGACCGAGTT |
| | | GCTCTTGCCCGGCGTCAATACGGGATAATACCGCGCCACA |
| | | TAGCAGAACTTTAAAAGTGCTCATCATTGGAAAACGTTCT |
| | | TCGGGGCGAAAACTCTCAAGGATCTTACCGCTGTTGAGAT |
| | | CCAGTTCGATGTAACCCACTCGTGCACCCAACTGATCTTC |
| | | AGCATCTTTTACTTTCACCAGCGTTTCTGGGTGAGCAAAA |
| | | ACAGGAAGGCAAAATGCCGCAAAAAAGGGAATAAGGGC |
| | | GACACGGAAATGTTGAATACTCATACTCTTCCTTTTTCAAT |
| | | ATTATTGAAGCATTTATCAGGGTTATTGTCTCATGAGCGG |
| | | ATACATATTTGAATGTATTTAGAAAAATAAACAAATAGGG |
| | | GTTCCGCGCACATTTCCCCGAAAAGTGCCACCTGAAATTG |
| | | TAAACGTTAATATTTTGTTAAAATTCGCGTTAAATTTTTGT |
| | | TAAATCAGCTCATTTTTTAACCAATAGGCCGAAATCGGCA |
| | | AAATCCCTTATAAATCAAAAGAATAGACCGAGATAGGGTT |
| | | GAGTGTTGTTCCAGTTTGGAACAAGAGTCCACTATTAAAG |
| | | AACGTGGACTCCAACGTCAAAGGGCGAAAAACCGTCTAT |
| | | CAGGGCGATGGCCCACTACGTGAACCATCACCCTAATCAA |
| | | GTTTTTTGGGGTCGAGGTGCCGTAAAGCACTAAATCGGAA |
| | | CCCTAAAGGGAGCCCCCGATTTAGAGCTTGACGGGGAAA |
| | | GCCGGCGAACGTGGCGAGAAAGGAAGGGAAGAAAGCGA |
| | | AAGGAGCGGGCGCTAGGGCGCTGGCAAGTGTAGCGGTCA |
| | | CGCTGCGCGTAACCACCACACCCGCCGCGCTTAATGCGCC |
| | | GCTACAGGGCGCGTC |
| AMI303 | 178 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGGGTACCGGGCCCCCCCTCGAGGTCGACGGTATCGAT |
| | | GGATCCGATATCGAATTCAAGCGCGCTCGCTCGCTCACTG |
| | | AGGCCGCCCGGGCAAAGCCCGGGCGTCGGGCGACCTTTG |
| | | GTCGCCCGGCCTCAGTGAGCGAGCGAGCGCGCAGAGAGG |
| | | GAGTGGCCAACTCCATCACTAGGGGTTCCTTGTAGTTAAT |
| | | GATTAACCCGCCATGCTACTTATCTACGTACTCTGGAGAC |
| | | GACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTGG |
| | | AGTATTTACGGTAAACTGCCCACTTGGCAGTACATCAAGT |
| | | GTATCATATGCCAAGTCCGCCCCCTATTGACGTCAATGAC |
| | | GGTAAATGGCCCGCCTGGCATTATGCCCAGTACATGACCT |
| | | TACGGGACTTTCCTACTTGGCAGTACATCTACGTATTAGTC |
| | | ATCGCTATTACCATGGTCGAGGTGAGCCCCACGTTCTGCT |
| | | TCACTCTCCCCATCTCCCCCCCCTCCCCACCCCCAATTTTG |
| | | TATTTATTTATTTTTTAATTATTTTGTGCAGCGATGGGGGC |
| | | GGGGGGGGGGGGGGGGCGCGCGCCAGGCGGGGCGGGGC |
| | | GGGGCGAGGGGGGGGCGGGGCGAGGCGGAGAGGTGCG |
| | | GCGGCAGCCAATCAGAGCGGCGCGCTCCGAAAGTTTCCTT |
| | | TTATGGCGAGGCGGCGGCGGCGGCGGCCCTATAAAAAGC |
| | | GAAGCGCGCGGCGGGCGGGAGTCGCTGCGACGCTGCCTT |
| | | CGCCCCGTGCCCCGCTCCGCCGCCGCCTCGCGCCGCCCGC |
| | | CCCGGCTCTGACTGACCGCGTTACTCCCACAGCTCACTCTC |
| | | TTCCGCATCGCTGTCTGCGAGGGCCAGCTGTTGGGGTGAG |
| | | TACTCCCTCTCAAAAGCGGGCATGACTTCTGCGCTAAGAT |
| | | TGTCAGTTTCCAAAAACGAGGAGGATTTGATATTCACCTG |
| | | GCCCGCGGTGATGCCTTTGAGGGTGGCCGCGTCCATCTGG |
| | | TCAGAAAAGACAATCTTTTTGTTGTCAAGCTTGGTGGCAA |
| | | ACGACCCGTAGAGGGCGTTGGACAGCAACTTGGCGATGG |
| | | AGCGCAGGGTTTGGTTTTTGTCGCGATCGGCGCGCTCCTT |
| | | GGCCGCGATGTTTAGCTGCACGTATTCGCGCGCAACGCAC |
| | | CGCCATTCGGGAAAGACGGTGGTGCGCTCGTCGGGCACCA |
| | | GGTGCACGCGCCAACCGCGGTTGTGCAGGGTGACAAGGT |
| | | CAACGCTGGTGGCTACCTCTCCGCGTAGGCGCTCGTTGGT |
| | | CCAGCAGAGGCGGCCGCCCTTGCGCGAACAGAATGGCGG |
| | | TAGTGGGTCTAGCTGCGTCTCGTCCGGGGGGTCTGCGTCC |
| | | ACGGTAAAGACCCCGGGCAGCAGGCGCGCGTCGAAGTAG |
| | | TCTATCTTGCATCCTTGCAAGTCTAGCGCCTGCTGCCATGC |
| | | GCGGGCGGCAAGCGCGCGCTCGTATGGGTTGAGTGGGGG |
| | | ACCCCATGGCATGGGGTGGGTGAGCGCGGAGGCGTACAT |
| | | GCCGCAAATGTCGTAAACGTAGAGGGGCTCTCTGAGTATT |
| | | CCAAGATATGTAGGGTAGCATCTTCCACCGCGGATGCTGG |
| | | CGCGCACGTAATCGTATAGTTCGTGCGAGGGAGCGAGGA |
| | | GGTCGGGACCGAGGTTGCTACGGGCGGGCTGCTCTGCTCG |
| | | GAAGACTATCTGCCTGAAGATGGCATGTGAGTTGGATGAT |
| | | ATGGTTGGACGCTGGAAGACGTTGAAGCTGGCGTCTGTGA |
| | | GACCTACCGCGTCACGCACGAAGGAGGCGTAGGAGTCGC |
| | | GCAGCTTGTTGACCAGCTCGGCGGTGACCTGCACGTCTAG |
| | | GGCGCAGTAGTCCAGGGTTTCCTTGATGATGTCATACTTA |
| | | TCCTGTCCCTTTTTTTTCCACAGCTCGCGGTTGAGGACAAA |
| | | CTCTTCGCGGTCTTTCCAGTACTCTTGGATCGGAAACCCGT |
| | | CGGCCTCCGAACGGTACTCCGCCACCGAGGGACCTGAGCG |
| | | AGTCCGCATCGACCGGATCGGAAAACCTCTCGAGAAAGG |
| | | CGTCTAACCAGTCACAGTCGCAAGGTAGGCTGAGCACCGT |
| | | GGCGGGCGGCAGCGGGTGGCGGTCGGGGTTGTTTCTGGCG |
| | | GAGGTGCTGCTGATGATGTAATTAAAGTAGGCGGTCTTGA |
| | | GACGGCGGATGGTCGAATCTGGCCATACACTTGAGTGACA |
| | | ATGACATCCACTTTGCCTTTCTCTCCACAGGTGTCCACTCC |
| | | CAGGTCCAAGTTTAAACGCCGCCACCATGGAGTTCGGCCT |
| | | GAGCTGGCTGTTCCTGGTGGCCATCCTTAAGGGCGTGCAG |
| | | TGCGAGCACCCCAACGCGCGCAAATACAAAGGAGCCAAC |
| | | AAGAAGGGCTTGTCCAAGGGCTGCTTCGGCCTCAAGCTGG |
| | | ACCGAATCGGCTCCATGAGCGGCCTGGGATGTTAGGGAG |
| | | GCGGAAGCGGCGGAGGCGGATCTGGCGGAGGCGGCAGCG |
| | | GCGGCGGCGGCTCTGACAAAACTCACACATGCCCACCGTG |
| | | CCCAGCACCTGAACTCCTGGGGGGACCGTCAGTCTTCCTC |
| | | TTCCCCCCAAAACCCAAGGACACCCTCATGATCTCCCGGA |
| | | CCCCTGAGGTCACATGCGTGGTGGTGGACGTGAGCCACGA |
| | | AGACCCTGAGGTCAAGTTCAACTGGTACGTGGACGGCGTG |
| | | GAGGTGCATAATGCCAAGACAAAGCCGCGGGAGGAGCAG |
| | | TACAACAGCACGTACCGTGTGGTCAGCGTCCTCACCGTCC |
| | | TGCACCAGGACTAGCTGAATGGCAAGGAGTACAAGTGCA |
| | | AGGTCTCCAACAAAGCCCTCCCAGCCCCCATCGAGAAAAC |
| | | CATCTCCAAAGCCAAAGGGCAGCCCCGAGAACCACAGGT |
| | | GTACACCCTGCCCCCATCCCGGGATGAGCTGACCAAGAAC |
| | | CAGGTCAGCCTGACCTGCCTGGTCAAAGGCTTCTATCCCA |
| | | GCGACATCGCCGTGGAGTGGGAGAGCAATGGGCAGCCGG |
| | | AGAACAACTACAAGACCACGCCTCCCGTGCTGGACTCCGA |
| | | CGGCTCCTTCTTCCTCTACAGCAAGCTCACCGTGGACAAG |
| | | AGCAGGTGGCAGCAGGGGAACGTCTTCTCATGCTCCGTGA |
| | | TGCATGAGGCTCTGCACAACCACTACACGCAGAAGAGCCT |
| | | CTCCCTGTCTCCGGGTAAATGATAACTCGAGAATCAACCT |
| | | CTGGATTACAAAATTTGTGAAAGATTGACTGGTATTCTTA |
| | | ACTATGTTGCTCCTTTTACGCTATGTGGATACGCTGCTTTA |
| | | ATGCCTTTGTATCATGCTATTGCTTCCCGTATGGCTTTCAT |
| | | TTTCTCCTCCTTGTATAAATCCTGGTTGCTGTCTCTTTATGA |
| | | GGAGTTGTGGCCCGTTGTCAGGCAACGTGGCGTGGTGTGC |
| | | ACTGTGTTTGCTGACGCAACCCCCACTGGTTGGGGCATTG |
| | | CCACCACCTGTCAGCTCCTTTCCGGGACTTTCGCTTTCCCC |
| | | CTCCCTATTGCCACGGCGGAACTCATCGCCGCCTGCCTTG |
| | | CCCGCTGCTGGACAGGGGCTCGGCTGTTGGGCACTGACAA |
| | | TTCCGTGGTCTGTTCTCATCACATCATATCAAGGTTATATA |
| | | CCATCAATATTGCCACAGATGTTACTTAGCCTTTTAATATT |
| | | TCTCTAATTTAGTGTATATGCAATGATAGTTCTCTGATTTC |
| | | TGAGATTGAGTTTCTCATGTGTAATGATTATTTAGAGTTTC |
| | | TCTTTCATCTGTTCAAATTTTTGTCTAGTTTTATTTTTTACT |
| | | GATTTGTAAGACTTCTTTTTATAATCTGCATATTACAATTC |
| | | TCTTTACTGGGGTGTTGCAAATATTTTCTGTCATTCTATGG |
| | | CCTGACTTTTCTTAATGGTTTTTTAATTTTAAAAATAAGTC |
| | | TTAATATTCATGCAATCTAATTAACAATCTTTTCTTTGTGG |
| | | TTAGGACTTTGAGTCATAAGAAATTTTTCTCTACACTGAA |
| | | GTCATGATGGCATGCTTCTATATTATTTTCTAAAAGATTTA |
| | | AAGTTTTGCCTTCTCCATTTAGACTTATAATTCACTGGAAT |
| | | TTTTTTGTGTGTATGGTATGACATATGGGTTCCCTTTTATTT |
| | | TTTACATATAAATATATTTCCCTGTTTTTCTAAAAAAGAAA |
| | | AAGATCATCATTTTCCCATTGTAAAATGCCATATTTTTTTC |
| | | ATAGGTCACTTACATATATCAATGGGTCTGTTTCTGAGCTC |
| | | TACTCTATTTTATCAGCCTCACTGTCTATCCCCACACATCT |
| | | CATGCTTTGCTCTAAATCTTGATATTTAGTGGAACATTCTT |
| | | TCCCATTTTGTTCTACAAGAATATTTTTGTTATTGTCTTTGG |
| | | GCTTTCTATATACATTTTGAAATGAGGTTGACAAGTTACCT |
| | | AGGAAAACTGTCTTCCTGCCCGGGTGGCATCCCTGTGACC |
| | | CCTCCCCAGTGCCTCTCCTGGCCCTGGAAGTTGCCACTCCA |
| | | GTGCCCACCAGCCTTGTCCTAATAAAATTAAGTTGCATCA |
| | | TTTTGTCTGACTAGGTGTCCTTCTATAATATTATGGGGTGG |
| | | AGGGGGGTGGTATGGAGCAAGGGGCCCAAGTTGGGAAGA |
| | | AACCTGTAGGGCCTGCGTACGTAGATAAGTAGCATGGCGG |
| | | GTTAATCATTAACTACAAGGAACCCCTAGTGATGGAGTTG |
| | | GCCACTCCCTCTCTGCGCGCTCGCTCGCTCACTGAGGCCG |
| | | GGCGACCAAAGGTCGCCCGACGCCCGGGCTTTGCCCGGGC |
| | | GGCCTCAGTGAGCGAGCGAGCGCGCAGCTGCTGGTACCG |
| | | GGCCCCCCCTCGAGGTCGACGGTATCGATGGATCCGATAT |
| | | CGAATTCAACCTGCAGGCATGCTCTGAGACAATAACCCTG |
| | | ATAAATGCTTCAATAATGTAGCCAACCACTAGAACTATAG |
| | | CTAGAGTCCTGGGCGAACAAACGATGCTCGCCTTCCAGAA |
| | | AACCGAGGATGCGAACCACTTCATCCGGGGTCAGCACCAC |
| | | CGGCAAGCGCCGCGACGGCCGAGGTCTTCCGATCTCCTGA |
| | | AGCCAGGGCAGATCCGTGCACAGCACCTTGCCGTAGAAG |
| | | AACAGCAAGGCCGCCAATGCCTGACGATGCGTGGAGACC |
| | | GAAACCTTGCGCTCGTTCGCCAGCCAGGACAGAAATGCCT |
| | | CGACTTCGCTGCTGCCCAAGGTTGCCGGGTGACGCACACC |
| | | GTGGAAACGGATGAAGGCACGAACCCAGTTGACATAAGC |
| | | CTGTTCGGTTCGTAAACTGTAATGCAAGTAGCGTATGCGC |
| | | TCACGCAACTGGTCCAGAACCTTGACCGAACGCAGCGGTG |
| | | GTAACGGCGCAGTGGCGGTTTTCATGGCTTGTTATGACTG |
| | | TTTTTTTGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGCAACGATGTTACGCAGCAG |
| | | GGCAGTCGCCCTAAAACAAAGTTAGGTGGCTCAAGTATGG |
| | | GCATCATTCGCACATGTAGGCTCGGCCCTGACCAAGTCAA |
| | | ATCCATGCGGGCTGCTCTTGATCTTTTCGGTCGTGAGTTCG |
| | | GAGACGTAGCCACCTACTCCCAACATCAGCCGGACTCCGA |
| | | TTACCTCGGGAACTTGCTCCGTAGTAAGACATTCATCGCG |
| | | CTTGCTGCCTTCGACCAAGAAGCGGTTGTTGGCGCTCTCG |
| | | CGGCTTACGTTCTGCCCAGGTTTGAGCAGCCGCGTAGTGA |
| | | GATCTATATCTATGATCTCGCAGTCTCCGGCGAGCACCGG |
| | | AGGCAGGGCATTGCCACCGCGCTCATCAATCTCCTCAAGC |
| | | ATGAGGCCAACGCGCTTGGTGCTTATGTGATCTACGTGCA |
| | | AGCAGATTACGGTGACGATCCCGCAGTGGCTCTCTATACA |
| | | AAGTTGGGCATACGGGAAGAAGTGATGCACTTTGATATCG |
| | | ACCCAAGTACCGCCACCTAACAATTCGTTCAAGCCGAGAT |
| | | CGGCTTCCCGGCCGCGGAGTTGTTCGGTAAATTGTCACAA |
| | | CGCCGCGAATATAGTCTTTACCATGCCCTTGGCCACGCCC |
| | | CTCTTTAATACGACGGGCAATTTGCACTTCAGAAAATGAA |
| | | GAGTTTGCTTTAGCCATAACAAAAGTCCAGTATGCTTTTTC |
| | | ACAGCATAACTGGACTGATTTCAGTTTACAACTATTCTGTC |
| | | TAGTTTAAGACTTTATTGTCATAGTTTAGATCTATTTTGTT |
| | | CAGTTTAAGACTTTATTGTCCGCCCACACCCGCTTACGCA |
| | | GGGCATCCATTTATTACTCAACCGTAACCGATTTTGCCAG |
| | | GTTACGCGGCTGGTCTGCGGTGTGAAATACCGCACAGATG |
| | | CGTAAGGAGAAAATACCGCATCAGGCGCTCTTCCGCTTCC |
| | | TCGCTCACTGACTCGCTGCGCTCGGTCGTTCGGCTGCGGC |
| | | GAGCGGTATCAGCTCACTCAAAGGCGGTAATACGGTTATC |
| | | CACAGAATCAGGGGATAACGCAGGAAAGAACATGTGAGC |
| | | AAAAGGCCAGCAAAAGGCCAGGAACCGTAAAAAGGCCGC |
| | | GTTGCTGGCGTTTTTCCATAGGCTCCGCCCCCCTGACGAGC |
| | | ATCACAAAAATCGACGCTCAAGTCAGAGGTGGCGAAACC |
| | | CGACAGGACTATAAAGATACCAGGCGTTTCCCCCTGGAAG |
| | | CTCCCTCGTGCGCTCTCCTGTTCCGACCCTGCCGCTTACCG |
| | | GATACCTGTCCGCCTTTCTCCCTTCGGGAAGCGTGGCGCTT |
| | | TCTCAATGCTCACGCTGTAGGTATCTCAGTTCGGTGTAGGT |
| | | CGTTCGCTCCAAGCTGGGCTGTGTGCACGAACCCCCCGTT |
| | | CAGCCCGACCGCTGCGCCTTATCCGGTAACTATCGTCTTG |
| | | AGTCCAACCCGGTAAGACACGACTTATCGCCACTGGCAGC |
| | | AGCCACTGGTAACAGGATTAGCAGAGCGAGGTATGTAGG |
| | | CGGTGCTACAGAGTTCTTGAAGTGGTGGCCTAACTACGGC |
| | | TACACTAGAAGGACAGTATTTGGTATCTGCGCTCTGCTGA |
| | | AGCCAGTTACCTTCGGAAAAAGAGTTGGTAGCTCTTGATC |
| | | CGGCAAACAAACCACCGCTGGTAGCGGTGGTTTTTTTGTT |
| | | TGCAAGCAGCAGATTACGCGCAGAAAAAAAGGATCTCAA |
| | | GAAGATCCTTTGATCTTTTCTACGTGGTCTGACGCTCAGTG |
| | | GAACGAAAACTCACGTTAAGGGATTTTGGTCATGAGATTA |
| | | TCAAAAAGGATCTTCACCTAGATCCTTTTAAATTAAAAAT |
| | | GAAGTTTTAAATCAATCTAAAGTATATATGAGTAAACTTG |
| | | GTCTGACGGTTACTCAGAAGAACTCGTCAAGAAGGCGATA |
| | | GAAGGCGATGCGCTGCGAATCGGGAGCGGCGATACCGTA |
| | | AAGCACGAGGAAGCGGTCAGCCCATTCGCCGCCAAGCTCT |
| | | TCAGCAATATCACGGGTAGCCAACGCTATGTCCTGATAGC |
| | | GGTCCGCCACACCCAGCCGGCCACAGTCGATGAATCCAGA |
| | | AAAGCGGCCATTTTCCACCATGATATTCGGCAAGCAGGCA |
| | | TCGCCATGGGTCACGACGAGATCCTCGCCGTCGGGCATGC |
| | | GCGCCTTGAGCCTGGCGAACAGTTCGGCTGGCGCGAGCCC |
| | | CTGATGCTCTTCGTCCAGATCATCCTGATCGACAAGACCG |
| | | GCTTCCATCCGAGTACGTGCTCGCTCGATGCGATGTTTCGC |
| | | TTGGTGGTCGAATGGGCAGGTAGCCGGATCAAGCGTATGC |
| | | AGCCGCCGCATTGCATCAGCCATGATGGATACTTTCTCGG |
| | | CAGGAGCAAGGTGAGATGACAGGAGATCCTGCCCCGGCA |
| | | CTTCGCCCAATAGCAGCCAGTCCCTTCCCGCTTCAGTGAC |
| | | AACGTCGAGCACAGCTGCGCAAGGAACGCCCGTCGTGGC |
| | | CAGCCACGATAGCCGCGCTGCCTCGTCCTGCAGTTCATTC |
| | | AGGGCACCGGACAGGTCGGTCTTGACAAAAAGAACCGGG |
| | | CGCCCCTGCGCTGACAGCCGGAACACGGCGGCATCAGAG |
| | | CAGCCGATTGTCTGTTGTGCCCAGTCATAGCCGAATAGCC |
| | | TCTCCACCCAAGCGGCCGGAGAACCTGCGTGCAATCCATC |
| | | TTGTTCAATCATACTCTTCCTTTTTCAATATTATTGAAGCA |
| | | TTTATCAGGGTTATTGTCTCATGAGCGGATACATATTTGAA |
| | | TGTATTTAGAAAAATAAACAAATAGGGGTTCCGCGCACAT |
| | | TTCCCCGAAAAGTGCCACCTGACGTCTAAGAAACCATTAT |
| | | TATCATGACATTAACCTATAAAAATAGGCGTATCACGAGG |
| | | CCCTTTCGTCTCGCGCGTTTCGGTGATGACGGTGAAAACC |
| | | TCTGACACATGCAGCTCCCGGAGACGGTCACAGCTTGTCT |
| | | GTAAGCGGATGCCGGGAGCAGACAAGCCCGTCAGGGCGC |
| | | GTCAGCGGGTGTTGGCGGGTGTCGGGGCTGGCTTAACTAT |
| | | GCGGCATCAGAGCAGATTGTACTGAGAGTGCACCATATGC |
| | | GGTGTGAAATACCGCACAGATGCGTAAGGAGAAAATACC |
| | | GCATCAGGAAATTGTAAACGTTAATATTTTGTTAAAATTC |
| | | GCGTTAAATTTTTGTTAAATCAGCTCATTTTTTAACCAATA |
| | | GGCCGAAATCGGCAAAATCCCTTATAAATCAAAAGAATA |
| | | GACCGAGATAGGGTTGAGTGTTGTTCCAGTTTGGAACAAG |
| | | AGTCCACTATTAAAGAACGTGGACTCCAACGTCAAAGGGC |
| | | GAAAAACCGTCTATCAGGGCGATGGCCCACTACGTGAACC |
| | | ATCACCCTAATCAAGTTTTTTGGGGTCGAGGTGCCGTAAA |
| | | GCACTAAATCGGAACCCTAAAGGGAGCCCCCGATTTAGA |
| | | GCTTGACGGGGAAAGCCGGCGAACGTGGCGAGAAAGGAA |
| | | GGGAAGAAAGCGAAAGGAGCGGGCGCTAGGGCGCTGGCA |
| | | AGTGTAGCGGTCACGCTGCGCGTAACCACCACACCCGCCG |
| | | CGCTTAATGCGCCGCTACAGGGCGCGTCC |
| AMI304 | 179 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGGGTACCGGGCCCCCCCTCGAGGTCGACGGTATCGAT |
| | | GGATCCGATATCGAATTCAAGCGCGCTCGCTCGCTCACTG |
| | | AGGCCGCCCGGGCAAAGCCCGGGCGTCGGGCGACCTTTG |
| | | GTCGCCCGGCCTCAGTGAGCGAGCGAGCGCGCAGAGAGG |
| | | GAGTGGCCAACTCCATCACTAGGGGTTCCTTGTAGTTAAT |
| | | GATTAACCCGCCATGCTACTTATCTACGTACTCTGGAGAC |
| | | GACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTGG |
| | | AGTATTTACGGTAAACTGCCCACTTGGCAGTACATCAAGT |
| | | GTATCATATGCCAAGTCCGCCCCCTATTGACGTCAATGAC |
| | | GGTAAATGGCCCGCCTGGCATTATGCCCAGTACATGACCT |
| | | TACGGGACTTTCCTACTTGGCAGTACATCTACGTATTAGTC |
| | | ATCGCTATTACCATGGTCGAGGTGAGCCCCACGTTCTGCT |
| | | TCACTCTCCCCATCTCCCCCCCCTCCCCACCCCCAATTTTG |
| | | TATTTATTTATTTTTTAATTATTTTGTGCAGCGATGGGGGC |
| | | GGGGGGGGGGGGGGGGCGCGCGCCAGGCGGGGCGGGGC |
| | | GGGGCGAGGGGCGGGGCGGGGCGAGGCGGAGAGGTGCG |
| | | GCGGCAGCCAATCAGAGCGGCGCGCTCCGAAAGTTTCCTT |
| | | TTATGGCGAGGCGGCGGCGGCGGCGGCCCTATAAAAAGC |
| | | GAAGCGCGCGGCGGGCGGGAGTCGCTGCGACGCTGCCTT |
| | | CGCCCCGTGCCCCGCTCCGCCGCCGCCTCGCGCCGCCCGC |
| | | CCCGGCTCTGACTGACCGCGTTACTCCCACAGCTCACTCTC |
| | | TTCCGCATCGCTGTCTGCGAGGGCCAGCTGTTGGGGTGAG |
| | | TACTCCCTCTCAAAAGCGGGCATGACTTCTGCGCTAAGAT |
| | | TGTCAGTTTCCAAAAACGAGGAGGATTTGATATTCACCTG |
| | | GCCCGCGGTGATGCCTTTGAGGGTGGCCGCGTCCATCTGG |
| | | TCAGAAAAGACAATCTTTTTGTTGTCAAGCTTGGTGGCAA |
| | | ACGACCCGTAGAGGGCGTTGGACAGCAACTTGGCGATGG |
| | | AGCGCAGGGTTTGGTTTTTGTCGCGATCGGCGCGCTCCTT |
| | | GGCCGCGATGTTTAGCTGCACGTATTCGCGCGCAACGCAC |
| | | CGCCATTCGGGAAAGACGGTGGTGCGCTCGTCGGGCACCA |
| | | GGTGCACGCGCCAACCGCGGTTGTGCAGGGTGACAAGGT |
| | | CAACGCTGGTGGCTACCTCTCCGCGTAGGCGCTCGTTGGT |
| | | CCAGCAGAGGCGGCCGCCCTTGCGCGAACAGAATGGCGG |
| | | TAGTGGGTCTAGCTGCGTCTCGTCCGGGGGGTCTGCGTCC |
| | | ACGGTAAAGACCCCGGGCAGCAGGCGCGCGTCGAAGTAG |
| | | TCTATCTTGCATCCTTGCAAGTCTAGCGCCTGCTGCCATGC |
| | | GCGGGCGGCAAGCGCGCGCTCGTATGGGTTGAGTGGGGG |
| | | ACCCCATGGCATGGGGTGGGTGAGCGCGGAGGCGTACAT |
| | | GCCGCAAATGTCGTAAACGTAGAGGGGCTCTCTGAGTATT |
| | | CCAAGATATGTAGGGTAGCATCTTCCACCGCGGATGCTGG |
| | | CGCGCACGTAATCGTATAGTTCGTGCGAGGGAGCGAGGA |
| | | GGTCGGGACCGAGGTTGCTACGGGCGGGCTGCTCTGCTCG |
| | | GAAGACTATCTGCCTGAAGATGGCATGTGAGTTGGATGAT |
| | | ATGGTTGGACGCTGGAAGACGTTGAAGCTGGCGTCTGTGA |
| | | GACCTACCGCGTCACGCACGAAGGAGGCGTAGGAGTCGC |
| | | GCAGCTTGTTGACCAGCTCGGCGGTGACCTGCACGTCTAG |
| | | GGCGCAGTAGTCCAGGGTTTCCTTGATGATGTCATACTTA |
| | | TCCTGTCCCTTTTTTTTCCACAGCTCGCGGTTGAGGACAAA |
| | | CTCTTCGCGGTCTTTCCAGTACTCTTGGATCGGAAACCCGT |
| | | CGGCCTCCGAACGGTACTCCGCCACCGAGGGACCTGAGCG |
| | | AGTCCGCATCGACCGGATCGGAAAACCTCTCGAGAAAGG |
| | | CGTCTAACCAGTCACAGTCGCAAGGTAGGCTGAGCACCGT |
| | | GGCGGGCGGCAGCGGGTGGCGGTCGGGGTTGTTTCTGGCG |
| | | GAGGTGCTGCTGATGATGTAATTAAAGTAGGCGGTCTTGA |
| | | GACGGCGGATGGTCGAATCTGGCCATACACTTGAGTGACA |
| | | ATGACATCCACTTTGCCTTTCTCTCCACAGGTGTCCACTCC |
| | | CAGGTCCAAGTTTAAACGCCGCCACCATGGAGTTCGGCCT |
| | | GAGCTGGCTGTTCCTGGTGGCCATCCTTAAGGGCGTGCAG |
| | | TGCGACAAGAGAGTGGAGAGCAAGTACGGCCCCCCCTGC |
| | | CCCCCCTGCCCCGCCCCCGAGTTCCTGGGCGGCCCCAGCG |
| | | TGTTCCTGTTCCCCCCCAAGCCCAAGGACACCCTGATGAT |
| | | CAGCAGAACCCCCGAGGTGACCTGCGTGGTGGTGGACGT |
| | | GAGCCAGGAGGACCCCGAGGTGCAGTTCAACTGGTACGT |
| | | GGACGGCGTGGAGGTGCACAACGCCAAGACCAAGCCCAG |
| | | AGAGGAGCAGTTCAACAGCACCTACAGAGTGGTGAGCGT |
| | | GCTGACCGTGCTGCACCAGGACTGGCTGAACGGCAAGGA |
| | | GTACAAGTGCAAGGTGAGCAACAAGGGCCTGCCCAGCAG |
| | | CATCGAGAAGACCATCAGCAAGGCCAAGGGCCAGCCCAG |
| | | AGAGCCCCAGGTGTACACCCTGCCCCCCAGCCAGGAGGA |
| | | GATGACCAAGAACCAGGTGAGCCTGACCTGCCTGGTGAA |
| | | GGGCTTCTACCCCAGCGACATCGCCGTGGAGTGGGAGAGC |
| | | AACGGCCAGCCCGAGAACAACTACAAGACCACCCCCCCC |
| | | GTGCTGGACAGCGACGGCAGCTTCTTCCTGTACAGCAGAC |
| | | TGACCGTGGACAAGAGCAGATGGCAGGAGGGCAACGTGT |
| | | TCAGCTGCAGCGTGATGCACGAGGCCCTGCACAACCACTA |
| | | CACCCAGAAGAGCCTGAGCCTGAGCCTGGGCAAGGGCGG |
| | | AGGCGGAAGCGGCGGAGGCGGATCTGGCGGAGGCGGAAG |
| | | CGGCGGAGGCGGCTCTGAGCACCCCAACGCGCGCAAATA |
| | | CAAAGGAGCCAACAAGAAGGGCTTGTCCAAGGGCTGCTT |
| | | CGGCCTCAAGCTGGACCGAATCGGCTCCATGAGCGGCCTG |
| | | GGATGTTAACTCGAGAATCAACCTCTGGATTACAAAATTT |
| | | GTGAAAGATTGACTGGTATTCTTAACTATGTTGCTCCTTTT |
| | | ACGCTATGTGGATACGCTGCTTTAATGCCTTTGTATCATGC |
| | | TATTGCTTCCCGTATGGCTTTCATTTTCTCCTCCTTGTATAA |
| | | ATCCTGGTTGCTGTCTCTTTATGAGGAGTTGTGGCCCGTTG |
| | | TCAGGCAACGTGGCGTGGTGTGCACTGTGTTTGCTGACGC |
| | | AACCCCCACTGGTTGGGGCATTGCCACCACCTGTCAGCTC |
| | | CTTTCCGGGACTTTCGCTTTCCCCCTCCCTATTGCCACGGC |
| | | GGAACTCATCGCCGCCTGCCTTGCCCGCTGCTGGACAGGG |
| | | GCTCGGCTGTTGGGCACTGACAATTCCGTGGTCTGTTCTCA |
| | | TCACATCATATCAAGGTTATATACCATCAATATTGCCACA |
| | | GATGTTACTTAGCCTTTTAATATTTCTCTAATTTAGTGTAT |
| | | ATGCAATGATAGTTCTCTGATTTCTGAGATTGAGTTTCTCA |
| | | TGTGTAATGATTATTTAGAGTTTCTCTTTCATCTGTTCAAA |
| | | TTTTTGTCTAGTTTTATTTTTTACTGATTTGTAAGACTTCTT |
| | | TTTATAATCTGCATATTACAATTCTCTTTACTGGGGTGTTG |
| | | CAAATATTTTCTGTCATTCTATGGCCTGACTTTTCTTAATG |
| | | GTTTTTTAATTTTAAAAATAAGTCTTAATATTCATGCAATC |
| | | TAATTAACAATCTTTTCTTTGTGGTTAGGACTTTGAGTCAT |
| | | AAGAAATTTTTCTCTACACTGAAGTCATGATGGCATGCTT |
| | | CTATATTATTTTCTAAAAGATTTAAAGTTTTGCCTTCTCCA |
| | | TTTAGACTTATAATTCACTGGAATTTTTTTGTGTGTATGGT |
| | | ATGACATATGGGTTCCCTTTTATTTTTTACATATAAATATA |
| | | TTTCCCTGTTTTTCTAAAAAAGAAAAAGATCATCATTTTCC |
| | | CATTGTAAAATGCCATATTTTTTTCATAGGTCACTTACATA |
| | | TATCAATGGGTCTGTTTCTGAGCTCTACTCTATTTTATCAG |
| | | CCTCACTGTCTATCCCCACACATCTCATGCTTTGCTCTAAA |
| | | TCTTGATATTTAGTGGAACATTCTTTCCCATTTTGTTCTAC |
| | | AAGAATATTTTTGTTATTGTCTTTGGGCTTTCTATATACAT |
| | | TTTGAAATGAGGTTGACAAGTTACCTAGGAAAACTGTCTT |
| | | CCTGCCCGGGTGGCATCCCTGTGACCCCTCCCCAGTGCCT |
| | | CTCCTGGCCCTGGAAGTTGCCACTCCAGTGCCCACCAGCC |
| | | TTGTCCTAATAAAATTAAGTTGCATCATTTTGTCTGACTAG |
| | | GTGTCCTTCTATAATATTATGGGGTGGAGGGGGGTGGTAT |
| | | GGAGCAAGGGGCCCAAGTTGGGAAGAAACCTGTAGGGCC |
| | | TGCGTACGTAGATAAGTAGCATGGCGGGTTAATCATTAAC |
| | | TACAAGGAACCCCTAGTGATGGAGTTGGCCACTCCCTCTC |
| | | TGCGCGCTCGCTCGCTCACTGAGGCCGGGCGACCAAAGGT |
| | | CGCCCGACGCCCGGGCTTTGCCCGGGCGGCCTCAGTGAGC |
| | | GAGCGAGCGCGCAGCTGCTGGTACCGGGCCCCCCCTCGAG |
| | | GTCGACGGTATCGATGGATCCGATATCGAATTCAACCTGC |
| | | AGGCATGCTCTGAGACAATAACCCTGATAAATGCTTCAAT |
| | | AATGTAGCCAACCACTAGAACTATAGCTAGAGTCCTGGGC |
| | | GAACAAACGATGCTCGCCTTCCAGAAAACCGAGGATGCG |
| | | AACCACTTCATCCGGGGTCAGCACCACCGGCAAGCGCCGC |
| | | GACGGCCGAGGTCTTCCGATCTCCTGAAGCCAGGGCAGAT |
| | | CCGTGCACAGCACCTTGCCGTAGAAGAACAGCAAGGCCG |
| | | CCAATGCCTGACGATGCGTGGAGACCGAAACCTTGCGCTC |
| | | GTTCGCCAGCCAGGACAGAAATGCCTCGACTTCGCTGCTG |
| | | CCCAAGGTTGCCGGGTGACGCACACCGTGGAAACGGATG |
| | | AAGGCACGAACCCAGTTGACATAAGCCTGTTCGGTTCGTA |
| | | AACTGTAATGCAAGTAGCGTATGCGCTCACGCAACTGGTC |
| | | CAGAACCTTGACCGAACGCAGCGGTGGTAACGGCGCAGT |
| | | GGCGGTTTTCATGGCTTGTTATGACTGTTTTTTTGTACAGT |
| | | CTATGCCTCGGGCATCCAAGCAGCAAGCGCGTTACGCCGT |
| | | GGGTCGATGTTTGATGTTATGGAGCAGCAACGATGTTACG |
| | | CAGCAGCAACGATGTTACGCAGCAGGGCAGTCGCCCTAA |
| | | AACAAAGTTAGGTGGCTCAAGTATGGGCATCATTCGCACA |
| | | TGTAGGCTCGGCCCTGACCAAGTCAAATCCATGCGGGCTG |
| | | CTCTTGATCTTTTCGGTCGTGAGTTCGGAGACGTAGCCACC |
| | | TACTCCCAACATCAGCCGGACTCCGATTACCTCGGGAACT |
| | | TGCTCCGTAGTAAGACATTCATCGCGCTTGCTGCCTTCGAC |
| | | CAAGAAGCGGTTGTTGGCGCTCTCGCGGCTTACGTTCTGC |
| | | CCAGGTTTGAGCAGCCGCGTAGTGAGATCTATATCTATGA |
| | | TCTCGCAGTCTCCGGCGAGCACCGGAGGCAGGGCATTGCC |
| | | ACCGCGCTCATCAATCTCCTCAAGCATGAGGCCAACGCGC |
| | | TTGGTGCTTATGTGATCTACGTGCAAGCAGATTACGGTGA |
| | | CGATCCCGCAGTGGCTCTCTATACAAAGTTGGGCATACGG |
| | | GAAGAAGTGATGCACTTTGATATCGACCCAAGTACCGCCA |
| | | CCTAACAATTCGTTCAAGCCGAGATCGGCTTCCCGGCCGC |
| | | GGAGTTGTTCGGTAAATTGTCACAACGCCGCGAATATAGT |
| | | CTTTACCATGCCCTTGGCCACGCCCCTCTTTAATACGACGG |
| | | GCAATTTGCACTTCAGAAAATGAAGAGTTTGCTTTAGCCA |
| | | TAACAAAAGTCCAGTATGCTTTTTCACAGCATAACTGGAC |
| | | TGATTTCAGTTTACAACTATTCTGTCTAGTTTAAGACTTTA |
| | | TTGTCATAGTTTAGATCTATTTTGTTCAGTTTAAGACTTTA |
| | | TTGTCCGCCCACACCCGCTTACGCAGGGCATCCATTTATTA |
| | | CTCAACCGTAACCGATTTTGCCAGGTTACGCGGCTGGTCT |
| | | GCGGTGTGAAATACCGCACAGATGCGTAAGGAGAAAATA |
| | | CCGCATCAGGCGCTCTTCCGCTTCCTCGCTCACTGACTCGC |
| | | TGCGCTCGGTCGTTCGGCTGCGGCGAGCGGTATCAGCTCA |
| | | CTCAAAGGCGGTAATACGGTTATCCACAGAATCAGGGGAT |
| | | AACGCAGGAAAGAACATGTGAGCAAAAGGCCAGCAAAAG |
| | | GCCAGGAACCGTAAAAAGGCCGCGTTGCTGGCGTTTTTCC |
| | | ATAGGCTCCGCCCCCCTGACGAGCATCACAAAAATCGACG |
| | | CTCAAGTCAGAGGTGGCGAAACCCGACAGGACTATAAAG |
| | | ATACCAGGCGTTTCCCCCTGGAAGCTCCCTCGTGCGCTCTC |
| | | CTGTTCCGACCCTGCCGCTTACCGGATACCTGTCCGCCTTT |
| | | CTCCCTTCGGGAAGCGTGGCGCTTTCTCAATGCTCACGCT |
| | | GTAGGTATCTCAGTTCGGTGTAGGTCGTTCGCTCCAAGCT |
| | | GGGCTGTGTGCACGAACCCCCCGTTCAGCCCGACCGCTGC |
| | | GCCTTATCCGGTAACTATCGTCTTGAGTCCAACCCGGTAA |
| | | GACACGACTTATCGCCACTGGCAGCAGCCACTGGTAACAG |
| | | GATTAGCAGAGCGAGGTATGTAGGCGGTGCTACAGAGTTC |
| | | TTGAAGTGGTGGCCTAACTACGGCTACACTAGAAGGACAG |
| | | TATTTGGTATCTGCGCTCTGCTGAAGCCAGTTACCTTCGGA |
| | | AAAAGAGTTGGTAGCTCTTGATCCGGCAAACAAACCACCG |
| | | CTGGTAGCGGTGGTTTTTTTGTTTGCAAGCAGCAGATTAC |
| | | GCGCAGAAAAAAAGGATCTCAAGAAGATCCTTTGATCTTT |
| | | TCTACGTGGTCTGACGCTCAGTGGAACGAAAACTCACGTT |
| | | AAGGGATTTTGGTCATGAGATTATCAAAAAGGATCTTCAC |
| | | CTAGATCCTTTTAAATTAAAAATGAAGTTTTAAATCAATCT |
| | | AAAGTATATATGAGTAAACTTGGTCTGACGGTTACTCAGA |
| | | AGAACTCGTCAAGAAGGCGATAGAAGGCGATGCGCTGCG |
| | | AATCGGGAGCGGCGATACCGTAAAGCACGAGGAAGCGGT |
| | | CAGCCCATTCGCCGCCAAGCTCTTCAGCAATATCACGGGT |
| | | AGCCAACGCTATGTCCTGATAGCGGTCCGCCACACCCAGC |
| | | CGGCCACAGTCGATGAATCCAGAAAAGCGGCCATTTTCCA |
| | | CCATGATATTCGGCAAGCAGGCATCGCCATGGGTCACGAC |
| | | GAGATCCTCGCCGTCGGGCATGCGCGCCTTGAGCCTGGCG |
| | | AACAGTTCGGCTGGCGCGAGCCCCTGATGCTCTTCGTCCA |
| | | GATCATCCTGATCGACAAGACCGGCTTCCATCCGAGTACG |
| | | TGCTCGCTCGATGCGATGTTTCGCTTGGTGGTCGAATGGG |
| | | CAGGTAGCCGGATCAAGCGTATGCAGCCGCCGCATTGCAT |
| | | CAGCCATGATGGATACTTTCTCGGCAGGAGCAAGGTGAGA |
| | | TGACAGGAGATCCTGCCCCGGCACTTCGCCCAATAGCAGC |
| | | CAGTCCCTTCCCGCTTCAGTGACAACGTCGAGCACAGCTG |
| | | CGCAAGGAACGCCCGTCGTGGCCAGCCACGATAGCCGCG |
| | | CTGCCTCGTCCTGCAGTTCATTCAGGGCACCGGACAGGTC |
| | | GGTCTTGACAAAAAGAACCGGGCGCCCCTGCGCTGACAG |
| | | CCGGAACACGGCGGCATCAGAGCAGCCGATTGTCTGTTGT |
| | | GCCCAGTCATAGCCGAATAGCCTCTCCACCCAAGCGGCCG |
| | | GAGAACCTGCGTGCAATCCATCTTGTTCAATCATACTCTTC |
| | | CTTTTTCAATATTATTGAAGCATTTATCAGGGTTATTGTCT |
| | | CATGAGCGGATACATATTTGAATGTATTTAGAAAAATAAA |
| | | CAAATAGGGGTTCCGCGCACATTTCCCCGAAAAGTGCCAC |
| | | CTGACGTCTAAGAAACCATTATTATCATGACATTAACCTA |
| | | TAAAAATAGGCGTATCACGAGGCCCTTTCGTCTCGCGCGT |
| | | TTCGGTGATGACGGTGAAAACCTCTGACACATGCAGCTCC |
| | | CGGAGACGGTCACAGCTTGTCTGTAAGCGGATGCCGGGA |
| | | GCAGACAAGCCCGTCAGGGCGCGTCAGCGGGTGTTGGCG |
| | | GGTGTCGGGGCTGGCTTAACTATGCGGCATCAGAGCAGAT |
| | | TGTACTGAGAGTGCACCATATGCGGTGTGAAATACCGCAC |
| | | AGATGCGTAAGGAGAAAATACCGCATCAGGAAATTGTAA |
| | | ACGTTAATATTTTGTTAAAATTCGCGTTAAATTTTTGTTAA |
| | | ATCAGCTCATTTTTTAACCAATAGGCCGAAATCGGCAAAA |
| | | TCCCTTATAAATCAAAAGAATAGACCGAGATAGGGTTGAG |
| | | TGTTGTTCCAGTTTGGAACAAGAGTCCACTATTAAAGAAC |
| | | GTGGACTCCAACGTCAAAGGGCGAAAAACCGTCTATCAG |
| | | GGCGATGGCCCACTACGTGAACCATCACCCTAATCAAGTT |
| | | TTTTGGGGTCGAGGTGCCGTAAAGCACTAAATCGGAACCC |
| | | TAAAGGGAGCCCCCGATTTAGAGCTTGACGGGGAAAGCC |
| | | GGCGAACGTGGCGAGAAAGGAAGGGAAGAAAGCGAAAG |
| | | GAGCGGGCGCTAGGGCGCTGGCAAGTGTAGCGGTCACGC |
| | | TGCGCGTAACCACCACACCCGCCGCGCTTAATGCGCCGCT |
| | | ACAGGGCGCGTCC |
| AMI305 | 180 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGGGTACCGGGCCCCCCCTCGAGGTCGACGGTATCGAT |
| | | GGATCCGATATCGAATTCAAGCGCGCTCGCTCGCTCACTG |
| | | AGGCCGCCCGGGCAAAGCCCGGGCGTCGGGCGACCTTTG |
| | | GTCGCCCGGCCTCAGTGAGCGAGCGAGCGCGCAGAGAGG |
| | | GAGTGGCCAACTCCATCACTAGGGGTTCCTTGTAGTTAAT |
| | | GATTAACCCGCCATGCTACTTATCTACGTACTCTGGAGAC |
| | | GACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTGG |
| | | AGTATTTACGGTAAACTGCCCACTTGGCAGTACATCAAGT |
| | | GTATCATATGCCAAGTCCGCCCCCTATTGACGTCAATGAC |
| | | GGTAAATGGCCCGCCTGGCATTATGCCCAGTACATGACCT |
| | | TACGGGACTTTCCTACTTGGCAGTACATCTACGTATTAGTC |
| | | ATCGCTATTACCATGGTCGAGGTGAGCCCCACGTTCTGCT |
| | | TCACTCTCCCCATCTCCCCCCCCTCCCCACCCCCAATTTTG |
| | | TATTTATTTATTTTTTAATTATTTTGTGCAGCGATGGGGGC |
| | | GGGGGGGGGGGGGGGGCGCGCGCCAGGCGGGGCGGGGC |
| | | GGGGCGAGGGGGGGGCGGGGCGAGGCGGAGAGGTGCG |
| | | GCGGCAGCCAATCAGAGCGGCGCGCTCCGAAAGTTTCCTT |
| | | TTATGGCGAGGCGGCGGCGGCGGCGGCCCTATAAAAAGC |
| | | GAAGCGCGCGGCGGGCGGGAGTCGCTGCGACGCTGCCTT |
| | | CGCCCCGTGCCCCGCTCCGCCGCCGCCTCGCGCCGCCCGC |
| | | CCCGGCTCTGACTGACCGCGTTACTCCCACAGCTCACTCTC |
| | | TTCCGCATCGCTGTCTGCGAGGGCCAGCTGTTGGGGTGAG |
| | | TACTCCCTCTCAAAAGCGGGCATGACTTCTGCGCTAAGAT |
| | | TGTCAGTTTCCAAAAACGAGGAGGATTTGATATTCACCTG |
| | | GCCCGCGGTGATGCCTTTGAGGGTGGCCGCGTCCATCTGG |
| | | TCAGAAAAGACAATCTTTTTGTTGTCAAGCTTGGTGGCAA |
| | | ACGACCCGTAGAGGGCGTTGGACAGCAACTTGGCGATGG |
| | | AGCGCAGGGTTTGGTTTTTGTCGCGATCGGCGCGCTCCTT |
| | | GGCCGCGATGTTTAGCTGCACGTATTCGCGCGCAACGCAC |
| | | CGCCATTCGGGAAAGACGGTGGTGCGCTCGTCGGGCACCA |
| | | GGTGCACGCGCCAACCGCGGTTGTGCAGGGTGACAAGGT |
| | | CAACGCTGGTGGCTACCTCTCCGCGTAGGCGCTCGTTGGT |
| | | CCAGCAGAGGCGGCCGCCCTTGCGCGAACAGAATGGCGG |
| | | TAGTGGGTCTAGCTGCGTCTCGTCCGGGGGGTCTGCGTCC |
| | | ACGGTAAAGACCCCGGGCAGCAGGCGCGCGTCGAAGTAG |
| | | TCTATCTTGCATCCTTGCAAGTCTAGCGCCTGCTGCCATGC |
| | | GCGGGCGGCAAGCGCGCGCTCGTATGGGTTGAGTGGGGG |
| | | ACCCCATGGCATGGGGTGGGTGAGCGCGGAGGCGTACAT |
| | | GCCGCAAATGTCGTAAACGTAGAGGGGCTCTCTGAGTATT |
| | | CCAAGATATGTAGGGTAGCATCTTCCACCGCGGATGCTGG |
| | | CGCGCACGTAATCGTATAGTTCGTGCGAGGGAGCGAGGA |
| | | GGTCGGGACCGAGGTTGCTACGGGCGGGCTGCTCTGCTCG |
| | | GAAGACTATCTGCCTGAAGATGGCATGTGAGTTGGATGAT |
| | | ATGGTTGGACGCTGGAAGACGTTGAAGCTGGCGTCTGTGA |
| | | GACCTACCGCGTCACGCACGAAGGAGGCGTAGGAGTCGC |
| | | GCAGCTTGTTGACCAGCTCGGCGGTGACCTGCACGTCTAG |
| | | GGCGCAGTAGTCCAGGGTTTCCTTGATGATGTCATACTTA |
| | | TCCTGTCCCTTTTTTTTCCACAGCTCGCGGTTGAGGACAAA |
| | | CTCTTCGCGGTCTTTCCAGTACTCTTGGATCGGAAACCCGT |
| | | CGGCCTCCGAACGGTACTCCGCCACCGAGGGACCTGAGCG |
| | | AGTCCGCATCGACCGGATCGGAAAACCTCTCGAGAAAGG |
| | | CGTCTAACCAGTCACAGTCGCAAGGTAGGCTGAGCACCGT |
| | | GGCGGGCGGCAGCGGGTGGCGGTCGGGGTTGTTTCTGGCG |
| | | GAGGTGCTGCTGATGATGTAATTAAAGTAGGCGGTCTTGA |
| | | GACGGCGGATGGTCGAATCTGGCCATACACTTGAGTGACA |
| | | ATGACATCCACTTTGCCTTTCTCTCCACAGGTGTCCACTCC |
| | | CAGGTCCAAGTTTAAACGCCGCCACCTAGGAGTTCGGCCT |
| | | GAGCTGGCTGTTCCTGGTGGCCATCCTTAAGGGCGTGCAG |
| | | TGCGACAAAACTCACACATGCCCACCGTGCCCAGCACCTG |
| | | AACTCCTGGGGGGACCGTCAGTCTTCCTCTTCCCCCCAAA |
| | | ACCCAAGGACACCCTCTAGATCTCCCGGACCCCTGAGGTC |
| | | ACATGCGTGGTGGTGGACGTGAGCCACGAAGACCCTGAG |
| | | GTCAAGTTCAACTGGTACGTGGACGGCGTGGAGGTGCATA |
| | | ATGCCAAGACAAAGCCGCGGGAGGAGCAGTACAACAGCA |
| | | CGTACCGTGTGGTCAGCGTCCTCACCGTCCTGCACCAGGA |
| | | CTGGCTGAATGGCAAGGAGTACAAGTGCAAGGTCTCCAA |
| | | CAAAGCCCTCCCAGCCCCCATCGAGAAAACCATCTCCAAA |
| | | GCCAAAGGGCAGCCCCGAGAACCACAGGTGTACACCCTG |
| | | CCCCCATCCCGGGATGAGCTGACCAAGAACCAGGTCAGCC |
| | | TGACCTGCCTGGTCAAAGGCTTCTATCCCAGCGACATCGC |
| | | CGTGGAGTGGGAGAGCAATGGGCAGCCGGAGAACAACTA |
| | | CAAGACCACGCCTCCCGTGCTGGACTCCGACGGCTCCTTC |
| | | TTCCTCTACAGCAAGCTCACCGTGGACAAGAGCAGGTGGC |
| | | AGCAGGGGAACGTCTTCTCATGCTCCGTGATGCATGAGGC |
| | | TCTGCACAACCACTACACGCAGAAGAGCCTCTCCCTGTCT |
| | | CCGGGTAAAGGCGGAGGCGGAAGCGGCGGAGGCGGATCT |
| | | GGCGGAGGCGGCAGCGGCGGCGGCGGCTCTTAGCACCCC |
| | | AACGCGCGCAAATACAAAGGAGCCAACAAGAAGGGCTTG |
| | | TCCAAGGGCTGCTTCGGCCTCAAGCTGGACCGAATCGGCT |
| | | CCATGAGCGGCCTGGGATGTTAACTCGAGAATCAACCTCT |
| | | GGATTACAAAATTTGTGAAAGATTGACTGGTATTCTTAAC |
| | | TATGTTGCTCCTTTTACGCTATGTGGATACGCTGCTTTAAT |
| | | GCCTTTGTATCATGCTATTGCTTCCCGTATGGCTTTCATTTT |
| | | CTCCTCCTTGTATAAATCCTGGTTGCTGTCTCTTTATGAGG |
| | | AGTTGTGGCCCGTTGTCAGGCAACGTGGCGTGGTGTGCAC |
| | | TGTGTTTGCTGACGCAACCCCCACTGGTTGGGGCATTGCC |
| | | ACCACCTGTCAGCTCCTTTCCGGGACTTTCGCTTTCCCCCT |
| | | CCCTATTGCCACGGCGGAACTCATCGCCGCCTGCCTTGCC |
| | | CGCTGCTGGACAGGGGCTCGGCTGTTGGGCACTGACAATT |
| | | CCGTGGTCTGTTCTCATCACATCATATCAAGGTTATATACC |
| | | ATCAATATTGCCACAGATGTTACTTAGCCTTTTAATATTTC |
| | | TCTAATTTAGTGTATATGCAATGATAGTTCTCTGATTTCTG |
| | | AGATTGAGTTTCTCATGTGTAATGATTATTTAGAGTTTCTC |
| | | TTTCATCTGTTCAAATTTTTGTCTAGTTTTATTTTTTACTGA |
| | | TTTGTAAGACTTCTTTTTATAATCTGCATATTACAATTCTC |
| | | TTTACTGGGGTGTTGCAAATATTTTCTGTCATTCTATGGCC |
| | | TGACTTTTCTTAATGGTTTTTTAATTTTAAAAATAAGTCTT |
| | | AATATTCATGCAATCTAATTAACAATCTTTTCTTTGTGGTT |
| | | AGGACTTTGAGTCATAAGAAATTTTTCTCTACACTGAAGT |
| | | CATGATGGCATGCTTCTATATTATTTTCTAAAAGATTTAAA |
| | | GTTTTGCCTTCTCCATTTAGACTTATAATTCACTGGAATTT |
| | | TTTTGTGTGTATGGTATGACATATGGGTTCCCTTTTATTTTT |
| | | TACATATAAATATATTTCCCTGTTTTTCTAAAAAAGAAAA |
| | | AGATCATCATTTTCCCATTGTAAAATGCCATATTTTTTTCA |
| | | TAGGTCACTTACATATATCAATGGGTCTGTTTCTGAGCTCT |
| | | ACTCTATTTTATCAGCCTCACTGTCTATCCCCACACATCTC |
| | | ATGCTTTGCTCTAAATCTTGATATTTAGTGGAACATTCTTT |
| | | CCCATTTTGTTCTACAAGAATATTTTTGTTATTGTCTTTGG |
| | | GCTTTCTATATACATTTTGAAATGAGGTTGACAAGTTACCT |
| | | AGGAAAACTGTCTTCCTGCCCGGGTGGCATCCCTGTGACC |
| | | CCTCCCCAGTGCCTCTCCTGGCCCTGGAAGTTGCCACTCCA |
| | | GTGCCCACCAGCCTTGTCCTAATAAAATTAAGTTGCATCA |
| | | TTTTGTCTGACTAGGTGTCCTTCTATAATATTATGGGGTGG |
| | | AGGGGGGTGGTATGGAGCAAGGGGCCCAAGTTGGGAAGA |
| | | AACCTGTAGGGCCTGCGTACGTAGATAAGTAGCATGGCGG |
| | | GTTAATCATTAACTACAAGGAACCCCTAGTGATGGAGTTG |
| | | GCCACTCCCTCTCTGCGCGCTCGCTCGCTCACTGAGGCCG |
| | | GGCGACCAAAGGTCGCCCGACGCCCGGGCTTTGCCCGGGC |
| | | GGCCTCAGTGAGCGAGCGAGCGCGCAGCTGCTGGTACCG |
| | | GGCCCCCCCTCGAGGTCGACGGTATCGATGGATCCGATAT |
| | | CGAATTCAACCTGCAGGCATGCTCTGAGACAATAACCCTG |
| | | ATAAATGCTTCAATAATGTAGCCAACCACTAGAACTATAG |
| | | CTAGAGTCCTGGGCGAACAAACGATGCTCGCCTTCCAGAA |
| | | AACCGAGGATGCGAACCACTTCATCCGGGGTCAGCACCAC |
| | | CGGCAAGCGCCGCGACGGCCGAGGTCTTCCGATCTCCTGA |
| | | AGCCAGGGCAGATCCGTGCACAGCACCTTGCCGTAGAAG |
| | | AACAGCAAGGCCGCCAATGCCTGACGATGCGTGGAGACC |
| | | GAAACCTTGCGCTCGTTCGCCAGCCAGGACAGAAATGCCT |
| | | CGACTTCGCTGCTGCCCAAGGTTGCCGGGTGACGCACACC |
| | | GTGGAAACGGATGAAGGCACGAACCCAGTTGACATAAGC |
| | | CTGTTCGGTTCGTAAACTGTAATGCAAGTAGCGTATGCGC |
| | | TCACGCAACTGGTCCAGAACCTTGACCGAACGCAGCGGTG |
| | | GTAACGGCGCAGTGGCGGTTTTCATGGCTTGTTATGACTG |
| | | TTTTTTTGTACAGTCTATGCCTCGGGCATCCAAGCAGCAA |
| | | GCGCGTTACGCCGTGGGTCGATGTTTGATGTTATGGAGCA |
| | | GCAACGATGTTACGCAGCAGCAACGATGTTACGCAGCAG |
| | | GGCAGTCGCCCTAAAACAAAGTTAGGTGGCTCAAGTATGG |
| | | GCATCATTCGCACATGTAGGCTCGGCCCTGACCAAGTCAA |
| | | ATCCATGCGGGCTGCTCTTGATCTTTTCGGTCGTGAGTTCG |
| | | GAGACGTAGCCACCTACTCCCAACATCAGCCGGACTCCGA |
| | | TTACCTCGGGAACTTGCTCCGTAGTAAGACATTCATCGCG |
| | | CTTGCTGCCTTCGACCAAGAAGCGGTTGTTGGCGCTCTCG |
| | | CGGCTTACGTTCTGCCCAGGTTTGAGCAGCCGCGTAGTGA |
| | | GATCTATATCTATGATCTCGCAGTCTCCGGCGAGCACCGG |
| | | AGGCAGGGCATTGCCACCGCGCTCATCAATCTCCTCAAGC |
| | | ATGAGGCCAACGCGCTTGGTGCTTATGTGATCTACGTGCA |
| | | AGCAGATTACGGTGACGATCCCGCAGTGGCTCTCTATACA |
| | | AAGTTGGGCATACGGGAAGAAGTGATGCACTTTGATATCG |
| | | ACCCAAGTACCGCCACCTAACAATTCGTTCAAGCCGAGAT |
| | | CGGCTTCCCGGCCGCGGAGTTGTTCGGTAAATTGTCACAA |
| | | CGCCGCGAATATAGTCTTTACCATGCCCTTGGCCACGCCC |
| | | CTCTTTAATACGACGGGCAATTTGCACTTCAGAAAATGAA |
| | | GAGTTTGCTTTAGCCATAACAAAAGTCCAGTATGCTTTTTC |
| | | ACAGCATAACTGGACTGATTTCAGTTTACAACTATTCTGTC |
| | | TAGTTTAAGACTTTATTGTCATAGTTTAGATCTATTTTGTT |
| | | CAGTTTAAGACTTTATTGTCCGCCCACACCCGCTTACGCA |
| | | GGGCATCCATTTATTACTCAACCGTAACCGATTTTGCCAG |
| | | GTTACGCGGCTGGTCTGCGGTGTGAAATACCGCACAGATG |
| | | CGTAAGGAGAAAATACCGCATCAGGCGCTCTTCCGCTTCC |
| | | TCGCTCACTGACTCGCTGCGCTCGGTCGTTCGGCTGCGGC |
| | | GAGCGGTATCAGCTCACTCAAAGGCGGTAATACGGTTATC |
| | | CACAGAATCAGGGGATAACGCAGGAAAGAACATGTGAGC |
| | | AAAAGGCCAGCAAAAGGCCAGGAACCGTAAAAAGGCCGC |
| | | GTTGCTGGCGTTTTTCCATAGGCTCCGCCCCCCTGACGAGC |
| | | ATCACAAAAATCGACGCTCAAGTCAGAGGTGGCGAAACC |
| | | CGACAGGACTATAAAGATACCAGGCGTTTCCCCCTGGAAG |
| | | CTCCCTCGTGCGCTCTCCTGTTCCGACCCTGCCGCTTACCG |
| | | GATACCTGTCCGCCTTTCTCCCTTCGGGAAGCGTGGCGCTT |
| | | TCTCAATGCTCACGCTGTAGGTATCTCAGTTCGGTGTAGGT |
| | | CGTTCGCTCCAAGCTGGGCTGTGTGCACGAACCCCCCGTT |
| | | CAGCCCGACCGCTGCGCCTTATCCGGTAACTATCGTCTTG |
| | | AGTCCAACCCGGTAAGACACGACTTATCGCCACTGGCAGC |
| | | AGCCACTGGTAACAGGATTAGCAGAGCGAGGTATGTAGG |
| | | CGGTGCTACAGAGTTCTTGAAGTGGTGGCCTAACTACGGC |
| | | TACACTAGAAGGACAGTATTTGGTATCTGCGCTCTGCTGA |
| | | AGCCAGTTACCTTCGGAAAAAGAGTTGGTAGCTCTTGATC |
| | | CGGCAAACAAACCACCGCTGGTAGCGGTGGTTTTTTTGTT |
| | | TGCAAGCAGCAGATTACGCGCAGAAAAAAAGGATCTCAA |
| | | GAAGATCCTTTGATCTTTTCTACGTGGTCTGACGCTCAGTG |
| | | GAACGAAAACTCACGTTAAGGGATTTTGGTCATGAGATTA |
| | | TCAAAAAGGATCTTCACCTAGATCCTTTTAAATTAAAAAT |
| | | GAAGTTTTAAATCAATCTAAAGTATATATGAGTAAACTTG |
| | | GTCTGACGGTTACTCAGAAGAACTCGTCAAGAAGGCGATA |
| | | GAAGGCGATGCGCTGCGAATCGGGAGCGGCGATACCGTA |
| | | AAGCACGAGGAAGCGGTCAGCCCATTCGCCGCCAAGCTCT |
| | | TCAGCAATATCACGGGTAGCCAACGCTATGTCCTGATAGC |
| | | GGTCCGCCACACCCAGCCGGCCACAGTCGATGAATCCAGA |
| | | AAAGCGGCCATTTTCCACCATGATATTCGGCAAGCAGGCA |
| | | TCGCCATGGGTCACGACGAGATCCTCGCCGTCGGGCATGC |
| | | GCGCCTTGAGCCTGGCGAACAGTTCGGCTGGCGCGAGCCC |
| | | CTGATGCTCTTCGTCCAGATCATCCTGATCGACAAGACCG |
| | | GCTTCCATCCGAGTACGTGCTCGCTCGATGCGATGTTTCGC |
| | | TTGGTGGTCGAATGGGCAGGTAGCCGGATCAAGCGTATGC |
| | | AGCCGCCGCATTGCATCAGCCATGATGGATACTTTCTCGG |
| | | CAGGAGCAAGGTGAGATGACAGGAGATCCTGCCCCGGCA |
| | | CTTCGCCCAATAGCAGCCAGTCCCTTCCCGCTTCAGTGAC |
| | | AACGTCGAGCACAGCTGCGCAAGGAACGCCCGTCGTGGC |
| | | CAGCCACGATAGCCGCGCTGCCTCGTCCTGCAGTTCATTC |
| | | AGGGCACCGGACAGGTCGGTCTTGACAAAAAGAACCGGG |
| | | CGCCCCTGCGCTGACAGCCGGAACACGGCGGCATCAGAG |
| | | CAGCCGATTGTCTGTTGTGCCCAGTCATAGCCGAATAGCC |
| | | TCTCCACCCAAGCGGCCGGAGAACCTGCGTGCAATCCATC |
| | | TTGTTCAATCATACTCTTCCTTTTTCAATATTATTGAAGCA |
| | | TTTATCAGGGTTATTGTCTCATGAGCGGATACATATTTGAA |
| | | TGTATTTAGAAAAATAAACAAATAGGGGTTCCGCGCACAT |
| | | TTCCCCGAAAAGTGCCACCTGACGTCTAAGAAACCATTAT |
| | | TATCATGACATTAACCTATAAAAATAGGCGTATCACGAGG |
| | | CCCTTTCGTCTCGCGCGTTTCGGTGATGACGGTGAAAACC |
| | | TCTGACACATGCAGCTCCCGGAGACGGTCACAGCTTGTCT |
| | | GTAAGCGGATGCCGGGAGCAGACAAGCCCGTCAGGGCGC |
| | | GTCAGCGGGTGTTGGCGGGTGTCGGGGCTGGCTTAACTAT |
| | | GCGGCATCAGAGCAGATTGTACTGAGAGTGCACCATATGC |
| | | GGTGTGAAATACCGCACAGATGCGTAAGGAGAAAATACC |
| | | GCATCAGGAAATTGTAAACGTTAATATTTTGTTAAAATTC |
| | | GCGTTAAATTTTTGTTAAATCAGCTCATTTTTTAACCAATA |
| | | GGCCGAAATCGGCAAAATCCCTTATAAATCAAAAGAATA |
| | | GACCGAGATAGGGTTGAGTGTTGTTCCAGTTTGGAACAAG |
| | | AGTCCACTATTAAAGAACGTGGACTCCAACGTCAAAGGGC |
| | | GAAAAACCGTCTATCAGGGCGATGGCCCACTACGTGAACC |
| | | ATCACCCTAATCAAGTTTTTTGGGGTCGAGGTGCCGTAAA |
| | | GCACTAAATCGGAACCCTAAAGGGAGCCCCCGATTTAGA |
| | | GCTTGACGGGGAAAGCCGGCGAACGTGGCGAGAAAGGAA |
| | | GGGAAGAAAGCGAAAGGAGCGGGCGCTAGGGCGCTGGCA |
| | | AGTGTAGCGGTCACGCTGCGCGTAACCACCACACCCGCCG |
| | | CGCTTAATGCGCCGCTACAGGGCGCGTCC |
| AMI312 | 181 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGGGTACCGGGCCCCCCCTCGAGGTCGACGGTATCGAT |
| | | GGATCCGATATCGAATTCAAGCGCGCTCGCTCGCTCACTG |
| | | AGGCCGCCCGGGCAAAGCCCGGGCGTCGGGCGACCTTTG |
| | | GTCGCCCGGCCTCAGTGAGCGAGCGAGCGCGCAGAGAGG |
| | | GAGTGGCCAACTCCATCACTAGGGGTTCCTTGTAGTTAAT |
| | | GATTAACCCGCCATGCTACTTATCTACGTACTCTGGAGAC |
| | | GACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTGG |
| | | AGTATTTACGGTAAACTGCCCACTTGGCAGTACATCAAGT |
| | | GTATCATATGCCAAGTCCGCCCCCTATTGACGTCAATGAC |
| | | GGTAAATGGCCCGCCTGGCATTATGCCCAGTACATGACCT |
| | | TACGGGACTTTCCTACTTGGCAGTACATCTACGTATTAGTC |
| | | ATCGCTATTACCATGGTCGAGGTGAGCCCCACGTTCTGCT |
| | | TCACTCTCCCCATCTCCCCCCCCTCCCCACCCCCAATTTTG |
| | | TATTTATTTATTTTTTAATTATTTTGTGCAGCGATGGGGGC |
| | | GGGGGGGGGGGGGGGGCGCGCGCCAGGCGGGGCGGGGC |
| | | GGGGCGAGGGGCGGGGCGGGGCGAGGCGGAGAGGTGCG |
| | | GCGGCAGCCAATCAGAGCGGCGCGCTCCGAAAGTTTCCTT |
| | | TTATGGCGAGGCGGCGGCGGCGGCGGCCCTATAAAAAGC |
| | | GAAGCGCGCGGCGGGCGGGAGTCGCTGCGACGCTGCCTT |
| | | CGCCCCGTGCCCCGCTCCGCCGCCGCCTCGCGCCGCCCGC |
| | | CCCGGCTCTGACTGACCGCGTTACTCCCACAGAGATCTCG |
| | | TTTAGTGAACCGTCAGATCCTCACTCTCTTCCGCATCGCTG |
| | | TCTGCGAGGGCCAGCTGTTGGGCTCGCGGTTGAGGACAAA |
| | | CTCTTCGCGGTCTTTCCAGTACTCTTGGATCGGAAACCCGT |
| | | CGGCCTCCGAACGGTACTCCGCCACCGAGGGACCTGAGCG |
| | | AGTCCGCATCGACCGGATCGGAAAACCTCTCGAGAAAGG |
| | | CGTCTAACCAGTCACAGTCGCAAGGTAGGCTGAGCACCGT |
| | | GGCGGGCGGCAGCGGGTGGCGGTCGGGGTTGTTTCTGGCG |
| | | GAGGTGCTGCTGATGATGTAATTAAAGTAGGCGGTCTTGA |
| | | GACGGCGGATGGTCGAGGTGAGGTGTGGCAGGCTTGAGA |
| | | TCCAGCTGTTGGGGTGAGTACTCCCTCTCAAAAGCGGGCA |
| | | TTACTTCTGCGCTAAGATTGTCAGTTTCCAAAAACGAGGA |
| | | GGATTTGATATTCACCTGGCCCGATCTGGCCATACACTTG |
| | | AGTGACAATGACATCCACTTTGCCTTTCTCTCCACAGGTGT |
| | | CCACTCCCAGGTCCAAGTTTAAACGCCGCCACCATGGTGA |
| | | GCTACTGGGACACCGGCGTGCTGCTGTGCGCCCTGCTGAG |
| | | CTGCCTGCTGCTGACCGGCAGCAGCAGCGGCAGCGACACC |
| | | GGCAGGCCCTTCGTGGAGATGTACTCCGAGATCCCCGAGA |
| | | TCATCCACATGACCGAGGGCAGGGAGCTGGTGATCCCCTG |
| | | CAGGGTGACCTCCCCCAACATCACCGTGACCCTGAAGAAG |
| | | TTCCCCCTGGACACCCTGATCCCCGACGGCAAGAGGATCA |
| | | TCTGGGACTCCAGGAAGGGCTTCATCATCTCCAACGCCAC |
| | | CTACAAGGAGATCGGCCTGCTGACCTGCGAGGCCACCGTG |
| | | AACGGCCACCTGTACAAGACCAACTACCTGACCCACAGGC |
| | | AGACCAACACCATCATCGACGTGGTGCTGTCCCCCTCCCA |
| | | CGGCATCGAGCTGTCCGTGGGCGAGAAGCTGGTGCTGAAC |
| | | TGCACCGCCAGGACCGAGCTGAACGTGGGCATCGACTTCA |
| | | ACTGGGAGTACCCCTCCTCCAAGCACCAGCACAAGAAGCT |
| | | GGTGAACAGGGACCTGAAGACCCAGTCCGGCTCCGAGAT |
| | | GAAGAAGTTCCTGTCCACCCTGACCATCGACGGCGTGACC |
| | | AGGTCCGACCAGGGCCTGTACACCTGCGCCGCCTCCTCCG |
| | | GCCTGATGACCAAGAAGAACTCCACCTTCGTGAGGGTGCA |
| | | CGAGAAGGACAAGAGAGTGGAGAGCAAGTACGGCCCCCC |
| | | CTGCCCCCCCTGCCCCGCCCCCGAGTTCCTGGGCGGCCCC |
| | | AGCGTGTTCCTGTTCCCCCCCAAGCCCAAGGACACCCTGA |
| | | TGATCAGCAGAACCCCCGAGGTGACCTGCGTGGTGGTGGA |
| | | CGTGAGCCAGGAGGACCCCGAGGTGCAGTTCAACTGGTA |
| | | CGTGGACGGCGTGGAGGTGCACAACGCCAAGACCAAGCC |
| | | CAGAGAGGAGCAGTTCAACAGCACCTACAGAGTGGTGAG |
| | | CGTGCTGACCGTGCTGCACCAGGACTGGCTGAACGGCAAG |
| | | GAGTACAAGTGCAAGGTGAGCAACAAGGGCCTGCCCAGC |
| | | AGCATCGAGAAGACCATCAGCAAGGCCAAGGGCCAGCCC |
| | | AGAGAGCCCCAGGTGTACACCCTGCCCCCCAGCCAGGAG |
| | | GAGATGACCAAGAACCAGGTGAGCCTGACCTGCCTGGTG |
| | | AAGGGCTTCTACCCCAGCGACATCGCCGTGGAGTGGGAG |
| | | AGCAACGGCCAGCCCGAGAACAACTACAAGACCACCCCC |
| | | CCCGTGCTGGACAGCGACGGCAGCTTCTTCCTGTACAGCA |
| | | GACTGACCGTGGACAAGAGCAGATGGCAGGAGGGCAACG |
| | | TGTTCAGCTGCAGCGTGATGCACGAGGCCCTGCACAACCA |
| | | CTACACCCAGAAGAGCCTGAGCCTGAGCCTGGGCAAGGG |
| | | CGGAGGCGGAAGCGGCGGAGGCGGATCTGGCGGAGGCGG |
| | | AAGCGGCGGAGGCGGCTCTGAGCACCCCAACGCGCGCAA |
| | | ATACAAAGGAGCCAACAAGAAGGGCTTGTCCAAGGGCTG |
| | | CTTCGGCCTCAAGCTGGACCGAATCGGCTCCATGAGCGGC |
| | | CTGGGATGTTAACTCGAGAATCAACCTCTGGATTACAAAA |
| | | TTTGTGAAAGATTGACTGGTATTCTTAACTATGTTGCTCCT |
| | | TTTACGCTATGTGGATACGCTGCTTTAATGCCTTTGTATCA |
| | | TGCTATTGCTTCCCGTATGGCTTTCATTTTCTCCTCCTTGTA |
| | | TAAATCCTGGTTGCTGTCTCTTTATGAGGAGTTGTGGCCCG |
| | | TTGTCAGGCAACGTGGCGTGGTGTGCACTGTGTTTGCTGA |
| | | CGCAACCCCCACTGGTTGGGGCATTGCCACCACCTGTCAG |
| | | CTCCTTTCCGGGACTTTCGCTTTCCCCCTCCCTATTGCCAC |
| | | GGCGGAACTCATCGCCGCCTGCCTTGCCCGCTGCTGGACA |
| | | GGGGCTCGGCTGTTGGGCACTGACAATTCCGTGGTGTTGT |
| | | CGGGGAAGCTGACGTCCTTTCCATGGCTGCTCGCCTGTGT |
| | | TGCCACCTGGATTCTGCGCGGGACGTCCTTCTGCTACGTCC |
| | | CTTCGGCCCTCAATCCAGCGGACCTTCCTTCCCGCGGCCTG |
| | | CTGCCGGCTCTGCGGCCTCTTCCGCGTCTTCGCCTCCGCCC |
| | | TCAGACGAGTCGGATCTCCCTTTGGGCCGCCTCCCCGCCT |
| | | GTTCTCATCACATCATATCAAGGTTATATACCATCAATATT |
| | | GCCACAGATGTTACTTAGCCTTTTAATATTTCTCTAATTTA |
| | | GTGTATATGCAATGATAGTTCTCTGATTTCTGAGATTGAGT |
| | | TTCTCATGTGTAATGATTATTTAGAGTTTCTCTTTCATCTGT |
| | | TCAAATTTTTGTCTAGTTTTATTTTTTACTGATTTGTAAGAC |
| | | TTCTTTTTATAATCTGCATATTACAATTCTCTTTACTGGGG |
| | | TGTTGCAAATATTTTCTGTCATTCTATGGCCTGACTTTTCTT |
| | | AATGGTTTTTTAATTTTAAAAATAAGTCTTAATATTCATGC |
| | | AATCTAATTAACAATCTTTTCTTTGTGGTTAGGACTTTGAG |
| | | TCATAAGAAATTTTTCTCTACACTGAAGTCATGATGGCAT |
| | | GCTTCTATATTATTTTCTAAAAGATTTAAAGTTTTGCCTTC |
| | | TCCATTTAGACTTATAATTCACTGGAATTTTTTTGTGTGTA |
| | | TGGTATGACATATGGGTTCCCTTTTATTTTTTACATATAAA |
| | | TATATTTCCCTGTTTTTCTAAAAAAGAAAAAGATCATCATT |
| | | TTCCCATTGTAAAATGCCATATTTTTTTCATAGGTCACTTA |
| | | CATATATCAATGGGTCTGTTTCTGAGCTCTACTCTATTTTA |
| | | TCAGCCTCACTGTCTATCCCCACACATCTCATGCTTTGCTC |
| | | TAAATCTTGATATTTAGTGGAACATTCTTTCCCATTTTGTT |
| | | CTACAAGAATATTTTTGTTATTGTCTTTGGGCTTTCTATAT |
| | | ACATTTTGAAATGAGGTTGACAAGTTACCTAGGAAAACTG |
| | | TCTTCCTGCCCGGGTGGCATCCCTGTGACCCCTCCCCAGTG |
| | | CCTCTCCTGGCCCTGGAAGTTGCCACTCCAGTGCCCACCA |
| | | GCCTTGTCCTAATAAAATTAAGTTGCATCATTTTGTCTGAC |
| | | TAGGTGTCCTTCTATAATATTATGGGGTGGAGGGGGGGG |
| | | TATGGAGCAAGGGGCCCAAGTTGGGAAGAAACCTGTAGG |
| | | GCCTGCGTACGTAGATAAGTAGCATGGCGGGTTAATCATT |
| | | AACTACAAGGAACCCCTAGTGATGGAGTTGGCCACTCCCT |
| | | CTCTGCGCGCTCGCTCGCTCACTGAGGCCGGGCGACCAAA |
| | | GGTCGCCCGACGCCCGGGCTTTGCCCGGGCGGCCTCAGTG |
| | | AGCGAGCGAGCGCGCAGCTGCTGGTACCGGGCCCCCCCTC |
| | | GAGGTCGACGGTATCGATGGATCCGATATCGAATTCAACC |
| | | TGCAGGCATGCTCTGAGACAATAACCCTGATAAATGCTTC |
| | | AATAATGTAGCCAACCACTAGAACTATAGCTAGAGTCCTG |
| | | GGCGAACAAACGATGCTCGCCTTCCAGAAAACCGAGGAT |
| | | GCGAACCACTTCATCCGGGGTCAGCACCACCGGCAAGCGC |
| | | CGCGACGGCCGAGGTCTTCCGATCTCCTGAAGCCAGGGCA |
| | | GATCCGTGCACAGCACCTTGCCGTAGAAGAACAGCAAGG |
| | | CCGCCAATGCCTGACGATGCGTGGAGACCGAAACCTTGCG |
| | | CTCGTTCGCCAGCCAGGACAGAAATGCCTCGACTTCGCTG |
| | | CTGCCCAAGGTTGCCGGGTGACGCACACCGTGGAAACGG |
| | | ATGAAGGCACGAACCCAGTTGACATAAGCCTGTTCGGTTC |
| | | GTAAACTGTAATGCAAGTAGCGTATGCGCTCACGCAACTG |
| | | GTCCAGAACCTTGACCGAACGCAGCGGTGGTAACGGCGC |
| | | AGTGGCGGTTTTCATGGCTTGTTATGACTGTTTTTTTGTAC |
| | | AGTCTATGCCTCGGGCATCCAAGCAGCAAGCGCGTTACGC |
| | | CGTGGGTCGATGTTTGATGTTATGGAGCAGCAACGATGTT |
| | | ACGCAGCAGCAACGATGTTACGCAGCAGGGCAGTCGCCC |
| | | TAAAACAAAGTTAGGTGGCTCAAGTATGGGCATCATTCGC |
| | | ACATGTAGGCTCGGCCCTGACCAAGTCAAATCCATGCGGG |
| | | CTGCTCTTGATCTTTTCGGTCGTGAGTTCGGAGACGTAGCC |
| | | ACCTACTCCCAACATCAGCCGGACTCCGATTACCTCGGGA |
| | | ACTTGCTCCGTAGTAAGACATTCATCGCGCTTGCTGCCTTC |
| | | GACCAAGAAGCGGTTGTTGGCGCTCTCGCGGCTTACGTTC |
| | | TGCCCAGGTTTGAGCAGCCGCGTAGTGAGATCTATATCTA |
| | | TGATCTCGCAGTCTCCGGCGAGCACCGGAGGCAGGGCATT |
| | | GCCACCGCGCTCATCAATCTCCTCAAGCATGAGGCCAACG |
| | | CGCTTGGTGCTTATGTGATCTACGTGCAAGCAGATTACGG |
| | | TGACGATCCCGCAGTGGCTCTCTATACAAAGTTGGGCATA |
| | | CGGGAAGAAGTGATGCACTTTGATATCGACCCAAGTACCG |
| | | CCACCTAACAATTCGTTCAAGCCGAGATCGGCTTCCCGGC |
| | | CGCGGAGTTGTTCGGTAAATTGTCACAACGCCGCGAATAT |
| | | AGTCTTTACCATGCCCTTGGCCACGCCCCTCTTTAATACGA |
| | | CGGGCAATTTGCACTTCAGAAAATGAAGAGTTTGCTTTAG |
| | | CCATAACAAAAGTCCAGTATGCTTTTTCACAGCATAACTG |
| | | GACTGATTTCAGTTTACAACTATTCTGTCTAGTTTAAGACT |
| | | TTATTGTCATAGTTTAGATCTATTTTGTTCAGTTTAAGACT |
| | | TTATTGTCCGCCCACACCCGCTTACGCAGGGCATCCATTTA |
| | | TTACTCAACCGTAACCGATTTTGCCAGGTTACGCGGCTGG |
| | | TCTGCGGTGTGAAATACCGCACAGATGCGTAAGGAGAAA |
| | | ATACCGCATCAGGCGCTCTTCCGCTTCCTCGCTCACTGACT |
| | | CGCTGCGCTCGGTCGTTCGGCTGCGGCGAGCGGTATCAGC |
| | | TCACTCAAAGGCGGTAATACGGTTATCCACAGAATCAGGG |
| | | GATAACGCAGGAAAGAACATGTGAGCAAAAGGCCAGCAA |
| | | AAGGCCAGGAACCGTAAAAAGGCCGCGTTGCTGGCGTTTT |
| | | TCCATAGGCTCCGCCCCCCTGACGAGCATCACAAAAATCG |
| | | ACGCTCAAGTCAGAGGTGGCGAAACCCGACAGGACTATA |
| | | AAGATACCAGGCGTTTCCCCCTGGAAGCTCCCTCGTGCGC |
| | | TCTCCTGTTCCGACCCTGCCGCTTACCGGATACCTGTCCGC |
| | | CTTTCTCCCTTCGGGAAGCGTGGCGCTTTCTCAATGCTCAC |
| | | GCTGTAGGTATCTCAGTTCGGTGTAGGTCGTTCGCTCCAA |
| | | GCTGGGCTGTGTGCACGAACCCCCCGTTCAGCCCGACCGC |
| | | TGCGCCTTATCCGGTAACTATCGTCTTGAGTCCAACCCGGT |
| | | AAGACACGACTTATCGCCACTGGCAGCAGCCACTGGTAAC |
| | | AGGATTAGCAGAGCGAGGTATGTAGGCGGTGCTACAGAG |
| | | TTCTTGAAGTGGTGGCCTAACTACGGCTACACTAGAAGGA |
| | | CAGTATTTGGTATCTGCGCTCTGCTGAAGCCAGTTACCTTC |
| | | GGAAAAAGAGTTGGTAGCTCTTGATCCGGCAAACAAACC |
| | | ACCGCTGGTAGCGGTGGTTTTTTTGTTTGCAAGCAGCAGA |
| | | TTACGCGCAGAAAAAAAGGATCTCAAGAAGATCCTTTGAT |
| | | CTTTTCTACGTGGTCTGACGCTCAGTGGAACGAAAACTCA |
| | | CGTTAAGGGATTTTGGTCATGAGATTATCAAAAAGGATCT |
| | | TCACCTAGATCCTTTTAAATTAAAAATGAAGTTTTAAATC |
| | | AATCTAAAGTATATATGAGTAAACTTGGTCTGACGGTTAC |
| | | TCAGAAGAACTCGTCAAGAAGGCGATAGAAGGCGATGCG |
| | | CTGCGAATCGGGAGCGGCGATACCGTAAAGCACGAGGAA |
| | | GCGGTCAGCCCATTCGCCGCCAAGCTCTTCAGCAATATCA |
| | | CGGGTAGCCAACGCTATGTCCTGATAGCGGTCCGCCACAC |
| | | CCAGCCGGCCACAGTCGATGAATCCAGAAAAGCGGCCAT |
| | | TTTCCACCATGATATTCGGCAAGCAGGCATCGCCATGGGT |
| | | CACGACGAGATCCTCGCCGTCGGGCATGCGCGCCTTGAGC |
| | | CTGGCGAACAGTTCGGCTGGCGCGAGCCCCTGATGCTCTT |
| | | CGTCCAGATCATCCTGATCGACAAGACCGGCTTCCATCCG |
| | | AGTACGTGCTCGCTCGATGCGATGTTTCGCTTGGTGGTCG |
| | | AATGGGCAGGTAGCCGGATCAAGCGTATGCAGCCGCCGC |
| | | ATTGCATCAGCCATGATGGATACTTTCTCGGCAGGAGCAA |
| | | GGTGAGATGACAGGAGATCCTGCCCCGGCACTTCGCCCAA |
| | | TAGCAGCCAGTCCCTTCCCGCTTCAGTGACAACGTCGAGC |
| | | ACAGCTGCGCAAGGAACGCCCGTCGTGGCCAGCCACGAT |
| | | AGCCGCGCTGCCTCGTCCTGCAGTTCATTCAGGGCACCGG |
| | | ACAGGTCGGTCTTGACAAAAAGAACCGGGCGCCCCTGCG |
| | | CTGACAGCCGGAACACGGCGGCATCAGAGCAGCCGATTG |
| | | TCTGTTGTGCCCAGTCATAGCCGAATAGCCTCTCCACCCA |
| | | AGCGGCCGGAGAACCTGCGTGCAATCCATCTTGTTCAATC |
| | | ATACTCTTCCTTTTTCAATATTATTGAAGCATTTATCAGGG |
| | | TTATTGTCTCATGAGCGGATACATATTTGAATGTATTTAGA |
| | | AAAATAAACAAATAGGGGTTCCGCGCACATTTCCCCGAAA |
| | | AGTGCCACCTGACGTCTAAGAAACCATTATTATCATGACA |
| | | TTAACCTATAAAAATAGGCGTATCACGAGGCCCTTTCGTC |
| | | TCGCGCGTTTCGGTGATGACGGTGAAAACCTCTGACACAT |
| | | GCAGCTCCCGGAGACGGTCACAGCTTGTCTGTAAGCGGAT |
| | | GCCGGGAGCAGACAAGCCCGTCAGGGCGCGTCAGCGGGT |
| | | GTTGGCGGGTGTCGGGGCTGGCTTAACTATGCGGCATCAG |
| | | AGCAGATTGTACTGAGAGTGCACCATATGCGGTGTGAAAT |
| | | ACCGCACAGATGCGTAAGGAGAAAATACCGCATCAGGAA |
| | | ATTGTAAACGTTAATATTTTGTTAAAATTCGCGTTAAATTT |
| | | TTGTTAAATCAGCTCATTTTTTAACCAATAGGCCGAAATC |
| | | GGCAAAATCCCTTATAAATCAAAAGAATAGACCGAGATA |
| | | GGGTTGAGTGTTGTTCCAGTTTGGAACAAGAGTCCACTAT |
| | | TAAAGAACGTGGACTCCAACGTCAAAGGGCGAAAAACCG |
| | | TCTATCAGGGCGATGGCCCACTACGTGAACCATCACCCTA |
| | | ATCAAGTTTTTTGGGGTCGAGGTGCCGTAAAGCACTAAAT |
| | | CGGAACCCTAAAGGGAGCCCCCGATTTAGAGCTTGACGG |
| | | GGAAAGCCGGCGAACGTGGCGAGAAAGGAAGGGAAGAA |
| | | AGCGAAAGGAGCGGGCGCTAGGGCGCTGGCAAGTGTAGC |
| | | GGTCACGCTGCGCGTAACCACCACACCCGCCGCGCTTAAT |
| | | GCGCCGCTACAGGGCGCGTCC |
| AMI315 | 182 | CATTCGCCATTCAGGCTGCAAATAAGCGTTGATATTCAGT |
| Plasmid | | CAATTACAAACATTAATAACGAAGAGATGACAGAAAAAT |
| | | TTTCATTCTGTGACAGAGAAAAAGTAGCCGAAGATGACGG |
| | | TTTGTCACATGGAGTTGGCAGGATGTTTGATTAAAAACAT |
| | | AACAGGAAGAAAAATGCCCCGCTGTGGGCGGACAAAATA |
| | | GTTGGGAACTGGGAGGGGTGGAAATGGAGTTTTTAAGGA |
| | | TTATTTAGGGAAGAGTGACAAAATAGATGGGAACTGGGT |
| | | GTAGCGTCGTAAGCTAATACGAAAATTAAAAATGACAAA |
| | | ATAGTTTGGAACTAGATTTCACTTATCTGGTTCGGATCTCC |
| | | TAGGGGTACCGGGCCCCCCCTCGAGGTCGACGGTATCGAT |
| | | GGATCCGATATCGAATTCAAGCGCGCTCGCTCGCTCACTG |
| | | AGGCCGCCCGGGCAAAGCCCGGGCGTCGGGCGACCTTTG |
| | | GTCGCCCGGCCTCAGTGAGCGAGCGAGCGCGCAGAGAGG |
| | | GAGTGGCCAACTCCATCACTAGGGGTTCCTTGTAGTTAAT |
| | | GATTAACCCGCCATGCTACTTATCTACGTACTCTGGAGAC |
| | | GACTTACGGTAAATGGCCCGCCTGGCTGACCGCCCAACGA |
| | | CCCCCGCCCATTGACGTCAATAATGACGTATGTTCCCATA |
| | | GTAACGCCAATAGGGACTTTCCATTGACGTCAATGGGTGG |
| | | AGTATTTACGGTAAACTGCCCACTTGGCAGTACATCAAGT |
| | | GTATCATATGCCAAGTCCGCCCCCTATTGACGTCAATGAC |
| | | GGTAAATGGCCCGCCTGGCATTATGCCCAGTACATGACCT |
| | | TACGGGACTTTCCTACTTGGCAGTACATCTACGTATTAGTC |
| | | ATCGCTATTACCATGGTCGAGGTGAGCCCCACGTTCTGCT |
| | | TCACTCTCCCCATCTCCCCCCCCTCCCCACCCCCAATTTTG |
| | | TATTTATTTATTTTTTAATTATTTTGTGCAGCGATGGGGGC |
| | | GGGGGGGGGGGGGGGGCGCGCGCCAGGCGGGGCGGGGC |
| | | GGGGCGAGGGGCGGGGCGGGGCGAGGCGGAGAGGTGCG |
| | | GCGGCAGCCAATCAGAGCGGCGCGCTCCGAAAGTTTCCTT |
| | | TTATGGCGAGGCGGCGGCGGCGGCGGCCCTATAAAAAGC |
| | | GAAGCGCGCGGCGGGCGGGAGTCGCTGCGACGCTGCCTT |
| | | CGCCCCGTGCCCCGCTCCGCCGCCGCCTCGCGCCGCCCGC |
| | | CCCGGCTCTGACTGACCGCGTTACTCCCACAGAGATCTCG |
| | | TTTAGTGAACCGTCAGATCCTCACTCTCTTCCGCATCGCTG |
| | | TCTGCGAGGGCCAGCTGTTGGGCTCGCGGTTGAGGACAAA |
| | | CTCTTCGCGGTCTTTCCAGTACTCTTGGATCGGAAACCCGT |
| | | CGGCCTCCGAACGGTACTCCGCCACCGAGGGACCTGAGCG |
| | | AGTCCGCATCGACCGGATCGGAAAACCTCTCGAGAAAGG |
| | | CGTCTAACCAGTCACAGTCGCAAGGTAGGCTGAGCACCGT |
| | | GGCGGGCGGCAGCGGGTGGCGGTCGGGGTTGTTTCTGGCG |
| | | GAGGTGCTGCTGATGATGTAATTAAAGTAGGCGGTCTTGA |
| | | GACGGCGGATGGTCGAGGTGAGGTGTGGCAGGCTTGAGA |
| | | TCCAGCTGTTGGGGTGAGTACTCCCTCTCAAAAGCGGGCA |
| | | TTACTTCTGCGCTAAGATTGTCAGTTTCCAAAAACGAGGA |
| | | GGATTTGATATTCACCTGGCCCGATCTGGCCATACACTTG |
| | | AGTGACAATGACATCCACTTTGCCTTTCTCTCCACAGGTGT |
| | | CCACTCCCAGGTCCAAGTTTAAACGCCGCCACCATGGAGT |
| | | TCGGCCTGAGCTGGCTGTTCCTGGTGGCCATCCTTAAGGG |
| | | CGTGCAGTGCGAGGTGCAGCTGGTGGAGAGCGGCGGCGG |
| | | CCTGGTGCAGCCCGGCGGCAGCCTGAGACTGAGCTGCGCC |
| | | GCCAGCGGCTACGACTTCACCCACTACGGCATGAACTGGG |
| | | TGAGACAGGCCCCCGGCAAGGGCCTGGAGTGGGTGGGCT |
| | | GGATCAACACCTACACCGGCGAGCCCACCTACGCCGCCGA |
| | | CTTCAAGAGAAGATTCACCTTCAGCCTGGACACCAGCAAG |
| | | AGCACCGCCTACCTGCAGATGAACAGCCTGAGAGCCGAG |
| | | GACACCGCCGTGTACTACTGCGCCAAGTACCCCTACTACT |
| | | ACGGCACCAGCCACTGGTACTTCGACGTGTGGGGCCAGGG |
| | | CACCCTGGTGACCGTGGGCGGAGGCGGAAGCGGCGGAGG |
| | | CGGATCTGGCGGAGGCGGCAGCGGCGGCGGCGGCTCTGA |
| | | CATCCAGCTGACCCAGAGCCCCAGCAGCCTGAGCGCCAGC |
| | | GTGGGCGACAGAGTGACCATCACCTGCAGCGCCAGCCAG |
| | | GACATCAGCAACTACCTGAACTGGTACCAGCAGAAGCCC |
| | | GGCAAGGCCCCCAAGGTGCTGATCTACTTCACCAGCAGCC |
| | | TGCACAGCGGCGTGCCCAGCAGATTCAGCGGCAGCGGCA |
| | | GCGGCACCGACTTCACCCTGACCATCAGCAGCCTGCAGCC |
| | | CGAGGACTTCGCCACCTACTACTGCCAGCAGTACAGCACC |
| | | GTGCCCTGGACCTTCGGCCAGGGCACCAAGGTGGAGATCA |
| | | AGAGAACCGTGGCCGCCGGCGGAGGCGGAAGCGGCGGAG |
| | | GCGGATCTGGCGGAGGCGGCAGCGGCGGCGGCGGCTCTG |
| | | AGCACCCCAACGCGCGCAAATACAAAGGAGCCAACAAGA |
| | | AGGGCTTGTCCAAGGGCTGCTTCGGCCTCAAGCTGGACCG |
| | | AATCGGCTCCATGAGCGGCCTGGGATGTTAACTCGAGAAT |
| | | CAACCTCTGGATTACAAAATTTGTGAAAGATTGACTGGTA |
| | | TTCTTAACTATGTTGCTCCTTTTACGCTATGTGGATACGCT |
| | | GCTTTAATGCCTTTGTATCATGCTATTGCTTCCCGTATGGC |
| | | TTTCATTTTCTCCTCCTTGTATAAATCCTGGTTGCTGTCTCT |
| | | TTATGAGGAGTTGTGGCCCGTTGTCAGGCAACGTGGCGTG |
| | | GTGTGCACTGTGTTTGCTGACGCAACCCCCACTGGTTGGG |
| | | GCATTGCCACCACCTGTCAGCTCCTTTCCGGGACTTTCGCT |
| | | TTCCCCCTCCCTATTGCCACGGCGGAACTCATCGCCGCCTG |
| | | CCTTGCCCGCTGCTGGACAGGGGCTCGGCTGTTGGGCACT |
| | | GACAATTCCGTGGTGTTGTCGGGGAAGCTGACGTCCTTTC |
| | | CATGGCTGCTCGCCTGTGTTGCCACCTGGATTCTGCGCGG |
| | | GACGTCCTTCTGCTACGTCCCTTCGGCCCTCAATCCAGCGG |
| | | ACCTTCCTTCCCGCGGCCTGCTGCCGGCTCTGCGGCCTCTT |
| | | CCGCGTCTTCGCCTCCGCCCTCAGACGAGTCGGATCTCCCT |
| | | TTGGGCCGCCTCCCCGCCTGTTCTCATCACATCATATCAAG |
| | | GTTATATACCATCAATATTGCCACAGATGTTACTTAGCCTT |
| | | TTAATATTTCTCTAATTTAGTGTATATGCAATGATAGTTCT |
| | | CTGATTTCTGAGATTGAGTTTCTCATGTGTAATGATTATTT |
| | | AGAGTTTCTCTTTCATCTGTTCAAATTTTTGTCTAGTTTTAT |
| | | TTTTTACTGATTTGTAAGACTTCTTTTTATAATCTGCATATT |
| | | ACAATTCTCTTTACTGGGGTGTTGCAAATATTTTCTGTCAT |
| | | TCTATGGCCTGACTTTTCTTAATGGTTTTTTAATTTTAAAA |
| | | ATAAGTCTTAATATTCATGCAATCTAATTAACAATCTTTTC |
| | | TTTGTGGTTAGGACTTTGAGTCATAAGAAATTTTTCTCTAC |
| | | ACTGAAGTCATGATGGCATGCTTCTATATTATTTTCTAAAA |
| | | GATTTAAAGTTTTGCCTTCTCCATTTAGACTTATAATTCAC |
| | | TGGAATTTTTTTGTGTGTATGGTATGACATATGGGTTCCCT |
| | | TTTATTTTTTACATATAAATATATTTCCCTGTTTTTCTAAAA |
| | | AAGAAAAAGATCATCATTTTCCCATTGTAAAATGCCATAT |
| | | TTTTTTCATAGGTCACTTACATATATCAATGGGTCTGTTTC |
| | | TGAGCTCTACTCTATTTTATCAGCCTCACTGTCTATCCCCA |
| | | CACATCTCATGCTTTGCTCTAAATCTTGATATTTAGTGGAA |
| | | CATTCTTTCCCATTTTGTTCTACAAGAATATTTTTGTTATTG |
| | | TCTTTGGGCTTTCTATATACATTTTGAAATGAGGTTGACAA |
| | | GTTACCTAGGAAAACTGTCTTCCTGCCCGGGTGGCATCCC |
| | | TGTGACCCCTCCCCAGTGCCTCTCCTGGCCCTGGAAGTTGC |
| | | CACTCCAGTGCCCACCAGCCTTGTCCTAATAAAATTAAGT |
| | | TGCATCATTTTGTCTGACTAGGTGTCCTTCTATAATATTAT |
| | | GGGGTGGAGGGGGGTGGTATGGAGCAAGGGGCCCAAGTT |
| | | GGGAAGAAACCTGTAGGGCCTGCGTACGTAGATAAGTAG |
| | | CATGGCGGGTTAATCATTAACTACAAGGAACCCCTAGTGA |
| | | TGGAGTTGGCCACTCCCTCTCTGCGCGCTCGCTCGCTCACT |
| | | GAGGCCGGGCGACCAAAGGTCGCCCGACGCCCGGGCTTT |
| | | GCCCGGGCGGCCTCAGTGAGCGAGCGAGCGCGCAGCTGC |
| | | TGGTACCGGGCCCCCCCTCGAGGTCGACGGTATCGATGGA |
| | | TCCGATATCGAATTCAACCTGCAGGCATGCTCTGAGACAA |
| | | TAACCCTGATAAATGCTTCAATAATGTAGCCAACCACTAG |
| | | AACTATAGCTAGAGTCCTGGGCGAACAAACGATGCTCGCC |
| | | TTCCAGAAAACCGAGGATGCGAACCACTTCATCCGGGGTC |
| | | AGCACCACCGGCAAGCGCCGCGACGGCCGAGGTCTTCCG |
| | | ATCTCCTGAAGCCAGGGCAGATCCGTGCACAGCACCTTGC |
| | | CGTAGAAGAACAGCAAGGCCGCCAATGCCTGACGATGCG |
| | | TGGAGACCGAAACCTTGCGCTCGTTCGCCAGCCAGGACAG |
| | | AAATGCCTCGACTTCGCTGCTGCCCAAGGTTGCCGGGTGA |
| | | CGCACACCGTGGAAACGGATGAAGGCACGAACCCAGTTG |
| | | ACATAAGCCTGTTCGGTTCGTAAACTGTAATGCAAGTAGC |
| | | GTATGCGCTCACGCAACTGGTCCAGAACCTTGACCGAACG |
| | | CAGCGGTGGTAACGGCGCAGTGGCGGTTTTCATGGCTTGT |
| | | TATGACTGTTTTTTTGTACAGTCTATGCCTCGGGCATCCAA |
| | | GCAGCAAGCGCGTTACGCCGTGGGTCGATGTTTGATGTTA |
| | | TGGAGCAGCAACGATGTTACGCAGCAGCAACGATGTTAC |
| | | GCAGCAGGGCAGTCGCCCTAAAACAAAGTTAGGTGGCTC |
| | | AAGTATGGGCATCATTCGCACATGTAGGCTCGGCCCTGAC |
| | | CAAGTCAAATCCATGCGGGCTGCTCTTGATCTTTTCGGTCG |
| | | TGAGTTCGGAGACGTAGCCACCTACTCCCAACATCAGCCG |
| | | GACTCCGATTACCTCGGGAACTTGCTCCGTAGTAAGACAT |
| | | TCATCGCGCTTGCTGCCTTCGACCAAGAAGCGGTTGTTGG |
| | | CGCTCTCGCGGCTTACGTTCTGCCCAGGTTTGAGCAGCCG |
| | | CGTAGTGAGATCTATATCTATGATCTCGCAGTCTCCGGCG |
| | | AGCACCGGAGGCAGGGCATTGCCACCGCGCTCATCAATCT |
| | | CCTCAAGCATGAGGCCAACGCGCTTGGTGCTTATGTGATC |
| | | TACGTGCAAGCAGATTACGGTGACGATCCCGCAGTGGCTC |
| | | TCTATACAAAGTTGGGCATACGGGAAGAAGTGATGCACTT |
| | | TGATATCGACCCAAGTACCGCCACCTAACAATTCGTTCAA |
| | | GCCGAGATCGGCTTCCCGGCCGCGGAGTTGTTCGGTAAAT |
| | | TGTCACAACGCCGCGAATATAGTCTTTACCATGCCCTTGG |
| | | CCACGCCCCTCTTTAATACGACGGGCAATTTGCACTTCAG |
| | | AAAATGAAGAGTTTGCTTTAGCCATAACAAAAGTCCAGTA |
| | | TGCTTTTTCACAGCATAACTGGACTGATTTCAGTTTACAAC |
| | | TATTCTGTCTAGTTTAAGACTTTATTGTCATAGTTTAGATC |
| | | TATTTTGTTCAGTTTAAGACTTTATTGTCCGCCCACACCCG |
| | | CTTACGCAGGGCATCCATTTATTACTCAACCGTAACCGAT |
| | | TTTGCCAGGTTACGCGGCTGGTCTGCGGTGTGAAATACCG |
| | | CACAGATGCGTAAGGAGAAAATACCGCATCAGGCGCTCTT |
| | | CCGCTTCCTCGCTCACTGACTCGCTGCGCTCGGTCGTTCGG |
| | | CTGCGGCGAGCGGTATCAGCTCACTCAAAGGCGGTAATAC |
| | | GGTTATCCACAGAATCAGGGGATAACGCAGGAAAGAACA |
| | | TGTGAGCAAAAGGCCAGCAAAAGGCCAGGAACCGTAAAA |
| | | AGGCCGCGTTGCTGGCGTTTTTCCATAGGCTCCGCCCCCCT |
| | | GACGAGCATCACAAAAATCGACGCTCAAGTCAGAGGTGG |
| | | CGAAACCCGACAGGACTATAAAGATACCAGGCGTTTCCCC |
| | | CTGGAAGCTCCCTCGTGCGCTCTCCTGTTCCGACCCTGCCG |
| | | CTTACCGGATACCTGTCCGCCTTTCTCCCTTCGGGAAGCGT |
| | | GGCGCTTTCTCAATGCTCACGCTGTAGGTATCTCAGTTCGG |
| | | TGTAGGTCGTTCGCTCCAAGCTGGGCTGTGTGCACGAACC |
| | | CCCCGTTCAGCCCGACCGCTGCGCCTTATCCGGTAACTAT |
| | | CGTCTTGAGTCCAACCCGGTAAGACACGACTTATCGCCAC |
| | | TGGCAGCAGCCACTGGTAACAGGATTAGCAGAGCGAGGT |
| | | ATGTAGGCGGTGCTACAGAGTTCTTGAAGTGGTGGCCTAA |
| | | CTACGGCTACACTAGAAGGACAGTATTTGGTATCTGCGCT |
| | | CTGCTGAAGCCAGTTACCTTCGGAAAAAGAGTTGGTAGCT |
| | | CTTGATCCGGCAAACAAACCACCGCTGGTAGCGGTGGTTT |
| | | TTTTGTTTGCAAGCAGCAGATTACGCGCAGAAAAAAAGGA |
| | | TCTCAAGAAGATCCTTTGATCTTTTCTACGTGGTCTGACGC |
| | | TCAGTGGAACGAAAACTCACGTTAAGGGATTTTGGTCATG |
| | | AGATTATCAAAAAGGATCTTCACCTAGATCCTTTTAAATT |
| | | AAAAATGAAGTTTTAAATCAATCTAAAGTATATATGAGTA |
| | | AACTTGGTCTGACGGTTACTCAGAAGAACTCGTCAAGAAG |
| | | GCGATAGAAGGCGATGCGCTGCGAATCGGGAGCGGCGAT |
| | | ACCGTAAAGCACGAGGAAGCGGTCAGCCCATTCGCCGCC |
| | | AAGCTCTTCAGCAATATCACGGGTAGCCAACGCTATGTCC |
| | | TGATAGCGGTCCGCCACACCCAGCCGGCCACAGTCGATGA |
| | | ATCCAGAAAAGCGGCCATTTTCCACCATGATATTCGGCAA |
| | | GCAGGCATCGCCATGGGTCACGACGAGATCCTCGCCGTCG |
| | | GGCATGCGCGCCTTGAGCCTGGCGAACAGTTCGGCTGGCG |
| | | CGAGCCCCTGATGCTCTTCGTCCAGATCATCCTGATCGAC |
| | | AAGACCGGCTTCCATCCGAGTACGTGCTCGCTCGATGCGA |
| | | TGTTTCGCTTGGTGGTCGAATGGGCAGGTAGCCGGATCAA |
| | | GCGTATGCAGCCGCCGCATTGCATCAGCCATGATGGATAC |
| | | TTTCTCGGCAGGAGCAAGGTGAGATGACAGGAGATCCTGC |
| | | CCCGGCACTTCGCCCAATAGCAGCCAGTCCCTTCCCGCTT |
| | | CAGTGACAACGTCGAGCACAGCTGCGCAAGGAACGCCCG |
| | | TCGTGGCCAGCCACGATAGCCGCGCTGCCTCGTCCTGCAG |
| | | TTCATTCAGGGCACCGGACAGGTCGGTCTTGACAAAAAGA |
| | | ACCGGGCGCCCCTGCGCTGACAGCCGGAACACGGCGGCA |
| | | TCAGAGCAGCCGATTGTCTGTTGTGCCCAGTCATAGCCGA |
| | | ATAGCCTCTCCACCCAAGCGGCCGGAGAACCTGCGTGCAA |
| | | TCCATCTTGTTCAATCATACTCTTCCTTTTTCAATATTATTG |
| | | AAGCATTTATCAGGGTTATTGTCTCATGAGCGGATACATA |
| | | TTTGAATGTATTTAGAAAAATAAACAAATAGGGGTTCCGC |
| | | GCACATTTCCCCGAAAAGTGCCACCTGACGTCTAAGAAAC |
| | | CATTATTATCATGACATTAACCTATAAAAATAGGCGTATC |
| | | ACGAGGCCCTTTCGTCTCGCGCGTTTCGGTGATGACGGTG |
| | | AAAACCTCTGACACATGCAGCTCCCGGAGACGGTCACAGC |
| | | TTGTCTGTAAGCGGATGCCGGGAGCAGACAAGCCCGTCAG |
| | | GGCGCGTCAGCGGGTGTTGGCGGGTGTCGGGGCTGGCTTA |
| | | ACTATGCGGCATCAGAGCAGATTGTACTGAGAGTGCACCA |
| | | TATGCGGTGTGAAATACCGCACAGATGCGTAAGGAGAAA |
| | | ATACCGCATCAGGAAATTGTAAACGTTAATATTTTGTTAA |
| | | AATTCGCGTTAAATTTTTGTTAAATCAGCTCATTTTTTAAC |
| | | CAATAGGCCGAAATCGGCAAAATCCCTTATAAATCAAAA |
| | | GAATAGACCGAGATAGGGTTGAGTGTTGTTCCAGTTTGGA |
| | | ACAAGAGTCCACTATTAAAGAACGTGGACTCCAACGTCAA |
| | | AGGGCGAAAAACCGTCTATCAGGGCGATGGCCCACTACG |
| | | TGAACCATCACCCTAATCAAGTTTTTTGGGGTCGAGGTGC |
| | | CGTAAAGCACTAAATCGGAACCCTAAAGGGAGCCCCCGA |
| | | TTTAGAGCTTGACGGGGAAAGCCGGCGAACGTGGCGAGA |
| | | AAGGAAGGGAAGAAAGCGAAAGGAGCGGGCGCTAGGGC |
| | | GCTGGCAAGTGTAGCGGTCACGCTGCGCGTAACCACCACA |
| | | CCCGCCGCGCTTAATGCGCCGCTACAGGGCGCGTCC |

TABLE 12
Yields of AAV vectors determined with ITR-qPCR

| | | | AAV | Total | Total | |
|---|---|---|---|---|---|---|
| | Vector | | titer | AAV | AAV | Yield |
| Construct | type | Lot No. | (vg/mL) | Vol (mL) | (vg) | (vg/L) |
| AMI061 | CMV- | 21-066 | 1.43E+13 | 2.0 | 2.86E+13 | 9.50E+13 |
| | scAAV | | | | | |
| AMI087 | CMV- | 21-071 | 3.75E+12 | 2.0 | 7.50E+12 | 2.50E+13 |
| | scAAV | | | | | |
| AMI088 | CMV- | 21-085 | 2.53E+13 | 4.6 | 1.16E+14 | 1.45E+14 |
| | scAAV | (AAVx) | | | | |
| AMI182 | CMV- | 21-084 | 2.21E+13 | 4.0 | 8.84E+13 | 1.10E+14 |
| | scAAV | (AAVx) | | | | |
| AMI189 | CMV- | 21-089 | 3.82E+13 | 4.5 | 1.72E+14 | 2.15E+14 |
| | scAAV | (AAVx) | | | | |
| AMI263 | CMV- | 22-005 | 2.69E+13 | 2.6 | 6.99E+13 | 8.70E+13 |
| | scAAV | (AAVx) | | | | |
| AMI269 | CMV- | 22-037 | 1.59E+13 | 2.1 | 3.34E+13 | 1.67E+14 |
| | scAAV | | | | | |
| AMI270 | CMV- | 22-031 | 2.94E+13 | 2.0 | 5.88E+13 | 2.94E+14 |
| | scAAV | | | | | |
| AMI273 | CBA- | 22-034 | 2.50E+13 | 5.3 | 1.33E+14 | 6.65E+13 |
| | scAAV | (AAVx) | | | | |
| AMI302 | CBA- | 22-029 | 2.62E+13 | 3.3 | 8.65E+13 | 4.33E+13 |
| | scAAV | (AAVx) | | | | |
| AMI303 | CBA- | NA | NA | NA | NA | NA |
| | ssAAV | | | | | |
| AMI304 | CBA- | NA | NA | NA | NA | NA |
| | ssAAV | | | | | |
| AMI305 | CBA- | NA | NA | NA | NA | NA |
| | ssAAV | | | | | |
| AMI312 | CMV- | 22-043 | 2.85E+13 | 2.2 | 6.27E+13 | 3.14E+14 |
| | scAAV | | | | | |
| AMI315 | CBA- | 22-044 | 3.33E+13 | 3.2 | 1.07E+14 | 5.35E+14 |
| | ssAAV | | | | | |

### Example 2. CNP-Fc ELISA Development and Standardization

C-type natriuretic peptide (CNP) has two isoforms, CNP-22 and CNP-53. Example 2 illustrates that CNP-36 fused with Fe increased the half-life of the CNP and also facilitated in purification and ELISA detection when compared to native (unfused) CNP. Transduction and CNP-Fc expression experiments were performed with three AAV vectors (AMI061, AMI087 and AMI0488). FIG. 1A is the pictorial depiction of the three vectors. FIGS. 1B-F illustrate additional vectors that can encode the CNP or the CNP fusion protein. Enzyme-linked immunoassay (ELISA) was developed for quantifying CNP-Fc fusion proteins in cell culture supernatants. The ELISA assay was used to quantify CNP-Fc expressed in HEK293 cells, human chondrocytes (HCH) cells, and human skeletal muscle cells (hSkMC) upon transduction with one of the three AAV constructs described herein. Table 13 illustrates material and instrument for performing the ELISA described herein.

TABLE 13
Material and instrument for performing ELISA for quantifying
CNP and CNP-Fc protein

| Product name | Vendor | Cat No. |
|---|---|---|
| 10X PBS | Fisher Scientific, Hyclone | SH3025802 |
| Human CNP Antibody | R&D systems | MAB31271 |
| (monoclonal) | | |
| Goat pAb Hu IgG (Biotin) | Abcam | ab98618 |
| Tween20 | Sigma Aldrich | P2287-500 mL |
| Nunc maxisorb Flat-bottom | Invitrogen | 44-2404-21 |
| 96-well plate | | |
| Blocker ™ Casein in PBS | Thermofisher Scientific | 37528 |
| Streptavidin-HRP | Abcam | ab7403 |
| TMB substrate | Abcam | ab171523 |
| (High Sensitivity) | | |
| Stop Reagent for TMB | Sigma Aldrich | 07102-1L-GL |
| Substrate (2N HCl) | | |

Buffer and substrate. Coating buffer: 3.7 g sodium bicarbonate, 0.64 g sodium carbonate in 1 L of Milli Q water, pH 9.6. Stored at RT; Blocking buffer: Commercial Casein blocking buffer in PBS+ added 0.1% Tween20. Stored at 4° C.; Standard diluent: same as blocking buffer; Wash buffer: 1×PBS with 0.1% tween 20 (30 days expiry from date of manufacture); Coating Antibody (Ab): Human CNP Antibody (monoclonal), coating concentration was 2 μg/mL; Detection Ab: Goat pAb anti-human Fc. Diluted up to 1:20,000 final dilution in well. Stored at 4° C.; HRP: Streptavidin-HRP stock of 1 mg/mL, diluted up to 1:10,000. Stored at 4° C.; Substrate: TMB. Stored at 4° C.; and Stop solution: 2 N HCl. Stored at ambient temperature.

### Procedure.

1. Coat 96-well plate with 2 μg/mL anti-CNP antibody final concentration per well in the coating buffer with a 50 μL volume and cover the plate with sealing cover. Put the plate at 4° C. overnight.
2. Next day, remove the plate, wash thrice with wash buffer and tap the plate on the paper towel to remove excess solution.
3. Add 300 μL of blocking buffer to each well using multichannel pipette. Put the plate with sealing cover into 37° C. incubator for 2 hours.
4. After incubation, discard the blocking buffer and tap on paper towel to remove excess buffer.
5. Prepare standards and sample dilutions in blocking buffer and add 50 μL into each well according to the scheme of the experiment.
Note: Reaction volume for every step is equal to the initial coating buffer volume. For example, if coating buffer was 50 μL then sample, capture, detection, TMB and stop solution all have to be 50 μL except for blocking and wash buffer (300 μL).
6. Cover the plate with sealing cover and place it back into the incubator for 1 hr.
7. After 1 hour, discard the solution and wash plate with 300 μL of wash buffer 6 times. Remove excess solution by tapping on paper towel as described above.
8. Add detection antibody at 1:20,000 dilution in blocking buffer and incubate at 37° C. for 1 hr.
9. Discard solution and repeat washing procedure as described in step 7.
10. Add Streptavidin-HRP at 1:10,000 dilution in blocking buffer at 37° C. for 45 mins.
11. Discard the solution and wash plate as described in step 7.
12. Add 50 μL TMB and avoid placing the plate in direct light source for 15-20 min (it could be <15 mins) or until you start seeing saturation at highest concentration wells.
13. Add 50 μL stop solution and read plate at 450 nm with 600 as reference within 15 min.

### ELISA Development.

1. Coating/Capture antibody titration: Different concentration of anti-CNP antibody was coated onto the wells (5, 2, 1 and 0.5 μg/mL) was plated on the 96-well plate. AMI061, AMI087 and AMI088 (CNP constructs) were also titrated from 0-200 ng/mL by serial dilution. Detection Ab was diluted till 1:4000 and Streptavidin HRP till 1:10,000.
2. Detection antibody titration: Results from the above experiment showed 2 μg/mL coating Ab concentration to be optimum for assay. However, all the constructs showed plateauing at 100 and 200 ng/mL concentration. Hence, along with detection antibody, standard concentration was reduced from 200 ng/mL to 50 ng/mL. The standards were now serially diluted from 50 ng/mL till 0.8 ng/mL.

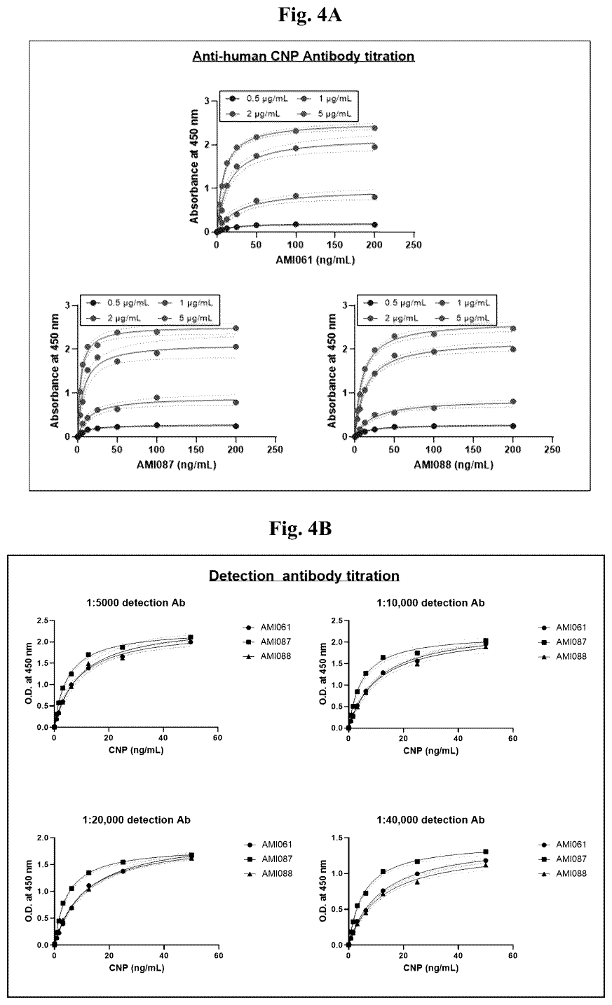

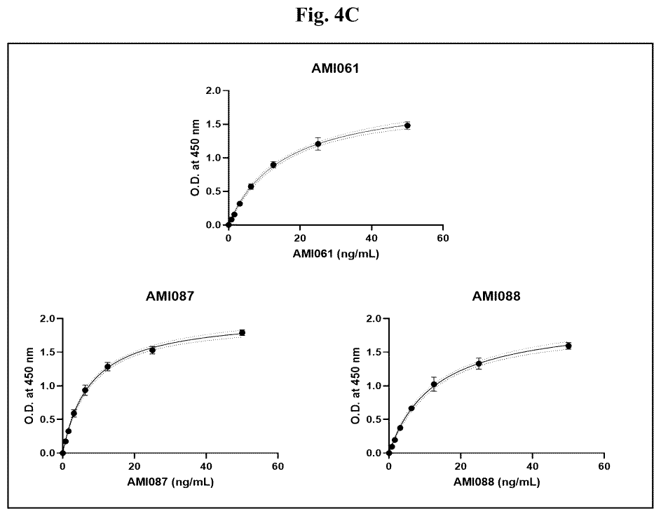

Results. Coating Ab titration: Following the protocol described in the ELISA Development section, wells were coated with different concentrations of anti-CNP antibody and different CNP constructs were added to the specified wells. The scheme of addition has been depicted in Table 14. The wells with numbers showed the concentration of CNP constructs added to the well in ng/mL. FIG. 4A illustrates the titration of coating Ab for each construct. The standard curve obtained was fitted using the hyperbolic fitting on GraphPad Prism software. At coating concentration of 0.5 and 1 μg/mL concentration the absorbance was less than 1 O.D. 5 μg/mL coating concentration showed saturation and 2 μg/mL showed O.D. greater than 1. Hence, 2 μg/mL might be an appropriate concentration to use for standard curve. Detection Ab was titrated to achieve better fitting of standard curves. Titrating detection antibody: At 1:4000 dilution of detection Ab, curve fitting wasn't optimal. This could lead to inaccurate sample estimation in future experiments. Therefore, detection Ab was diluted to 1:5000, 1:10,000, 1:20,000 and 1:40,000. The coating Ab concentration was 2 μg/mL, titration of CNP constructs was reduced from 200 ng/mL to 50 ng/mL and was serially diluted to give concentrations of 50, 25, 12.5, 6.25, 3.125, 1.56, 0.76 ng/mL along with blank, which was assay diluent. Streptavidin-TRP was diluted 1;10,000 as used in previous experiment. FIG. 4B shows the standard curves for all three CNP constructs with hyperbolic fitting. 1:20,000 detection Ab titration gave a good fitting compared to other dilutions. Anti-CNP-Fc ELISA to quantify the expression of CNP in HEK293 cell supernatant: 1E+06 HEK293 cells were plated on the 6-well plate. Cells were transduced with 100,000 MOI of AVV2 (N54-AMI061, N54-AMI087 and N54-AMI088) constructs carrying CNP fused with Fc. Constructs were designed and optimized for GC content which facilitates better expression. qPCR was used to determine the viral vector titer after purification. The titer and other parameters are shown in Table 15. 5 days after transduction, cell supernatant was collected and CNP-Fc ELISA was performed at different sample dilutions (no dilution, 1:5, 1:10, 1:50, 1:100, 1;200, 1:500, 1:1000, 1:2000 and 1:4000 dilutions). Experiment was performed in duplicates. The standard curves obtained were plotted and fitted using the hyperbolic equation using GraphPad Prism software as mentioned above (FIG. 4C). Intra-assay precision for CNP-Fc ELISA had been calculated and is shown in Table 16. Percentage coefficient of variance (CV) value for all the standards is less than 10%. Indicating good precision of the assay. Samples with no dilution showed saturating signal. For AMI061, dilutions 1:500, 1:1000 and 1:2000 showed readings that were in detectable range. For AMI087 and AMI088, the O.D. values were in detectable range in 1:4000 dilution. Also, AMI088 showed highest expression when compared to other CNP constructs. These results have been summarized in Table 17. The cells that have not been transduced using the CNP constructs showed very little expression with 6.2, 2.5 and 6.4 ng/mL for AMI061, AMI087 and AMI088 respectively.

TABLE 14
Schematic representation of the addition of coating Ab and CNP constructs in 96-well plate

| 0.5 μg/mL | | | 1 μg/mL | | | 2 μg/mL | | | 5 μg/mL | | |
|---|---|---|---|---|---|---|---|---|---|---|---|
| coating Ab | | | coating Ab | | | coating Ab | | | coating Ab | | |
| AMI | AMI | AMI | AMI | AMI | AMI | AMI | AMI | AMI | AMI | AMI | AMI |
| 061 | 087 | 088 | 061 | 087 | 088 | 061 | 087 | 088 | 061 | 087 | 088 |
| 200 | 200 | 200 | 200 | 200 | 200 | 200 | 200 | 200 | 200 | 200 | 200 |
| 100 | 100 | 100 | 100 | 100 | 100 | 100 | 100 | 100 | 100 | 100 | 100 |
| 50 | 50 | 50 | 50 | 50 | 50 | 50 | 50 | 50 | 50 | 50 | 50 |
| 25 | 25 | 25 | 25 | 25 | 25 | 25 | 25 | 25 | 25 | 25 | 25 |
| 12.5 | 12.5 | 12.5 | 12.5 | 12.5 | 12.5 | 12.5 | 12.5 | 12.5 | 12.5 | 12.5 | 12.5 |
| 6.25 | 6.25 | 6.25 | 6.25 | 6.25 | 6.25 | 6.25 | 6.25 | 6.25 | 6.25 | 6.25 | 6.25 |
| 3.25 | 3.25 | 3.25 | 3.25 | 3.25 | 3.25 | 3.25 | 3.25 | 3.25 | 3.25 | 3.25 | 3.25 |
| 0 | 0 | 0 | 0 | 0 | 0 | 0 | 0 | 0 | 0 | 0 | 0 |

TABLE 15
CNP-construct titers calculated using qPCR

| Lot# | Construct | Titer (vg/mL) | Production Date | Buffer |
|---|---|---|---|---|
| 20-004 | N54-AMI061 | 1.40E+13 | Jul. 28, 2021 | AVMX |
| | | | | AAV2 |
| | | | | Formulation |
| 20-070 | N54-AMI087 | 3.75E+12 | Jul. 28, 2021 | AVMX |
| | | | | AAV2 |
| | | | | Formulation |
| 21-005 | N54-AMI088 | 3.28E+12 | Jul. 28, 2021 | AVMX |
| | | | | AAV2 |
| | | | | Formulation |

TABLE 16
Intra-assay precision of CNP-Fc ELISA for AMI061,
AMI087 and AMI088

| CNP | | | |
|---|---|---|---|
| constructs | % CV for | % CV for | |
| (ng/ml) | AMI061 | AMI087 | % CV for AMI088 |
| 50 | 2.70 | 1.51 | 2.12 |
| 25.00 | 5.15 | 2.41 | 4.69 |
| 12.50 | 3.71 | 3.52 | 7.34 |
| 6.25 | 5.87 | 5.89 | 2.84 |
| 3.13 | 7.27 | 6.58 | 3.85 |
| 1.56 | 8.88 | 4.02 | 6.36 |
| 0.78 | 7.07 | 2.59 | 1.98 |

TABLE 17
Expression of AMI061, AMI087 and AMI088 in HEK293 cells

| CNP-Fc construct | Concentration (μg/mL) | Std. Dev. |
|---|---|---|
| AMI061 | 6.5 | 0.3 |
| AMI087 | 33 | 4.5 |
| AMI088 | 75 | 9.2 |

The standard curve of CNP-Fc ELISA was hyperbolic and need to be fit using the hyperbolic fitting. The ELISA method described herein could be used to quantify CNP-Fc in cell culture supernatants, pre-clinical and clinical samples. The concise summary of the conditions at which this ELISA was been standardized includes: 2 μg/mL Anti-CNP antibody coating concentration was used; CNP-Fc purified proteins were serially diluted from 50 ng/mL till 0.78 ng/mL; and detection Ab was diluted until 1:20,000 and Streptavidin-HRP until 1:10,000.

### Example 3. Evaluation of Efficacy in NMDA Excitotoxicity Mouse Model of Retinal Degeneration

This study was for evaluating the effect of adeno-associated viral (AAV) vectors on retinal ganglion cell protection following damage caused via N-methyl-D-aspartate (NMDA) administration in the mouse. Injection volumes and treatment options can be found in Table 18. Groups 7 and 8 were added to test a higher concentration of MK-801. The tissues for bioanalysis were snap frozen whole instead of being homogenized. The methods of adding IBMX to whole blood were established and clarified. This study was conducted in wild type mice (Mus musculus, C57BL/6).

TABLE 18
Experimental design for the NMDA mouse study

| | | OU Treatment | | OD | |
|---|---|---|---|---|---|
| | | ROA, | | Induction | |
| | No. of | Volume and | | (Day 0) | |
| Group | Animals | Study Day | Treatment | Agent and ROA | Experimental Endpoints |
| 1 | 12 | IVT | PBS | NMDA | Day 7 |
| | | 0.5 μL | | (2.5 mM; | n = 2 OD/group for |
| 2 | 12 | Day 0 | MK-801 | 0.5 μL) | histopathology/cell |
| | | | 1.5 nM | IVT | counts; n = 2 OS |
| | | | | | for controls |
| | | | | | n = 8 OD/group for |
| | | | | | retinal flat mounts |
| | | | | | (DAPI + TUJ-1 |
| | | | | | Alexa488); n = 8 OS |
| | | | | | from Group I were |
| | | | | | included as controls |
| | | | | | n = 2 OD/group for |
| | | | | | IHC; n = 2 OS for |
| | | | | | controls |
| 3 | 8 | IVT | Sham AMI | NMDA | Day 7 |
| | | 1 μL | 189 | (2.5 mM; | Blood collection |
| 4 | 8 | 4e+8 | AMI 169 | 1 μL) | (serum): n = 8/group |
| | | vg/μL | | IVT | n = 8 OD/group for |
| 5 | 8 | Day −28 | AMI 182 | | retinal flat mounts |
| 6 | 8 | | AMI 088 | | (DAPI + CNP) |
| | | | | | n = 8 OS/group for |
| | | | | | assays |
| 7 | 8 | IVT | PBS | NMDA | Day 7 |
| 8 | 8 | 0.5 μL | MK-801 | (2.5 mM; | n = 8 OD/group for |
| | | Day 0 | 100 μM | 0.5 μL) | retinal flat mounts |
| | | | | IVT | (DAPI); n = 8 OS |
| | | | | | from Group I were |
| | | | | | included as controls |

Abbreviations: IVT—Intravitreal; OU—both eyes; OD—Right eye; OS—Left eye; PBS—Phosphate buffered saline
Identification of AAV2.N54 vector and gene of interest:
AMI189: the same vector contains all DNA sequences but open reading frame (ORF) interrupted
AMI263 encoding IgG4 CNP-Fc36. No protein was expected to produce in vitro and in vivo;
AMI169: AAV2.N54 vector encoding anti-VEGF scFv CNP36 fusion protein
AMI088: AAV2.N54 vector encoding Fc-VNP36.
AAV constructs were resuspended in formulation buffer 1, PBS, 0.01%

### NMDA Formulation

The molecular weight of NMDA is 147.3 g/mol. On the day of dosing, 20 mg of NMDA was weighed and added to 2.7 mL of PBS for a 50 mM solution [Stock A]. Stock A solution was diluted 1:9 in PBS (100 μL of Stock A+900 μL of PBS) for a final 5 mM NMDA solution. The final solution was syringe filtered with a 0.22 μm filter into a sterile vial. Solution was made the day of injections, protected from light, and stored refrigerated until dosing.

### MK-801 Formulation

The molecular weight of MK-801 is 337.37 g/mol. The MK-801 container received had 5 mg of MK-801. On the day of dosing, 1.0 mL of PBS was added to the MK-801 for a concentration of 14.82 mM solution [Stock A]. The stock was serially diluted as follows: 10 μL of Stock A+731 μL PBS: 0.2 mM (200 μM) MK-801 [Stock B]. The final solution was syringe filtered with a 0.22 μm filter into a sterile vial. Solution was made the day of injections, protected from light, and stored refrigerated until dosing.

### Group 1 Dosing

300 μL of the 5 mM NMDA stock was combined with 300 μL of sterile PBS and 1 μL as injected on Day 0 for a final concentration of 2.5 mM NMDA.

### Group 2 Dosing

300 μL of the 5 mM NMDA stock as combined with 300 μL of sterile-filtered MK-801 and 1 μL was injected on Day 0 for a final concentration of 2.5 mM NMDA/100 μM MK-801.

### Groups 3-6 Dosing

300 μL of the 5 mM NMDA stock was combined with 300 μL of sterile PBS and 1 μL was injected on Day 0 for a final concentration of 2.5 mM NMDA.

### Intravitreal Injection

On Day −28 or Day 0 based on the experimental design, mice were given buprenorphine 0.01-0.05 mg/kg subcutaneously (SQ). Animals were then tranquilized for the intravitreal injections with ketamine/xylazine or inhaled isoflurane and one drop of 0.5% proparacaine HCl was applied to both eyes. The conjunctiva was gently grasped with Dumont #4 forceps, and the injection was made using a 33 G needle and a Hamilton syringe. After dispensing the syringe contents, the syringe needle was slowly withdrawn. Following the injection procedure, 1 drop of Ofloxacin ophthalmic solution was applied topically to the ocular surface with eye lube.

During the Day 0 NMDA induction, animal 409 (Group 4) was noted to have inflammation and synechiae in the OD, animal 519 OD (Group 5) was noted to have a cataract, and animal 524 (Group 5) was noted to have cataracts OU. In Group 6, animals 625 and 626 were noted to have a cataract OS, and animals 629, 630, and 632 were noted to have a cataract OD. No other abnormalities were noted in the study records.

### Cage Side Observations

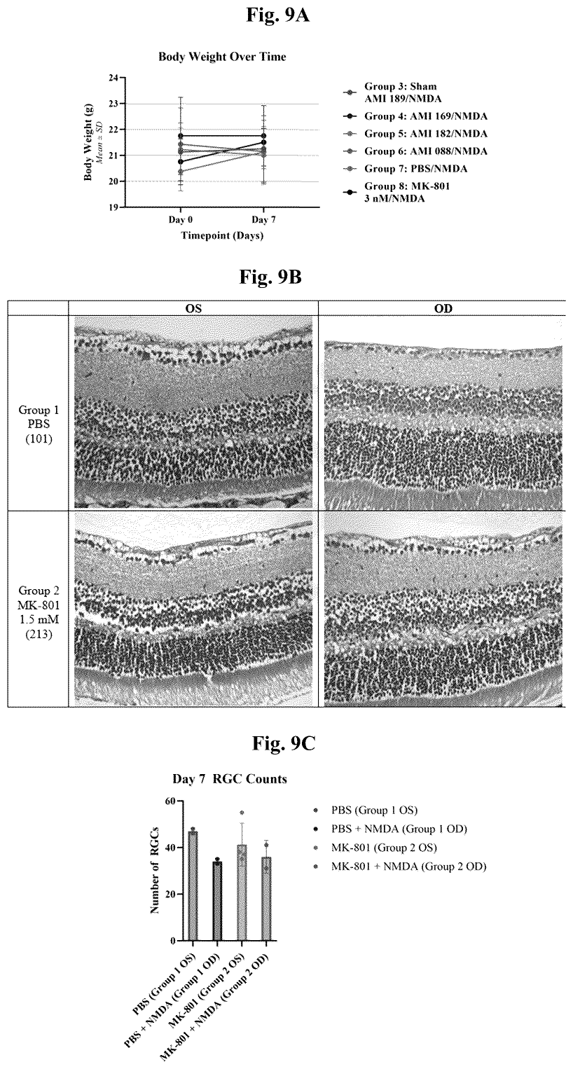

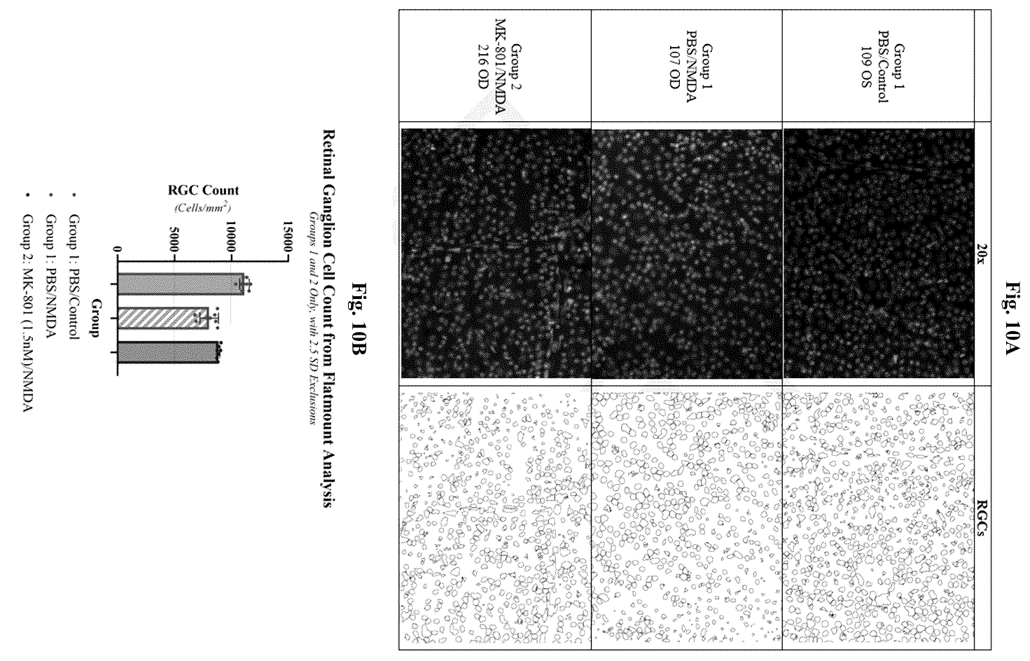

Morbidity and mortality were observed daily along with cage-side observations, with particular attention paid to both eyes. All animals were bright, alert, and responsive at all observation timepoints, with no additional findings noted in the study records. On the day of IVT injection, the body weight of all animals was 20-22 g, and all animals generally maintained body weight over the course of the study (FIG. 9A).

### Euthanasia and Terminal Blood Collection (Groups 3-6)

Animals in Groups 3-6 had terminal blood collected on Day 7. IBMX was used during blood collection to inhibit phosphodiesterase activity. Prior to necropsy on Day 7 but after dosing on Day −28, all remaining spare animals were utilized to ensure that addition of IBMX to whole blood did not interfere with clotting and extraction of serum. Briefly, each syringe for cardiac puncture was pre-rinsed with a stock solution of 10 mM IBMX prior to drawing blood. All mice were sedated to a deep plane of sedation under isoflurane and euthanized via exsanguination. After each syringe was pre-rinsed with 10 mM IBMX, a 25 G needle was inserted into the heart and the animal was exsanguinated and euthanized, and blood was collected. The volume of blood collected was noted on the paperwork and the appropriate volume of IBMX was added into a 1.5 mL RNAse/DNAse-free microfuge tube. For every 85 μL of whole blood collected, 15 μL of 10 mM IBMX was added for a final concentration of 1.5 mM IBMX in whole blood. Then, the blood was expelled into the tube, mixed gently by inversion with the IBMX, and allowed to clot at room temperature for at least 20 minutes prior to serum processing. The samples were centrifuged at room temperature for 10 minutes at 4,000×g in a benchtop microfuge. Following centrifugation, the clear serum was transferred to a prelabelled polypropylene tube, snap frozen on liquid nitrogen and stored frozen at −80° C.

### Ocular Tissue Collection and Processing

Following euthanasia, eyes of elected animals were processed for histological or immunological examination. The ODs of animal 519 (Group 5) and 629 (Group 6) were noted to have a very small lens, blood in the retina, and the eye was filled with a gel-like substance. No other abnormalities in necropsy and tissue processing were noted in the study records. For Groups 1 and 2, immediately following euthanasia, selected eyes were collected into 10% neutral buffered formalin. The eyes were placed in 70% ethanol the following day. The eyes were then processed to paraffin blocks for sectioning. Sagittal sections of each eye (5 μm thickness) were prepared for all animals. At least 3 slides containing a ribbon of approximately 5 sections were collected sequentially. The optic nerve was included in the sectioning. The slides were stained for hematoxylin and eosin (H&E) and examined using light microscopy. Representative images from the central retina from the H&E histopathology are shown in FIG. 9B. Retinal ganglion cells (RGC) were counted (Table 19 and FIG. 9C). The mean RGC count of the right eyes (ODs; experimental eyes) was lower than the mean RGC count of the left eyes (OSs; control eyes), and there were no differences in the mean RGC count in the ODs and OSs between Groups 1 and 2.

TABLE 19
RGC counts of Groups 1 and 2

| Animal | | | Histopathology |
|---|---|---|---|
| ID | Eye | Group | Comments |
| 101 | L | 1 PBS | RGC #/HPF (400X) = 46 |
| 101 | R | | RGC #/HPF (400X) = 33 |
| 102 | L | | RGC #/HPF (400X) = 48 |
| 102 | R | | RGC #/HPF (400X) = 35 |
| 213 | L | 2 MK- | RGC #/HPF (400X) = 37 |
| 213 | R | 801 | RGC #/HPF (400X) = 41 |
| 214 | L | 1.5 | RGC #/HPF (400X) = 35 |
| 214 | R | mM | RGC #/HPF (400X) = 31 |
| 223 | L | | RGC #/HPF (400X) = 55 |
| 224 | L | | RGC #/HPF (400X) = 38 |

### Ocular Tissue Collection for Retinal Flatmount Analysis (All Groups)

Eyes were enucleated and immediately fixed in 4% paraformaldehyde in phosphate-buffered saline (PBS) and stored overnight at 4° C. The following day, the eyes were transferred to cold immunocytochemistry (ICC) buffer (PBS containing 0.5% BSA and 0.2% Tween 20) until processing. Using a dissecting microscope, the eye was carefully trimmed of extraneous tissue at the limbus and, using fine curved scissors, the anterior chamber was removed. Retinas in the eye cup were rinsed with cold ICC buffer. Eye cups were placed in cold ICC buffer containing 1/100 rat anti-CNP36 (Groups 3-6 only; resuspended at 0.5 mg/mL in PBS) for 3 hours at 4° C. Eye cups were then washed extensively and stained with 1/200 donkey anti-rat Cy3 and 1/1,000 DAPI for 2-3 hours at 4° C. Groups 1 and 2 were stained with 1/500 TUJ-1 and 1/1000 DAPI, while Groups 7 and 8 were incubated with 1/1,000 DAPI alone. Eye cups were washed extensively but gently with cold ICC buffer. Using fine curved scissors and an eyelash knife, the retina was detached from the optic nerve head and removed from the RPE/choroid. Radial cuts were then made toward the center and retinas were flat mounted, covered, and sealed. 2D fluorescent microscopy images were acquired using an Olympus Bx63 upright fluorescent microscope. The TUJ-1 signal was highly variable across Groups 1 and 2 and provided little additional information so the Cy2 channel was not imaged. Images at 20× were acquired in 4 quadrants and a central 248.1×325.7 μm region was analyzed for RGC counts using Olympus cellSens and ImageJ software.

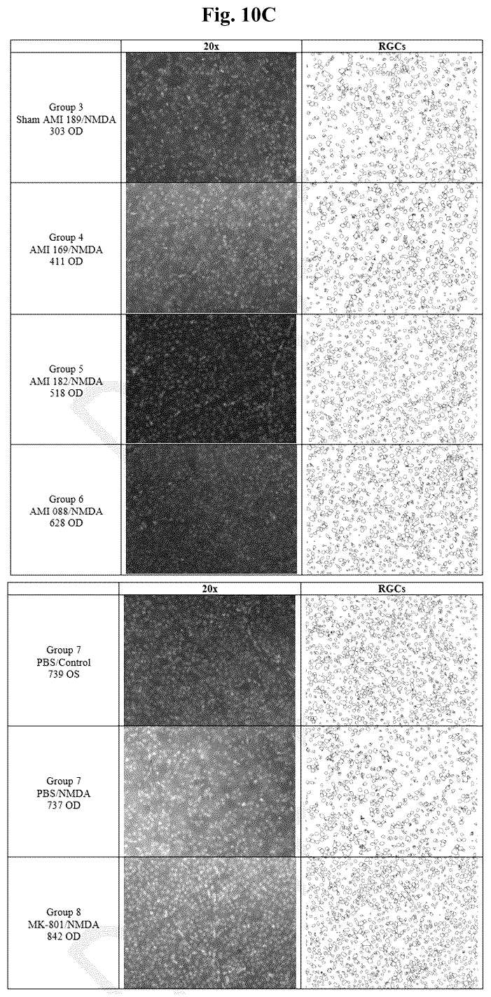

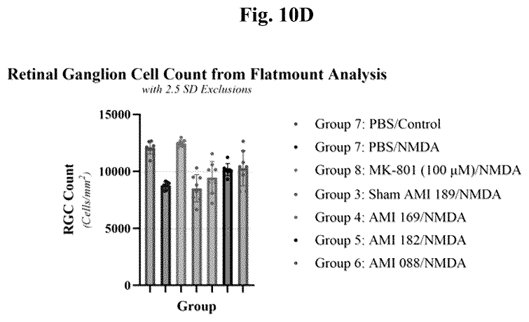

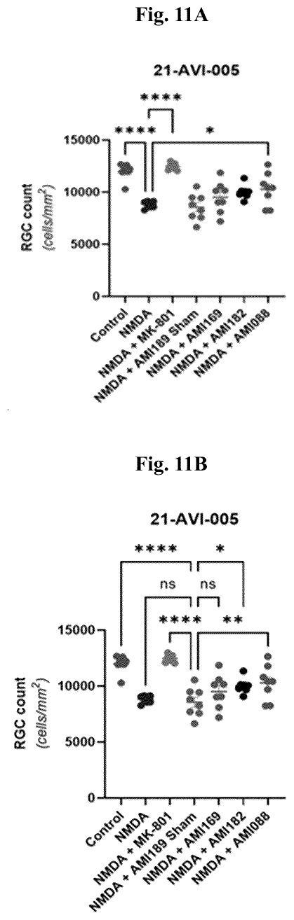

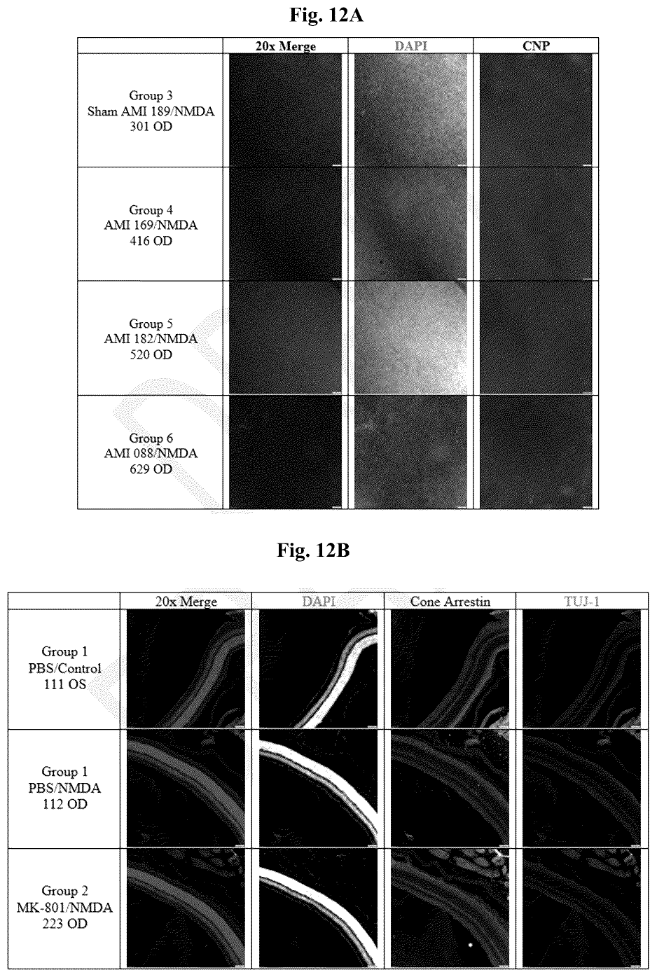

Groups 1 and 2 were stained with DAPI and TUJ-1 and images were captured at 20× (FIG. 10A). RGCs were counted in four quadrants, and cells/mm2 were averaged across each group. When the RGC count was quantified across groups (FIG. 10B), the control group (Group 1 OS; PBS, no NMDA) had an average count of 11,000±700 cells/mm2. The NMDA control group (Group 1 OD; PBS+NMDA) had a decreased RGC count of 8,000±1,000 cells/mm2. The Group 2 ODs had an RGC count slightly increased over the Group 1 ODs, at 9,000±500 cells/mm2.

Groups 3-8 were stained for DAPI and four images were captured for each retina at 20× approximately 600 μm from the optic nerve head. RGCs were counted in a central 248.1×325.7 μm region and cells/mm2 were averaged over each group (FIG. 10C). When the RGC count was calculated, the control group (Group 7 OS; PBS, no NMDA) had an average count of 12,000±700 cells/mm2 (FIG. 10D). The NMDA control group (Group 7 OD; PBS+NMDA) had a decreased RGC count of 9,000±300 cells/mm2. The Group 8 ODs had an RGC count similar to the Group 7 OSs, at 12,000±300 cells/mm2. Group 3 ODs had an RGC count similar to the Group 7 ODs, at 9,000±1,000 cells/mm2. Group 4 ODs had an RGC count of 10,000±1,400 cells/mm2. Group 5 ODs had an RGC count of 10,000±600 cells/mm2. Group 6 ODs had an RGC count of 10,000±1,500 cells/mm2.

The statistical analyses were performed on the Group 3-8 RGC data (one-way ANOVA followed by multiple comparisons using the Dunnett statistical hypothesis comparison). Treatment of PBS-injected eyes with NMDA caused a statistically significant decrease in RGC counts (P<0.0001). Increasing the dose of MK-801 to 100 μM rescued the Group 8 RGC counts to the Group 7 OS control level and the difference between NMDA-treated (Group 7 OD) and NMDA+MK-801-treated (Group 8) eyes was statistically significant (P<0.0001). When Groups 4-6 were compared to the Group 3 NMDA/sham vector controls, both the AMI 182 (Group 5) and AMI 088 (Group 6) reached statistical significance (FIG. 11A; P=0.0332 for Group 5 versus Group 3; P=0.0021 for Group 6 versus Group 3). When the RGC count of the AAV-treated groups were compared to the NMDA/PBS controls (Group 7 OD), only the AMI 088 construct maintained statistical significance (P=0.002; FIG. 11B). Groups 3-6 were stained for CNP36 in addition to DAPI to evaluate the expression location and levels of the AAV constructs, and images (4× and 20×) were captured (FIG. 12A).

### Ocular Tissue Collection for Immunohistochemistry (Groups 1 and 2 Only)

All eyes designated for immunohistochemistry (IHC) were enucleated, the approximate site of injection was marked, and eyes were fixed at 4° C. in 4% paraformaldehyde in separately labeled vials overnight. Eyes were then transferred into 0.1M phosphate buffer (PB), brought through a sequential sucrose gradient (10-30%, 1 hour each) followed by embedding in OCT medium and freezing on dry ice. The entire eye was cryosectioned (14 μm sections) and stained with the following antibodies shown in Table 20.

TABLE 20
Antibodies for ocular tissue immunohistochemistry
Day 7 IHC

| | | | Species/ | | Company | | | |
|---|---|---|---|---|---|---|---|---|
| | Primary | | clonality/ | | Name & | Secondary | | |
| Cocktail | Antibody | Cell type | Isotype | Dilution | cat # | Antibody | Dilution | Company |
| 1 | | | | | | Mouse | 1:500 | Bio |
| | | | | | | TUJ1 | | Legend |
| | | | | | | Alexa 488 | | |
| | Cone | Photo- | Rabbit | 1:500 | EMD | Donkey | 1:200 | Jackson |
| | arrestin | receptor | polyclonal | | Millipore | anti-rabbit | | Immuno |
| | | cones | IgG | | (AB15282) | Cy3 | | |
| | | | | | | DAPI | 1:1,000 | Thermo |

Negative controls were performed for the staining cocktail by utilizing only the secondary antibody. Five slides spanning the retina for each eye were stained. Two pictures per retinal cross-section—one at or near the injection site and one from a central region—were taken using an Olympus Bx63 upright fluorescent microscope and cellSens software. Qualitatively, there did not appear to be a difference in number of TUJ-1+ and cone arrestin+ cells between groups. All groups had clear cone arrestin staining with some TUJ-1+ cells observed (FIG. 12B). No further staining or analysis was performed on cross-sections as the staining was not an informative endpoint.

### Ocular Tissue Collection (Groups 3-6)

Eyes allocated for assays were enucleated, snap frozen, and stored at ≤−70° C. until shipment on dry ice. The tissues were placed into appropriate pre-weighed labeled analytical vials, immediately reweighed to determine sample weight, and placed on dry ice until being transferred to a freezer. Samples were weighed on a balance capable of measuring out to 4 decimal places. Samples collected included: serum (2 mL polypropylene screw cap tube); and whole globes with lens (2 mL polypropylene screw cap tube).

### Conclusions

The objective of this non-clinical study was to evaluate the effect of the AAV constructs on retinal ganglion cell protection following damage caused via NMDA excitotoxicity in the mouse. Mice were pretreated with the AAV constructs on Day −28 at dose of 4e+8 vg/eye, and then retinal degeneration was induced with NMDA on Day 0 into the right eye only. Groups 1, 2, 7 and 8 received a co-injection of NMDA with either PBS (Groups 1, 7) or MK-801 (Groups 2, 8).

Animals maintained a normal body weight over the course of the study. When the RGC count of Groups 1 and 2 was measured via histopathology, the experimental eyes (ODs) had a lower RGC count than the control eyes (OSs). When RGC counts were measured via Flatmount immunohistochemistry, the NMDA-treated eyes (Groups 1 and 7 OD) had significantly lower RGC count than the control eyes (Groups 1 and 7 OS). Pretreatment with 100 μM MK-801 (Group 8) mitigated the damage from NMDA administration. Amongst the AAV treatments, pretreatment with AMI 088 resulted in a statistically significantly higher RGC count compared to NMDA controls, approximately 20% more RGC counts, but it was not as effective as the 100 μM MK-801. When the AAV treatment groups were compared to a sham AAV+NMDA group both AMI 182 and AMI 088 reached statistical significance of higher RGC counts.

Overall, 100 μM MK-801 was an adequate positive control for retinal ganglion cell protection following damage caused by NMDA-induced excitotoxicity as it fully rescued the NMDA RGC loss phenotype. Future studies can investigate different dose levels of AMI 182 and AMI 088 and different timepoints post-NMDA administration.

This study was designed to determine the efficacy of a proposed method to cause retinal degeneration similar to that seen in humans. One method to cause this retinal degeneration is via an intravitreal injection of NMDA. The number of animals, data collection time points and parameters for measurement were chosen based on the minimum required to meet the objectives of the study.

Bioanalytical work and the accompanying analyses were performed at the conclusion of the study. Ocular samples (e.g., whole eye globes) from Groups 3-6 were enucleated, frozen after isolation. All samples were received in frozen condition upon receipt. Left eyes (OS) from all animals (n=8) in each group receiving the different AAV constructs were analyzed. Table 21 shows documentation of tissue weights and the volume of RIPA lysis and extraction buffer with protease inhibitor added to each sample prior to homogenization. Samples were placed on ice and homogenized using a sonicator as the following: a 20 second pulse followed by a 20 second rest for three cycles. Following sonication, the samples were rested on ice.

TABLE 21
Tissue weight and RIPA buffer volume

| | | | | Pre- | Post- | Tissue | RIPA + |
|---|---|---|---|---|---|---|---|
| Animal | Group | | | weight | weight | Weight | PI |
| # | # | Eye | Tissue | (g) | (g) | (g) | (μL) |
| 301 | 3 | OS | Whole | 1.58 | 1.61 | 0.022 | 221.0 |
| | | | Globe | | | | |
| 302 | | OS | Whole | 1.58 | 1.60 | 0.024 | 240.0 |
| | | | Globe | | | | |
| 303 | | OS | Whole | 1.57 | 1.60 | 0.023 | 231.0 |
| | | | Globe | | | | |
| 304 | | OS | Whole | 1.58 | 1.60 | 0.021 | 210.0 |
| | | | Globe | | | | |
| 305 | | OS | Whole | 1.58 | 1.60 | 0.021 | 210.0 |
| | | | Globe | | | | |
| 306 | | OS | Whole | 1.58 | 1.60 | 0.021 | 205.0 |
| | | | Globe | | | | |
| 307 | | OS | Whole | 1.57 | 1.59 | 0.021 | 214.0 |
| | | | Globe | | | | |
| 308 | | OS | Whole | 1.58 | 1.60 | 0.021 | 215.0 |
| | | | Globe | | | | |
| 409 | 4 | OS | Whole | 1.58 | 1.61 | 0.030 | 299.0 |
| | | | Globe | | | | |
| 410 | | OS | Whole | 1.57 | 1.59 | 0.021 | 214.0 |
| | | | Globe | | | | |
| 411 | | OS | Whole | 1.57 | 1.59 | 0.021 | 209.0 |
| | | | Globe | | | | |
| 412 | | OS | Whole | 1.58 | 1.60 | 0.023 | 232.0 |
| | | | Globe | | | | |
| 413 | | OS | Whole | 1.58 | 1.61 | 0.024 | 240.0 |
| | | | Globe | | | | |
| 414 | | OS | Whole | 1.60 | 1.62 | 0.021 | 212.0 |
| | | | Globe | | | | |
| 415 | | OS | Whole | 1.59 | 1.61 | 0.022 | 217.0 |
| | | | Globe | | | | |
| 416 | | OS | Whole | 1.58 | 1.60 | 0.023 | 233.0 |
| | | | Globe | | | | |
| 517 | 5 | OS | Whole | 1.59 | 1.61 | 0.021 | 210.0 |
| | | | Globe | | | | |
| 518 | | OS | Whole | 1.58 | 1.60 | 0.021 | 209.0 |
| | | | Globe | | | | |
| 519 | | OS | Whole | 1.56 | 1.58 | 0.022 | 223.0 |
| | | | Globe | | | | |
| 520 | | OS | Whole | 1.58 | 1.61 | 0.021 | 207.0 |
| | | | Globe | | | | |
| 521 | | OS | Whole | 1.59 | 1.61 | 0.020 | 195.0 |
| | | | Globe | | | | |
| 522 | | OS | Whole | 1.57 | 1.59 | 0.021 | 206.0 |
| | | | Globe | | | | |
| 523 | | OS | Whole | 1.58 | 1.60 | 0.021 | 207.0 |
| | | | Globe | | | | |
| 524 | | OS | Whole | 1.59 | 1.61 | 0.021 | 212.0 |
| | | | Globe | | | | |
| 625 | 6 | OS | Whole | 1.56 | 1.59 | 0.022 | 217.0 |
| | | | Globe | | | | |
| 626 | | OS | Whole | 1.58 | 1.61 | 0.027 | 267.0 |
| | | | Globe | | | | |
| 627 | | OS | Whole | 1.58 | 1.60 | 0.023 | 229.0 |
| | | | Globe | | | | |
| 628 | | OS | Whole | 1.57 | 1.59 | 0.021 | 211.0 |
| | | | Globe | | | | |
| 629 | | OS | Whole | 1.57 | 1.60 | 0.023 | 229.0 |
| | | | Globe | | | | |
| 630 | | OS | Whole | 1.57 | 1.59 | 0.021 | 210.0 |
| | | | Globe | | | | |
| 631 | | OS | Whole | 1.58 | 1.60 | 0.021 | 209.0 |
| | | | Globe | | | | |
| 632 | | OS | Whole | 1.60 | 1.62 | 0.023 | 225.0 |
| | | | Globe | | | | |

The CNP36 was the peptide produced by the vector AAV2.N54-CNP36 (AMI182). AMI182 was the same DNA transgene that was derived from AMI087 with multiple stop codon at the last cysteine residue of CNP36. Therefore, the Fe open reading frame was disrupted.

The CNP36 and CNP-Fc36 expression in ocular (whole globes) and serum samples was quantified using the commercial CNP36 ELISA kit and in-house CNP36-Fc ELISA (Example 2), respectively. The commercial ELISA was performed by following the stepwise procedure described in the user's manual. For in-house ELISA, 2 μg/mL anti-CNP36 antibody was coated onto a 96-well plate and incubated overnight at 4° C. The plate was washed with wash buffer and blocked with blocking buffer. Ocular and serum samples were delivered to the specified wells directly without any dilution. Samples were incubated for 1 hour. Plates were washed, and the detection antibody was added into each well at a 1:20,000 dilution. After a 1 hour incubation, plates were washed and Streptavidin-horse radish peroxidase (HRP) was added at a 1:10,000 dilution. After a 45 min incubation, plates were washed and CNP36-Fc was detected by the addition of 3,3′,5,5′-tetramethylbenzidine (TMB) substrate. The reaction was stopped by the addition of stop solution and the plate was read immediately at 450 nm along with the reference wavelength of 600 nm.

### CNP36-Fc Quantification in Ocular and Serum Samples

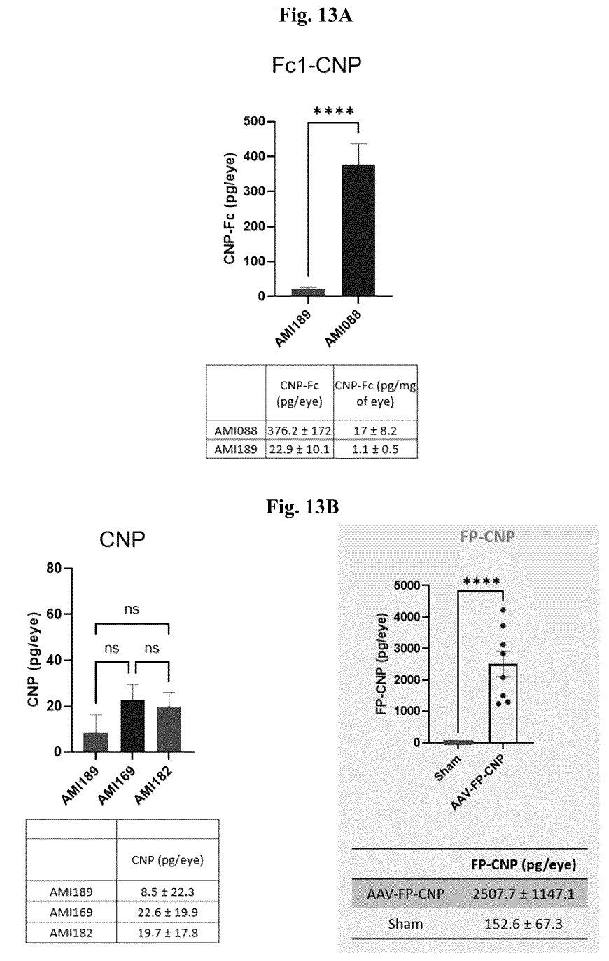

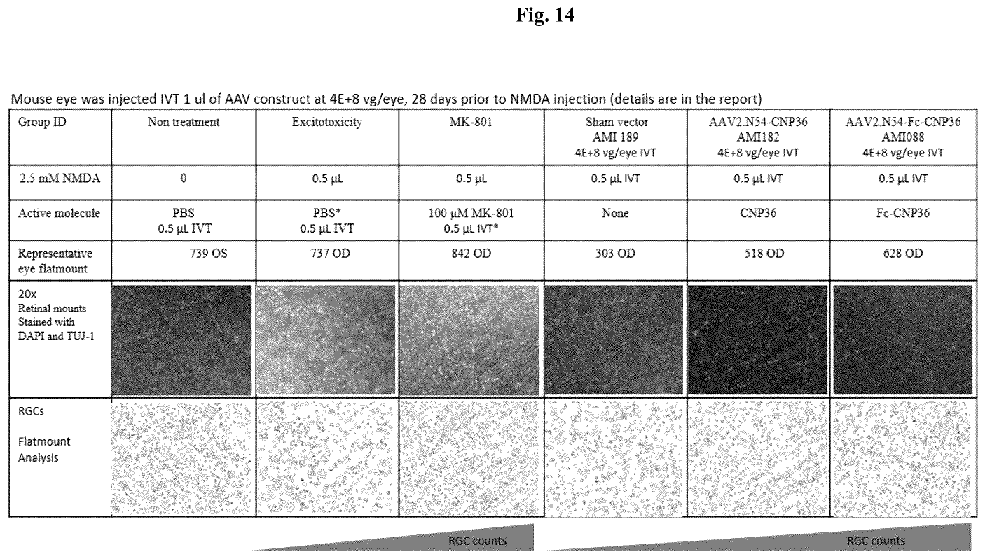

Animals from Groups 3 (AMI189) and 6 (AMI088) were analyzed using the CNP36-Fc ELISA. Group 3 (sham vector) animals served as a negative control (FIG. 13A). Group 6 animals were injected with AMI088, which was an AAV2 vector with N-terminal CNP36 fused to the C-terminus of human IgG1Fc fragment (CNP36-Fc). The data were analyzed in GraphPad Prism software using one-way ANOVA followed by Dunnett's multiple comparison (****=<0.0001). A statistically significant difference was observed between Groups 3 and 6. In the samples where the Fc fusion was not present, however, CNP36 levels were quite low, likely due to CNP36's very short half-life. Ocular samples from Groups 3, 4 and 5 were analyzed using the commercial ELISA kit described above (FIG. 13B, left graph). Although Groups 4 and 5 showed a slightly higher level of CNP36 compared to Group 3 (sham vector; negative control), the observed differences were not significant. The low levels observed on the ELISA likely indicated that only a small fraction of the total expressed CNP36 was being quantified as most of the protein may have been proteolyzed prior to quantification. Since the test articles for Groups 4 and especially 5 showed efficacy, CNP36 was expressed but the optimal conditions for protection from proteolysis and subsequent detection could be difficult. Neither CNP36-Fc nor CNP36 were expressed in the serum samples. FIG. 14 illustrates RGC protection by AAV vectors encoding CNP. Mouse eye was injected via IVT 1 μl of AAV construct at 4E+8 vg/eye, 28 days prior to NMDA injection. Images indicated NMDA induced RGC #reduction, which was rescued by MK-801. Sham vector showed reduced RGCs in the NMDA only treatment group, but the AMI182 and AMI088 groups showed higher RGC counts than the groups of sham vector and NMDA treatments. Table 22 shows the expression summary of detection of CNP36 and CNP36-Fc in ocular homogenate samples.

TABLE 22
CNP36 and CNP36-Fc expression in ocular homogenate samples

| | | Dose/eye | CNP36 | CNP36-Fc |
|---|---|---|---|---|
| Product | Entity | (IVT) | (pg/eye) | (pg/eye) |
| AAV2.N54-189 | Sham vector | 4E+08 vg/eye | 8.5 ± 22.3 | 22.9 ± 10.1 |
| | control | | | |
| AAV2.N54-169 | Lucentis | 4E+08 vg/eye | 22.6 ± 19.9 | — |
| | scFv + CNP36 | | | |
| AAV2.N54-182 | CNP36 | 4E+08 vg/eye | 19.7 ± 17.8 | — |
| AAV2.N54-088 | CNP36-Fc | 4E+08 vg/eye | — | 376.2 ± 172 |

AAV2 constructs showed efficacy via retinal ganglion cell recovery in a pilot NMDA excitoxicity study. CNP36-Fc was stable and thus quantifiable in the ocular samples. CNP36 without the Fc fusion was difficult to quantify due to its short half-life from rapid proteolysis. Expression of CNP36 and CNP36-Fc was not detected. For CNP36-Fc, this indicated that the protein was not leaking systemically but acted locally, which would be a distinct therapeutic advantage. The sham vector showed no expression CNP36 acts as good negative control for the study.

TABLE 23
Amino acid sequence of CNP, CNP fusion protein, and antibody or fragment
thereof

| | | |
|---|---|---|
| SEQ | Full | MHLSQLLACALLLTLLSLRPSEAKPGAPPKVPRTPPAEELAEPQAAGGG |
| ID | length | QKKGDKAPGGGGANLKGDRSRLLRDLRVDTKSRAAWARLLQEHPNAR |
| No: | CNP | KYKGANKKGLSKGCFGLKLDRIGSMSGLGC |
| 1 | | |
| SEQ | CNP | GDRSRLLR DLRVDTKSRAAWARLL QEHPNA |
| ID | precursor | RKYKGANKKGLSKGCFGLKLDRIGSMSGLGC |
| No: | (CNP-61) | |
| 2 | | |
| SEQ | CNP-53 | DL RVDTKSRAAW ARLLQ EHPNA RKYKGANKK |
| ID | | GLSKGCFGLKLDRIGSMSGLGC |
| No: | | |
| 3 | | |
| SEQ | CNP-36 | EHPNA RKYKGANKKGLSKGCFGLKLDRIGSMSGLGC |
| ID | | |
| No: | | |
| 4 | | |
| SEQ | CNP-22 | GLSKGCFGLKLDRIGSMSGLGC |
| ID | | |
| No: | | |
| 5 | | |
| SEQ | Antibody | MEFGLSWLFLVAILKGVQC |
| ID | signal | |
| No: | peptide | |
| 6 | | |
| SEQ | IgG1 | DKTHTCPPCPAPELLGGPSVFLFPPKPKDTLMISRTP |
| ID | | EVTCVVVDVSHEDPEVKFNWYVDGVEVHNAKTKP |
| No: | | REEQYNSTYRVVSVLTVLHQDWLNGKEYKCKVSN |
| 7 | | KALPAPIEKTISKAKGQPREPQVYTLPPSRDELTKN |
| | | QVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGK |
| SEQ | IgG4 | DKRVESKYGPPCPPCPAPEFLGGPSVFLFPPKPKDTLMISRTPEVTCVVV |
| ID | | DVSQEDPEVQFNWYVDGVEVHNAKTKPREEQFNSTYRVVSVLTVLHQ |
| No: | | DWLNGKEYKCKVSNKGLPSSIEKTISKAKGQPREPQVYTLPPSQEEMTK |
| 8 | | NQVSLTCLVKGFYPSDIAVEWESNGQPENNYKTTPPVLDSDGSFFLYSR |
| | | LTVDKSRWQEGNVFSCSVMHEALHNHYTQKSLSLSLGK |
| SEQ | SP-CNP36 | MEFGLWLFLVAILKGVQC EHPNA RKYKGANKKG LSKGCFGLKL |
| ID | | DRIGSMSGLG C |
| No: | | |
| 9 | | |
| SEQ | SP-CNP22 | MEFGLWLFLVAILKGVQC LSKGCFGLKL DRIGSMSGLG C |
| ID | | |
| No: | | |
| 10 | | |
| SEQ | SPVh- | MEFGLSWLFLVAILKGVQCDKTHTCPPCPAPELLGG |
| ID | CNP36 | PSVFLFPPKPKDTLMISRTPEVTCVVVDVSHEDPEV |
| No: | | KFNWYVDGVEVHNAKTKPREEQYNSTYRVVSVLT |
| 11 | | VLHQDWLNGKEYKCKVSNKALPAPIEKTISKAKGQ |
| | | PREPQVYTLPPSRDELTKNQVSLTCLVKGFYPSDIA |
| | | VEWESNGQPENNYKTTPPVLDSDGSFFLYSKLTVD |
| | | KSRWQQGNVFSCSVMHEALHNHYTQKSLSLSPGKG |
| | | GGGSGGGGSGGGGSGGGGSEHPNARKYKGANKKG |
| | | LSKGCFGLKLDRIGSMSGLGC |
| SEQ | SPVh- | MEFGLSWLFLVAILKGVQCDKTHTCPPCPAPELLGG |
| ID | CNP22 | PSVFLFPPKPKDTLMISRTPEVTCVVVDVSHEDPEV |
| No: | | KFNWYVDGVEVHNAKTKPREEQYNSTYRVVSVLT |
| 12 | | VLHQDWLNGKEYKCKVSNKALPAPIEKTISKAKGQ |
| | | PREPQVYTLPPSRDELTKNQVSLTCLVKGFYPSDIA |
| | | VEWESNGQPENNYKTTPPVLDSDGSFFLYSKLTVD |
| | | KSRWQQGNVFSCSVMHEALHNHYTQKSLSLSPGKG |
| | | GGGSGGGGSGGGGSGGGGSLSKGCFGLKLDRIGSM |
| | | SGLGC |

### Example 4. Treatment of Rat Model of Partial Optic Nerve Transection (pONT)

### Aims

Using an experimental rat model of partial optic nerve transection (pONT), effects of Adeno-associated virus (AAV) CNP peptide (AAV-P) and protein Fc fusion (AAV-FP) on: retinal ganglion cell (RGC) apoptosis/stress in vivo was assessed using detection of apoptosing retinal cells (DARC) imaging; intraocular inflammation in vivo using Optical Coherence Tomography (OCT) imaging; intraocular pressure (IOP) in vivo using Tonolab tonometry; RGC survival histologically using immunohistochemistry on retinal whole mounts; and retinal microglial activity histologically using immunohistochemistry on retinal whole mounts.

### Materials and Methods: Test Article Information

Table 24 illustrates the final calculation of dosage per eye: 4 μL/eye.

TABLE 24
Final calculation of dosage per eye: 4 μL/eye

| | | Animal | | | Dose vg/per | | Test Article | Ready for |
|---|---|---|---|---|---|---|---|---|
| Category | Test Article | No. | Concentration | | eye (IVT) | | Vol × vial# | injection |
| Vehicle | PBS-F68 | 6 | | | | | 50 μl × 2 | yes |
| Positive | AMI088 | 6 | 2.5E13 | vg/mL | 1e+11 | vg | 50 μl × 2 | yes |
| Control AAV | | | | | | | | |
| Negative | AMI189 | 6 | 2.5E13 | vg/mL | 1e+11 | vg | 50 μl × 2 | yes |
| Control AAV | (Sham*) | | | | | | | |
| Peptide (P) | AMI302 | 6 | 2.5E13 | vg/mL | 1e+11 | vg | 50 μl × 2 | yes |
| | AMI302 | 6 | 2.5E12 | vg/mL | 1e+10 | vg | 50 μl × 2 | yes |
| | AMI302 | 6 | 2.5E11 | vg/mL | 1e+9 | vg | 50 μl × 2 | yes |
| Fc-peptide (FP) | AMI273 | 6 | 2.5E13 | vg/mL | 1e+11 | vg | 50 μl × 2 | yes |
| | AMI273 | 6 | 2.5E12 | vg/mL | 1e+10 | vg | 50 μl × 2 | yes |
| | AMI273 | 6 | 2.5E11 | vg/mL | 1e+9 | vg | 50 μl × 2 | yes |
| Pure Fc-peptide | Fc4-263 | 6 | 0.5 | mg/mL | 2 | μg | 50 μl × 2 | yes |
| Protein | Fc4-263 | 6 | 5 | mg/mL | 20 | μg | 50 μl × 2 | yes |
| | Fc4-263 | 6 | 20 | mg/mL | 80 | μg | 50 μl × 2 | yes |
| Shipping | AMI088 | 0 | 1.25E12 | vg/mL | | | 25 μl × 6 | Prepared for 2-3 |
| control | | | | | | | | return shipment |
| | Fc4-263 | 0 | 5 | mg/mL | 20 | μg | 25 μl × 6 | Prepared for 2-3 |
| | | | | | | | | return shipment |
| | Total vials | | | | | | 36 vials | |

*sham: contains same capsid and the transgene cassette covering 5′ITR to ITR3′ DNA content are identical to the AMI273 except the disruption of the open reading frames (ORF). No protein product produced once administrated to animals.

On the −21 or −28 days, AAV-P, AAV-FP, vehicle, negative and positive AAV controls: thawed 1 vial (50 μL) of diluted vials of each test article above at ambient temperature for at least 24 hours and centrifugated in a minifuge/Eppendorf centrifuge for 30 seconds in order to spin down the condensed water on vial wall and pellet the potential aggregates. The pure Fc-peptide Protein vials would be thawed at ambient temperature for about 60 minutes and then spun briefly in a Eppendorf centrifuge to bring down the condensed water and to remove aggregates prior to injection.

### Procedure for Eye Ball Homogenization

Make a mixture of RIPA and the protease inhibitor was prepared by adding one tablet of protease inhibitor in 10 mL of RIPA buffer. Ocular tissue was measured for the amount of buffer needed. For every 1 mg of tissue, 10 μL of buffer was added. The tube containing the tissue was sonicated for 20 seconds on ice followed by 20 seconds of rest on ice. The sonication and chilling on ice was repeated two more times making 3 cycles in total. These cycles could be repeated according to requirement (if the tissue still did not homogenize and lumps were visible).

After complete homogenization, the tubes were placed on an orbital shaker in cold room for two hours. After two hours, the tubes were centrifuged for five minutes at 13,000 rpm. The supernatants were collected and stored store at −80° C.

### Randomized Experimental Design

In total, seventy-two male Dark Agouti (DA) rats aged 8-10 weeks were used in this study. The seventy-two animals were randomly divided into 3 categories. Category 1 (Table 25): AAV-P (AMI302) and sham vector-only (AMI189) (n=24); Category 2 (Table 26): AAV-FP (AMI273), buffer-only (PBS-F68), and positive control AAV (AMI088) (n=30); and Category 3 (Table 27): pure fusion protein (FP-263) (n=18).

Each category contained 6 blocks with each block covering different treatments, e.g., n=6 (see Categories 1, 2, and 3). The rat ID for each treatment was shown in the left column of each block. L: lower dose; M: medium dose; and H: higher dose.

Intravitreal injections (IVT, 4 μL) were given under general anesthesia (GA), according to the categories, and administered to the left eye (pONT eye) only once using a 34-gauge Hamilton needle at day 0 after BL imaging. pONT was performed at week 3 in Categories 1 and 2. In Category 3, pONT was performed soon after baseline imaging.

Partial optic nerve transection (pONT) surgery was performed under GA at week 3 in categories 1 and 2. In category 3, pONT surgery was performed on day 0 after BL imaging. Briefly, an incision was made in the superior conjunctiva, and the optic nerve sheath was exposed. A longitudinal slit was made in the dura mater and a 0.2-mm cross-cut was performed in the dorsal optic nerve at a distance of 2 mm behind the eye. An ophthalmic scalpel with a steel cutting guard of 0.2 mm was used in this procedure. Damage to major ophthalmic blood vessels was avoided and verified at the end of surgery by ophthalmoscopy.

Animals were culled at week 4 in categories 1 and 2, and at week 1 in category 3. Before culling, a blood sample (at least 2 mL) was collected by cardiac puncture under the terminal procedure, centrifuged to collect serum, and stored at −80° C.

TABLE 25
Category 1. AAV-P (AMI302) and Vector-only (AMI189) (n = 24)

| Block 1 | | Block 2 | | Block 3 | | Block 4 | | Block 5 | | Block 6 | |
|---|---|---|---|---|---|---|---|---|---|---|---|
| | treat- | | treat- | | treat- | | treat- | | treat- | | treat- |
| rat | ment | rat | ment | rat | ment | rat | ment | rat | ment | rat | ment |
| 3663* | Vector | 3667* | Vector | 3671 | Vector | 3675 | Vector | 3679 | Vector | 3683 | Vector |
| | only | | only | | only | | only | | only | | only |
| 3664 | AAV-P | 3668 | AAV-P | 3672 | AAV-P | 3676 | AAV-P | 3680 | AAV-P | 3684 | AAV-P |
| | (L) | | (L) | | (L) | | (L) | | (L) | | (L) |
| 3665 | AAV-P | 3669 | AAV-P | 3673 | AAV-P | 3677 | AAV-P | 3681 | AAV-P | 3685 | AAV-P |
| | (M) | | (M) | | (M) | | (M) | | (M) | | (M) |
| 3666 | AAV-P | 3670 | AAV-P | 3674 | AAV-P | 3678 | AAV-P | 3682 | AAV-P | 3686 | AAV-P |
| | (H) | | (H) | | (H) | | (H) | | (H) | | (H) |

TABLE 26
Category 2: AAV-FP (AMI273), buffer-only (PBS-F68), and positive control AAV (AMI088) (n = 30)

| Block 1 | | Block 2 | | Block 3 | | Block 4 | | Block 5 | | Block 6 | |
|---|---|---|---|---|---|---|---|---|---|---|---|
| | treat- | | treat- | | treat- | | treat- | | treat- | | treat- |
| rat | ment | rat | ment | rat | ment | rat | ment | rat | ment | rat | ment |
| 3687 | Buffer- | 3692 | Buffer- | 3697 | Buffer- | 3702 | Buffer- | 3707 | Buffer- | 3712 | Buffer- |
| | only | | only | | only | | only | | only | | only |
| 3688 | AAV-FP | 3693 | AAV-FP | 3698 | AAV-FP | 3703 | AAV-FP | 3708* | AAV-FP | 3713 | AAV-FP |
| | (L) | | (L) | | (L) | | (L) | | (L) | | (L) |
| 3689 | AAV-FP | 3694 | AAV-FP | 3699 | AAV-FP | 3704 | AAV-FP | 3709 | AAV-FP | 3714 | AAV-FP |
| | (M) | | (M) | | (M) | | (M) | | (M) | | (M) |
| 3690 | AAV-FP | 3695 | AAV-FP | 3700 | AAV-FP | 3705 | AAV-FP | 3710 | AAV-FP | 3715 | AAV-FP |
| | (H) | | (H) | | (H) | | (H) | | (H) | | (H) |
| 3691 | AAV | 3696 | AAV | 3701 | AAV | 3706 | AAV | 3711 | AAV | 3716 | AAV |
| | positive | | positive | | positive | | positive | | positive | | positive |

TABLE 27
Category 3: Pure FP-peptide Protein (FP-263) (n = 18)

| Block 1 | | Block 2 | | Block 3 | | Block 4 | | Block 5 | | Block 6 |
|---|---|---|---|---|---|---|---|---|---|---|
| treat- | | treat- | | treat- | | treat- | | treat- | | treat- |
| ment | rat | ment | rat | ment | rat | ment | rat | ment | rat | ment |
| FP-263 | 3720 | FP-263 | 3723 | FP-263 | 3746 | FP-263 | 3749 | FP-263 | 3752 | FP-263 |
| (L) | | (L) | | (L) | | (L) | | (L) | | (L) |
| FP-263 | 3721 | FP-263 | 3724 | FP-263 | 3747 | FP-263 | 3750 | FP-263 | 3753 | FP-263 |
| (M) | | (M) | | (M) | | (M) | | (M) | | (M) |
| FP-263 | 3722 | FP-263 | 3725 | FP-263 | 3748 | FP-263 | 3751 | FP-263 | 3754 | FP-263 |
| (H) | | (H) | | (H) | | (H) | | (H) | | (H) |

*Animal died during in vivo experiments (see details in Exclusions below)

### In Vivo Assessments

DARC/OCT. under GA, DARC/OCT imaging was conducted at baseline (BL), weeks 1, 2, and 4 in categories 1 and 2. pONT was performed at week 3. In category 3, DARC/OCT was conducted at baseline and week 1 before termination with pONT performed soon after baseline imaging. Briefly, fluorescently labelled annexin 776 (6 mg/mL, 40 μL) was intranasally administrated 2 hours before DARC imaging and then assessed with a confocal scanning laser ophthalmoscope (cSLO). OCT imaging was performed of the posterior pole centering on the optic nerve disc using a Spectralis cSLO.

IOP. Intraocular pressure (IOP) was measured at BL and three times (Monday, Wednesday, and Friday) a week at weeks 1, 2, and 4 in categories 1 and 2 and at week 1 in category 3 using a Tonolab tonometer under inhalational anesthesia. Ten IOP readings were collected from each eye of each animal at each time point.

### Histological Assessments

Three blocks (blocks 1-3) of animals from each category (36 rats in total) were used for immunohistochemistry study. After culling, both eyes of each animal were enucleated and retinal whole mounts dissected. Immunostaining was performed with anti-RBPMS and anti-Iba-1 antibodies to assess RGC survival and microglial activity, respectively. The immunostained retinal whole-mounts were then imaged under a fluorescence microscope.

### Protein Analysis

The remaining three blocks (blocks 4-6) of animals from each category (36 rats in total) were used for protein analysis. After culling, both eyes from each animal were enucleated and snap-frozen with liquid nitrogen, cryogenically homogenized with mortar and pestle and stored at −80° C. until protein analysis.

### ELISA

ELISA can be used for the concentration of CNP-Fc in process intermediates and drug substance using adeno-associated vector for gene therapy or CNP-Fc determination in analytical samples (cell culture supernatants), pre-clinical (plasma, homogenized tissues, vitreous humor etc. from monkey, pigs and rodents) and clinical samples. CNP-Fc comprises a recombinant fusion protein consisting of CNP-36 fused with human IgG. CNP-Fc can be affinity purified from HEK293 cells transduced with AMI088, which is rAAV2 carrying CNP gene that can make only 36 amino acid long peptide fused with Fc. A serial dilution of CNP-Fc can be used in the assay. The detection range of CNP-Fc concentration in this assay can be from 0.78 to 50 ng/mL. Material and equipment can include: CNP-Fc stored at −80° C. in 1×PBS (concentration can be determined by BCA); CNP coating/capture antibody: 100 μg/mL, antigen expressed in HEK293 cells and purified (GeneScript, Cat #Z03073); goat anti-human IgG Fc (Biotin) preadsorbed (Abcam, Cat #ab98618); HRP-streptavidin conjugate (Abcam, Cat #ab7403); TMB substrate: 1-Step™ Ultra TMB-ELISA substrate solution (ThermoFisher, Cat #34028); 96-well microplate reader (Molecular Device: VERSAmax tunable Microplate reader); coating buffer: 3.7 g sodium bicarbonate (NaHCO₃), 0.64 g sodium carbonate (Na₂CO₃), and 1 L of Milli Q water, pH 9.60 with storage condition at room temperature for one month; 1×PBS (phosphate buffered saline): 8.0 g sodium chloride, 1.3 g dibasic sodium phosphate, 0.2 g monobasic sodium phosphate, and 1.0-liter Milli-Q water, pH 7.4 with storage condition at room temperature for one year; washing buffer (PBST): 1×phosphate buffered saline and 0.1% Tween 20 (v/v) with storage condition at room temperature for 30 days expiration from date of preparation; blocking buffer (BB): 1× phosphate buffered saline (PBS) with 0.1% Tween 20 (v/v) and with 1% casein with storage condition at 4° C. for 30 days from date of preparation; dilution buffer (DB): same as blocking buffer; stop solution for TMB substrate: 2 N HCl, diluted in-house from the stock HCl purchased from Millipore Sigma, Cat #1003172510; and 96-well microplate.

ELISA procedure can include: diluting the CNP antibody (500 μg/mL) stock to 2 μg/mL with coating buffer (20 μL of VEGF stock to 5 mL of coating buffer); adding coating antigen to a 96-well microplate at 50 μL/well and covering and placing the plate at 2-8° C. for approximately 12 hours or overnight; discarding the coating antigen and wash the plate thrice with 300 μL/well of PBS-T wash buffer; adding 300 μL/well of blocking buffer, cover and incubate the plate at 37±1° C. for 120 minutes; diluting CNP-Fc standard (8.3 mg/mL) with dilution buffer to 50 ng/mL as the following: 8.3 mg/mL was diluted 83-fold to 100 μg/mL, 100 μg/mL was diluted 10-fold to 10 μg/mL, 10 μg/mL was diluted 10-fold to 1 μg/mL, and adding 25 μL of 1 μg/mL solution in 475 μL of dilution buffer as the first standard point of 50 ng/mL; preparing the rest of CNP-Fc standard using 1:2 serial dilution scheme in duplicates in Table 28; or diluting process intermediate samples, e.g., cell culture supernatants HEK293 with dilution buffer to 1:2000, followed by 1:4000, 1:8000 and 1:16000. For ARPE19 cell culture supernatant 1:250, 1:500, 1:1000 and 1:2000 dilutions can be made in separate plates. Table 29 is schematic representation of the HEK293 expressed spent medium quantification plate indicating the known CNP-Fc concentration (row 1A-1G and row 2A-2G) to construct the standard curve, blank with buffer only used as negative control (blank) and unknown samples (rows 3-10) for CNP-Fc concentration determination. Wells in white represent empty wells in 96-well plate. Unknown samples were tested in duplicates and the ratio in the brackets is the dilution factor.

TABLE 28
Exemplary CNP-Fc standard serial dilution

| Dilution | 2⁰ | 2⁻¹ | 2⁻² | 2⁻³ | 2⁻⁴ | 2⁻⁴ | 2⁻⁶ | |
|---|---|---|---|---|---|---|---|---|
| ng/mL | 50 | 25 | 12.5 | 6.3 | 3.1 | 1.6 | 0.78 | Blank |
| CNP-Fc (μL) | 25 | 200 | 200 | 200 | 200 | 200 | 200 | 0 |
| DB (μL) | 475 | 200 | 200 | 200 | 200 | 200 | 200 | 200 |

TABLE 29
Exemplary schematic representation of the HEK293 expressed spent medium quantification plate

| | 1 | 2 | 3 | 4 | 5 | 6 | 7 | 8 | 9 | 10 | 11 | 12 |
|---|---|---|---|---|---|---|---|---|---|---|---|---|
| A | 50 | 50 | 1.1 | 1.2 | 1.1 | 1.2 | 1.1 | 1.2 | 1.1 | 1.2 | — | — |
| | | | (1:2000 | (1:2000) | (1:4000) | (1:4000) | (1:8000) | (1:8000) | (1:16000) | (1:16000) | | |
| B | 25 | 25 | 2.1 | 2.2 | 2.1 | 2.2 | 2.1 | 2.2 | 2.1 | 2.2 | — | — |
| | | | (1:2000 | (1:2000 | (1:4000) | (1:4000) | (1:8000) | (1:8000) | (1:16000) | (1:16000) | | |
| C | 12.5 | 12.5 | 3.1 | 3.2 | 3.1 | 3.2 | 3.1 | 3.2 | 3.1 | 3.2 | — | — |
| | | | (1:2000) | (1:2000) | (1:4000) | (1:4000) | (1:8000) | (1:8000) | (1:16000) | (1:16000) | | |
| D | 6.3 | 6.3 | 4.1 | 4.2 | 4.1 | 4.2 | 4.1 | 4.2 | 4.1 | 4.2 | — | — |
| | | | (1:2000) | (1:2000) | (1:4000) | (1:4000) | (1:8000) | (1:8000) | (1:16000) | (1:16000) | | |
| E | 3.1 | 3.1 | 5.1 | 5.2 | 5.1 | 5.2 | 5.1 | 5.2 | 5.1 | 5.2 | — | — |
| | | | (1:2000) | (1:2000) | (1:4000) | (1:4000) | (1:8000) | (1:8000) | (1:16000) | (1:16000) | | |
| F | 1.6 | 1.6 | — | — | — | — | — | — | — | — | — | — |
| G | 0.8 | 0.8 | — | — | — | — | — | — | — | — | — | — |
| H | Blank | Blank | — | — | — | — | — | — | — | — | — | — |

Additional ELISA procedure can include: transferring the diluted standard samples and unknown samples to the plate according to Table 29 with 50 μL per well in duplicates for each dilution; covering the plate and incubating the plate at 37±1° C. for 60 minutes; discarding the reactants in the plate and washing 6 times with 300 μL/well of wash buffer; diluting goat anti-human IgG Fc (Biotin) preadsorbed at 1:20,000 with dilution buffer and adding 50 μL/well; covering and incubating the plate at 37±1° C. for 60 minutes; discarding the reaction mix, wash the plate 6 times, 300 μL/well with wash buffer; diluting streptavidin-TRP with dilution buffer at 1:10,000 and add 50 μL/well. Cover and incubated the plate at 37±1° C. for 60 minutes; discarding the reactants in the plate and wash 6 times, 300 μL/well, with wash buffer; adding TMB substrate, 50 μL/well; covering the plate and incubating at 37±1° C. for 15 minutes; stopping the reaction by adding stop solution 50 μL/well; reading the plate at 450 nm wavelength filter with 600 nm as reference wavelength in a microplate reader; or copying the data to the Excel spread sheet and constructing the standard curve for CNP-Fc. Unknown samples can be analyzed by first taking the mean of duplicates. Concentration of the unknown samples can be determined by incorporating the OD values obtained into the equation generated from the standard curve. At the end the concentration is adjusted for the dilution factor. The assay is considered valid when the following criteria are met: negative control gives an A₄₅₀ for TMB substrate essentially similar to the readings of blank control; or the standard curve is linear with R value of ≥0.975.

### Data Analysis

IOP measurements were performed three times a week at weeks 1, 2, and 4. DARC spots on in vivo images were automatically counted by an algorithm developed and validated in the Cordeiro lab. The DARC count was defined as the number of annexin-positive spots seen in the retinal image at 120 minutes at each time point after baseline spot subtraction. A single DARC score was generated for each retina by subtracting the DARC spots observed at the baseline timepoint from the DARC spots observed at the terminal timepoint. A linear transformation of +22 was applied to all DARC counts for visualisation purposes only, with no effect on the statistical properties of the data. OCT images were used to assess inflammation by manual counting of vitreal inflammatory cells. RBPMS⁺ RGCs in retinal whole mounts were automatically counted and analyzed by a recently developed and validated algorithm in the Cordeiro lab. To assess whether the treatments had a regional difference, the retinal wholemount was semi-segmented into the superior and inferior halves. The morphology of microglia in retinal whole mounts was automatically analyzed by a recently developed and validated machine learning approach in the Cordeiro lab.

### Statistical Analysis

Statistical analysis and graphing of data were completed on IBM SPSS and GraphPad Prism 9. Data were presented as means±SEM, and p<0.05 was considered as statistical significance. Where appropriate, ANOVAs and t-tests were used to analyse data. Where this was inappropriate, the non-parametric Mann-Whitney U test was used instead. Multiple pair-wise comparisons are always corrected for using the Tukey adjustment. To overcome small n numbers per treatment dose, and to improve statistical power, Medium and High concentration conditions were combined to assess the effect of treatments as single groups. Hence, FP, AAV-P, and AAV-FP consist of both medium and high concentrations only.

### Exclusions

Four rats (R3663, R3667, R3708, and R3719) died during the in vivo experimental course. Two of them (R3663 and R3667) were treated with vector-only, one (R3663) died before week 4 and the other (R3667) died just after week 4 imaging. Thus, there was no data available for DARC/OCT, histology, and blood sample from R3663. However, because R3667 died after week 4 imaging (just before culling), all data of DARC/OCT and histology was available except the blood sample. One rat (R3708), treated with AAV-FP low dose, died before week 4. So, no data on DARC/OCT, blood samples, and protein was available. One rat (R6719), treated with pure FP peptide protein high dose, died before week 1. Thus, no data on DARC/OCT, histology, and the blood sample was available. Several rats were excluded from the data analysis for various reasons listed in Table 30.

TABLE 30
A list of animals that were excluded from data analysis

| Analysis | Rat | Exclusion Reason |
|---|---|---|
| DARC | 3719 | Died |
| | 3663 | Died |
| | 3748 | Poor image quality |
| OCT | 3683 | Impossible to image |
| | 3688 | Impossible to image |
| | 3708 | Died |
| | 3663 | Died |
| | 3678 | Missing OCT data |
| | 3682 | Missing OCT data |
| | 3685 | Missing OCT data |
| | 3709 | Missing OCT data |
| RBPMS | 3719 | Died |
| | 3663 | Died |
| | 3722 | Anomalous data |
| | 3696 | Anomalous data |
| IBA1 | 3719 | Died |
| | 3663 | Died |
| | 3689 | Anomalous data |

### Results

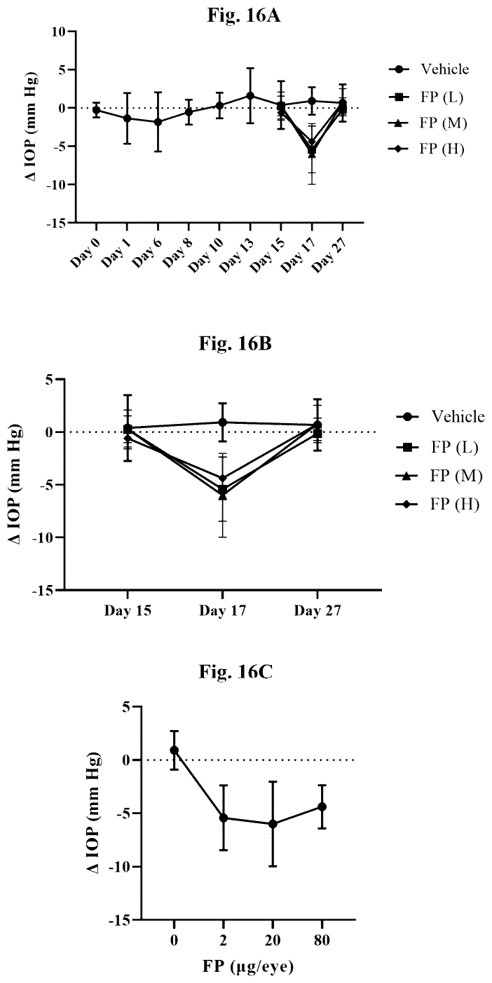

IOP analysis in the both Fc4-CNP36 (FP) and AAV treatment. To assess if intravitreal administration of treatments in the left eye affects IOP profiles, IOP measurements were performed three times a week at weeks 1, 2, and 4. The IOP data was analyzed by subtracting OD (the right eye) from OS (the left eye) in each animal at each time point. The results showed that FP had significant effect on IOP reduction after intravitreal injection of 2, 20 and 80 μg/eye of the affinity purified FP. Analysis of the FP group alone did show a significant effect (p<0.01) of FP on IOP, after pONT. FIGS. 16A-C illustrates effect of Fc4-CNP36 (FP) on intraocular pressure (IOP) change in rat partial optic nerve transection (pONT) model. FIG. 16A illustrates animal IOP monitored during the course of the study. FIG. 16B illustrates animal IOP change after Fc4-CNP36 administration intravitreally (IVT). FIG. 16C illustrates effect of Fc4-CNP36 concentration on IOP change post administration intravitreally. Vehicle: 10 mM phosphate, pH7.3, 180 mM NaCl, 0.001% Pluronic F68; FP (L)=2 μg/eye IVT; FP (M)=20 μg/eye IVT; and FP (H)=80 μg/eye IVT. A significant reduction in the IOP (p<0.01) of all FP concentrations was observed at Day 1 compared to baseline (Day 0) or Day 7 after pONT surgery. The IOP reduction was not significantly affected within the 2 μg/eye-80 μg/eye IVT of FP4-CNP36 (FP), indicating IPO reduction reached maximal at 2 μg/eye. Table 31 illustrates calculated delivery dose per eye.

TABLE 31
Calculated delivery dose per eye

| | nmoles/eye | |
|---|---|---|
| ug/eye/4 ul | Monomer | Dimer |
| 2 | 0.076 | 0.038 |
| 20 | 0.76 | 0.38 |
| 80 | 3.05 | 1.53 |

FP-CNP, MW: 26 kDa

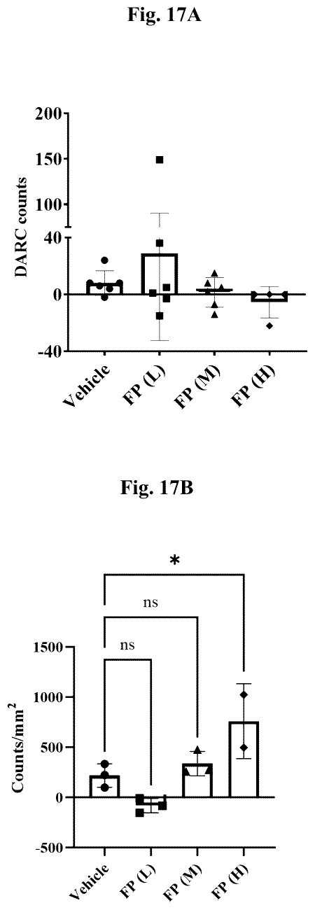

Effect of FP on DARC count and retina ganglion cell (RGC) protection. DARC (Detection of Apoptosing Retinal Cells) is a retinal imaging technology that has been developed within the last 2 decades from basic laboratory science to Phase 2 clinical trials. The higher the DARC counts, the severe apoptosis of retinal cells. FIGS. 17A-B illustrates effects of effect of Fc4-CNP36 on RGC protection in rat pONT model. FIG. 17A illustrates Fc4-CNP36 concentration detection of apoptosing retinal cells (DARC) reduction. FIG. 17B illustrates Fc4-CNP36 concentration on RGC count. Vehicle: 10 mM phosphate, pH7.3, 180 mM NaCl, 0.001% Pluronic F68; FP (L)=2 μg/eye IVT; FP (M)=20 μg/eye IVT; and FP (H)=80 μg/eye IVT. FP conditions showed the lowest DARC count with FP (High) concertation significantly, 80 μg/eye. FP conditions showed the lowest DARC count with FP (High) concertation significantly lower than AAV-Negative (p=0.0078). DARC counts were analyzed by subtracting the baseline of each animal and results shown DARC reduction showed a correlation to dose intravitreally injected as 80 mg/eye IVT showed complete inhibition of DARC formation (FIG. 17A). The RGC counts increased with dose increase (FIG. 17B).

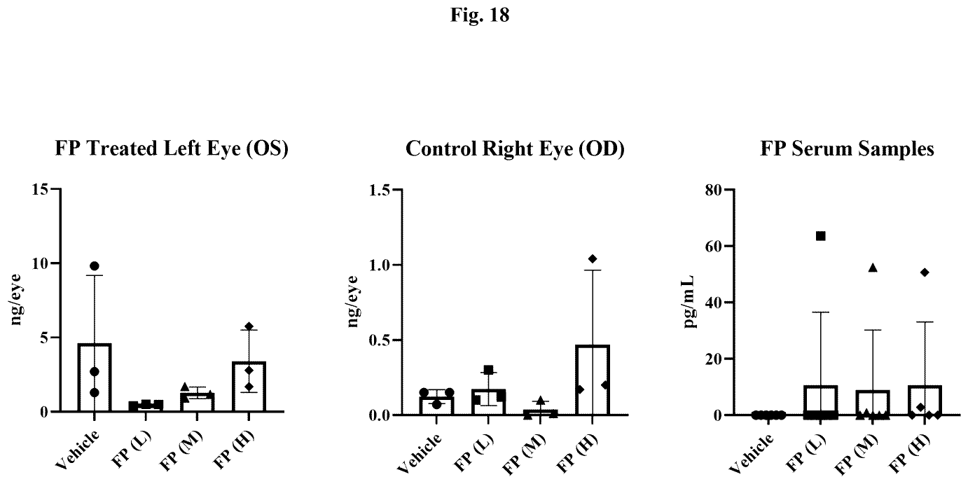

The protein level of FP4-CNP36 (FP) in the animal eye and serum samples were analyzed by ELISA assays and results are shown in FIG. 18 illustrating FP4-CNP36 levels detected by ELISA in the treated eye and control right eyes of animals. The levels of FP4-CNP36 in treated eyes (OS) were correlated to the dose amount.

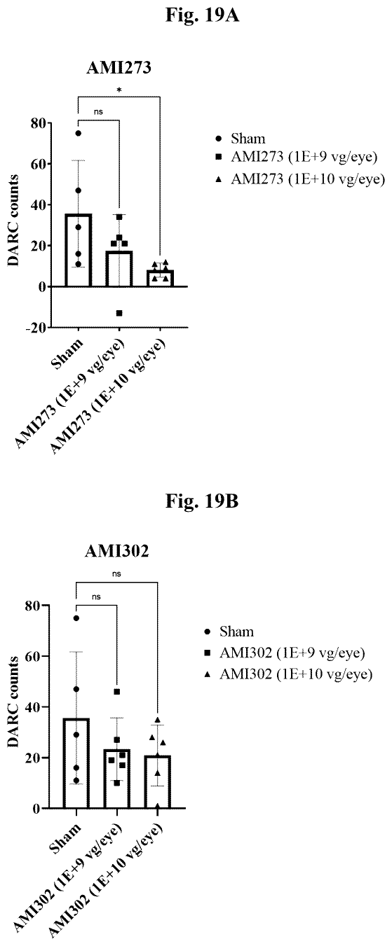

Effects of FP on DARC count and RGC count. DARC count analysis revealed that AMI273 is the test article of AAV2.N54-Fc4-CNP36 (AAV-FP) encoding Fc4-CNP36 and AMI302, AAV2.N54-CNP36 (AAV-P) encoding CNP36 peptide only, showed DARC reduction at 10⁹ and 10¹⁰ vg/eye 3 weeks after intravitreally administration for AMI273 and AMI302 compared to sham vector (FIG. 19). FIG. 19A-B illustrate effect of AAV2.N54-Fc4-CNP36 and AAV2.N54-CPNP36 on retina DARC reduction in rat pONT model. DARC values had been subtracted from the baseline. Terminal DARC counts were plotted. FIG. 19A: AMI273 was the vector of AAV2.N54-Fc4-CNP36 which expressed Fc4-CNP36 protein. FIG. 19B: AMI302 was the vector of AAV2.N54-CNP36 which expressed CNP36 peptide. For AMI273, animals IVT injected with 10¹⁰ vg/eye, DARC counts were reduced significantly while the animals received 10¹⁰ vg/eye IVT of AMI302, no significant reduction of DARC, indicating the high level of bioavailable level of Fc4-CNP36 was higher than CNP36, the peptide only. There was a clear effect of the Fc4-CNP36 (FP) protein on reducing levels of RGC apoptosis in this model in vivo, as seen in FIG. 18 and FIG. 19. All AAV treatments compared to FP showed an increased DARC count, indicating AAV groups raised baseline level. This could be related to the timing of DARC imaging following intravitreal administration of AAV, suggesting that persistent inflammation in the AAV groups was associated with a higher DARC count. Comparison of individual concentrations of treatment groups is shown in FIG. 19. It is important to note that there appeared to be a dose-dependent effect of FP on reducing the DARC count, with the highest concentration, 80 μg/eye of FP showing a significant reduction compared to Sham control which was the same vector AMI89 that contained the same DNA sequence but disrupted open reading frame (ORF) and resuspended in the same buffer.

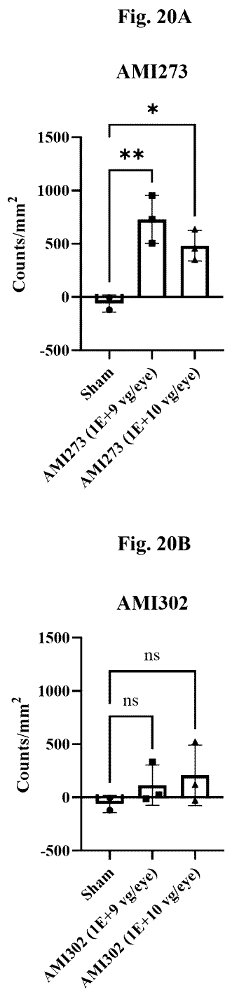

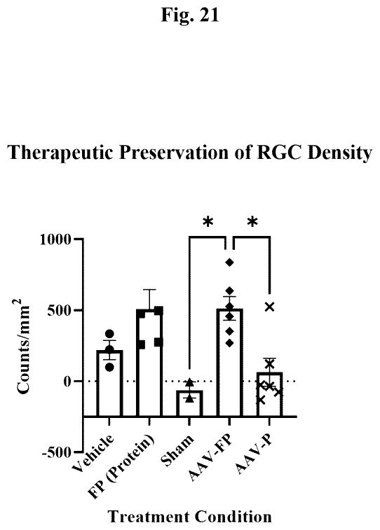

Effects on RGC survival. In this surgical model of pONT, it has been shown an initial injury in the superior retina causes primary degeneration whilst secondary effects are seen in the inferior retina due to secondary degeneration. Whilst the primary damage is unavoidable, the secondary damage can be preventable if therapeutic intervention is successful. To assess the effects of AAV-P and AAV-FP on RGC survival, regional differences were studied. The density of RBPMS⁺ RGCs (cells/mm²) in retinal whole mounts was analyzed by semi-segmentation of the superior and inferior retina (FIG. 20 and FIG. 21). FIG. 20A-B illustrates effect of AAV2.N54-Fc4-CNP36 (AAV-FP) and AAV2.N54-CPNP36 (AAV-P) on RGC protection in Rat pONT model. FIG. 20A: AMI273 was the vector of AAV2.N54-Fc4-CNP36 which expressed Fc4-CNP36 protein. FIG. 20B: AMI302 was the vector of AAV2.N54-CNP36 which expressed CNP36 peptide. The difference in cell density between the superior and inferior retina was then computed. By looking at the superior RGC count subtracted from the inferior RGC count (subtracted data) as an indication of protection against secondary degeneration, it was found that the AAV-FP condition resulted in significantly higher protection than the AAV-P (p=0.002), and the sham vector (p=0.005). No other comparisons were significant. p values were adjusted for five comparisons. By the subtraction of the superior from the inferior, RGC density was significantly higher in the AAV-FP condition than the conditions of AAV-P (p=0.002), and sham vector (AAV-negative, p=0.005) (FIG. 20). FIG. 21 illustrates RGC counts treated eyes of animals dosed with Fc4-CNP36 protein (FP) and AAV vectors in rat pONT model, where RGC density (cell/mm²) was presented in the superior and inferior regions with different treatment conditions in an order of Buffer, FP, sham vector (AAV-negative), AAV-positive, AAV-FP, and AAV-P.

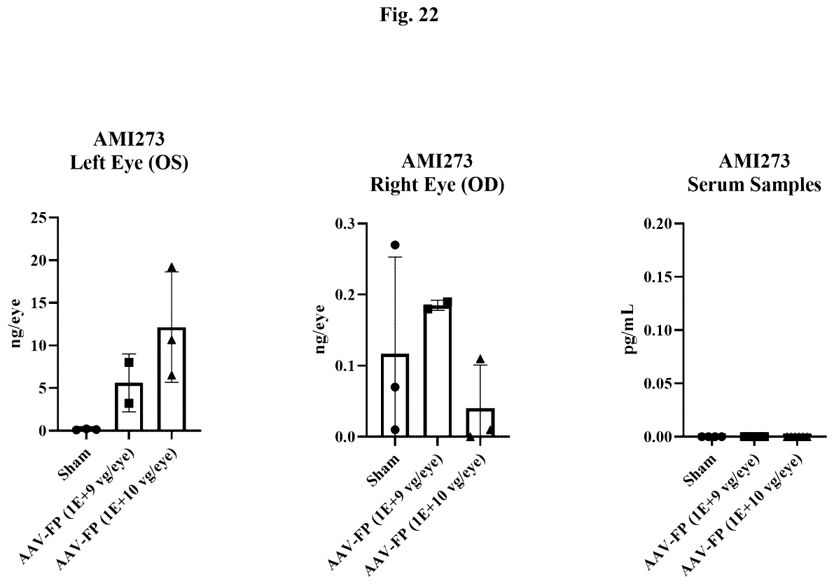

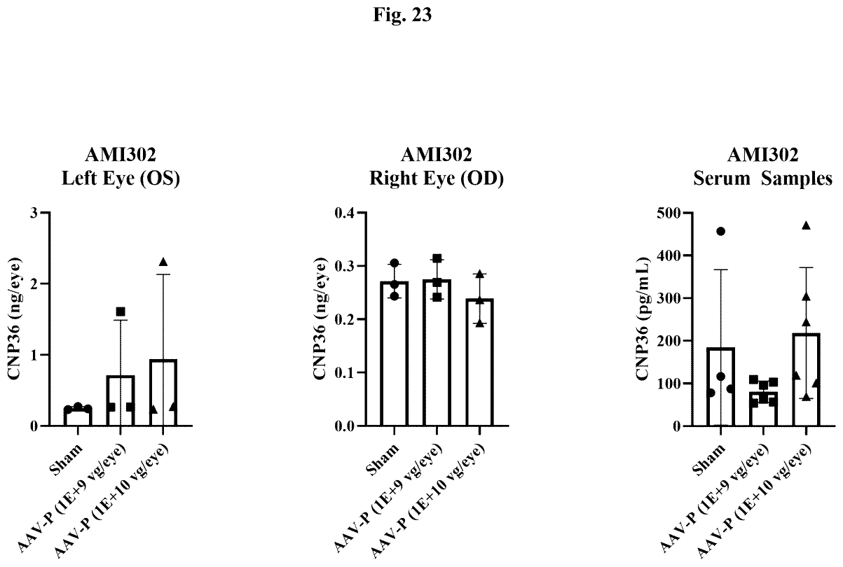

The expression in ocular tissue and serum of Fc4-CNP36 (FIG. 22) and CNP36 (FIG. 23) was detected by ELISA for the AAV dosed eyes (OS) and untreated (right) eyes. Fc4-CNP36 concentration in ocular homogenate was correlated well with the increase of AAV dose level in the left eyes (OS) while the transgene was not detected expression in the right eyes (OD). FIG. 22 illustrates Fc4-CNP36 concentration in ocular and serum samples after administration of AAV vectors (AAV-FP) IVT. The GOI of Fc4-CNP36 expressed in the AMI273 dosed eyes but no expression in un-dosed eyes. No transgene product, Fc4-CNP36, or CNP36 was detected in the no treated eyes, the right (OD) eyes. No transgene was detected either in serum samples of animals dosed either with AAV-FP or AAV-P independently. FIG. 23 illustrates CNP36 concentration in ocular and serum samples after administration of AAV vectors (AAV-P) IVT.

### Summary and Conclusion

DARC analysis revealed that the FP condition exhibited the lowest DARC count, followed by AAV-FP group. The RGC counts increased in eyes after treatment with FP, AAV-FP and AAV-P separately. IOP reduction was also observed in animals after IVT injection of FP (Fc4-CNP36) and reached maximal IOP lowering effect at 2 μg/eye and no further reduction was detectable with increase of FP.

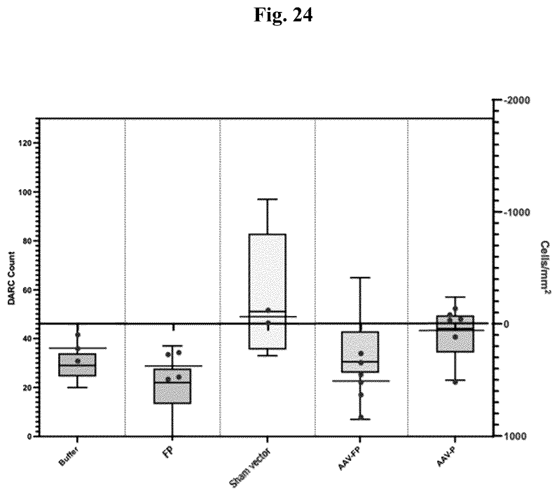

However, the DARC count appeared to correlate closely with RGC survival despite secondary neurodegenerative effects. The protective effects of the AAV-FP and FP on RGC survival in the inferior retina was consistent with the DARC data. A comparison of DARC count and RGC regional density is shown in FIG. 24, where reduced DARC count correlated with an increased RGC density (subtracting superior from inferior) in the FP and AAV-FP conditions in the analysis of secondary degeneration. Such data supports that FP and AAV-FP promotes neuroprotection, which led to secondary neurodegenerative effects.

### Example 5. EC₅₀ for CNP-Fc Constructs

EC₅₀ for CNP-Fc described herein were measured by ELISA. 2.5E+05 NIH3T3 cells/well were plated. After 2 days, −10 M to −5 M different CNP-Fcs (Fc4-CNP22; Fc1-CNP36; Fc4-CNP36; or Aflibercept-Fc4-CNP36) or comparable natriuretic peptide (ANP or CNP-22 as controls) were added into the specified wells in presence of 1.8 mM IBMX (inhibitor of cyclic nucleotide phosphodiesterases). After 30 minutes, supernatant was collected for cGMP determined via ELISA. FIG. 25A illustrates a standard curve of cGMP. FIG. 25B illustrates data fitting and calculations of EC₅₀ of various CNP fusion (e.g., CNP-Fc) and comparable natriuretic peptide described herein (EC₅₀ values: CNP-22 ≡Fc4-CNP36>Aflibercept-Fc4-CNP36>Fc1-CNP36>>Fc4-CNP22) based on cGMP secreted by the cells.

### Example 6. Assessment of Neuroprotective Effects and Treatment Efficacy of AAV-FP in Experimental Glaucoma Model

Glaucoma is a leading cause of irreversible blindness worldwide and is characterized by degeneration and loss of retinal ganglion cells (RGCs) and their axons, via apoptosis. Elevated intraocular pressure (IOP) is currently the only modifiable risk factor, but a proportion of glaucoma patients continue to lose their vision despite effective IOP control. Therefore, IOP independent risk factors are increasingly thought to play a role in glaucoma pathology and targeting those factors may have a potential in treatment of glaucoma. Neuroprotection and reduction in neuroinflammation have gained substantial interest in recent years as therapeutic approaches to prevent loss of function in glaucoma.

DARC (detection of apoptosing retinal cells) is a biomarker that binds to the exposed phosphatidylserine that allows identification of sick, stressed, and apoptotic cells. DARC can be used as a platform for evaluating the neuroprotective effects and treatment efficacy of a drugs, in both pre-clinical and clinical trials. DARC can be used to assess gene therapies delivered by a method or an engineered polynucleotide described herein in a model of partial optic nerve transection.

This study aims to investigate the efficacy of 2 doses of AAV-FP relative to sham AAV and NGF positive control, using well-established and translatable endpoints including DARC on an ocular hypertensive model. Using a well-established rat model of glaucoma (ocular hypertension or OHT), the following can be investigated: whether AAV-FP can prevent or reduce RGC (retinal ganglion cells) apoptosis in vivo by DARC imaging and evaluate dose-response relationship using DARC imaging; and whether AAV-FP can promote RGC survival and reduce inflammation by histological labelling and evaluation of microglial morphometry through disease induction to AAV intervention.

### Experimental Design

### Treatment Groups (Table 32):

Group 1: OHT only (n=6)
Group 2: OHT+Vehicle (n=6)
Group 3: OHT+IVT NGF (n=6)
Group 4: OHT+IVT Sham AAV-FP (n=6)
Group 5: OHT+IVT Dose 1 AAV-FP (n=6)
Group 6: OHT+IVT Dose 2 AAV-FP (n=6)

TABLE 32
Animal groups for OHT study

| | Time | | | | | | | | |
|---|---|---|---|---|---|---|---|---|---|
| Grp | −4 wk | −3 wk | −2 wk | −1 wk | BL | 1 d | 1 wk | 2 wk | 3 wk |

| | | | | | | |
|---|---|---|---|---|---|---|
| 1 | | IOP, DARC/OCT, OHT | IOP | IOP | IOP | IOP, DARC/OCT |
| 2 | IVT | IOP, DARC/OCT, OHT | IOP | IOP | IOP | IOP, DARC/OCT |
| 3 | | IOP, DARC/OCT, OHT, IVT | IOP | IOP | IOP | IOP, DARC/OCT |
| 4 | IVT | IOP, DARC/OCT, OHT | IOP | IOP | IOP | IOP, DARC/OCT |
| 5 | IVT | IOP, DARC/OCT, OHT | IOP | IOP | IOP | IOP, DARC/OCT |
| 6 | IVT | IOP, DARC/OCT, OHT | IOP | IOP | IOP | IOP, DARC/OCT |

### In Vivo Schedule (4 Months):

Animals: 36 Dark Agouti (DA) male rats at 150-200 g weight (n=6, 6 groups).
Treatments: animals can be randomly grouped into two groups (see above).
Groups 2, 4 5 and 6 can receive intravitreal injections 4 weeks in advance of IOP elevation.
Group 3 can receive IVT NGF at the time of surgery.
OHT surgery: intraocular pressure (IOP) can be raised on the left eye only, by injection of hypertonic saline into the episcleral veins, as well-established in the group.
IOP measurements: IOP can be measured before surgery (baseline) and day 1, week 1 and week 3 after surgery using a tonometer. Only animals who have elevated IOP at Day 1 (>5 mmHg compared to baseline) in the induced eye will be enrolled in treatment groups.
In vivo DARC imaging: RGC apoptosis in both eyes of each animal can be assessed with DARC at baseline and 3 weeks after OHT induction, by intranasal injection of fluorescently labelled annexinV 2 hours before imaging with a confocal scanning laser ophthalmoscopy (cSLO) [1, 2, 3]. The retinal images can be then collected, and the number of apoptotic RGCs counted.
In vivo OCT images can also be recorded at the same timepoints as DARC to assess vitritis.

### Histology Assessments (2 Months):

Animals can be culled at 3 weeks after OHT induction, and both eyes enucleated and wholemount retinas dissected.
Wholemount retinas can be stained with RBPMS and IBA-1 to label surviving RGCs and microglial cells, and imaged under confocal microscopy.

### Data Analysis (1 Month):

RGC apoptosis in DARC images in each eye can be analyzed.
RGC survival in each eye can be assessed in whole retina mounts.
Primary and secondary degeneration in each eye can be analyzed accordingly.
Analysis of microglia morphology in each eye can be assessed.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. An engineered polynucleotide comprising an AAV vector, said AAV vector comprises an expression cassette, wherein the expression cassette encodes an engineered polypeptide comprising a natriuretic peptide covalently connected to an antibody or fragment thereof.

2. The engineered polynucleotide of claim 1, wherein the natriuretic peptide comprises an amino acid sequence that is at least 80%, at least 90%, at least 95%, or at least 99% identical to any one of SEQ ID NOs: 1-5.

3-4. (canceled)

5. The engineered polynucleotide of claim 1, wherein the natriuretic peptide comprises an amino acid sequence that is at least 80%, at least 90%, at least 95%, or at least 99% identical to SEQ ID NO: 4.

6-7. (canceled)

8. The engineered polynucleotide of claim 1, wherein the antibody or fragment thereof comprises a fragment crystallizable (Fc) region.

9. The engineered polynucleotide of claim 1, wherein the antibody or fragment thereof comprises an amino acid sequence that is at least 80% at least 90%, at least 95%, or at least 99% identical to any one of SEQ ID NOs: 6-8.

10-13. (canceled)

14. The engineered polynucleotide of claim 1, wherein the natriuretic peptide is covalently connected to the antibody or fragment thereof by a peptide linker.

15-16. (canceled)

17. The engineered polynucleotide of claim 1, wherein the engineered polypeptide comprises a protease cleavage site.

18. The engineered polynucleotide of claim 17, wherein the proteases cleavage site comprises a Furin protease site.

19. The engineered polynucleotide of claim 1, wherein the engineered polypeptide comprises an amino acid sequence that is at least 80%, at least 90%, at least 95%, or at least 99% identical to any one of SEQ ID NOs: 131-140.

20-21. (canceled)

22. The engineered polynucleotide of claim 1, wherein the natriuretic peptide comprises a C-type natriuretic peptide (CNP) or fragment thereof.

23-29. (canceled)

30. The engineered polynucleotide of claim 1 the AAV vector comprises an AAV serotype comprising AAV1, AAV2, AAV3, AAV4, AAV5, AAV6, AAV7, AAV8, AAV9, AAV10, AAV11, AAV12, or any combination thereof.

31. The engineered polynucleotide of claim 30, wherein the AAV serotype comprises the AAV2.

32. The engineered polynucleotide of claim 1, wherein the AAV vector encodes an modified AAV capsid.

33. The engineered polynucleotide of claim 1, wherein the AAV vector comprises a second expression cassette for encoding a therapeutic.

34. (canceled)

35. The engineered polynucleotide of claim 34, wherein the therapeutic comprises a hormone, an agonist of a natriuretic peptide receptor (NPR), an agonist of a cyclic GMP (cGMP) signaling pathway, or an VEGF inhibitor.

36-71. (canceled)

72. A pharmaceutical composition comprising the engineered polynucleotide of claim 1.

73. The pharmaceutical composition of claim 72, wherein pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

74. (canceled)

75. The pharmaceutical composition of claim 73, wherein the pharmaceutical composition is for treating an ocular disease or condition.

76-85. (canceled)

86. A method of treating a disease or condition in a subject, comprising administering an engineered polynucleotide to the subject, wherein the engineered polynucleotide comprises a viral vector comprising an expression cassette for expressing an engineered polypeptide comprising an antibody or a fragment thereof operatively coupled to a natriuretic peptide in a cell of the subject, and wherein the cell expresses the engineered polypeptide, thereby treating the disease or condition.

87-127. (canceled)

128. The method of claim 86, wherein the disease or condition comprises an ocular disease.

129-168. (canceled)
